(19) United States
(12) Patent Application Publication
(10) Pub. No.: US 2014/0234359 A1
(43) Pub. Date: Aug. 21, 2014

(54) PLANT VIRAL VACCINES AND THERAPEUTICS

(71) Applicants: Martha Karen Newell, Holland, TX (US); Richard Tobin, Aurora, CO (US); Susannah K. Rogers, Holland, TX (US)

(72) Inventors: Martha Karen Newell, Holland, TX (US); Richard Tobin, Aurora, CO (US); Susannah K. Rogers, Holland, TX (US)

(73) Assignees: Viral Genetics, Inc., San Marino, CA (US); Scott & White Healthcare, Temple, TX (US); The Texas A&M University System, College Station, TX (US)

(21) Appl. No.: 14/346,214

(22) PCT Filed: Sep. 21, 2012
(86) PCT No.: PCT/US12/56621

§ 371 Date: Mar. 20, 2014

Related U.S. Application Data
(60) Provisional application No. 61/537,306, filed on Sep. 21, 2011.

(51) Int. Cl. A61K 39/12; A61K 39/39; C12Q 1/70; C07K 14/005; C12N 15/113

(52) U.S. Cl. CPC A61K 39/12; C07K 14/005; C12N 15/1131; C12Q 1/70; A61K 39/39
USPC 424/186.1; 424/204.1; 424/199.1; 514/44 A; 435/5

(57) ABSTRACT

The invention relates to methods and related products for preventing and treating disease, based on the use of plant viral vaccines and plant viral defense strategies. The methods also involve the identification of appropriate therapeutic strategies for diseases such as cancers.

### RELATED APPLICATIONS

This application claims priority under 35 U.S.C. §119(e) to U.S. Provisional Application Ser. No. 61/537,306, entitled “PLANT VIRAL VACCINES AND THERAPEUTICS” filed on Sep. 21, 2011, which is herein incorporated by reference in its entirety.

### BACKGROUND OF INVENTION

Mammalian viruses have recently been shown to play a critical role in the development of certain types of tumors in animals or humans. At least six families of viruses appear to be involved in tumor development. These include five families of viruses having DNA genomes, which are referred to as DNA tumor viruses and a single family of tumor viruses referred to as retroviruses. Retroviruses have viral particles with RNA genomes and replicate through the synthesis of a DNA provirus in infected cells. Known tumor causing viruses include Hepatitis B virus (HBV, Liver Cancer), Human Papilloma virus (HPV, cervical and other anogenital cancers), Epstein-barr virus (EBV, Burkitt's Lymphoma and Nasopharyngeal carcinoma), Kaposi's sarcoma-associated herpes virus (Kaposi's sarcoma), Human T-cell Lymphotropic virus (adult T-cell leukemia), and Human Immunodeficiency virus (HIV, aids associated cancers).

Although these viruses have each been linked with cancer it is believed that the tumor viruses work through distinct mechanisms. For instance, HBV is believed to cause chronic tissue damage in the liver which drives the continual proliferation of liver cells resulting in a tumor. SV40 and Polyoma virus are believed to produce factors during lytic infection which stimulate host cell gene expression and DNA synthesis. Since most animal cells are non-proliferating they must be stimulated to divide in order to induce the enzymes needed for viral DNA replication. Cell proliferation stimulated in this way can lead to transformation if the viral DNA becomes stably integrated. One common feature of tumor-causing viruses is that these viruses cause changes to the cells by integrating their genetic material within the host cell DNA. DNA viruses can directly insert the DNA into the host DNA. RNA viruses, however, must first transcribe RNA to DNA and then insert the genetic material into the host cell.

Human papilloma virus (HPV) has been implicated in many tumors. HPV infections often persist for extended periods of time and persistent infections with HPVs have been demonstrated to be the primary cause of cervical cancer. The discovery of HPV as an etiologic agent of many human tumors provided the rationale for the development of a vaccine, now sold as either Gardasil® or Cervarix®, both of which have been reported to prevent cervical and potentially other tumors, such as anal cell carcinoma and genital warts. Gardasil®, sold by Merck, is a prophylactic vaccine designed to avoid the development of cervical and other cancers. Gardasil® does not treat existing infections and must be given prior to HPV infection in order to be effective. Gardasil® is typically provided in three 0.5 ml injections over six months. The second injection is two months after the first and the third injection is four months after the second. Gardasil® is composed of recombinant viral like particles (VLPs) assembled from the L1 proteins of HPV. It has been shown that genes encoding the L1 protein in recombinant form are capable of assembling into HPV VLPs when expressed that are morphologically similar to native HPV virions.

A review article on HPV and therapeutic vaccines (Mo et al. Current cancer therapy reviews, 2010, 6, 81-103), notes that HPV, a non-enveloped double-stranded circular DNA virus, may integrate viral DNA into the host genome.

### SUMMARY OF INVENTION

It has been discovered that plant viruses play an important role in the development of human disease. The invention, in some aspects, is directed to novel prophylactic and therapeutic modalities for treating human disease and related products based on the targeting of plant viruses.

In some aspects the invention is directed to a vaccine of an isolated plant viral antigen, wherein the plant viral antigen is immunogenic, and a pharmaceutically acceptable carrier. In some embodiments the plant viral antigen is an immunogenic peptide. Optionally, the vaccine may include an adjuvant.

In other embodiments the plant viral antigen is a nucleic acid comprising at least one gene encoding a plant viral peptide. The vaccine may be a replication defective vector comprising the nucleic acid, which optionally may be an adenoviral vector. In some embodiments the gene is operably linked to a heterologous promoter and transcription terminator.

The plant viral antigen, in some embodiments, is a plant virus selected from the group consisting of Maize chlorotic mottle virus; Maize rayado fino virus; Oat chlorotic stunt virus; Chayote mosaic tymovirus; Grapevine asteroid mosaic-associated virus; Grapevine fleck virus; Grapevine Red Globe virus; Grapevine rupestris vein feathering virus; Melon necrotic spot virus; Physalis mottle tymovirus; Prunus necrotic ringspot; Nigerian tobacco latent virus; Tobacco mild green mosaic virus; Tobacco mosaic virus; Tobacco necrosis virus; Eggplant mosaic virus; Kennedya yellow mosaic virus; Lycopersicon esculentum TVM viroid; Oat blue dwarf virus; Obuda pepper virus; Olive latent virus 1; Paprika mild mottle virus; PMMV; Tomato mosaic virus; Turnip vein-clearing virus; Carnation mottle virus; Cocksfoot mottle virus; Galinsoga mosaic virus; Johnsongrass chlorotic stripe mosaic virus; Odontoglossum ringspot virus; Ononis yellow mosaic virus; Panicum mosaic virus; Poinsettia mosaic virus; Pothos latent virus; Banana bunchy top virus, and Ribgrass mosaic virus.

In other aspects the invention is a method of modulating gastrointestinal plant viral levels in a subject, by administering to the subject an amount of a plant virus vaccine effective to modulate the plant virus levels in the gastrointestinal tract of the subject. In some embodiments the levels of plant virus in the gastrointestinal system of the subject corresponding to the plant virus vaccine are decreased in the gastrointestinal system of the subject relative to the levels that are observed in the absence of the administration of the plant virus vaccine. In other embodiments the levels of plant virus in the gastrointestinal system of the subject are measured in a fecal sample or a blood sample.

Methods involving administering to a subject at risk of having a plant virus associated cancer, a plant virus vaccine in an effective amount to inhibit infection with the plant virus in the subject are provided according to other aspects of the invention. In some embodiments the subject has been exposed to a plant virus.

The invention also relates to a method for treating a subject, wherein the subject has a disease associated with a plant virus, with an anti-viral compound in an effective amount to reduce infection with the plant virus in the subject.

In other aspects of the invention a method is provided. The method comprises determining whether a subject having a virally caused disease has been exposed to a plant virus that causes the disease, and treating the subject with a compound that is a plant defense mechanism against the plant virus in an effective amount to reduce infection of the subject with the plant virus. The disease may optionally be cancer. The method may also include the step of administering a TLR agonist.

In other embodiments the step of determining whether the subject has been exposed to the plant virus involves analyzing a biological sample of the subject for the presence of the plant virus. The biological sample may be, for instance, a fecal or blood sample.

In some embodiments the compound is a naturally occurring substance found in a plant susceptible to the plant virus or is an analog, homolog, or derivative thereof. In other embodiments the compound is a plant defense mechanism against the plant virus selected from the group consisting of flavonoids, anthocyanins, phytoalexins, medicarpin, rishitin, camalexin, capsaisin, glucosinolate, defensins, alpha-amylase, protease inhibitors, lignin and furanocoumarins.

According to yet other aspects, the invention involves a method for silencing plant virus gene expression in a mammal needing relief from the gene expression. The method involves administering to the mammal an inhibitory nucleic acid that targets the genome of an essential plant virus in an effective amount to reduce infection of the mammal with the plant virus.

In some embodiments the inhibitory nucleic acid comprises double stranded nucleic acid of 15 to 30 nucleotides in length. The double stranded nucleic acid may have a first nucleotide sequence that targets the genome of the essential plant virus and a second nucleotide sequence that is a complement of the first nucleotide sequence.

The inhibitory nucleic acid in some embodiments comprises a nucleotide sequence having sufficient complementarity to a target sequence of about 15 to about 30 contiguous nucleotides in an RNA of a virus for the inhibitory nucleic acid to direct cleavage of the RNA via RNA interference. The virus may be selected from the group consisting of Maize chlorotic mottle virus; Maize rayado fino virus; Oat chlorotic stunt virus; Chayote mosaic tymovirus; Grapevine asteroid mosaic-associated virus; Grapevine fleck virus; Grapevine Red Globe virus; Grapevine rupestris vein feathering virus; Melon necrotic spot virus; Physalis mottle tymovirus; Prunus necrotic ringspot; Nigerian tobacco latent virus; Tobacco mild green mosaic virus; Tobacco mosaic virus; Tobacco necrosis virus; Eggplant mosaic virus; Kennedya yellow mosaic virus; Lycopersicon esculentum TVM viroid; Oat blue dwarf virus; Obuda pepper virus; Olive latent virus 1; Paprika mild mottle virus; PMMV; Tomato mosaic virus; Turnip vein-clearing virus; Carnation mottle virus; Cocksfoot mottle virus; Galinsoga mosaic virus; Johnsongrass chlorotic stripe mosaic virus; Odontoglossum ringspot virus; Ononis yellow mosaic virus; Panicum mosaic virus; Poinsettia mosaic virus; Pothos latent virus; and Ribgrass mosaic virus, wherein the target sequence is in a gene essential for infectivity or replication of the virus. In some embodiments the gene essential for infectivity or replication of the virus is selected from a group consisting of plant virus genome-linked protein (VPg), VPg-Pro, the 3′UTR, the 5′ UTR, zinc finger region of the capsid protein, and tRNA like domain.

A vector composition comprising a nucleic acid encoding an inhibitory nucleic acid that targets the genome of an essential plant virus operably linked to a mammalian promoter is provided according to other aspects of the invention.

A method is also provided for performing a physical analytical step on a biological sample of a subject, identifying the presence of plant virus in the biological sample based on the physical analytical step, and determining a course of treatment for the subject based on the presence of the plant virus. In some embodiments the presence of the plant virus is indicative of a predisposition to cancer. In other embodiments the biological sample is a fecal sample. In yet other embodiments the plant virus is tobacco mosaic virus, Maize chlorotic mottle virus; Maize rayado fino virus; Oat chlorotic stunt virus; Chayote mosaic tymovirus; Grapevine asteroid mosaic-associated virus; Grapevine fleck virus; Grapevine Red Globe virus; Grapevine rupestris vein feathering virus; Melon necrotic spot virus; Physalis mottle tymovirus; Prunus necrotic ringspot; Nigerian tobacco latent virus; Tobacco mild green mosaic virus; Tobacco necrosis virus; Eggplant mosaic virus; a yellow mosaic virus; Lycopersicon esculentum TVM viroid; Oat blue dwarf virus; Obuda pepper virus; Olive latent virus 1; Paprika mild mottle virus; PMMV; Tomato mosaic virus; Turnip vein-clearing virus; Carnation mottle virus; Cocksfoot mottle virus; Galinsoga mosaic virus; Johnsongrass chlorotic stripe mosaic virus; Odontoglossum ringspot virus; Ononis yellow mosaic virus; Panicum mosaic virus; Poinsettia mosaic virus; Pothos latent virus; or Ribgrass mosaic virus.

The method may also involve analyzing the status of inflammation in the subject.

The course of treatment in the method may be the administration of a plant virus vaccine.

According to other aspects of the invention, a method for treating a plant virus associated cancer is provided. The method involves administering to a subject having a plant virus associated cancer an inhibitor of plant specific RNA dependent RNA polymerase in an effective amount to treat the cancer.

In some embodiments the inhibitor is an RNA dependent RNA polymerase antagonist. The RNA dependent RNA polymerase antagonist may be an inhibitory peptide, such as an antibody. In other embodiments the RNA dependent RNA polymerase antagonist is an inhibitory nucleic acid such as siRNA, shRNA, or miRNA.

A method for identifying an anti-cancer agent is provided according to other aspects of the invention. The method involves performing a physical analytical step on a plant to determine a plant defense mechanism for preventing infection with a plant virus, identifying an association of the plant virus with a mammalian cancer, and selecting the plant defense mechanism as an anti-cancer agent for the mammalian cancer.

A kit including a set of primers for detecting plant viruses, a reagent for processing the primers to detect plant viruses, and instructions for analyzing a human or animal biological sample to detect the presence of plant viruses using the set of primers and reagent is provided in other aspects of the invention.

A method for determining the presence of a plant virus in a human gut capable of inducing a virally caused disease is provided according to yet another aspect of the invention. The method involves conducting an analytic test for such plant virus in the blood or fecal matter of the human using a set of first reagents for detecting plant viruses, and using a second reagent for processing the first reagents to detect plant viruses. In some embodiments the set of first reagents comprises a set of antibodies against a plurality of said plant viruses.

According to other aspects of the invention, a method for treating HIV is provided. The method involves administering to a subject having or at risk of having HIV a plant viral vaccine in an effective amount to treat or prevent HIV infection in the subject. In some embodiments the plant viral vaccine is banana bunchy virus.

In other aspects, a composition for modulating gastrointestinal plant viral levels in a subject is provided. The composition is formulated in amount sufficient for administering to the subject an amount of a plant virus vaccine effective to modulate the plant virus levels in the gastrointestinal tract of the subject, wherein the plant virus vaccine is optionally a vaccine as described herein.

In other aspects a composition of a plant virus vaccine in an effective amount to inhibit infection with the plant virus in a subject at risk of having a plant virus associated cancer is provided.

A composition comprising an anti-viral compound for use in the treatment of a subject having a disease associated with a plant virus is provided according to other aspects of the invention.

A composition comprising a compound that is a plant defense mechanism against a plant virus for use in the treatment of a subject who has been identified as having a virally caused disease, such as cancer, and has been exposed to the plant virus that causes the disease.

A composition comprising an inhibitory nucleic acid that targets the genome of an essential plant virus for use in silencing plant virus gene expression in a mammal needing relief from the gene expression and in an effective amount to reduce infection of the mammal with the plant virus.

A composition comprising an anti-viral compound for use in the treatment of a subject having a plant virus associated cancer, wherein the anti-viral compound is a compound that interferes with viral synthesis.

This invention is not limited in its application to the details of construction and the arrangement of components set forth in the following description or illustrated in the drawings. The invention is capable of other embodiments and of being practiced or of being carried out in various ways. Each of the above embodiments and aspects may be linked to any other embodiment or aspect. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 is a blot of a genomic DNA PCR analysis. Gnomic DNA from T-24 human bladder cancer cell line was amplified using 4 primer sets specific for amplifying tobacco mosaic virus (TMV). Lane 1. 1/1000 BP size markers. Lane 2. Genomic DNA amplified with TMV primer 1. Lane 3. Genomic DNA amplified with TMV primer 3. Lane 4. Genomic DNA amplified with TMV primer 6. Lane 5. Genomic DNA amplified with TMV primer 8. Foreword and reverse primer sequences can be found in Example 1.

FIG. 2 is a data set depicting the effect of antiviral treatment on T-24 human bladder cancer cells. FIG. 2a is a set of dot plots of flow cytometric data. Forward scatter on the Y-axis vs side scatter on the X-axis. Data shows increased death in T-24 human bladder cancer cells treated with anti-viral agent efavirenz, a nonnucleoside reverse transcriptase inhibitor. FIG. 2b is a bar graph showing increased cell death after treatment with efavirenz. Cell death was measured by flow cytometry.

FIG. 3 demonstrates that TLR activation results in transcription of the integrated viral genes in several human bladder cancer cells. FIG. 3 is a series of bar graphs depicting the results of the PCR assays using primers 1-8, under the following cellular conditions: 3a is CpG treated spleen cells, 3b is untreated T24 cells, 3c is CpG treated T24 cells; 3d is LPS treated T24 cells, 3e is CpG+efavirenz treated T24, and 3f is LPS+efavirenz treated T24.

FIG. 4 is a ClustalX 2.1 sequence alignment of plant virus protein sequences versus viral anti-apoptotic protein sequences.

FIG. 5 is a ClustalX 2.1 sequence alignment of Plant Virus Protein Sequences vs. Human Proteins from Cell Death Pathways. FIG. 5A depicts amino acids 1051-1200. FIG. 5B depicts amino acids 1201-1350.

FIG. 6 is a ClustalX 2.1 sequence alignment of HIV versus Banana Bunchy Top Virus (BBTV).

### DETAILED DESCRIPTION

A group of researchers recently analyzed the enteric RNA viral community present in healthy humans (Zhang et al. PLOS Biology, January 2006, v. 4, p. 108) and discovered that the majority of the viral sequences present in human fecal samples were similar to plant RNA viruses. Upon further analysis of the viruses taken from these samples, it was discovered that these viruses were active and still capable of infecting plants. Traditionally plant viruses were believed to be harmless in humans. Although plant viruses have long been, and are currently, considered non-pathogenic for animals, our discoveries (that lead to the invention) prompt us to consider that plant viruses may infect animal cells and that they may be causally related to human disease.

It has now been discovered that these active viruses present in many human subjects, which were previously thought to be harmless, play critical roles in the development of disease. A number of diseases, including tumors, in humans and animals are associated with plant virus infection. The ability to prevent plant viral infection and/or to treat plant viral infection has profound implications for the treatment of a wide array of diseases. As such, the invention relates to preventative and therapeutic vaccines which are specific for plant viruses as well as compounds that are effective in reducing or eliminating the activity of plant viruses, in order to treat diseases in which plant viruses play a role. The invention also encompasses diagnostic, prognostic and drug discovery based methods.

Plant viruses are structurally similar to mammalian viruses in many respects. Two families of plant viruses are characterized as single-stranded DNA viruses, both having small circular genome components. A single family of plant viruses is categorized as a reverse-transcribing virus, having a single circular double-stranded DNA structure. The replication of the reverse-transcribing virus is through an RNA intermediate. Several plant viruses and many mycoviruses are characterized as double-stranded RNA viruses. A few plant viruses are negative sense single-stranded RNA. They are characterized as such because some or all of their genes are translated into a protein from an RNA strand complementary to that of the genome. Finally, the majority of plant viruses are positive sense single-stranded RNA. Some viruses use host reverse transcriptase or that from co-infectious agents.

Many of the plant viruses reported to be present in the gut or nasal passages are RNA viruses whose genomes encode RNA dependent RNA polymerase that can bind to “permissive” factors or proteins that make a host, a plant or even a mammalian cell, permissive for plant virus infection. In a recent study, investigators reported that Pepper Mild Mottled Virus (PMMV) can infect mammalian cells and the report suggested for the first time that mammalian cells may be hosts to plant (Colson et al. POLF1, v. 5, April 2010, p. 1).

The data presented in the Examples is the first demonstration of a direct link between a plant virus and a mammalian disease, such as cancer. It was discovered that viral DNA from tobacco mosaic virus is stably incorporated into genomic DNA from human bladder cancer. The development of bladder cancer is strongly linked to exposure to smokeless tobacco. The discovery that tobacco mosaic virus is stably incorporated into genomic DNA from human bladder cancer strongly supports the assertion that the virus creates a susceptibility to the development of cancer, similar to the role played by papilloma virus in cervical cancer. Additionally, human bladder cancer cells treated with a plant anti-viral agent showed significantly less proliferation than control (untreated or methanol treated) cells. The data indicate that plant viruses play a role in cancer such as bladder cancer and that treatment of the viral infection can reduce cellular proliferation and, thus, such compounds are useful therapeutics. Additionally, after the priority date of the instant application Li et al (Biosci. Rep. 32, p. 174, 2012) published a study demonstrating that TMV induces autophagy in HeLa cells, confirming Applicant's work.

Although Applicant is not bound by mechanism of action it is believed that the plant virus contributes to mammalian disease by integrating plant viral DNA into the host genome in an oncogenic manner or transcriptionally silent manner or alternatively by remaining independent of the host DNA by altering the function of the host cells by utilizing a mechanism which is similar to RNA interference and can regulate host gene expression. When the viral DNA is integrated in an oncogenic manner it may be integrated into the chromosome near an oncogene or in another site that would cause it to be expressed in a dysregulated fashion. The dysregulated expression of the viral DNA causes increased expression, leading to the proliferation of the host cell. Plant viral DNA that is incorporated in transcriptionally silent manner may also result in the development of cancer or other disease when the host cell is exposed to a trigger event. Once the plant viral DNA is silently integrated into the genome it may lay dormant for a period of time, and later be reactivated under conditions of stress, such as inflammation or TLR activation. The reactivation in response to conditions of stress can activate new gene transcription from the integrated viral DNA sequences, resulting in cellular proliferation. Thus, TLR agonists can be administered together with the vaccines or other therapeutics of the invention in order to activate viral transcription, to enhance the therapy.

“Plant viruses” as used herein refers to a group of viruses that have been identified as being pathogenic to plants. These viruses rely on the host for replication, as they lack the molecular machinery to replicate without the host. Plant viruses include but are not limited to tobacco mosaic virus, Maize chlorotic mottle virus; Maize rayado fino virus; Oat chlorotic stunt virus; Chayote mosaic tymovirus; Grapevine asteroid mosaic-associated virus; Grapevine fleck virus; Grapevine Red Globe virus; Grapevine rupestris vein feathering virus; Melon necrotic spot virus; Physalis mottle tymovirus; Prunus necrotic ringspot; Nigerian tobacco latent virus; Tobacco mild green mosaic virus; Tobacco necrosis virus; Eggplant mosaic virus; Kennedya yellow mosaic virus; Lycopersicon esculentum TVM viroid; Oat blue dwarf virus; Obuda pepper virus; Olive latent virus 1; Paprika mild mottle virus; PMMV; Tomato mosaic virus; Turnip vein-clearing virus; Carnation mottle virus; Cocksfoot mottle virus; Galinsoga mosaic virus; Johnsongrass chlorotic stripe mosaic virus; Odontoglossum ringspot virus; Ononis yellow mosaic virus; Panicum mosaic virus; Poinsettia mosaic virus; Pothos latent virus; or Ribgrass mosaic virus. An extensive listing of plant viruses, which can be treated or prevented according to the invention, is set forth in Brunt, A. A. et al (eds.) (1996), Plant Viruses Online Descriptions and Lists from the VIDE Database. Version: 20 Aug. 1996 (URL:http://biology.anu.edu.au/Groups/MES/vide/) and Dallwitz (1980) and Dallwitz, Paine and Zurcher (1993). These viruses include all of those listed on Appendix A of U.S. Patent Application Ser. No. 61/537,306, to which the instant application claims priority and which is specifically incorporated by reference and in Brunt, A. A. et al (eds.) (1996), Plant Viruses Online: Descriptions and Lists from the VIDE Database. Version: 20 Aug. 1996 (URL:http://biology.anu.edu.au/Groups/MES/vide/) and Dallwitz (1980) and Dallwitz, Paine and Zurcher (1993). Exemplary plant viruses and the plants they infect are presented below in Table 1.

TABLE 1

| Virus | Plant | Type of Host Plant |
|---|---|---|
| Maize chlorotic mottle virus | Zea mays | Corn |
| Maize rayado fino virus | Zea mays | Corn |
| Oat chlorotic stunt virus | Avena sativa | Oat |
| Chayote mosaic tymovirus | Sechium edule | Chayote or vegetable pear |
| Grapevine asteroid mosaic- | Vitis rupetris | Grape |
| associated virus | | |
| Grapevine fleck virus | Vitis vinifera | Grape |
| Grapevine Red Globe virus | Vitis rupestris | Grape |
| Grapevine rupestris vein feathering | Vitis rupestris | Grape |
| virus | | |
| Melon necrotic spot virus | Cucumis melo, C. sativus | Melon and cucumber |
| Physalis mottle tymovirus | Solanaceous plants | Datura (Jimson weed), |
| | | Mandragora (mandrake), |
| | | belladonna (deadly nightshade), |
| | | Lycium barbarum (Wolfberry), |
| | | Physalis philadelphica |
| | | (Tomatillo), Physalis peruviana |
| | | (Cape gooseberry flower), |
| | | Capsicum (paprika, chili pepper), |
| | | Solanum (potato, tomato, |
| | | eggplant), Nicotiana (tobacco), |
| | | and Petunia. With the exception |
| | | of tobacco (Nicotianoideae) and |
| | | petunia (Petunioideae) |
| Prunus necrotic ringspot | Dicotyledonous plants | Fruit |
| Nigerian tobacco latent virus | Nigerian tobacco | Tobacco |
| Tobacco mild green mosaic virus | Nicotiana glauca, N. tabacum, | Tobacco |
| | Capsicum annum, Eryngium | |
| | aquaticum | |
| Tobacco mosaic virus | Nicotiana tobacum, | Tobacco |
| | Chenopodium quinoa, N. | |
| | glutinosa | |
| Tobacco necrosis virus | Nicotiana tabacum, | Tobacco |
| | Chenopodium amaranticolor, | |
| | Cucumis sativus, N. clevelandii | |
| Eggplant mosaic virus | Chenopodium amaranticolor, C. | Vegetable |
| | quinoa, Cucumis sativus, | |
| | Nicotiana clevelandii, N. | |
| | glutinosa, eggplant, and tomato | |
| Kennedya yellow mosaic virus | Kennedya rubicunda, | Vegetable |
| | Desmodium triflorum, D. | |
| | scorpiurus, Indigofera australis, | |
| | red Kennedy pea, dusky coral | |
| | pea, mung bean, French bean, pea | |
| Lycopersicon esculentum TVM | Lycopersicon esculentum | Vegetable |
| viroid | | |
| Oat blue dwarf virus | Avena sativa, Hordeum vulgare, | Vegetable |
| | Linum usitatissimum | |
| Obuda pepper virus | Nicotiano glutinosa, | Vegetable |
| | Chenopodium amaranticolor, N. | |
| | tabacum, and pepper | |
| Olive latent virus 1 | Oleo europaea | Vegetable |
| Paprika mild mottle virus | Capsicum annuum, Nicotiana | Vegetable |
| | benthamiana, N. clevelandii | |
| PMMV | Capsicum frutescens, C. annuum | Vegetable |
| Tomato mosaic virus | Lycopersicon esculentum | Vegetable |
| Turnip vein-clearing virus | Crucifers | Vegetable |
| Carnation mottle virus | Dianthaus caryophyllus | Others |
| Cocksfoot mottle virus | Avena sativa, Dactylis glomerata, | Others |
| | Hordeium vulgare, Triticum | |
| | aestivum, cocksfoot, and wheat | |
| Galinsoga mosaic virus | Galinsoga parviflora | Others |
| Johnsongrass chlorotic stripe | Sorghum halepense | Others |
| mosaic virus | | |
| Odontoglossum ringspot virus | Chenopodium quinoa (L), | Others |
| | Nicotiana tabacum cv. Xanthi-nc | |
| | (L) | |
| Ononis yellow mosaic virus | Ononis repens | Others |
| Panicum mosaic virus | Panicum vigatum | Others |
| Poinsettia mosaic virus | Euphorbia pulcherrima, E. | Others |
| | fulgens, Nicotiana benthamiana, | |
| | E. cyathophora | |
| Pothos latent virus | Nicotiana clevelandii, N. | Others |
| | benthamiana, N. hispens | |
| Ribgrass mosaic virus | Plantago lanceolata | Others |

The invention relates to the use of novel vaccines to prevent plant viruses from transforming mammalian host cells into cancerous lesions. Additionally, by following the mechanisms of effective plant host defenses, therapeutic modalities for the plant virus-induced tumors may be derived from an understanding of known plant host-defense mechanisms that have evolved to protect the plant from the plant virus. Further stress conditions such as inflammation or TLR activation that would lead to increase viral replication may be monitored and treated in patients that have been exposed to plant viruses.

The methods are useful for treating disease in a subject. As used herein, a subject is a mammal such as a human, non-human primate, cow, horse, pig, sheep, goat, dog, cat, or rodent. In all embodiments human subjects are preferred. A disease treatable according to the methods of the invention is any disease in which a plant virus plays a role in the development, maintenance or advancement of the disease. Such diseases are referred to as disease associated with a plant virus and include, for instance proliferative disorders, such as cancer, and neurodegenerative diseases. A disease associated with a plant virus is not a disease known to be associated with a mammalian virus, such as, for instance, HIV or HBV infection.

It was discovered according to the invention that Tobacco Mosaic Virus (TMV) is present in human bladder cancer cells. Inhibition of the virus using an anti-viral agent resulted in a reduction in proliferation of the infected cancer cells. As a result TMV is implicated in the development and progression of human bladder cancer. In addition to bladder cancer, several serious cancers are linked to the use of tobacco, including cancers of the lung, esophagus, larynx (voice box), mouth, throat, kidney, bladder, pancreas, stomach, and cervix, as well as acute myeloid leukemia. Even smokeless tobacco, including snuff and chewing tobacco, increase the risks of oral, facial, and bladder cancer. Furthermore, tobacco field workers have a significantly higher incidence of bladder and other cancers. Bladder cancers have very distinct morphological appearances and individual tumors appear as “tree-like” growths along the bladder wall.

The incidence of different types of cancer vary based on geographical areas, as do different plant viruses that infect food ingested by humans. For instance, the incidence of stomach cancer is highest in Asia and South America and the incidence of cervical cancer is highest in Latin America, Africa, India and Australia. Cancers with the highest incidence in the more developed countries such as North America and Europe include breast cancer and prostate cancer. Gastrointestinal cancers are highest in Japan and Southeast Asia. In India, the leading cancer, oral maxillo-facial tumors, are significantly linked to chewing leaves of the Betel plant that is frequently infected with the plant virus, badnavirus. These differences may reflect the impact of lifestyle or foods. Importantly, food groups that are ingested in regional areas include plants that are well documented to be infected with plant viruses. Thus, plant viruses are a significant etiologic factor in the majority of cancers, including but not limited to Tobacco Mosaic Virus with bladder and other tobacco-associated tumors; Rice Virus with stomach and gastro-intestinal tumors; Pepper viruses with other regional stomach tumors, etc. One class of virus, found in food, spice and medicine, that is extensively used by humans is Solanaceae. It is believed that the presence of the Cauliflower mosaic virus is associated with gastrointestinal, colon, and head and neck cancers.

The invention involves in some aspects methods of modulating gastrointestinal plant viral levels in a subject by administering to the subject a plant virus vaccine. The level of plant virus in the gastrointestinal tract of a subject can be determined using a number of known techniques in the art. For instance, Zhang et al 2006, supra, describes methods for determining levels of plant virus in human gastrointestinal tracts. Plant virus levels van be determined in human fecal or blood samples, for instance. Exemplary assays are provided below.

The levels of plant virus in the gastrointestinal system may be compared to a control. For instance, the levels may be compared to standard known levels or ranges of levels for normal or diseased subjects. Alternatively, the levels may be compared in the same or different subjects before and/or after vaccine administration. In other embodiments the levels may be compared to prior levels measured in the same subject to assess changes over time.

Additionally, it has been discovered that a plant virus vaccine and other anti-viral therapeutics described herein can be used to treat a subject at risk of having a plant virus associated cancer. A subject at risk of having a plant virus associated cancer as used herein is a subject who is at risk of coming into contact with a plant virus associated with a disease. The subject could come into contact with the plant virus by being exposed to a plant, by residing in or traveling to a geographical region associated with a particular plant, by being in a particular age group that might be exposed to a plant or any other factor determined to be a risk factor for exposure to a plant associated with a virus. In some embodiments the subject has been exposed to a plant virus.

The plant virus vaccine and other anti-viral therapeutics described herein can also be used to treat a subject having a plant virus associated neurodegenerative disease. A subject having a plant virus associated neurodegenerative disease as used herein is a subject who is at risk of or who has come into contact with a plant virus associated with a neurodegenerative disease. Plant virus associated with a neurodegenerative diseases include for instance amytrophic lateral sclerosis (ALS) and Parkinson's disease. A link between consumption of the plant Cycas micronesica, for example by the people of Guam, and the development of ALS/Parkinsonism Demensia Complex has been established (Shen, W. et al, Ann Neurol, 2010; 68, p. 70-80.) Others have proposed an epidemiologic connection between consumption of castor bean plants, which may be infected with viruses such as Olive latent virus 2, and ALS.

In some aspects the invention is directed to a vaccine that is composed of an isolated plant viral antigen. A plant viral “antigen” or “immunogen” as used herein refers to a non-infectious plant virus or immunogenic portion, fragment or derivative thereof. The antigen may be a nucleic acid antigen and/or a peptide antigen and optionally may include lipids, such as those found in viral lipid envelopes. For instance an antigen or immunogen may comprise a viral like particle (VLP), whole organism, killed, attenuated or live; a subunit or portion of an organism; a recombinant vector containing an insert with immunogenic properties; a piece or fragment of DNA capable of inducing an immune response upon presentation to a host animal; a protein, a glycoprotein, a lipoprotein, a polypeptide, a peptide, an epitope, a hapten, or any combination thereof.

The plant viral antigen is immunogenic. The term “immunogenic” as used herein refers to the specific biological immune response to a substance i.e. antigen or immunogen in a host animal. An immunogenic peptide is a viral peptide that elicits an immune response specific for the virus or viruses. Immunogenic peptides of viruses are well known in the art. Exemplary plant viral peptides are shown in Example 5. These peptides include but are not limited to SEQ ID NOs 1-429. The immunogenic peptides in some embodiments are the peptides of Example 5, immunogenic variants or fragments thereof.

In some instances the antigen, and thus the vaccine, is composed of attenuated virus. The virus, may be, for instance, heat killed intact virus.

The TMV peptides presented in Example 5 are those identified by Moudallal et al, A major part of the polypeptide chain of tobacco mosaic virus protein is antigenic, EMBO J. 1985 May; 4(5): 1231-1235. Moudallal et al, identified a number of conformation-dependent epitopes in the viral protein. In their assays Moudallal et al, concluded that “virtually the entire sequence of TMVP possessed antigenic activity.”

The plant viral antigen may also be a nucleic acid of at least one gene encoding a plant viral peptide. Examples of nucleic acids encoding plant viruses and plant virus genes are set forth in Example 6. These nucleic acid sequences include but are not limited to SEQ ID NOS: 430-438, as well as fragments and functional variants thereof.

In order to effect expression of the gene the nucleic acid may be delivered in a vector and/or operably linked to a heterologous promoter and transcription terminator. As used herein, a “vector” may be any of a number of nucleic acid molecules into which a desired sequence may be inserted by restriction and ligation for transport between different genetic environments or for expression in a host cell. Vectors are typically composed of DNA although RNA vectors are also available. Vectors include, but are not limited to, plasmids, phagemids, and virus genomes.

A cloning vector is one which is able to replicate in a host cell, and which is further characterized by one or more endonuclease restriction sites at which the vector may be cut in a determinable fashion and into which a desired DNA sequence may be ligated such that the new recombinant vector retains its ability to replicate in the host cell. An expression vector is one into which a desired DNA sequence may be inserted by restriction and ligation such that it is operably joined to regulatory sequences and may be expressed as an RNA transcript.

As used herein, a coding sequence and regulatory sequences are said to be “operably joined” when they are covalently linked in such a way as to place the expression or transcription of the coding sequence under the influence or control of the regulatory sequences. As used herein, “operably joined” and “operably linked” are used interchangeably and should be construed to have the same meaning. If it is desired that the coding sequences be translated into a functional protein, two DNA sequences are said to be operably joined if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region is operably joined to a coding sequence if the promoter region is capable of effecting transcription of that DNA sequence such that the resulting transcript can be translated into the desired protein or polypeptide.

The precise nature of the regulatory sequences needed for gene expression may vary between species or cell types, but shall in general include, as necessary, 5′ non-transcribed and 5′ non-translated sequences involved with the initiation of transcription and translation respectively, such as a TATA box, capping sequence, CAAT sequence, and the like. Often, such 5′ non-transcribed regulatory sequences will include a promoter region which includes a promoter sequence for transcriptional control of the operably joined gene. Regulatory sequences may also include enhancer sequences or upstream activator sequences as desired. The vectors of the invention may optionally include 5′ leader or signal sequences. The choice and design of an appropriate vector is within the ability and discretion of one of ordinary skill in the art.

The vector may be a replication defective vector. These types of vectors include but are not limited to adenoviral vectors.

The antigen in the vaccine may be an antigenic determinant. An “antigenic determinant” or “epitope” as used herein refers to a portion of an antigen that contacts a particular antibody. When a protein or fragment of a protein is used to immunize a host animal, numerous regions of the protein may induce the production of antibodies that bind specifically to a given region or three-dimensional structure on the protein; these regions or structures are referred to as antigenic determinants.

As used herein, the term “vaccine composition” includes at least one immunogenic antigen or immunogen in a pharmaceutically acceptable carrier useful for inducing an immune response in a host. Vaccine compositions can be administered in dosages and by techniques well known to those skilled in the medical or veterinary arts, taking into consideration such factors as the age, sex, weight, species and condition of the recipient animal, and the route of administration. As used herein, the term “host cell” refers to any mammalian cell, whether located in vitro or in vivo. For example, host cells may be located in a transgenic animal.

The vaccine composition may be formulated with or co-administered with an adjuvant. An “adjuvant” as used herein refers to a substance added to a vaccine to increase a vaccine's immunogenicity by stimulating the humoral and/or cellular immune response and/or functioning as a depo. Known vaccine adjuvants include, but are not limited to, oil and water emulsions, oil-in-water emulsions, water-in-oil emulsions, water-in-oil-in-water emulsions, saponin, aluminum hydroxide, dextran sulfate, carbomer, sodium alginate, (N,N-dioctadecyl-N′,N-bis(2-hydroxyethyl)-propanediamine), paraffin oil, muramyl dipeptide, cationic lipids, DMRIE, DOPE, and TLR ligands such as CpG oligonucleotides.

Before the instant invention, plant viruses were utilized as carriers or drug delivery reagents in vaccines. For instance, the prior art has shown the use of inactivated virus like particles derived from plants as carriers for non-plant based antigens in vaccines. These viral like particles can be loaded with DNA encoding foreign peptides which will produce the antigen of interest or they could be loaded with drugs. Modified plant viruses have also been used as smart bombs to deliver chemical payloads. These modified plant viruses have a viral shell with DNA removed leaving a cargo space of 17 nanometers which can be filled with drugs to deliver to cells. The viral shell may be coated in small proteins called signal peptides, which target the complex to a particular tissue. When administered to a subject the virus presumably travels to the target tissue and injects the payload into the cell. These prior art constructs differ from the plant viral vaccines of the invention in several important ways.

The vaccines of the invention are designed such that the antigen is part of the plant virus. In other words the vaccine includes components which elicit a specific immune response against a plant virus in the host. In addition to the plant viral antigen, the vaccine can include other foreign antigens in some embodiments, as long as it includes an immunogenic plant virus antigen. In some embodiments the vaccine does not include any nucleic acid and/or protein other than the plant viral nucleic acid and/or protein. Thus in some embodiments the plant viral antigen is an immunogenic nucleic acid or peptide of a plant virus, and is not a plant viral particle having a foreign peptide or nucleic acid incorporated therein.

Recombinant immunogenic proteins of plant viruses can be assembled into VLPs for use as vaccines. VLPs can be assembled from naturally expressed or recombinantly produced viral proteins. Disulfide bonds, including inter-capsomeric disulfide bonds have been demonstrated to be important for VLPs stability and possibly assembly. Typically, the recombinant proteins can be produced in many different types of host cells. The host cells are transformed with the appropriate genetic constructs and once the proteins are produced, they may be harvested and purified using any known procedures. It is possible that parts of the VLP can be fused to proteins of interest to help increase the immunogenicity of the vaccine.

The invention also relates to a method for treating a subject, wherein the subject has a disease associated with a plant virus, with an antiviral compound in an effective amount to reduce infection of the subject with the plant virus. An effective amount to reduce infection of the subject with the plant virus refers to an amount of an antiviral compound that increases the resistance of the subject to infection with the virus, in other words, decreases the likelihood that the subject will develop the disease resulting from the virus, as well as reducing the viral levels to treat the disease, maintain the viral levels to prevent the disease from becoming worse, or to slow the progressive infection with the virus compared to in the absence of the therapy.

An anti-viral compound, as used herein is any compound that inhibits or interferes with viral development, infectivity or replication. A number of anti-viral compounds are known in the art. For instance, anti-viral compounds include but are not limited to, compounds which interfere with cell entry, compounds that interfere with viral synthesis, compounds that interfere with transcription and translation and compounds that inhibit viral assembly.

Compounds which interfere with cell entry include, for instance, agents which mimic the virus-associated protein (VAP) and bind to the cellular receptors, such as VAP anti-idiotypic antibodies, natural ligands of the receptor and anti-receptor antibodies and agents which mimic the cellular receptor and bind to the VAP, including anti-VAP antibodies, receptor anti-idiotypic antibodies, extraneous receptor and synthetic receptor mimics.

Compounds that interfere with viral synthesis, include but are not limited to agents that block reverse transcription such as nucleotide or nucleoside analogues and inhibitors of RNA dependent RNA polymerase. Inhibitors of RNA dependent RNA polymerase are particularly interesting plant anti-viral compounds. It has previously been shown that replication of a plant virus and infection of the host cell by the virus resulted from the binding of the plant RNA dependent RNA polymerase to a host factor that allowed infection. Our analysis demonstrates that the plant virus host factor has sequence homology to an analogous factor that may be necessary for lysogenic infection with papilloma viruses. The factor may be associated with release from dead cells or conditions of inflammation in the host.

Compounds that interfere with transcription and translation include, for instance, agents that block transcription factor binding and inhibitory nucleic acids such as antisense and siRNA.

Compounds that inhibit viral assembly include protease inhibitors.

Exemplary anti-viral compounds include but are not limited to Tenofovir

Disoproxil Fumarate, Abacavir, Emtricitabine, Lamivudine, Zidovudine, Atazanavir Sulfate, Nevirapine, Stavudine, Didanosine, Efavirenz, Lopinavir, Zalcitabine, Entecavir, Apricitabine, Adefovir, Nevirapine, Delavirdine, Etravirine, Rilpivirine, portmanteau inhibitors, and Ritonavir.

Another anti-viral compound useful according to the invention is melittin and analogs thereof. Such compounds are described in Marcos et al PNAS v. 92, p. 12466, 1995. Melittin is a 26 amino acid amphipathic peptide.

A recently developed antiviral strategy, also encompassed by anti-viral compounds according to the invention is double-stranded RNA activated caspase oligomerizer (DRACO) methods. DRACO involves the destruction of dsRNA inside infected cells while sending a signal to the cell to begin apoptosis.

A number of these anti-viral compounds are naturally occurring plant viral defense mechanisms. These are chemicals or other mechanisms developed by plants to avoid infection or treat infection by viruses. Naturally occurring plant viral defense mechanisms include but are not limited to chloroquine, Resistance (R) proteins, salicylic acid, jasmonic acid, inhibitory nucleic acids specific for essential plant genes, such as argonaute (e.g., AGO1, AGO2, flavonoids, anthocyanins, phytoalexins, medicarpin, rishitin, camalexin, capsaisin, glucosinolate, defensins, alpha-amylase, protease inhibitors, lignin and furanocoumarins. Medicinal plants have been described previously. For instance, Mukhtar et al (Virus Research, v. 131, p. 111-120 (2008)) which is incorporated by reference is a review article on medicinal plants having anti-viral activities. Such plants fall within the anti-viral compounds of the invention.

Anti-viral compounds of the invention also include inhibitory nucleic acids that target the plant virus. Previous studies have shown that administration of siRNA in animal models is useful for preventing infection. These same mechanisms are useful in treating plant viruses that have infected mammalian cells. Preferably, the virus is selected from any of the viruses listed in Appendix A of U.S. Patent Application Ser. No. 61/537,306 which is incorporated by reference or Table 1. A target nucleic acid is any nucleic acid sequence whose expression or activity is to be modulated. The target nucleic acid can be DNA or RNA.

The inhibitory nucleic acids target nucleic acids that are part of a viral genome and, in particular, nucleic acids comprising essential genes. More specifically, the inhibitory nucleic acid inhibit expression of the target viral sequence. “Essential genes” refer to genes whose expression is required for infection and/or replication functions of the virus. The viral genome may be selected, for example, from the genomes of a virus noted in Appendix A of U.S. Patent Application Ser. No. 61/537,306 and/or Table 1. Essential genes in the genomes of the viruses noted above are known to the skilled artisan. The gene essential for infectivity or replication of the virus may be for instance plant virus genome-linked protein (VPg), VPg-Pro, the 3′UTR, the 5′ UTR, zinc finger region of the capsid protein, or tRNA like domain.

Thus, the invention also features the use of small nucleic acid molecules, referred to as short interfering nucleic acid (siNA) that include, for example: microRNA (miRNA), short interfering RNA (siRNA), double-stranded RNA (dsRNA), and short hairpin RNA (shRNA) molecules to knockdown expression of viral proteins. An siNA of the invention can be unmodified or chemically-modified. An siNA of the instant invention can be chemically synthesized, expressed from a vector or enzymatically synthesized. The instant invention also features various chemically-modified synthetic siNA molecules capable of modulating gene expression or activity in cells by, for instance, RNA interference (RNAi). The use of chemically-modified siNA improves various properties of native siNA molecules through, for example, increased resistance to nuclease degradation in vivo and/or through improved cellular uptake. Furthermore, siNA having multiple chemical modifications may retain its RNAi activity. The siNA molecules of the instant invention provide useful reagents and methods for a variety of therapeutic applications.

Chemically synthesizing nucleic acid molecules with modifications (base, sugar and/or phosphate) that prevent their degradation by serum ribonucleases can increase their potency (see e.g., Eckstein et al., International Publication No. WO 92/07065; Perrault et al, 1990 Nature 344, 565; Pieken et al., 1991, Science 253, 314; Usman and Cedergren, 1992, Trends in Biochem. Sci. 17, 334; Usman et al., International Publication No. WO 93/15187; and Rossi et al., International Publication No. WO 91/03162; and Sproat, U.S. Pat. No. 5,334,711; all of these describe various chemical modifications that can be made to the base, phosphate and/or sugar moieties of the nucleic acid molecules herein). Modifications which enhance their efficacy in cells, and removal of bases from nucleic acid molecules to shorten oligonucleotide synthesis times and reduce chemical requirements are desired.

There are several examples in the art describing sugar, base and phosphate modifications that can be introduced into nucleic acid molecules with significant enhancement in their nuclease stability and efficacy. For example, oligonucleotides are modified to enhance stability and/or enhance biological activity by modification with nuclease resistant groups, for example, 2′ amino, 2′-C-allyl, 2′-fluoro, 2′-O-methyl, 2′-H, nucleotide base modifications (for a review see Usman and Cedergren, 1992, TIBS. 17, 34; Usman et al., 1994, Nucleic Acids Symp. Ser. 31, 163; Burgin et al., 1996, Biochemistry, 35, 14090). Sugar modification of nucleic acid molecules have been extensively described in the art (see Eckstein et al., International Publication PCT No. WO 92/07065; Perrault et al. Nature, 1990, 344, 565 568; Pieken et al. Science, 1991, 253, 314317; Usman and Cedergren, Trends in Biochem. Sci., 1992, 17, 334 339; Usman et al. International Publication PCT No. WO 93/15187; Sproat, U.S. Pat. No. 5,334,711 and Beigelman et al., 1995, J. Biol. Chem., 270, 25702; Beigelman et al., International PCT publication No. WO 97/26270; Beigelman et al., U.S. Pat. No. 5,716,824; Usman et al.).

In one embodiment, one of the strands of the double-stranded siRNA molecule comprises a nucleotide sequence that is complementary to a nucleotide sequence of a target RNA or a portion thereof, and the second strand of the double-stranded siRNA molecule comprises a nucleotide sequence identical to the nucleotide sequence or a portion thereof of the targeted RNA. In another embodiment, one of the strands of the double-stranded siRNA molecule comprises a nucleotide sequence that is substantially complementary to a nucleotide sequence of a target RNA or a portion thereof, and the second strand of the double-stranded siRNA molecule comprises a nucleotide sequence substantially similar to the nucleotide sequence or a portion thereof of the target RNA. In another embodiment, each strand of the siRNA molecule comprises about 19 to about 23 nucleotides, and each strand comprises at least about 19 nucleotides that are complementary to the nucleotides of the other strand.

In another aspect the nucleic acid molecules comprise a 5′ and/or a 3′-cap structure. By “cap structure” is meant chemical modifications, which have been incorporated at either terminus of the oligonucleotide (see for example Wincott et al, WO 97/26270). Other useful RNA derivatives incorporate nucleotides having modified carbohydrate moieties, such as 2′O-alkylated residues or 2′-O-methyl ribosyl derivatives and 2′-O-fluoro ribosyl derivatives. The RNA bases may also be modified. Any modified base useful for inhibiting or interfering with the expression of a target sequence may be used. For example, halogenated bases, such as 5-bromouracil and 5-iodouracil can be incorporated. The bases may also be alkylated, for example, 7-methylguanosine can be incorporated in place of a guanosine residue. Non-natural bases that yield successful inhibition can also be incorporated.

For example the siRNA can be a double-stranded polynucleotide molecule comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof. The siRNA can be assembled from two separate oligonucleotides, where one strand is the sense strand and the other is the antisense strand, wherein the antisense and sense strands are self-complementary (i.e. each strand comprises nucleotide sequence that is complementary to nucleotide sequence in the other strand; such as where the antisense strand and sense strand form a duplex or double stranded structure, for example wherein the double stranded region is about 15 to about 30, e.g., about 15, 16, 17, 18, 19, 20, 21, 22, 23, 24, 25, 26, 27, 28, 29 or 30 base pairs; the antisense strand comprises nucleotide sequence that is complementary to nucleotide sequence in a target nucleic acid molecule or a portion thereof and the sense strand comprises nucleotide sequence corresponding to the target nucleic acid sequence or a portion thereof (e.g., about 15 to about 25 or more nucleotides of the siRNA molecule are complementary to the target nucleic acid or a portion thereof). Alternatively, the siRNA is assembled from a single oligonucleotide, where the self-complementary sense and antisense regions of the siRNA are linked by means of a nucleic acid based or non-nucleic acid-based linker(s). The siRNA can be a polynucleotide with a duplex, asymmetric duplex, hairpin or asymmetric hairpin secondary structure, having self-complementary sense and antisense regions, wherein the antisense region comprises nucleotide sequence that is complementary to nucleotide sequence in a separate target nucleic acid molecule or a portion thereof and the sense region having nucleotide sequence corresponding to the target nucleic acid sequence or a portion thereof. The siRNA can be a circular single-stranded polynucleotide having two or more loop structures and a stem comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof, and wherein the circular polynucleotide can be processed either in vivo or in vitro to generate an active siRNA molecule capable of mediating RNAi. The siRNA can also comprise a single stranded polynucleotide having nucleotide sequence complementary to nucleotide sequence in a target nucleic acid molecule or a portion thereof (for example, where such siRNA molecule does not require the presence within the siRNA molecule of nucleotide sequence corresponding to the target nucleic acid sequence or a portion thereof), wherein the single stranded polynucleotide can further comprise a terminal phosphate group, such as a 5′-phosphate (see for example Martinez et al., 2002, Cell., 110, 563-574 and Schwarz et al., 2002, Molecular Cell, 10, 537-568), or 5′,3′-diphosphate. In certain embodiments, the siRNA molecule of the invention comprises separate sense and antisense sequences or regions, wherein the sense and antisense regions are covalently linked by nucleotide or non-nucleotide linkers molecules as is known in the art, or are alternately non-covalently linked by ionic interactions, hydrogen bonding, van der waals interactions, hydrophobic interactions, and/or stacking interactions.

The siNA are composed of nucleotide sequences that are complementary to nucleotide sequences of a target gene. “Complementarity” as used herein refers to the degree to which a nucleic acid can form hydrogen bonds with another nucleic acid sequence by either traditional Watson-Crick or other non-traditional bonds. The binding free energy for a nucleic acid molecule with its complementary sequence is sufficient to allow the relevant function of the nucleic acid to proceed, e.g., RNAi activity. Methods for determining binding free energies for nucleic acid molecules is well known in the art (see, e.g., Turner et al., 1987, CSH Symp. Quant. Biol. LII pp. 123-133; Frier et al., 1986, Proc. Nat. Acad. Sci. USA 83:9373-9377; Turner et al., 1987, J. Am. Chem. Soc. 109:3783-3785). A percent complementarity indicates the percentage of contiguous residues in a nucleic acid molecule that can form hydrogen bonds (e.g., Watson-Crick base pairing) with a second nucleic acid sequence (e.g., 5, 6, 7, 8, 9, or 10 nucleotides out of a total of 10 nucleotides in the first oligonucleotide being based paired to a second nucleic acid sequence having 10 nucleotides represents 50%, 60%, 70%, 80%, 90%, and 100% complementary respectively).

“Perfectly complementary” as used herein means that all the contiguous residues of a nucleic acid sequence will hydrogen bond with the same number of contiguous residues in a second nucleic acid sequence. In one embodiment, an siNA molecule of the invention comprises about 15 to about 30 or more (e.g., about 15, 16, 17, 18, 19, 20, 21, 22, 23, 24, 25, 26, 27, 28, 29, or 30 or more) nucleotides that are complementary to one or more target nucleic acid molecules or a portion thereof.

The siNA molecules modulate gene expression. The term “modulate” as used herein refers to change in the expression of the gene, or level of RNA molecule or equivalent RNA molecules encoding one or more proteins or protein subunits, or activity of one or more proteins or protein subunits such that it is up regulated or down regulated, and such that expression, level, or activity is greater than or less than that observed in the absence of the modulator.

Inhibition of gene expression indicates that the expression of the gene, or level of RNA molecules or equivalent RNA molecules encoding one or more proteins or protein subunits, or activity of one or more proteins or protein subunits, is reduced below that observed in the absence of the nucleic acid molecules (e.g., siRNA) of the invention. In one embodiment, inhibition, down-regulation or reduction with an siNA molecule is below that level observed in the presence of an inactive or attenuated molecule. In another embodiment, inhibition, down-regulation, or reduction with siNA molecules is below that level observed in the presence of, for example, an siNA molecule with scrambled sequence or with mismatches. A therapeutically or prophylactically significant reduction is about 30%, about 40%, about 50%, about 60%, about 70%, about 80%, about 90%, about 100%, about 125%, about 150% or more compared to a control.

A gene is a nucleic acid that encodes an RNA, for example, nucleic acid sequences including, but not limited to, structural genes encoding a polypeptide. A gene can also encode a functional RNA (fRNA) or non-coding RNA (ncRNA), such as small temporal RNA (stRNA), micro RNA (miRNA), small nuclear RNA (snRNA), short interfering RNA (siRNA), small nucleolar RNA (snRNA), ribosomal RNA (rRNA), transfer RNA (tRNA) and precursor RNAs thereof.

In some embodiments an siNA is an shRNA, shRNA-mir, or microRNA molecule encoded by and expressed from a genomically integrated transgene or a plasmid-based expression vector. Thus, in some embodiments a molecule capable of inhibiting mRNA expression, or microRNA activity, is a transgene or plasmid-based expression vector that encodes a small-interfering nucleic acid. Such transgenes and expression vectors can employ either polymerase II or polymerase III promoters to drive expression of these shRNAs and result in functional siNAs in cells. The former polymerase permits the use of classic protein expression strategies, including inducible and tissue-specific expression systems. In some embodiments, transgenes and expression vectors are controlled by tissue specific promoters. In other embodiments transgenes and expression vectors are controlled by inducible promoters, such as tetracycline inducible expression systems.

In another embodiment, a short interfering nucleic acid of the invention is expressed in mammalian cells using a mammalian expression vector. The recombinant mammalian expression vector may be capable of directing expression of the nucleic acid preferentially in a particular cell type (e.g., tissue-specific regulatory elements are used to express the nucleic acid). Tissue specific regulatory elements are known in the art. Non-limiting examples of suitable tissue-specific promoters include the myosin heavy chain promoter, albumin promoter, lymphoid-specific promoters, neuron specific promoters, pancreas specific promoters, and mammary gland specific promoters. Developmentally-regulated promoters are also encompassed, for example the murine hox promoters and the a-fetoprotein promoter.

Viral-mediated delivery mechanisms to deliver siNAs to cells in vitro and in vivo have been described in Xia, H. et al. (2002) Nat Biotechnol 20(10):1006). Plasmid- or viral-mediated delivery mechanisms of shRNA may also be employed to deliver shRNAs to cells in vitro and in vivo as described in Rubinson, D. A., et al. ((2003) Nat. Genet. 33:401-406) and Stewart, S. A., et al. ((2003) RNA 9:493-501). Other methods of introducing siNA molecules of the present invention to target cells include a variety of art-recognized techniques including, but not limited to, calcium phosphate or calcium chloride co-precipitation, DEAE-dextran-mediated transfection, lipofection, or electroporation as well as a number of commercially available transfection kits (e.g., OLIGOFECTAMINE® Reagent from Invitrogen) (see, e.g. Sui, G. et al. (2002) Proc. Natl. Acad. Sci. USA 99:5515-5520; Calegari, F. et al. (2002) Proc. Natl. Acad. Sci., USA Oct. 21, 2002; J-M Jacque, K. Triques and M. Stevenson (2002) Nature 418:435-437).

In another embodiment of the invention, the siNA may be transported or conducted across biological membranes using carrier polymers which comprise, for example, contiguous, basic subunits, at a rate higher than the rate of transport of siNA molecules which are not associated with carrier polymers. Combining a carrier polymer with siNA, with or without a cationic transfection agent, results in the association of the carrier polymer and the siNA. The carrier polymer may efficiently deliver the siNA, across biological membranes both in vitro and in vivo. Accordingly, the invention provides methods for delivery of an siNA, across a biological membrane, e.g., a cellular membrane including, for example, a nuclear membrane, using a carrier polymer. The invention also provides compositions comprising an siNA in association with a carrier polymer.

Other inhibitor molecules that can be used include sense and antisense nucleic acids (single or double stranded), ribozymes, peptides, DNAzymes, peptide nucleic acids (PNAs), triple helix forming oligonucleotides, antibodies, and aptamers and modified form(s) thereof directed to sequences in gene(s), RNA transcripts, or proteins. Antisense and ribozyme suppression strategies have led to the reversal of a tumor phenotype by reducing expression of a gene product or by cleaving a mutant transcript at the site of the mutation (Carter and Lemoine Br. J. Cancer. 67(5):869-76, 1993; Lange et al., Leukemia. 6(11):1786-94, 1993; Valera et al., J. Biol. Chem. 269(46):28543-6, 1994; Dosaka-Akita et al., Am. J. Clin. Pathol. 102(5):660-4, 1994; Feng et al., Cancer Res. 55(10):2024-8, 1995; Quattrone et al., Cancer Res. 55(1):90-5, 1995; Lewin et al., Nat Med. 4(8):967-71, 1998). For example, neoplastic reversion was obtained using a ribozyme targeted to an H-Ras mutation in bladder carcinoma cells (Feng et al., Cancer Res. 55(10):2024-8, 1995). Ribozymes have also been proposed as a means of both inhibiting gene expression of a mutant gene and of correcting the mutant by targeted trans-splicing (Sullenger and Cech Nature 371(6498):619-22, 1994; Jones et al., Nat. Med. 2(6):643-8, 1996). Ribozyme activity may be augmented by the use of, for example, non-specific nucleic acid binding proteins or facilitator oligonucleotides (Herschlag et al., Embo J. 13(12):2913-24, 1994; Jankowsky and Schwenzer Nucleic Acids Res. 24(3):423-9,1996). Multitarget ribozymes (connected or shotgun) have been suggested as a means of improving efficiency of ribozymes for gene suppression (Ohkawa et al., Nucleic Acids Symp Ser. (29):121-2, 1993).

Anti-sense oligonucleotides may be designed to hybridize to the complementary sequence of nucleic acid, pre-mRNA or mature mRNA, interfering with the production of an viral protein encoded by a given DNA sequence (e.g. either native polypeptide or a mutant form thereof), so that its expression is reduce or prevented altogether. Anti-sense techniques may be used to target a coding sequence; a control sequence of a gene, e.g. in the 5′ flanking sequence, whereby the anti-sense oligonucleotides can interfere with control sequences. Anti-sense oligonucleotides may be DNA or RNA and may be of around 14-23 nucleotides, particularly around 15-18 nucleotides, in length. The construction of antisense sequences and their use is described in Peyman and Uhlmann, Chemical Reviews, 90:543-584, (1990), and Crooke, Ann. Rev. Pharmacol. Toxicol., 32:329-376, (1992).

It may be preferable that there is complete sequence identity in the sequence used for down-regulation of expression of a target sequence, and the target sequence, though total complementarity or similarity of sequence is not essential. One or more nucleotides may differ in the sequence used from the target gene. Thus, a sequence employed in a down-regulation of gene expression in accordance with the present invention may be a wild-type sequence (e.g. gene) selected from those available, or a mutant, derivative, variant or allele, by way of insertion, addition, deletion or substitution of one or more nucleotides, of such a sequence.

The sequence need not include an open reading frame or specify an RNA that would be translatable. It may be preferred for there to be sufficient homology for the respective sense RNA molecules to hybridize. There may be down regulation of gene expression even where there is about 5%, 10%, 15% or 20% or more mismatch between the sequence used and the target gene.

Triple helix approaches have also been investigated for sequence-specific gene suppression. Triple helix forming oligonucleotides have been found in some cases to bind in a sequence-specific manner (Postel et al., Proc. Natl. Acad. Sci. U.S.A. 88(18):8227-31, 1991; Duval-Valentin et al., Proc. Natl. Acad. Sci. U.S.A. 89(2):504-8, 1992; Hardenbol and Van Dyke Proc. Natl. Acad. Sci. U.S.A. 93(7):2811-6, 1996; Porumb et al., Cancer Res. 56(3):515-22, 1996). Similarly, peptide nucleic acids have been shown to inhibit gene expression (Hanvey et al., Antisense Res. Dev. 1(4):307-17, 1991; Knudsen and Nielson Nucleic Acids Res. 24(3):494-500, 1996; Taylor et al., Arch. Surg. 132(11):1177-83, 1997). Minor-groove binding polyamides can bind in a sequence-specific manner to DNA targets and hence may represent useful small molecules for future suppression at the DNA level (Trauger et al., Chem. Biol. 3(5):369-77, 1996). In addition, suppression has been obtained by interference at the protein level using dominant negative mutant peptides and antibodies (Herskowitz Nature 329(6136):219-22, 1987; Rimsky et al., Nature 341(6241):453-6, 1989; Wright et al., Proc. Natl. Acad. Sci. U.S.A. 86(9):3199-203, 1989). In some cases suppression strategies have led to a reduction in RNA levels without a concomitant reduction in proteins, whereas in others, reductions in RNA have been mirrored by reductions in protein.

The diverse array of suppression strategies that can be employed includes the use of DNA and/or RNA aptamers that can be selected to target, for example, a viral protein of interest.

The siNA that targets a viral target may be a single siNA or multiple siNA. Thus, a mixture of siNAs targeting either the same viral gene or at least 2, 3, 4, 5 or up to at least 10 different viral genes may be used. Each of the siNAs, can be screened for potential off-target effects may be analyzed using, for example, expression profiling. Such methods are known to one skilled in the art and are described, for example, in Jackson et al. Nature Biotechnology 6:635-637, 2003. In addition to expression profiling, one may also screen the potential target sequences for similar sequences in the sequence databases to identify potential sequences which may have off-target effects. One may initially screen the proposed siNAs to avoid potential off-target silencing using the sequence identity analysis by any known sequence comparison methods, such as BLAST. Design of siNAs is known to the skilled artisan, see for example, Dykxhoorn & Lieberman 2006 “Running interference: prospects and obstacles to using small interfering RNAs as small molecule drugs” Annu Rev Biomed Eng.

The dose of the siNA will be in an amount necessary to effect RNA interference, e.g., post translational gene silencing, of the particular target gene, thereby leading to inhibition of target gene expression or inhibition of activity or level of the protein encoded by the target gene. Assays to determine expression of the target sequence are known in the art. In one embodiment, a reporter gene, e.g., GFP, may be fused to the target sequence in a test cell, e.g., in a test animal. Effectiveness of silencing can then be measured by examining the reporter gene expression. Target cells which have been transfected with the siNA molecules can be identified by routine techniques such as immunofluorescence, phase contrast microscopy and fluorescence microscopy. In one embodiment, reduced levels of target gene mRNA may be measured by in situ hybridization (Montgomery et al., (1998) Proc. Natl. Acad. Sci., USA 95:15502-15507) or Northern blot analysis (Ngo, et al. (1998)) Proc. Natl. Acad. Sci., USA 95:14687-14692). Preferably, target gene transcription is measured using quantitative real-time PCR (Gibson et al., Genome Research 6:995-1001, 1996; Heid et al., Genome Research 6:986-994, 1996).

As used herein, “inhibition of target gene expression” includes any decrease in expression or protein activity or level of the target gene or protein encoded by the target gene as compared to a situation wherein no RNA interference has been induced. The decrease may be of at least about 30%, about 40%, about 50%, about 60%, about 70%, about 80%, about 90%, about 95% or about 99% or more as compared to the expression of a target gene or the activity or level of the protein encoded by a target gene which has not been targeted by an siNA.

The molecules useful herein are isolated molecules. As used herein, the term “isolated” means that the referenced material is removed from its native environment, e.g., a cell. Thus, an isolated biological material can be free of some or all cellular components, i.e., components of the cells in which the native material is occurs naturally (e.g., cytoplasmic or membrane component). The isolated molecules may be substantially pure and essentially free of other substances with which they may be found in nature or in vivo systems to an extent practical and appropriate for their intended use. In particular, the molecules are sufficiently pure and are sufficiently free from other biological constituents of their hosts cells so as to be useful in, for example, producing pharmaceutical preparations or sequencing. Because an isolated peptide of the invention may be admixed with a pharmaceutically acceptable carrier in a pharmaceutical preparation, the peptide may comprise only a small percentage by weight of the preparation. The peptide is nonetheless substantially pure in that it has been substantially separated from the substances with which it may be associated in living systems. In some embodiments, the peptide is a synthetic peptide.

The term “purified” in reference to a protein or a nucleic acid, refers to the separation of the desired substance from contaminants to a degree sufficient to allow the practitioner to use the purified substance for the desired purpose. Preferably this means at least one order of magnitude of purification is achieved, more preferably two or three orders of magnitude, most preferably four or five orders of magnitude of purification of the starting material or of the natural material. In specific embodiments, a purified thymus derived peptide is at least 60%, at least 80%, or at least 90% of total protein or nucleic acid, as the case may be, by weight. In a specific embodiment, a purified thymus derived peptide is purified to homogeneity as assayed by, e.g., sodium dodecyl sulfate polyacrylamide gel electrophoresis, or agarose gel electrophoresis.

The therapeutic compounds described herein can be administered in combination with other therapeutic agents and such administration may be simultaneous or sequential. When the other therapeutic agents are administered simultaneously they can be administered in the same or separate formulations, but are administered at the same time. The administration of the other therapeutic agent, including chemotherapeutics and TLR activators/agonists and the compounds of the invention can also be temporally separated, meaning that the therapeutic agents are administered at a different time, either before or after, the administration of the therapeutics described herein. The separation in time between the administration of these compounds may be a matter of minutes or it may be longer.

Thus, in some instances, the invention also involves administering another cancer treatment (e.g., radiation therapy, chemotherapy or surgery) to a subject. Examples of conventional cancer therapies include treatment of the cancer with agents such as All-trans retinoic acid, Actinomycin D, Adriamycin, anastrozole, Azacitidine, Azathioprine, Alkeran, Ara-C, Arsenic Trioxide (Trisenox), BiCNU Bleomycin, Busulfan, CCNU, Carboplatin, Capecitabine, Cisplatin, Chlorambucil, Cyclophosphamide, Cytarabine, Cytoxan, DTIC, Daunorubicin, Docetaxel, Doxifluridine, Doxorubicin, 5-fluorouracil, Epirubicin, Epothilone, Etoposide, exemestane, Erlotinib, Fludarabine, Fluorouracil, Gemcitabine, Hydroxyurea, Herceptin, Hydrea, Ifosfamide, Irinotecan, Idarubicin, Imatinib, letrozole, Lapatinib, Leustatin, 6-MP, Mithramycin, Mitomycin, Mitoxantrone, Mechlorethamine, megestrol, Mercaptopurine, Methotrexate, Mitoxantrone, Navelbine, Nitrogen Mustard, Oxaliplatin, Paclitaxel, pamidronate disodium, Pemetrexed, Rituxan, 6-TG, Taxol, Topotecan, tamoxifen, taxotere, Teniposide, Tioguanine, toremifene, trimetrexate, trastuzumab, Valrubicin, Vinblastine, Vincristine, Vindesine, Vinorelbine, Velban, VP-16, and Xeloda.

Other therapeutics for cancer involve antibodies or other binding proteins conjugated to a cytotoxic agents. The conjugates include an antibody conjugated to a cytotoxic agent such as a chemotherapeutic agent, toxin (e.g. an enzymatically active toxin of bacterial, fungal, plant or animal origin, or fragments thereof, or a small molecule toxin), or a radioactive isotope (i.e., a radioconjugate). Other antitumor agents that can be conjugated to the antibodies of the invention include BCNU, streptozoicin, vincristine and 5-fluorouracil, the family of agents known collectively LL-E33288 complex described in U.S. Pat. Nos. 5,053,394, 5,770,710, as well as esperamicins (U.S. Pat. No. 5,877,296). Enzymatically active toxins and fragments thereof which can be used in the conjugates include diphtheria A chain, nonbinding active fragments of diphtheria toxin, exotoxin A chain (from Pseudomonas aeruginosa), abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin and the tricothecenes.

For selective destruction of the cell, the antibody may comprise a highly radioactive atom. A variety of radioactive isotopes are available for the production of radioconjugated antibodies. Examples include At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu. When the conjugate is used for detection, it may comprise a radioactive atom for scintigraphic studies, for example tc⁹⁹m or I¹²³, or a spin label for nuclear magnetic resonance (NMR) imaging (also known as magnetic resonance imaging, mri), such as iodine-123, iodine-131, indium-111, fluorine-19, carbon-13, nitrogen-15, oxygen-17, gadolinium, manganese or iron.

The radio- or other labels may be incorporated in the conjugate in known ways. For example, the peptide may be biosynthesized or may be synthesized by chemical amino acid synthesis using suitable amino acid precursors involving, for example, fluorine-19 in place of hydrogen. Labels such as tc^{99m} or I¹²³, Re¹⁸⁶, Re¹⁸⁸ and In¹¹¹ can be attached via a cysteine residue in the peptide. Yttrium-90 can be attached via a lysine residue. The IODOGEN method (Fraker et al (1978) Biochem. Biophys. Res. Commun. 80: 49-57 can be used to incorporate iodine-123. “Monoclonal Antibodies in Immunoscintigraphy” (Chatal, CRC Press 1989) describes other methods in detail.

Conjugates of the antibody and cytotoxic agent may be made using a variety of bifunctional protein coupling agents such as N-succinimidyl-3-(2-pyridyldithio)propionate (SPDP), succinimidyl-4-(N-maleimidomethyl)cyclohexane-1-carboxylate, iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis(p-azidobenzoyl)hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026. The linker may be a “cleavable linker” facilitating release of the cytotoxic drug in the cell. For example, an acid-labile linker, peptidase-sensitive linker, photolabile linker, dimethyl linker or disulfide-containing linker (Chari et al., Cancer Research 52:127-131 (1992); U.S. Pat. No. 5,208,020) may be used.

TLR activation causes plant viral gene transcription. Therefore, the compositions of the invention can be combined with a TLR activation therapy, in order to induce viral transcription. TLR activators or agonists include but are not limited to TLR 3, 7, 8, and 9 agonists.

The term “TLR3 agonist” refers to a molecule that interacts with (directly or indirectly) and is capable of activating a TLR3 polypeptide to induce a full or partial receptor-mediated response (i.e. induces TLR3-mediated signaling). A TLR3 agonist, thus, may or may not bind to a TLR3 polypeptide, and may or may not interact directly with the TLR3 polypeptide. TLR3 agonists include for instance, naturally-occurring double-stranded RNA (dsRNA); synthetic ds RNA; and synthetic dsRNA analogs, such as those described in Alexopoulou et al. (2001) Nature 413:732-738. An exemplary, non-limiting example of a synthetic ds RNA analog is poly(I:C).

“TLR7 agonist” and “TLR8 agonists” include single stranded RNA having specific motifs as well as other molecules that interact with (directly or indirectly) and are capable of activating a TLR7 and/or TLR8 polypeptide to induce a full or partial receptor-mediated response (i.e. induces TLR7 and/or 8-mediated signaling).

A “TLR9 agonist” as used herein is a molecule that interacts with (directly or indirectly) and is capable of activating a TLR9 polypeptide to induce a full or partial receptor-mediated response (i.e. induces TLR9-mediated signaling). TLR9 agonists include but are not limited to CpG oligonucleotides.

The therapeutics of the invention may also be combined with CLIP inhibitors. CLIP inhibitors are described extensively in US2011/0118175 and US2010/0166782, each of which are incorporated by reference. CLIP inhibitors include, for instance, but are not limited to FRIMAVLAS (SEQ ID NO. 439).

The invention also involves combinations of the active agents described herein with compounds that make cells more immunogenic, such as autophagy inhibitors and/or a fatty acid metabolism inhibitors. Thus, in some embodiments the invention involves the co-administration of a vaccine or anti-viral therapy of the invention with an autophagy inhibitor and/or a fatty acid metabolism inhibitor. Autophagy inhibitors and fatty acid metabolism inhibitors have been described extensively in U.S. Provisional Application No. 61/511,289 and U.S. patent application Ser. No. 13/054,147 and WO2010/008554 each of which is incorporated by reference.

When used in combination with the therapies of the invention the dosages of known therapies may be reduced in some instances, to avoid side effects.

Cancer therapies and their dosages, routes of administration and recommended usage are known in the art and have been described in such literature as the Physician's Desk Reference (56^{th} ed., 2002). In some embodiments, the therapeutic compounds of the invention are formulated into a pharmaceutical composition that further comprises one or more additional anticancer agents.

The compounds of the invention are administered in prophylactically or therapeutically effective amounts. A prophylactically or therapeutically effective amount means that amount necessary to attain, at least partly, the desired effect, or to delay the onset of, inhibit the progression of, prevent the reoccurrence of, or halt altogether, the onset or progression of the viral infection and/or the resultant disease being treated, i.e. cancer. Such amounts will depend, of course, on the particular condition being treated, the severity of the condition and individual patient parameters including age, physical condition, size, weight and concurrent treatment. These factors are well known to those of ordinary skill in the art and can be addressed with no more than routine experimentation. It is preferred generally that a maximum dose be used, that is, the highest safe dose according to sound medical judgment. It will be understood by those of ordinary skill in the art; however, that a lower dose or tolerable dose may be administered for medical reasons, psychological reasons or for virtually any other reason.

The term “preventing” or “reducing” or “inhibiting” as used herein refers to preventing plant viral infection in an individual susceptible for infection or re-infection. Accordingly, administration of a prophylactic agent can occur prior to the manifestation of symptoms characteristic of the infection or the resultant disease, such that the disease or infection is prevented or, alternatively, delayed in its progression. Any mode of administration of the therapeutic agents of the invention, as described herein or as known in the art, including topical administration or mucosal administration of the compounds of the instant invention, may be utilized for the prophylactic treatment of the plant infection or resultant disease.

An effective amount for treating precancerous or cancerous tissue may be an amount sufficient to prevent, delay or inhibit the development of a tumor or slow the growth or reverse the growth of a tumor in the subject compared to the levels in the absence of treatment. According to some aspects of the invention, an effective amount is that amount of a compound of the invention alone or in combination with another medicament, which when combined or co-administered or administered alone, results in a biological affect associated with treating the precancerous or cancerous tissue. Prevention or inhibition as used in this context refers to any reduction or delay in tumor formation as a result of the treatment when compared to an untreated subject.

As defined herein, a therapeutically effective amount of an active compound of the invention (i.e., an effective dosage) ranges from about 0.001 to 3000 mg/kg body weight, preferably about 0.01 to 2500 mg/kg body weight, more preferably about 0.1 to 2000 mg/kg body weight, and even more preferably about 1 to 1000 mg/kg, 2 to 900 mg/kg, 3 to 800 mg/kg, 4 to 700 mg/kg, or 5 to 600 mg/kg body weight. In one embodiment, the average adult is 60 kg and is administered about 0.5 to 50 mg, about 1 to 45 mg, about 2 to 40, about 3 to 35 mg, about 4 to 30 mg, about 5 to 25 mg, about 6 to 20 mg of compound. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of an active compound can include a single treatment or, preferably, can include a series of treatments.

Toxicity and efficacy of the prophylactic and/or therapeutic protocols of the present invention can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Prophylactic and/or therapeutic agents that exhibit large therapeutic indices are preferred. While prophylactic and/or therapeutic agents that exhibit toxic side effects may be used, care should be taken to design a delivery system that targets such agents to the site of affected tissue in order to minimize potential damage to uninfected cells and, thereby, reduce side effects.

The data obtained from the cell culture assays, animal studies and human studies can be used in formulating a range of dosage of the prophylactic and/or therapeutic agents for use in humans. The dosage of such agents lies preferably within a range of circulating concentrations that include the ED50 with little or no toxicity. The dosage may vary within this range depending upon the dosage form employed and the route of administration utilized. For any agent used in the method of the invention, the therapeutically effective dose can be estimated initially from cell culture assays. A dose may be formulated in animal models to achieve a circulating plasma concentration range that includes the IC50 (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

Multiple doses of the molecules of the invention are also contemplated. In some instances, when the molecules of the invention are administered with another therapeutic, for instance, an anti-cancer agent a sub-therapeutic dosage of either or both of the molecules may be used. A “sub-therapeutic dose” as used herein refers to a dosage which is less than that dosage which would produce a therapeutic result in the subject if administered in the absence of the other agent.

Pharmaceutical compositions of the present invention comprise an effective amount of one or more agents, dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. Moreover, for animal (e.g., human) administration, it will be understood that preparations should meet sterility, pyrogenicity, general safety and purity standards as required by FDA Office of Biological Standards. The compounds are generally suitable for administration to humans. This term requires that a compound or composition be nontoxic and sufficiently pure so that no further manipulation of the compound or composition is needed prior to administration to humans.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences (1990), incorporated herein by reference). Except insofar as any conventional carrier is incompatible with the active ingredient, its use in the therapeutic or pharmaceutical compositions is contemplated. The compounds may be sterile or non-sterile.

The agent may comprise different types of carriers depending on whether it is to be administered in solid, liquid or aerosol form, and whether it need to be sterile for such routes of administration as injection. The present invention can be administered intravenously, intradermally, intraarterially, intralesionally, intratumorally, intracranially, intraarticularly, intraprostaticaly, intrapleurally, intratracheally, intranasally, intravitreally, intravaginally, intrarectally, topically, intratumorally, intramuscularly, intraperitoneally, subcutaneously, subconjunctival, intravesicularlly, mucosally, intrapericardially, intraumbilically, intraocularally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences (1990), incorporated herein by reference). In a particular embodiment, intraperitoneal injection is contemplated.

In any case, the composition may comprise various antioxidants to retard oxidation of one or more components. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

The agent may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups also can be derived from inorganic bases such as for example, sodium, potassium, ammonium, calcium or ferric hydroxides; or such organic bases as isopropylamine, trimethylamine, histidine or procaine.

In embodiments where the composition is in a liquid form, a carrier can be a solvent or dispersion medium comprising but not limited to, water, ethanol, polyol (e.g., glycerol, propylene glycol, liquid polyethylene glycol, etc.), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. In many cases, it will be preferable to include isotonic agents, such as, for example, sugars, sodium chloride or combinations thereof.

The compounds of the invention may be administered directly to a tissue. Direct tissue administration may be achieved by direct injection. The compounds may be administered once, or alternatively they may be administered in a plurality of administrations. If administered multiple times, the compounds may be administered via different routes. For example, the first (or the first few) administrations may be made directly into the affected tissue while later administrations may be systemic.

The formulations of the invention are administered in pharmaceutically acceptable solutions, which may routinely contain pharmaceutically acceptable concentrations of salt, buffering agents, preservatives, compatible carriers, adjuvants, and optionally other therapeutic ingredients.

According to the methods of the invention, the compound may be administered in a pharmaceutical composition. In general, a pharmaceutical composition comprises the compound of the invention and a pharmaceutically-acceptable carrier. Pharmaceutically-acceptable carriers for the compounds of the invention are well-known to those of ordinary skill in the art. As used herein, a pharmaceutically-acceptable carrier means a non-toxic material that does not interfere with the effectiveness of the biological activity of the active ingredients.

Pharmaceutically acceptable carriers include diluents, fillers, salts, buffers, stabilizers, solubilizers and other materials which are well-known in the art. Exemplary pharmaceutically acceptable carriers for peptides in particular are described in U.S. Pat. No. 5,211,657. Such preparations may routinely contain salt, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

The compounds of the invention may be formulated into preparations in solid, semi-solid, liquid or gaseous forms such as tablets, capsules, powders, granules, ointments, solutions, depositories, inhalants and injections, and usual ways for oral, parenteral or surgical administration. The invention also embraces pharmaceutical compositions which are formulated for local administration, such as by implants.

Compositions suitable for oral administration may be presented as discrete units, such as capsules, tablets, lozenges, each containing a predetermined amount of the active agent. Other compositions include suspensions in aqueous liquids or non-aqueous liquids, such as a syrup, an elixir or an emulsion.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers for neutralizing internal acid conditions or may be administered without any carriers.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For administration by inhalation, the compounds for use according to the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch. Techniques for preparing aerosol delivery systems are well known to those of skill in the art. Generally, such systems should utilize components which will not significantly impair the biological properties of the active agent (see, for example, Sciarra and Cutie, “Aerosols,” in Remington's Pharmaceutical Sciences, 18th edition, 1990, pp 1694-1712; incorporated by reference). Those of skill in the art can readily determine the various parameters and conditions for producing aerosols without resort to undue experimentation.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. Lower doses will result from other forms of administration, such as intravenous administration. In the event that a response in a subject is insufficient at the initial doses applied, higher doses (or effectively higher doses by a different, more localized delivery route) may be employed to the extent that patient tolerance permits. Multiple doses per day are contemplated to achieve appropriate systemic levels of compounds.

In yet other embodiments, the preferred vehicle is a biocompatible microparticle or implant that is suitable for implantation into the mammalian recipient. Exemplary biodegradable implants that are useful in accordance with this method are described in PCT International Application No. PCT/US/03307 (Publication No. WO 95/24929, entitled “Polymeric Gene Delivery System”, claiming priority to U.S. patent application serial no. 213,668, filed Mar. 15, 1994). PCT/US/0307 describes a biocompatible, preferably biodegradable polymeric matrix for containing a biological macromolecule. The polymeric matrix may be used to achieve sustained release of the agent in a subject. In accordance with one aspect of the instant invention, the agent described herein may be encapsulated or dispersed within the biocompatible, preferably biodegradable polymeric matrix disclosed in PCT/US/03307. The polymeric matrix preferably is in the form of a microparticle such as a microsphere (wherein the agent is dispersed throughout a solid polymeric matrix) or a microcapsule (wherein the agent is stored in the core of a polymeric shell). Other forms of the polymeric matrix for containing the agent include films, coatings, gels, implants, and stents. The size and composition of the polymeric matrix device is selected to result in favorable release kinetics in the tissue into which the matrix device is implanted. The size of the polymeric matrix device further is selected according to the method of delivery which is to be used, typically injection into a tissue or administration of a suspension by aerosol into the nasal and/or pulmonary areas. The polymeric matrix composition can be selected to have both favorable degradation rates and also to be formed of a material which is bioadhesive, to further increase the effectiveness of transfer when the device is administered to a vascular, pulmonary, or other surface. The matrix composition also can be selected not to degrade, but rather, to release by diffusion over an extended period of time.

Both non-biodegradable and biodegradable polymeric matrices can be used to deliver the agents of the invention to the subject. Biodegradable matrices are preferred. Such polymers may be natural or synthetic polymers. Synthetic polymers are preferred. The polymer is selected based on the period of time over which release is desired, generally in the order of a few hours to a year or longer. Typically, release over a period ranging from between a few hours and three to twelve months is most desirable. The polymer optionally is in the form of a hydrogel that can absorb up to about 90% of its weight in water and further, optionally is cross-linked with multivalent ions or other polymers.

In general, the agents of the invention may be delivered using the biodegradable implant by way of diffusion, or more preferably, by degradation of the polymeric matrix. Exemplary synthetic polymers which can be used to form the biodegradable delivery system include: polyamides, polycarbonates, polyalkylenes, polyalkylene glycols, polyalkylene oxides, polyalkylene terepthalates, polyvinyl alcohols, polyvinyl ethers, polyvinyl esters, poly-vinyl halides, polyvinylpyrrolidone, polyglycolides, polysiloxanes, polyurethanes and co-polymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene, poly(ethylene glycol), poly(ethylene oxide), poly(ethylene terephthalate), poly(vinyl alcohols), polyvinyl acetate, poly vinyl chloride, polystyrene and polyvinylpyrrolidone.

Examples of non-biodegradable polymers include ethylene vinyl acetate, poly(meth)acrylic acid, polyamides, copolymers and mixtures thereof.

Examples of biodegradable polymers include synthetic polymers such as polymers of lactic acid and glycolic acid, polyanhydrides, poly(ortho)esters, polyurethanes, poly(butic acid), poly(valeric acid), and poly(lactide-cocaprolactone), and natural polymers such as alginate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water in vivo, by surface or bulk erosion.

Bioadhesive polymers of particular interest include biodegradable hydrogels described by H. S. Sawhney, C. P. Pathak and J. A. Hubell in Macromolecules, 1993, 26, 581-587, the teachings of which are incorporated herein, polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

Other delivery systems can include time-release, delayed release or sustained release delivery systems. Such systems can avoid repeated administrations of the compound, increasing convenience to the subject and the physician. Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids or neutral fats such as mono- di- and tri-glycerides; hydrogel release systems; silastic systems; peptide based systems; wax coatings; compressed tablets using conventional binders and excipients; partially fused implants; and the like. Specific examples include, but are not limited to: (a) erosional systems in which the platelet reducing agent is contained in a form within a matrix such as those described in U.S. Pat. Nos. 4,452,775, 4,675,189, and 5,736,152 and (b) diffusional systems in which an active component permeates at a controlled rate from a polymer such as described in U.S. Pat. Nos. 3,854,480, 5,133,974 and 5,407,686. In addition, pump-based hardware delivery systems can be used, some of which are adapted for implantation.

Therapeutic formulations of the compounds of the invention or other therapeutic may be prepared for storage by mixing a compounds of the invention having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

The compounds of the invention may be administered directly to a cell or a subject, such as a human subject alone or with a suitable carrier. Alternatively, a peptide may be delivered to a cell in vitro or in vivo by delivering a nucleic acid that expresses the peptide to a cell. Various techniques may be employed for introducing nucleic acid molecules of the invention into cells, depending on whether the nucleic acid molecules are introduced in vitro or in vivo in a host. Such techniques include transfection of nucleic acid molecule-calcium phosphate precipitates, transfection of nucleic acid molecules associated with DEAE, transfection or infection with the foregoing viruses including the nucleic acid molecule of interest, liposome-mediated transfection, and the like.

The invention also relates to assays for identifying therapeutics and therapeutic courses of treatment. The presence of plant viral DNA in a tumor cell may be assessed, for instance, in order to determine an appropriate therapeutic regimen against the tumor. For example one method involves performing a physical analytical step on a biological sample of a subject, identifying the presence of plant virus in the biological sample based on the physical analytical step, and determining a course of treatment for the subject based on the presence of the plant virus. Another method involves identifying an anti-cancer agent, by performing a physical analytical step on a plant to determine a plant defense mechanism for preventing infection with a plant virus, identifying an association of the plant virus with a mammalian cancer, and selecting the plant defense mechanism as an anti-cancer agent for the mammalian cancer.

The expression of plant viral genes in the tumor cell is determined using methods known to the skilled artisan. The detection methods generally involve contacting a plant viral binding molecule with a sample in or from a subject or in an in vitro cell. Preferably, the sample is first harvested from the subject, although in vivo detection methods are also envisioned. The sample may include any body tissue or fluid that is suspected of harboring the cancer cells. For example, the cancer cells are commonly found in or around a tumor mass for solid tumors. The binding molecules are referred to herein as isolated molecules that selectively bind to plant viral DNA, such as DNA, RNA or antibodies.

In aspects of the invention pertaining to cancers, the subject is a human either suspected of having the cancer, or having been diagnosed with cancer. Methods for identifying subjects suspected of having cancer may include physical examination, subject's family medical history, subject's medical history, biopsy, or a number of imaging technologies such as ultrasonography, computed tomography, magnetic resonance imaging, magnetic resonance spectroscopy, or positron emission tomography. Diagnostic methods for cancer and the clinical delineation of cancer diagnoses are well known to those of skill in the medical arts.

As used herein, a tissue sample is tissue obtained from a tissue biopsy, a surgically resected tumor, or any other tumor cell mass removed from the body using methods well known to those of ordinary skill in the related medical arts. The phrase “suspected of being cancerous” as used herein means a cancer tissue sample believed by one of ordinary skill in the medical arts to contain cancerous cells. Methods for obtaining the sample from a biopsy include gross apportioning of mass, microdissection, laser-based microdissection, or other art-known cell-separation methods.

Because of the variability of the cell types in diseased-tissue biopsy material, and the variability in sensitivity of the predictive methods used, the sample size required for analysis may range from 1, 10, 50, 100, 200, 300, 500, 1000, 5000, 10,000, to 50,000 or more cells. The appropriate sample size may be determined based on the cellular composition and condition of the biopsy and the standard preparative steps for this determination and subsequent isolation of the nucleic acid for use in the invention are well known to one of ordinary skill in the art.

The methods may involve the steps of isolating nucleic acids from the sample and/or an amplification step. Typically, a nucleic acid comprising a sequence of interest can be obtained from a biological sample, more particularly from a sample comprising DNA (e.g. gDNA or cDNA) or RNA (e.g. mRNA). Release, concentration and isolation of the nucleic acids from the sample can be done by any method known in the art. Various commercial kits are available such as the High pure PCR Template Preparation Kit (Roche Diagnostics, Basel, Switzerland) or the DNA purification kits (PureGene, Gentra, Minneapolis, US). Other, well-known procedures for the isolation of DNA or RNA from a biological sample are also available (Sambrook et al., Cold Spring Harbor Laboratory Press 1989, Cold Spring Harbor, N.Y., USA; Ausubel et al., Current Protocols in Molecular Biology 2003, John Wiley & Sons).

When the quantity of the nucleic acid is low or insufficient for the assessment, the nucleic acid of interest may be amplified. Such amplification procedures can be accomplished by those methods known in the art, including, for example, the polymerase chain reaction (PCR), ligase chain reaction (LCR), nucleic acid sequence-based amplification (NASBA), strand displacement amplification, rolling circle amplification, T7-polymerase amplification, and reverse transcription polymerase reaction (RT-PCR).

Polymerase chain reaction (PCR) technology is practiced routinely by those having ordinary skill in the art and its uses in diagnostics are well known and accepted. Methods for practicing PCR technology are disclosed in “PCR Protocols: A Guide to Methods and Applications”, Innis, M. A., et al. Eds. Academic Press, Inc. San Diego, Calif. (1990) which is incorporated herein by reference. Applications of PCR technology are disclosed in “Polymerase Chain Reaction” Erlich, H. A., et al., Eds. Cold Spring Harbor Press, Cold Spring Harbor, N.Y. (1989) which is incorporated herein by reference. U.S. Pat. No. 4,683,202, U.S. Pat. No. 4,683,195, U.S. Pat. No. 4,965,188 and U.S. Pat. No. 5,075,216, which are each incorporated herein by reference describe methods of performing PCR. PCR technology allows for the rapid generation of multiple copies of DNA sequences by providing 5′ and 3′ primers that hybridize to sequences present in an RNA or DNA molecule, and further providing free nucleotides and an enzyme which fills in the complementary bases to the nucleotide sequence between the primers with the free nucleotides to produce complementary strand of DNA.

PCR primers can be designed routinely by those having ordinary skill in the art using sequence information. The mRNA or cDNA is combined with the primers, free nucleotides and enzyme following standard PCR protocols. The mixture undergoes a series of temperature changes. If the test gene transcript or cDNA generated therefrom is present, that is, if both primers hybridize to sequences on the same molecule, the molecule comprising the primers and the intervening complementary sequences will be exponentially amplified. The amplified DNA can be easily detected by a variety of well-known means. If no gene transcript or cDNA generated therefrom is present, no PCR product will be exponentially amplified.

PCR product may be detected by several well-known means. One method for detecting the presence of amplified DNA is to separate the PCR reaction material by gel electrophoresis and stain the gel with ethidium bromide in order to visual the amplified DNA if present. A size standard of the expected size of the amplified DNA is preferably run on the gel as a control.

In some instances, such as when unusually small amounts of RNA are recovered and only small amounts of cDNA are generated therefrom, it is desirable to perform a PCR reaction on the first PCR reaction product. The second PCR can be performed to make multiple copies of DNA sequences of the first amplified DNA. A nested set of primers are used in the second PCR reaction. The nested set of primers hybridize to sequences downstream of the 5′ primer and upstream of the 3′ primer used in the first reaction.

Branched chain oligonucleotide hybridization may be performed as described in U.S. Pat. No. 5,597,909, U.S. Pat. No. 5,437,977 and U.S. Pat. No. 5,430,138, which are each incorporated herein by reference. Northern blot analysis methods are well known by those having ordinary skill in the art and are described in Sambrook, J. et al., (1989) Molecular Cloning: A Laboratory Manual, Cold Spring Harbor Laboratory Press, Cold Spring Harbor, N.Y. Additionally, mRNA extraction, electrophoretic separation of the mRNA, blotting, probe preparation and hybridization are all well-known techniques that can be routinely performed using readily available starting material.

Hybridization methods for nucleic acids are well known to those of ordinary skill in the art (see, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York). The nucleic acid molecules hybridize under stringent conditions to nucleic acid markers expressed in cancer cells. The tissue may be obtained from a subject or may be grown in culture.

In the assays of the invention, the presence of the plant virus may be indicative of a predisposition to cancer. As such, the discovery of the presence of a plant virus may lead to the recommendation for a particular therapeutic regimen to avoid development of a disease such as cancer. Additionally it may lead to a further analysis of the status of inflammation in the subject. It is believed that a triggering event such as the induction of inflammation may lead to the activation of a dormant virus and development of cancer.

The invention also includes articles, which refers to any one or collection of components. In some embodiments the articles are kits. The articles include pharmaceutical or diagnostic grade compounds of the invention in one or more containers. The article may include instructions or labels promoting or describing the use of the compounds of the invention. One kit includes a set of primers for detecting plant viruses, a reagent for processing the primers to detect plant viruses, and instructions for analyzing a human or animal biological sample to detect the presence of plant viruses using the set of primers and reagent.

In one embodiment, a kit comprises antibodies against the starvation markers being measured in a method of the invention. The kit may further comprise assay diluents, standards, controls and/or detectable labels. The assay diluents, standards and/or controls may be optimized for a particular sample matrix.

As used herein, “promoted” includes all methods of doing business including methods of education, hospital and other clinical instruction, pharmaceutical industry activity including pharmaceutical sales, and any advertising or other promotional activity including written, oral and electronic communication of any form, associated with compositions of the invention in connection with treatment of infections, cancer, and autoimmune disease.

“Instructions” can define a component of promotion, and typically involve written instructions on or associated with packaging of compositions of the invention. Instructions also can include any oral or electronic instructions provided in any manner.

Thus the agents described herein may, in some embodiments, be assembled into pharmaceutical or diagnostic or research kits to facilitate their use in therapeutic, diagnostic or research applications. A kit may include one or more containers housing the components of the invention and instructions for use. Specifically, such kits may include one or more agents described herein, along with instructions describing the intended therapeutic application and the proper administration of these agents. In certain embodiments agents in a kit may be in a pharmaceutical formulation and dosage suitable for a particular application and for a method of administration of the agents.

The kit may be designed to facilitate use of the methods described herein by physicians and can take many forms. Each of the compositions of the kit, where applicable, may be provided in liquid form (e.g., in solution), or in solid form, (e.g., a dry powder). In certain cases, some of the compositions may be constitutable or otherwise processable (e.g., to an active form), for example, by the addition of a suitable solvent or other species (for example, water or a cell culture medium), which may or may not be provided with the kit. As used herein, “instructions” can define a component of instruction and/or promotion, and typically involve written instructions on or associated with packaging of the invention. Instructions also can include any oral or electronic instructions provided in any manner such that a user will clearly recognize that the instructions are to be associated with the kit, for example, audiovisual (e.g., videotape, DVD, etc.), Internet, and/or web-based communications, etc. The written instructions may be in a form prescribed by a governmental agency regulating the manufacture, use or sale of pharmaceuticals or biological products, which instructions can also reflects approval by the agency of manufacture, use or sale for human administration.

The kit may contain any one or more of the components described herein in one or more containers. As an example, in one embodiment, the kit may include instructions for mixing one or more components of the kit and/or isolating and mixing a sample and applying to a subject. The kit may include a container housing agents described herein. The agents may be prepared sterilely, packaged in syringe and shipped refrigerated. Alternatively it may be housed in a vial or other container for storage. A second container may have other agents prepared sterilely. Alternatively the kit may include the active agents premixed and shipped in a syringe, vial, tube, or other container.

The following examples are provided to illustrate specific instances of the practice of the present invention and are not intended to limit the scope of the invention. As will be apparent to one of ordinary skill in the art, the present invention will find application in a variety of compositions and methods.

### EXAMPLES

### Example 1

### Detection of Plant Viral DNA in Human Bladder Cancer Cells

Methods:

Genomic DNA was extracted from T-24 human bladder cells using the Qiagen DNeasy Blood and Tissue Kit (Cat#69504) according to the manufacturer's directions. 1 μg of DNA, 1 μL of 10 μM forward primer (table below), and 1 μL of 10 μM reverse primer (table below), were used with the USB Taq PCR Master Mix Plus Kit according to the manufacturer's directions. Using a BioRad iCycler thermo cycler, 30 cycles of 1 min at 940 C, 1 min 520 C, 1 min at 720 C. Finally one 10 min elongation at 720 C was performed. PCR products were run on a polyacrylamide gel and analyzed on a Licor Odyssey Infrared Imager.

The following primers corresponding to SEQ ID NOs:486-493 were used in the study:

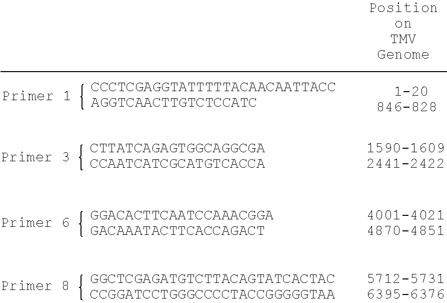

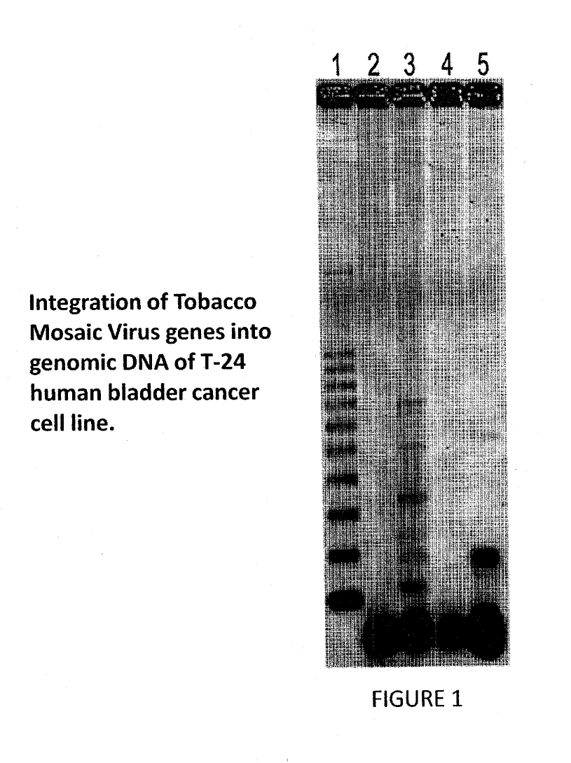

Results: PCR was performed on T24 bladder cancer cell DNA using TMV primers to detect the presence of plant viral DNA. The data is shown in FIG. 1. FIG. 1 is a blot of a genomic DNA PCR analysis. Gnomic DNA from T-24 human bladder cancer cell line was amplified using 4 primer sets specific for amplifying tobacco mosaic virus (TMV). Lane 1. 1/1000 BP size markers. Lane 2. Genomic DNA amplified with TMV primer 1. Lane 3. Genomic DNA amplified with TMV primer 3. Lane 4. Genomic DNA amplified with TMV primer 6. Lane 5. Genomic DNA amplified with TMV primer 8. Foreword and reverse primer sequences can be found in the table above. As shown in FIG. 1, TMV DNA is present in T24 bladder cancer cell DNA samples.

### Example 2

### Effect of Anti-Viral Compound on Human Bladder Cancer Cells

Methods:

T-24 Efavirenz Culture:

T-24 human bladder cells were grown in a 12 well plate in a total volume of 2 mL of 10% FBS complete RPMI. Cells were left untreated or treated with 2 μL of methanol (Sigma-Aldrich) or treated with 10 μM efavirenz (Toronto Research Chemicals Cat# E425000). Cells were grown in CO₂ incubator at 37° C. for 48 hours. After 48 hours, cells were harvested and counted using trypan blue on a hemocytometer.

MitoTracker Red:

Mitochondrial membrane potential was assessed using Mitotracker Red (CM-H₂XROS, Invitrogen). The cells were resuspended in warm (37° C. PBS containing a final concentration of 0.5 μM dye. The cells were incubated for 20 minutes, pelleted, and resuspended in PBS for analysis.

Results:

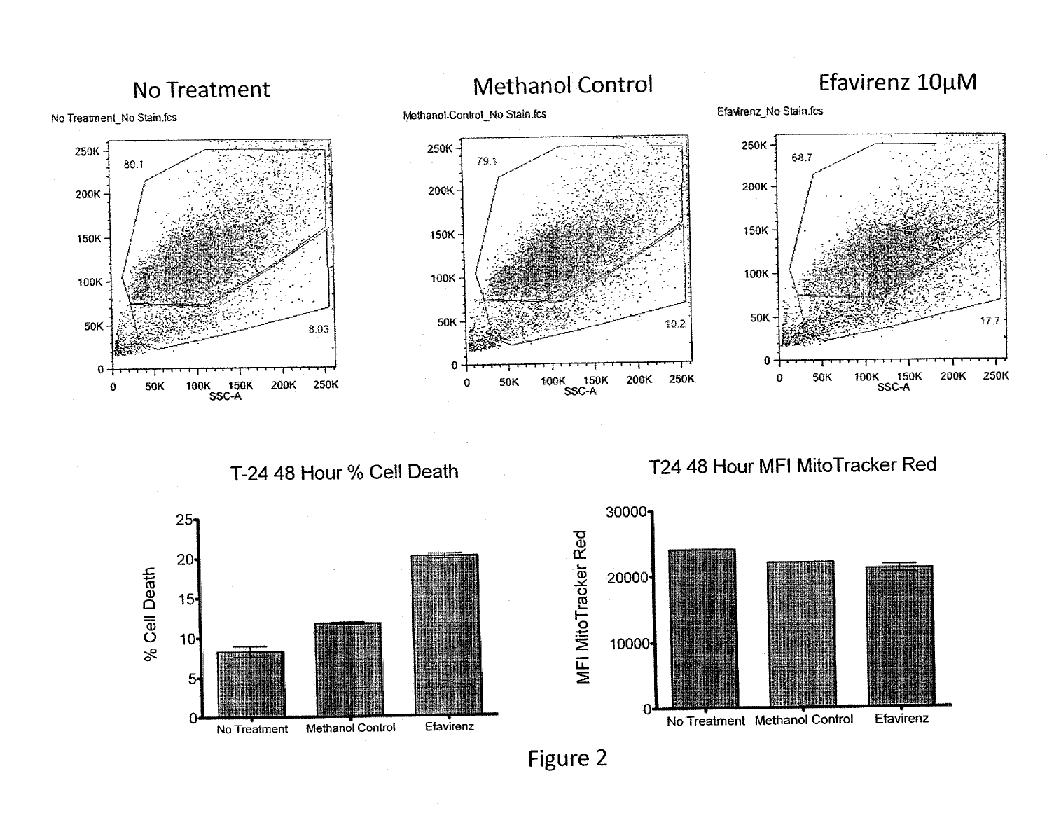

The human bladder cancer T24 cell line was used to determine the effects of and anti-viral treatment on human tumor cells infected with plant virus. The T24 cells were grown in culture and then treated or not with the anti-reverse transcriptase drug, efavirenz, for twenty four or forty eight hours. Cell death assays were performed in triplicate. Efavirenz was effective in killing a percentage of the cells, presumably the subset of the population that are producing viruses or reverse transcribing. It is expected that treatment of the bladder cancer cells with a TLR activator to activate new virus replication in combination with the anti-viral drug will be useful in increasing cell death further. FIG. 2a depicts flow cytometer results on T-24 Human bladder cancer cells treated with efavirenz or methanol control for 48 hours. FIG. 2b is a bar graph depiction of the data.

### Example 3

### TLR Activation Results in Transcription of the Integrated Viral Genes in Several of the Human Bladder Cancer Cells

Methods:

Total RNA was extracted from T-24 human bladder cells and C57B/6 mouse splenocytes using the Qiagen RNeasy Minit Kit (Cat#74104) according to the manufacturer's directions. cDNA was synthesized with the BioRad iScript cDNA Synthesis Kit (1708891) using a BioRad iCycler thermo cycler according to the manufacturer's directions. The following primer sets were used with iTaq SYBER Green Super Mix with ROX (BioRad 172-5850) on an Agilent Technologies Stratagene Mx3005P real time PCR machine.

Primer sets were used according to Zhou, X. et al. Complete nucleotide sequence and genome organization of tobacco mosaic virus isolated from Vicia faba. Sci. China C Life Sci. 2000 Vol. 43 No. 2.

The primers corresponding to SEQ ID NOs:494-507, 233 and 344 are presented below:

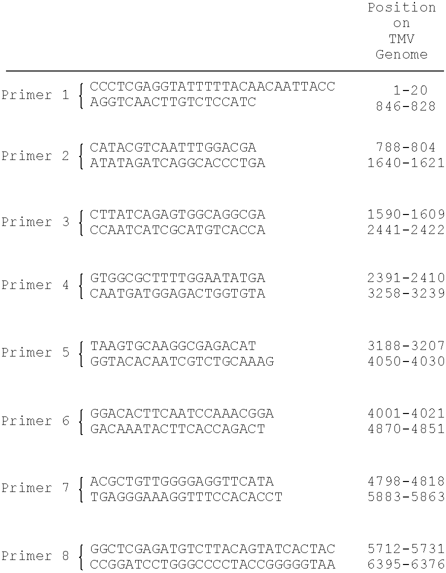

Results:

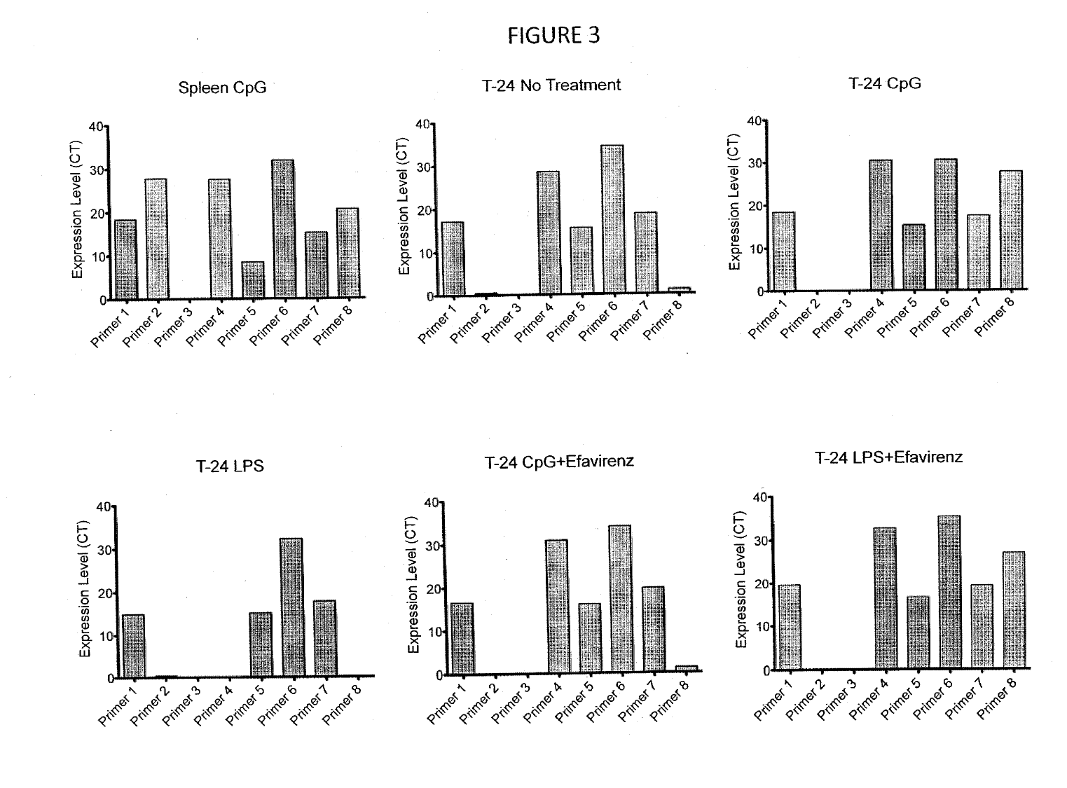

The impact of TLR activation on viral gene transcription in a human bladder cancer cell was examined. The results are shown in FIG. 3. A series of bar graphs depicting the results of the PCR assays using primers 1-8 are shown. The following conditions were used: 3a is CpG treated spleen cells, 3b is untreated T24 cells, 3c is CpG treated T24 cells; 3d is LPS treated T24 cells, 3e is CpG+efavirenz treated T24, and 3f is LPS+efavirenz treated T24. The results demonstrate that TLR activation, particularly CpG causes increased transcription of at least one of the integrated viral genes in human bladder cancer cells. In particular, primer 8 showed increased expression in T24 cells.

### Example 4

### Sequence Alignment

Methods:

Using the software package ClustalX 2.1, the protein sequences from tobacco mosaic virus (TMV), pepper mild mottled virus (PMMV), rice grassy stunt virus (RGSV), cauliflower mosaic virus (CMV), and banana bunchy top virus (BBTV) were aligned with protein sequences of either known anti-apoptotic proteins from other viruses or human proteins associated with cell death pathways. Homologies are indicated by the bar graphs below the sequence information and indicate significant relationships.

Results:

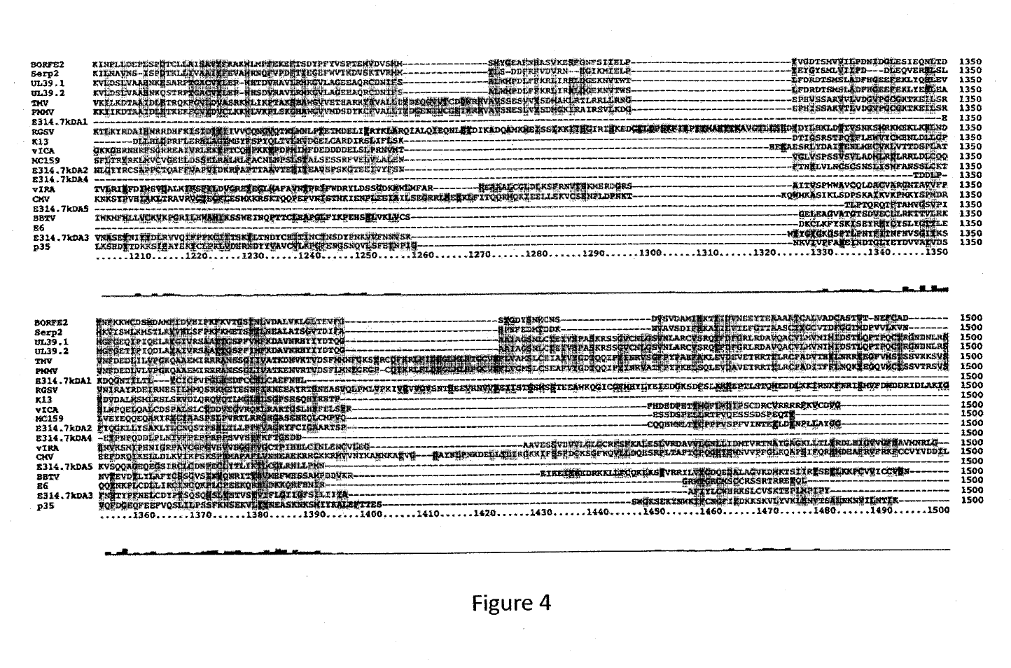

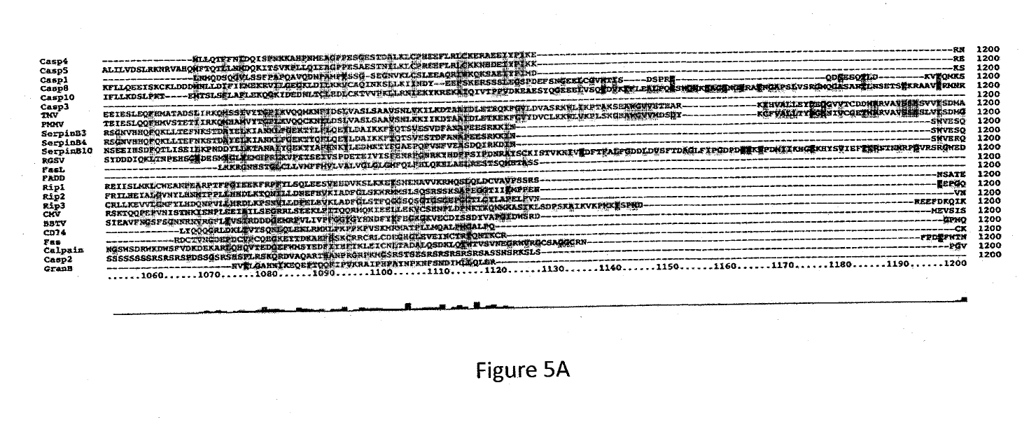

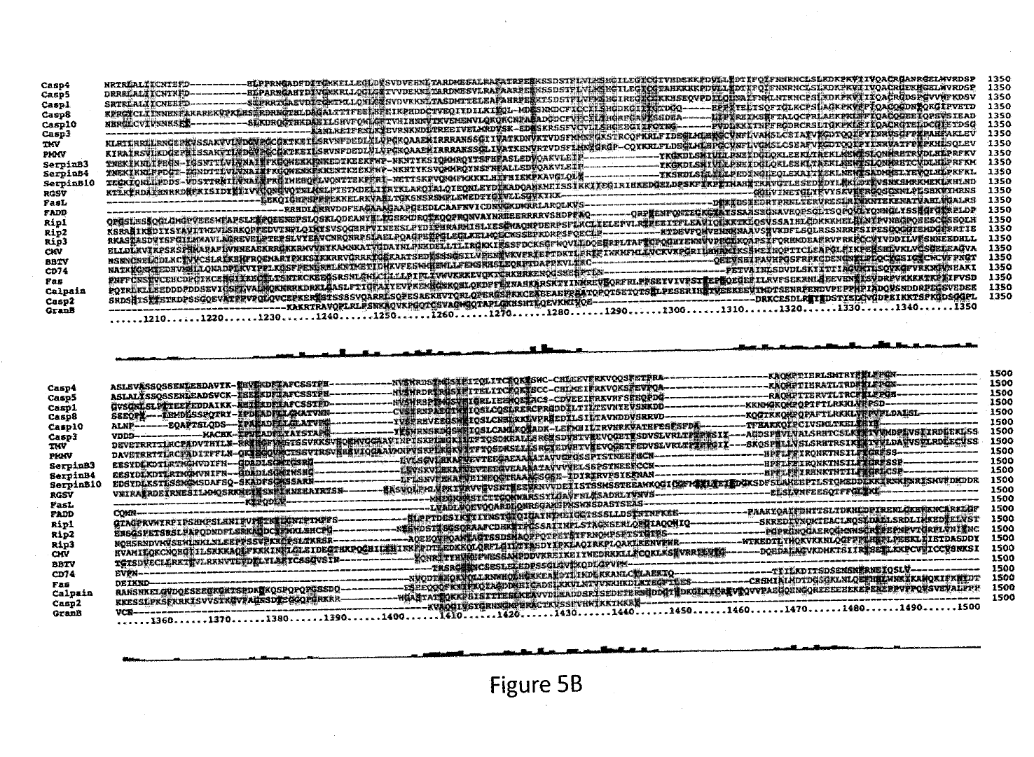

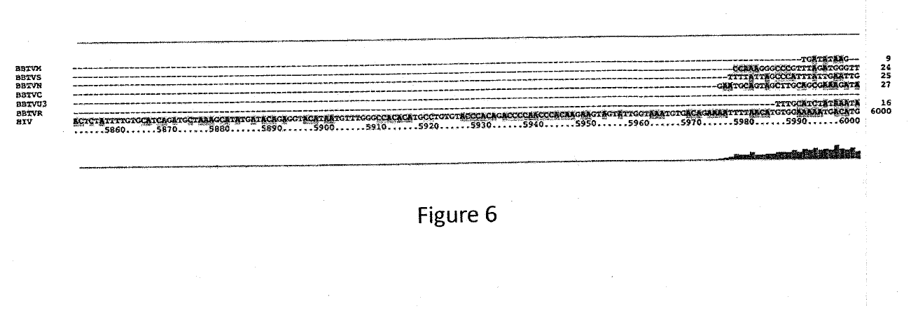

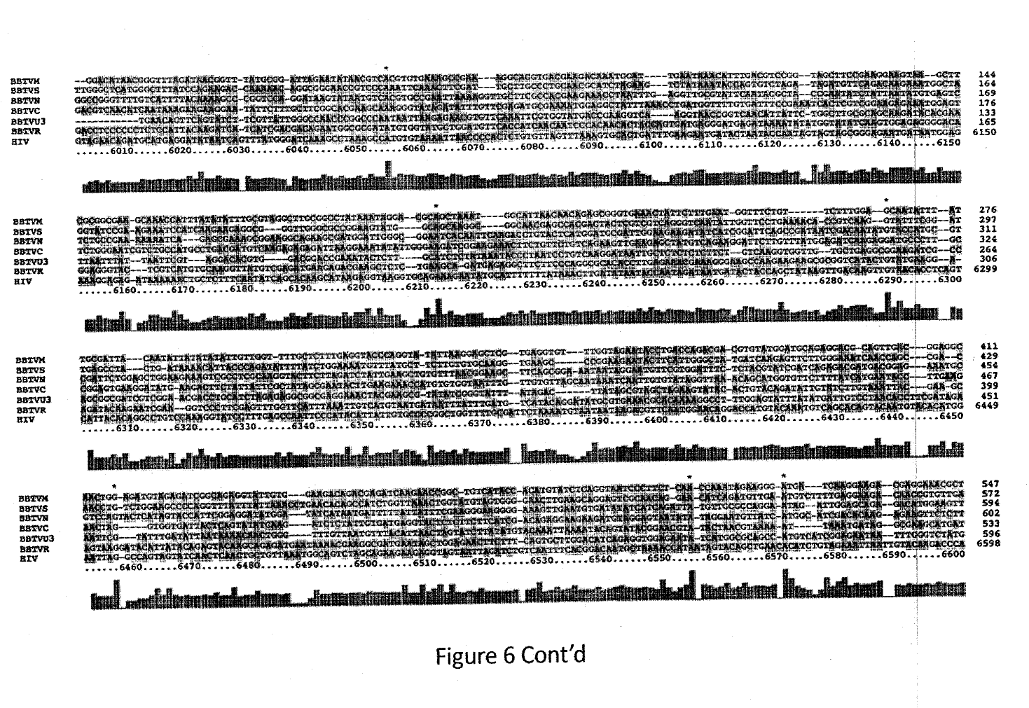

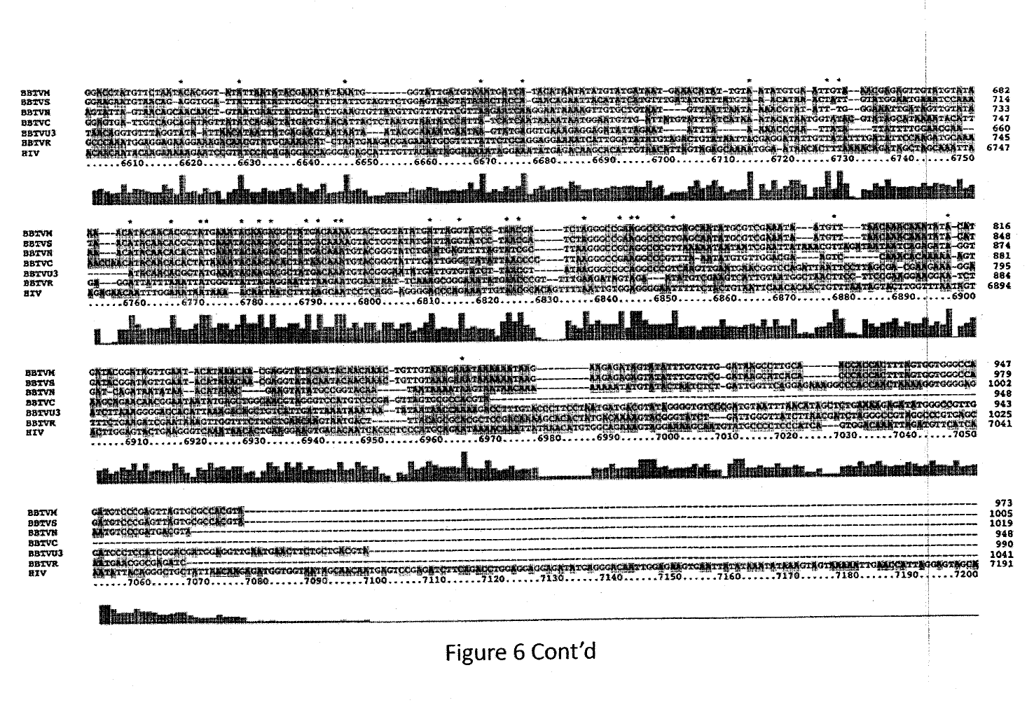

The ClustalX 2.1 alignment of plant virus protein sequences versus known viruses was generated and the results are shown in FIGS. 4-6. Specifically the ClustalX 2.1 alignment of plant virus protein sequences versus viral anti-apoptotic protein sequences is shown in FIG. 4. The ClustalX 2.1 Alignment of Plant Virus Protein Sequences vs. Human Proteins from Cell Death Pathways is shown in FIGS. 5A & 5B. The ClustalX 2.1 alignment of HIV versus Banana Bunchy Top Virus (BBTV) is shown in FIG. 6.

The sequence alignments show striking homology between a number of plant viruses and mammalian viruses, suggesting a possible common origin. The high sequence homology provides a guide for selecting the appropriate plant viral vaccine or anti-viral strategy for a particular disease. Interestingly, the significant homology between HIV and Banana bunchy top virus (BBTV), suggests the use of a new plant viral vaccine for the treatment of HIV infection. The BBTV may be used as a prophylactic or therapeutic vaccine for the treatment of HIV infection.

### Example 5

### Sequences and Accession Numbers for Plant Viral Peptides Tobacco Mosaic Virus Protein Sequence

| | | SEQ | |
|---|---|---|---|
| Protein | | ID | |
| Name | Accession # | NO. | Sequence |
| Coat | NP_597750.1 | 1 | SYSITTPSQFVFLSSAWADPIELINLCTNALGNQFQTQQARTVVQRQFSEVWKPSPQVTVRFP |
| Protein | | | DSDFKVYRYNAVLDPLVTALLGAFDTRNRIIEVENQANPTTAETLDATRRVDDATVAIRSAI |
| | | | NNLIVELIRGTGSY NRSSFESSSGLVWTSGPAT |
| Replicase | NP_597746.1 | 2 | AYTQTATTSALLDTVRGNNSLVNDLAKRRLYDTAVEEFNARDRRPKVNFSKVISEEQTLIAT |
| | | | RAYPEFQITFYNTQNAVHSLAGGLRSLELEYLMMQIPYGSLTYDIGGNFASHLFKGRAYVH |
| | | | CCMPNLDVRDIMRHEGQKDSIELYLSRLERGGKTVPNFQKEAFDRYAEIPEDAVCHNTFQT |
| | | | MRHQPMQQSGRVYAIALHSIYDIPADEFGAALLRKNVHTCYAAFHFSENLLLEDSYVNLDEI |
| | | | NACFSRDGDKLTFSFASESTLNYCHSYSNILKYVCKTYFPASNREVYMKEFLVTRVNTWFC |
| | | | KFSRIDTFLLYKGVAHKSVDSEQFYTAMEDAWHYKKTLAMCNSERILLEDSSSVNYWFPK |
| | | | MRDMVIVPLFDISLETSKRTRKEVLVSKDFVFTVLNHIRTYQAKALTYANVLSFVESIRSRVII |
| | | | NGVTARSEWDVDKSLLQSLSMTFYLHTKLAVLKDDLLISKFSLGSKTVCQHVWDEISLAFG |
| | | | NAFPSVKERLLNRKLIRVAGDALEIRVPDLYVTFHDRLVTEYKASVDMPALDIRKKMEETE |
| | | | VMYNALSELSVLRESDKFDVDVFSQMCQSLEVDPMTAAKVIVAVMSNESGLTLTFERPTEA |
| | | | NVALALQDQEKASEGALVVTSREVEEPSMKGSMARGELQLAGLAGDHPESSYSKNEEIESL |
| | | | EQFHMATADSLIRKQMSSIVYTGPIKVQQMKNFIDSLVASLSAAVSNLVKILKDTAAIDLETR |
| | | | QKFGVLDVASRKWLIKPTAKSHAWGVVETHARKYHVALLEYDEQGVVTCDDWRRVAVSS |
| | | | ESVVYSDMAKLRTLRRLLRNGEPHVSSAKVVLVDGVPGCGKTKEILSRVNFDEDLILVPGK |
| | | | QAAEMIRRRANSSGIIVATKDNVKTVDSFMMNFGKSTRCQFKRLFIDEGLMLHTGCVNFLV |
| | | | AMSLCEIAYVYGDTQQIPYINRVSGFPYPAHFAKLEVDEVETRRTTLRCPADVTHYLNRRYE |
| | | | GFVMSTSSVKKSVSQEMVGGAAVINPISKPLHGKILTFTQSDKEALLSRGYSDVHTVHEVQG |
| | | | ETYSDVSLVRLTPTPVSIIAGDSPHVLVALSRHTCSLKYYTVVMDPLVSIIRDLEKLSSYLLD |
| | | | MYKVDAGTQXQLQIDSVFKGSNLFVAAPKTGDISDMQFYYDKCLPGNSTMMNNFDAVTM |
| | | | RLTDISLNVKDCILDMSKSVAAPKDQIKPLIPMVRTAAEMPRQTGLLENLVAMIKRNFNAPE |
| | | | LSGIIDIENTASLVVDKFFDSYLLKEKRKPNKNVSLFSRESLNRWLEKQEQVTIGQLADFDFV |
| | | | DLPAVDQYRHMIKAQPKQKLDTSIQTEYPALQTIVYHSKKINAIFGPLFSELTRQLLDSVDSS |
| | | | RFLFFTRKTPAQIEDFFGDLDSHVPMDVLELDISKYDKSQNEFHCAVEYEIWRRLGFEDFLG |
| | | | EVWKQGHRKTTLKDYTAGIKTCIWYQRKSGDVTTFIGNTVIIAACLASMLPMEKIIKGAFCG |
| | | | DDSLLYFPKGCEFPDVQHSANLMWNFEAKLFKKQYGYFCGRYVIHHDRGCIVYYDPLKLIS |
| | | | KLGAKHIKDWEHLEEFRRSLCDVAVSLNNCAYYTQLDDAVWEVHKTAPPGSFVYKSLVKY |
| | | | LSDKVLFRSLFIDGSSC |
| RNA | NP_597747.1 | 3 | QFYYDKCLPGNSTMMNNFDAVTMRLTDISLNVKDCILDMSKSVAAPKDQIKPLIPMVRTAA |
| Polymerase | | | EMPRQTGLLENLVAMIKRNFNAPELSGIIDIENTASLVVDKFFDSYLLKEKRKPNKNVSLFSR |
| | | | ESLNRWLEKQEQVTIGQLADFDFVDLPAVDQYRHMIKAQPKQKLDTSIQTEYPALQTIVYH |
| | | | SKKINAIFGPLFSELTRQLLDSVDSSRFLFFTRKTPAQIEDFFGDLDSHVPMDVLELDISKYDK |
| | | | SQNEFHCAVEYEIWRRLGFEDFLGEVWKQGHRKTTLKDYTAGIKTCIWYQRKSGDVTTFIG |
| | | | NTVIIAACLASMLPMEKIIKGAFCGDDSLLYFPKGCEFPDVQHSANLMWNFEAKLFKKQYG |
| | | | YFCGRYVIHHDRGCIVYYDPLKLISKLGAKHIKDWEHLEEFRRSLCDVAVSLNNCAYYTQL |
| | | | DDAVWEVHKTAPPGSFVYKSLVKYLSDKVLFRSLFIDGSSC |
| Movement | NP_597748.1 | 4 | ALVVKGKVNINEFIDLTKMEKILPSMFTPVKSVMCSKVDKIMVHENESLSEVNLLKGVKLID |
| Protein | | | SGYVCLAGLVVTGEWNLPDNCRGGVSVCLVDKRMERADEATLGSYYTAAAKKRFQFKVV |
| | | | PNYAITTQDAMKNVWQVLVNIRNVKMSAGFCPLSLEFVSVCIVYRNNIKLGLREKITNVRD |
| | | | GGPMELTEEVVDEFMEDVPMSIRLAKFRSRTGKKSDVRKGKNSSNDRSVPNKNYRNVKDF |
| | | | GGMSFKKNNLIDDDSEATVAESDSF |
| Charged | NP_597749.1 | 5 | MIRRLLSPNRIRFKYVLQYHYSISVRVLVISVGRPNRVN |
| Protein | | | |

TMV Examplary Peptides:

| Amino | | SEQ ID |
|---|---|---|
| Acid number | Sequence | NO. |
| 1-11 | acetyl-SYSITTPSQFV(GK)^{a} | 6 |
| 19-32 | (KG)DPIELINLCTNALG^{a} | 7 |
| 18-25 | ADPIELIN | 8 |
| 22-29 | ELINLCTN | 9 |
| 27-33 | CTNALGN | 10 |
| 28-42 | TNALGNQFQTQQART | 11 |
| 34-39 | QFQTQQ | 12 |
| 39-51 | QARTVVQRQFSEV | 13 |
| 53-74 | KPSPQVTVRFPDSDFKVYRYNA | 14 |
| 61-74 | RFPDSDFKVYRYNA | 15 |
| 72-77 | YNAVLD | 16 |
| 76-88 | (KG)LDPLVTALLGAFD^{a} | 17 |
| 90-117 | RNRIIEVENQANPTTAETLDATRRVDDA | 18 |
| 95-117 | EVENQANPTTAETLDATRRVDDA | 19 |
| 115-134 | DDATVAIRSAINNLIVELIR | 20 |
| 129-134 | IVELIR | 21 |
| 134-146 | RGTGSYNRSSFES | 22 |
| 142-147 | SSFESS | 23 |
| 149-158 | GLVWTSGPAT | 24 |

A: alanine;
R: arginine;
D: aspartic acid;
N: asparagine;
C: cysteine;
E: glutamic acid;
Q: glutamine;
G: glycine;
I: isoleucine;
L: leucine;
K: lysine;
F: phenylalanine;
P: proline;
S: serine;
T: threonine;
W: tryptophan;
Y: tyrosine;
V: valine.
sequence (KG) raises the hydrophilicity of particularly hydrophobic peptides.

Relicase 1a

| HLADRB1*0101 | Predicted −logIC50 | Predicted IC50 | Confidence of | |
|---|---|---|---|---|
| Amino acid groups | (M) | Value (nM) | prediction (Max = 1) | SEQ ID NO |
| FDEDLILVP | 9.234 | 0.58 | 0.33 | 25 |
| YLHTKLAVL | 9.22 | 0.6 | 0.38 | 26 |
| FIDSLVASL | 9.154 | 0.7 | 0.38 | 27 |
| FYLHTKLAV | 9.116 | 0.77 | 0.29 | 28 |
| RVYAIALHS | 9.101 | 0.79 | 0.29 | 29 |
| HLADRB*0401 | Predicted −logIC50 | Predicted IC50 | Confidence of | |
| Amino acid groups | (M) | Value (nM) | prediction (Max = 1) | SEQ ID NO |
| VSSAKVVLV | 7.403 | 39.54 | 0.38 | 30 |
| VRGNNSLVN | 7.379 | 41.78 | 0.38 | 31 |
| DSLVASLSA | 7.327 | 47.1 | 0.33 | 32 |
| VSGFPYPAH | 7.263 | 54.58 | 0.33 | 33 |
| FSQMCQSLE | 7.242 | 57.28 | 0.29 | 34 |
| HLADRB*0701 | Predicted −logIC50 | Predicted IC50; | Confidence of | |
| Amino acid groups | (M) | Value (nM) | prediction (Max = 1) | SEQ ID NO |
| GAALLRKNV | 8.036 | 9.2 | 0.38 | 35 |
| IIVATKDNV | 7.858 | 13.87 | 0.38 | 36 |
| AKVIVAVMS | 7.738 | 18.28 | 0.38 | 37 |
| YVNLDEINA | 7.714 | 19.32 | 0.33 | 38 |
| EFLVTRVNT | 7.679 | 20.94 | 0.38 | 39 |

RNA Polymerase

| HLADRB1*0101 | Predicted −logIC50 | Predicted IC50 | Confidence of | |
|---|---|---|---|---|
| Amino acid groups | (M) | Value (nM) | prediction (Max = 1) | SEQ ID NO |
| YYDPLKLIS | 9.635 | 0.23 | 0.33 | 40 |
| FVDLPAVDQ | 9.034 | 0.92 | 0.33 | 41 |
| FFDSYLLKE | 9.034 | 0.92 | 0.38 | 42 |
| DIENTASLV | 8.993 | 1.02 | 0.29 | 43 |
| YYTQLDDAV | 8.989 | 1.03 | 0.29 | 44 |
| HLADRB*0401 | Predicted −logIC50 | Predicted IC50 | Confidence of | |
| Amino acid groups | (M) | Value (nM) | prediction (Max = 1) | SEQ ID NO |
| KVLFRSLFI | 7.378 | 41.88 | 0.33 | 45 |
| VYYDPLKLI | 7.366 | 43.05 | 0.38 | 46 |
| WYQRKSGDV | 7.285 | 51.88 | 0.33 | 47 |
| VDLPAVDQY | 7.28 | 52.48 | 0.29 | 48 |
| PRQTGLLEN | 7.24 | 57.54 | 0.29 | 49 |
| HLADRB*0701 | Predicted −logIC50 | Predicted IC50 | Confidence of | |
| Amino acid groups | (M) | Value (nM) | prediction (Max = 1) | SEQ ID NO |
| FIGNTVIIA | 8.002 | 9.95 | 0.38 | 50 |
| PMVRTAAEM | 7.616 | 24.21 | 0.29 | 51 |
| YPALQTIVY | 7.482 | 32.96 | 0.38 | 52 |
| RQLLDSVDS | 7.46 | 34.67 | 0.33 | 53 |

Charged Protein

| HLADRB1*0101 | Predicted −logIC50 | Predicted IC50 | Confidence of | |
|---|---|---|---|---|
| Amino acid groups | (M) | Value (nM) | prediction (Max = 1) | SEQ ID NO |
| MIRRLLSPN | 8.644 | 2.27 | 0.33 | 54 |
| SISVRVLVI | 8.336 | 4.61 | 0.33 | 55 |
| FKYVLQYHY | 8.226 | 5.94 | 0.33 | 56 |
| QYHYSISVR | 8.103 | 7.89 | 0.38 | 57 |
| MMIRRLLSP | 8.015 | 9.66 | 0.29 | 58 |
| HLADRB*0401Amino | Predicted −logIC50 | Predicted IC50 | Confidence of | |
| acid groups | (M) | Value (nM) | prediction (Max = 1) | SEQ ID NO |
| RVLVISVGR | 7.126 | 74.82 | 0.33 | 59 |
| YVLQYHYSI | 6.884 | 130.62 | 0.33 | 60 |
| RIRFKYVLQ | 6.626 | 236.59 | 0.29 | 61 |
| YHYSISVRV | 6.605 | 248.31 | 0.38 | 62 |
| YSISVRVLV | 6.604 | 248.89 | 0.38 | 63 |
| HLADRB*0701Amino | Predicted −logIC50 | Predicted IC50 | Confidence of | |
| acid groups | (M) | Value (nM) | prediction (Max = 1) | SEQ ID NO |
| KYVLQYHYS | 7.45 | 35.48 | 0.38 | 64 |
| IRRLLSPNR | 7.231 | 58.75 | 0.38 | 65 |
| YSISVRVLV | 7.007 | 98.4 | 0.38 | 66 |
| VRVLVISVG | 6.881 | 131.52 | 0.38 | 67 |
| LLSPNRIRF | 6.876 | 133.05 | 0.38 | 68 |

CaMV Proteins:

Cauliflower mosaic virus peptides obtained from UniPro (with UniPro accession number; http://www.uniprot.org/uniprot):

| Accession # | Protein names | Seq | |
|---|---|---|---|
| Entry | Gene names | ID | |
| name | Organism | NO | Sequence |
| P03551 | Virion-associated protein | 69 | MANLNQIQKE VSEILSDQKS MKADIKAILE LLGSQNPIKE SLETVAAKIV |
| VAP_CAMVS | ORF III | | NDLTKLINDC PCNKEILEAL GTQPKEQLIE QPKEKGKGLN LGKYSYPNYG |
| | Cauliflower mosaic virus | | VGNEELGSSG NPKALTWPFK APAGWPNQF |
| | (strain Strasbourg) (CaMV) | | |
| P03545 | Movement protein | 70 | MDLYPEENTQ SEQSQNSENN MQIFKSENSD GFSSDLMISN DQLKNISKTQ |
| MVP_CAMVS | ORF I | | LTLEKEKIFK MPNVLSQVMK KAFSRKNEIL YCVSTKELSV DIHDATGKVY |
| | Cauliflower mosaic virus | | LPLITKEEIN KRLSSLKPEV RKTMSMVHLG AVKILLKAQF RNGIDTPIKI |
| | (strain Strasbourg) (CaMV) | | ALIDDRINSR RDCLLGAAKG NLAYGKFMFT VYPKFGISLN TQRLNQTLSL |
| | | | IHDFENKNLM NKGDKVMTIT YVVGYALTNS HHSIDYQSNA TIELEDVFQE |
| | | | IGNVQQSEFC TIQNDECNWA IDIAQNKALL GAKTKTQIGN NLQIGNS ASS |
| | | | SNTENELARV SQNIDLLKNK LKEICGE |
| P03542 | Capsid protein | 71 | MAESILDRTI NRFWYNLGED CLSESQFDLM IRLMEESLDG DQIIDLTSLP |
| CAPSD_CAMVS | ORF IV | | SDNLQVEQVM TTTEDSISEE ESEFLLAIGE TSEEESDSGE EPEFEQVRMD |
| | Cauliflower mosaic virus | | RTGGTEIPKE EDGEGPSRYN ERKRKTPEDR YFPTQPKTIP GQKQTSMGML |
| | (strain Strasbourg) (CaMV) | | NIDCQTNRRT LIDDWAAEIG LIVKTNREDY LDPETILLLM EHKTSGIAKE |
| | | | LIRNTRWNRT TGDIIEQVID AMYTMFLGLN YSDNKVAEKI DEQEKAKIRM |
| | | | TKLQLCDICY LEEFTCDYEK NMYKTELADF PGYINQYLSK IPIIGEKALT |
| | | | RFRHEANGTS IYSLGFAAKI VKEELSKICD LSKKQKKLKK FNKKCCSIGE |
| | | | ASTEYGCKKT STKKYHKKRY KKKYKAYKPY KKKKKFRSGK |
| | | | YFKPKEKKGS KQKYCPKGKK DCRCWICNIE GHYANECPNR QSSEKAHILQ |
| | | | QAEKLGLQPI EEPYEGVQEV FILEYKEEEE ETSTEESDGS STSEDSDSD |
| P03554 | Enzymatic polyprotein | 72 | MDHLLLKTQT QTEQVMNVTN PNSIYIKGRL YFKGYKKIEL HCFVDTGASL |
| POL_CAMVS | ORF V | | CIASKFVIPE EHWVNAERPI MVKIADGSSI TISKVCKDID LIIAGEIFRI |
| | Cauliflower mosaic virus | | PTVYQQESGI DFIIGNNFCQ LYEPFIQFTD RVIFTKNKSY VHIAKLTRA |
| | (strain Strasbourg) (CaMV) | | VRVGTEGFLE SMKKRSKTQQ PEPVNISTNK IENPLEEIAI LSEGRRLSEE |
| | | | KLFITQQRMQ KIEELLEKVC SENPLDPNKT KQWMKASIKL SDPSKAIKVK |
| | | | PMKYSPMDRE EFDKQIKELL DLKVIKPSKS PHMAPAFLVN NEAEKRRGKK |
| | | | RMVVNYKAMN KATVGDAYNL PNKDELLTLI RGKKIFSSFD CKSGFWQVLL |
| | | | DQESRPLTAF TCPQGHYEWN VVPFGLKQAP SIFQRHMDEA FRVFRKFCCV |
| | | | YVDDILVFSN NEEDHLLHVA MILQKCNQHG IILSKKKAQL FKKKINFLGL |
| | | | EIDEGTHKPQ GHILEHINKF PDTLEDKKQL QRFLGILTYA SDYIPKLAQI |
| | | | RKPLQAKLKE NVPWRWTKED TLYMQKVKKN LQGFPPLHHP LPEEKLIIET |
| | | | DASDDYWGGM LKAIKINEGT NTELICRYAS GSFKAAEKNY HSNDKETLAV |
| | | | INTIKKFSIY LTPVHFLIRT DNTHFKSFVN LNYKGDSKLG RNIRWQAWLS |
| | | | HYSFDVEHIK GTDNHFADFL SREFNKVNS |
| P03559 | Transactivator/viroplasmin | 73 | MENIEKLLMQ EKILMLELDL VRAKISLARA NGSSQQGDLS LHRETPEKEE |
| IBMP_CAMVS | protein | | AVHSALATFT PSQVKAIPEQ TAPGKESTNP LMANILPKDM NSVQTEIRPV |
| | ORF VI | | KPSDFLRPHQ GIPIPPKPEP SSSVAPLRDE SGIQHPHTNY YVVYNGPHAG |
| | Cauliflower mosaic virus (strain | | IYDDWGCTKA ATNGVPGVAH KKFATITEAR AAADAYTTSQ QTDRLNFIPK |
| | Strasbourg) (CaMV) | | GEAQLKPKSF AKALTSPPKQ KAHWLMLGTK KPSSDPAPKE ISFAPEITMD |
| | | | DFLYLYDLVR KFDGEGDDTM FTTDNEKISL FNFRKNANPQ MVREAYAAGL |
| | | | IKTIYPSNNL QEIKYLPKKV KDAVKRFRTN CIKNTEKDIF LKIRSTIPVW |
| | | | TIQGLLHKPR QVIEIGVSKK VVPTESKAME SKIQIEDLTE LAVKTGEQFI |
| | | | QSLLRLNDKK KIFVNMVEHD TLVYSKNIKD TVSEDQRAIE TFQQRVISGN |
| | | | LLGFHCPAIC HFIVKIVEKE GGSYKCHHCD KGKAIVEDAS ADSGPKDGPP |
| | | | PTRSIVEKED VPTTSSKQVD |
| Q02954 | Transactivator/viroplasmin | 74 | MENIEKLLMQ EKILMLELDL VRAKISLARA NGSSQQGDLS LHRETPVKEE |
| IBMP_CAMVE | protein | | AVHSALATFT PTQVKAIPEQ TAPGKESTNP LMASILPKDM NPVQTGIRLA |
| | ORF VI | | VPGDFLRPHQ GIPIPQKSEL SSTVVPLRDE SGIQHPHINY YVVYNGPHAG |
| | Cauliflower mosaic virus (strain | | IYDDWGCTKA ATNGVPGVAH KKFATITEAR AAADAYTTSQ QTDRLNFIPK |
| | BBC) (CaMV) | | GEAQLKPKSF REALTSPPKQ KAHWLTLGTK RPSSDPAPKE ISFAPEITMD |
| | | | DFLYLYDLGR KFDGEGDDTM FTTDNEKISL FNFRKNADPQ MVREAYAAGL |
| | | | IKTIYPSNNL QEIKYLPKKV KDAVKRFRTN CIKNTEKDIF LKIRSTIPVW |
| | | | TIQGLLHKPR QVIEIGVSKK VVPTESKAME SKIQIEDLTE LAVKTGEQFI |
| | | | QSLLRLNDKK KIFVNMVEDD TLVYSKNIKD TVSEDQRAIE TFQQRVISGN |
| | | | LLGFHCPAIC HFIERTVEKE GGSYKVHHCD KGKAIVQDAS ADSGPKDGPP |
| | | | PTRSIVEKED VPTTSSKQVD |
| P03546 | Movement protein | 75 | MDLYPEENTQ SEQSQNSENN MQIFKSENSD GFSSDLMISN DQLKNISKTQ |
| MVP_CAMVC | ORF I | | LTLEKEKIFK MPNVLSQVMK KAFSRKNEIL YCVSTKELSV DIHDATGKVY |
| | Cauliflower mosaic virus (strain | | LPLITREEIN KRLSSLKPEV RKIMSMVHLG AVKILLKAQF RNGIDTPIKI |
| | CM-1841) (CaMV) | | ALIDDRINSR RDCLLGAAKG NLAYGKFMFT VYPKFGISLN TQRLNQTLSL |
| | | | IHDFENKNLM NKGDKVMTIT YIVGYALTNS HHSIDYQSNA TIELEDVFQE |
| | | | IGNVQQSDFC TIQNDECNWA IDIAQNKALL GAKTQSQIGN SLQIGNSASS |
| | | | SNTENELARV SQNIDLLKNK LKEICGE |
| P16666 | Transactivator/viroplasmin | 76 | MEDIEKLLLQ EKILMLELDL VRAKISLARA KGSMQQGGNS LHRETPVKEE |
| IBMP_CAMVB | protein | | AVHSALATFA PIQAKAIPEQ TAPGKESTNP LMVSILPKDM KSVQTEKKRL |
| | ORF VI | | VTPMDFLRPN QGIQIPQKSE PNSSVAPNRA ESGIQHPHSN YYVVYNGPHA |
| | Cauliflower mosaic virus (strain | | GIYDDWGSAK AATNGVPGVA HKKFATITEA RAAADVYTTA QQAERLNFIP |
| | Bari 1) (CaMV) | | KGEAQLKPKS FVKALTSPPK QKAQWLTLGV KKPSSDPAPK EVSFDQETTM |
| | | | DDFLYLYDLG RRFDGEGDDT VFTTDNESIS LFNFRKNANP EMIREAYNAG |
| | | | LIRTIYPSNN LQEIKYLPKK VKDAVKKFRT NCIKNTEKDI FLKIKSTIPV |
| | | | WQDQGLLHKP KHVIEIGVSK KIVPKESKAM ESKDHSEDLI ELATKTGEQF |
| | | | IQSLLRLNDK KKIFVNLVEH DTLVYSKNTK ETVSEDQRAI ETFQQRVITP |
| | | | NLLGFHCPSI CHFIKRTVEK EGGAYKCHHC DKGKAIVQDA SADSKVADKE |
| | | | GPPLTTNVEK EDVSTTSSKA SG |
| P03558 | Transactivator/viroplasmin | 77 | MENIEKLLMQ EKILMLELDL VRAKISLARA NGSSQQGDLP LHRETPVKEE |
| IBMP_CAMVC | protein | | AVHSALATFT PTQVKAIPEQ TAPGKESTNP LMASILPKDM NPVQTGIRLA |
| | ORF VI | | VPGDFLRPHQ GIPIPQKSEL SSIVAPLRAE SGIHHPHINY YVVYNGPHAG |
| | Cauliflower mosaic virus (strain | | IYDDWGCTKA ATNGVPGVAY KKFATITEAR AAADAYTTSQ QTDRLNFIPK |
| | CM-1841) (CaMV) | | GEAQLKPKSF AKALTSPPKQ KAHWLTLGTK RPSSDPAPKE ISFAPEITMD |
| | | | DFLYLYDLGR KFDGEGDDTM FTTDNEKISL FNFRKNADPQ MVREAYAAGL |
| | | | IKTIYPSNNL QEIKYLPKKV KDAVKRFRTN CIKNTEKDIF LKIRSTIPVW |
| | | | TIQGLLHKPR QVIEIGVSKK VVPTESKAME SKIQIEDLTE LAVKTGEQFI |
| | | | QSLLRLNDKK KIFVNMVEHD TLVYSKNIKD TVSEDQRAIE TFQQRVISGN |
| | | | LLGFHCPAIC HFIKRTVEKE GGTYKCHHCD KGKAIVQDAS ADSGPKDGPP |
| | | | PTRSIVEKED VPTTSSKQVD |
| P03557 | Transactivator/viroplasmin | 78 | MENIEKLLMQ EKILMLELDL VRAKISLARA NGSSQQGELS LHRETPEKEV |
| IBMP_CAMVD | protein | | AVHSALVTFT PTQVKAIPEQ TAPGKESTNP LMASILPKDM NPVQTGTRLA |
| | ORF VI | | VPSDFLRPHQ GIPIPQKSEL SSTVVPLRAE SGIQHPHINY YVVYNGPHAG |
| | Cauliflower mosaic virus (strain | | IYDDWGCTKA ATNGVPGVAH KKFATITEAR AAADAYTTRQ QTDRLNFIPK |
| | D/H) (CaMV) | | GEAQLKPKSF AEALTSPPKQ KAHWLTLGTK KPSSDPAPKE ISFAPEITMD |
| | | | DFLYLYDLVR KFDGEGDDTM FTTDNEKISL FNFRKNANPQ MVREAYAAGL |
| | | | IKTIYPSNNL QEIKYLPKKV KDAVKRFRTN CIKNTEKDIF LKIRSTIPVW |
| | | | TIQGLLHKPR QVIEIGVSKK VIPTESKAME SRIQIEDLTE LAVKTGEQFI |
| | | | QSLLRLNDKK KIFVNMVEHD TLVYSKNIKE TDSEDQRAIE TFQQRVISGN |
| | | | LLGFHCPAIC HFIMKTVEKE GGAYKCHHCD KGKAIVQDAS ADEGTTDKSG |
| | | | PPPTRSIVEK EDVPNTSSKQ VD |
| P13218 | Transactivator/viroplasmin | 79 | MENIEKLLMQ EKILMLELDL VRAKISLARA NGSSQQGDLS LHRETPVKEE |
| IBMP_CAMVJ | protein | | AVHSALATFT PTQVKAIPEQ TAPGKESTNP LMASILPKDM NSVQTENRLV |
| | ORF VI | | KPLDFLRPHQ GIPIPQKSEP NSSVTLHRVE SGIQHPHTNY YVVYNGPHAG |
| | Cauliflower mosaic virus (strain | | IYDDWGCTKA ATNGVPGVAH KKFATITEAR AAADAYTTNQ QTGRLNFIPK |
| | S-Japan) (CaMV) | | GEAQLKPKSF AKALISPPKQ KAHWLTLGTK KPSSDPAPKE ISFDPEITMD |
| | | | DFLYLYDLAR KFDGEDDGTI FTTDNEKISL FNFRKNANPQ MVREAYTAGL |
| | | | IKTIYPSNNL QEIKYLPKKV KDAVKRFRTN CIKNTEKDIF LKIRSTIPVW |
| | | | TIQGLLHKPR QVIEIGVSKK IVPTESKAME SKIQIEDLTE LAVKSGEQFI |
| | | | QSLLRLNDKK KIFVNMVEHD TLVYSKNIKD TVSEDQRAIE TFQQRVISGN |
| | | | LLGFHCPAIC HFIMKTVEKE GGAYKCHHCE KGKAIVKDAS TDRGTTDKDG |
| | | | PPPTRSIVEK EDVPTTSSKQ VD |
| P03543 | Capsid protein | 80 | MAESILDRTI NRFWYNLGED CLSESQFDLM IRLMEESLDG DQIIDLTSLP |
| CAPSD_CAMVC | ORF IV | | SDNLQVEQVM TTTDDSISEE SEFLLAIGEI SEDESDSGEE PEFEQVRMDR |
| | Cauliflower mosaic virus (strain | | TGGTEIPKEE DGEGPSRYNE RKRKTPEDRY FPTQPKTIPG QKQTSMGMLN |
| | CM-1841) (CaMV) | | IDCQINRRTL IDDWAAEIGL IVKTNREDYL DPETILLLME HKTSGIAKEL |
| | | | IRNTRWNRTT GDIIEQVINA MYTMFLGLNY SDNKVAEKID EQEKAKIRMT |
| | | | KLQLFDICYL EEFTCDYEKN MYKTEMADFP GYINQYLSKI PIIGEKALTR |
| | | | FRHEANGTSI YSLGFAAKIV KEELSKICDL SKKQKKLKKF NKKCCSIGEA |
| | | | SVEYGGKKTS KKKYHKRYKK RYKVYKPYKK KKKFRSGKYF |
| | | | KPKEKKGSKR KYCPKGKKDC RCWICNIEGH YANECPNRQS SEKAHILQQA |
| | | | ENLGLQPVEE PYEGVQEVFI LEYKEEEEET STEESDDESS TSEDSDSD |
| P03544 | Capsid protein | 81 | MAESILDRTI NRFWYKLGDD CLSESQFDLM IRLMEESLDG DQIIDLTSLP |
| CAPSD_CAMVD | ORF IV | | SDNLQVEQVM TTTEDSISEE ESEFLLAIGE TSEEESDSGE EPEFEQVRMD |
| | Cauliflower mosaic virus (strain | | RTGGTEIPKE EDGGEPSRYN ERKRKTTEDR YFPTQPKTIP GQKQTTMGML |
| | D/H) (CaMV) | | NIDCQANRRT LIDDWAAEIG LIVKTNREDY |
| | | | LDPETILLLM EHKTSGIAKE LIRNTRWNRT TGDIIEQVID AMYTMFLGLN |
| | | | YSDNKVAEKI EEQEKAKIRM TKLQLCDICY LEEFTCDYEK NMYKTELADF |
| | | | PGYINQYLSK IPIIGEKALT RFRHEANGTS IYSLGFAAKI VKEELSKICD |
| | | | LTKKQKKLKK FNKKCCSIGE ASVEYGCKKT SKKKYHKRYK |
| | | | KKYKAYKPYK KKKKFRSGKY FKPKEKKGSK QKYCPKGKKD |
| | | | CRCWICNIEG HYANECPNRQ SSEKAHILQQ AEKLGLQPIE EPYEGVQEVF |
| | | | ILEYKEEEEE TSTEEDDGSS TSEDSDSESD |
| P03556 | Enzymatic polyprotein | 82 | MDHLLQKTQI QNQTEQVMNI TNPNSIYIKG RLYFKGYKKI ELHCFVDTGA |
| POL_CAMVD | ORF V | | SLCIASKFVI PEEHWINAER PIMVKIADGS SITINKVCRD IDLIIAGEIF |
| | Cauliflower mosaic virus (strain | | HIPTVYQQES GIDFIIGNNF CQLYEPFIQF TDRVIFTKDR TYPVHIAKLT |
| | D/H) (CaMV) | | RAVRVGTEGF LESMKKRSKT QQPEPVNIST NKIAILSEGR RLSEEKLFIT |
| | | | QQRMQKIEEL LEKVCSENPL DPNKTKQWMK ASIKLSDPSK AIKVKPMKYS |
| | | | PMDREEFDKQ IKELLDLKVI KPSKSPHMAP AFLVNNEAEK RRGKKRMVVN |
| | | | YKAMNKATVG DAYNPPNKDE LLTLIRGKKI FSSFDCKSGF WQVLLDQESR |
| | | | PLTAFTCPQG HYEWNVVPFG LKQAPSIFQR HMDEAFRVFR KFCCVYVDDI |
| | | | LVFSNNEEDH LLHVAMILQK CNQHGIILSK KKAQLFKKKI NFLGLEIDEG |
| | | | THKPQGHILE HINKFPDTLE DKKQLQRFLG ILTYASDYIP KLAQIRKPLQ |
| | | | AKLKENVPWK WTKEDTLYMQ KVKKNLQGFP PLHHPLPEEK LIIETDASDD |
| | | | YWGGMLKAIK INEGTNTELI CRYASGSFKA AEKNYHSNDK ETLAVINTIK |
| | | | KFSIYLTPVH FLIRTDNTHF KSFVNLNYKG DSKLGRNIRW QAWLSHYSFD |
| | | | VEHIKGTDNH FADFLSREFN RVNS |
| Q02964 | Enzymatic polyprotein | 83 | MDHLLLKTQT QTEQVMNVTN PNSIYIKGRL YFKGYKKIEL HCFVDTGASL |
| POL_CAMVE | ORF V | | CIASKFVIPE EHWVNAERPI MVKIADGSSI TISKVCKDID LIIAREIFKI |
| | Cauliflower mosaic virus (strain | | PTVYQQESGI DFIIGNNFCQ LYEPFIQFTD RVIFTKNKSY PVHIAKLTRA |
| | BBC) (CaMV) | | VRVGTEGFLE SMKKRSKTQQ PEPVNISTNK IENPLKEIAI LSEGRRLSEE |
| | | | KLFITQQRMQ KIEELLEKVC SENPLDPNKT KQWMKASIKL SDPSKAIKVK |
| | | | PMKYSPMDRE EFDKQIKELL DLKVIKPSKS PHMAPAFLVN NEAEKRRGKK |
| | | | RMVVNYKAMN KATIGDAYNL PNKDELLTLI RGKKIFSSFD CKSGFWQVLL |
| | | | DQESRPLTAF TCPQGHYEWN VVPFGLKQAP SIFQRHMDEA FRVFRKFCCV |
| | | | YVDDILVFSN NEEDHLLHVA MILQKCNQHG IILSKKKAQL FKKKINFLGL |
| | | | EIDEGTHKPQ GHILEHINKF PDTLEDKKQL QRFLGILTYA SDYIPKLAQI |
| | | | RKPLQAKLKE NVPWKWTKED TLYMQKVKKN LQGFPPLHHP LPEEKLIIET |
| | | | DASDDYWGGM LKAIKINEGT NTELICRYAS GSFKAAERNY HSNDKETLAV |
| | | | INTIKKFSIY LTPVHFLIRT DNTHFKSFVN LNYKGDSKLG RNIRWQAWLS |
| | | | HYSFDVEHIK GTDNHFADFL SREFNKVNS |
| Q02951 | Capsid protein | 84 | MAESILDRTI NRFWYNLGED CLSESQFDLM IRLMEESLDG DQIIDLTSLP |
| CAPSD_CAMVE | ORF IV | | SDNLQVEQVM TTTDDSISEE SEFLLAIGET SEDESDSGEE PEFEQVRMDR |
| | Cauliflower mosaic virus (strain | | TGGTEIPKKE DGAEPSRYNE RKRKTTEDRY FPTQPKTIPG QKQTSMGILN |
| | BBC) (CaMV) | | IDCQTNRRTL IDDWAAEIGL IVKTNREDYL DPETILLLME HKTSGIAKEL |
| | | | IRNTRWNRTT GDIIEQVIDA MYTMFLGLNY SDNKVAEKID EQEKAKIRMT |
| | | | KLQLCDICYL EEFTCDYEKN MYKTELADFP GYINQYLSKI PIIGEKALTR |
| | | | FRHEANGTSI YSLGFAAKIV KEELSKICAL SKKQKKLKKF NKKCCSIGEA |
| | | | SVEYGCKKTS KKKYHNKRYK KKYKVYKPYK KKKKFRSGKY |
| | | | FKPKEKKGSK QKYCPKGKKD CRCWISNIEG HYANECPNRQ SSEKAHILQQ |
| | | | AEKLGLQPIE EPYEGVQEVF ILEYKEEEEE TSTEESDGSS TSEDSDSD |
| Q00956 | Capsid protein | 85 | MAESILDRTI NRFWYNLGED CLSESQFDLM IRLMEESLSG DQIIDLTSLP |
| CAPSD_CAMVN | ORF IV | | SDNLQVEQVM TTTEDSISEE SEFLLAIGET SEDESDSGEE PEFEQVRMDR |
| | Cauliflower mosaic virus (strain | | TGGTEIPKEE DGEPSRYNER KRKTTEDRYF PTQPKTIPRQ KQTSMGMLNI |
| | NY8153) (CaMV) | | DCQTNRRTLI DDWAAEIGLI VKTNREDYLN PETILLLMEH KTSGIAKELI |
| | | | RNTRWNRTTG DIIEQVIDRM YTMFLGLNYS DNKVAEKIDE QEKAKIRMTK |
| | | | LQLCDICYLE EFTCDYEKNM YKTELADFPG YINQYLSKIP IIGEKALTRF |
| | | | RHEANGTSIY SLGFERKICK EELSKIRDLS KNEKKLKKFN KKCCSIEEAS |
| | | | AEYGCKKTST KKYHKKRYKK KYKAYKPYKK KKKFRSGKYF |
| | | | KPKEKKGSKQ KYCPKGKKDC RCWICNIEGH YANECPNRQS SEKAHILQQA |
| | | | EKVGLQPIEA PYEGVQEVFI LEYKEEEEET STEESDDESS TSEDSDSD |
| P03548 | Aphid transmission protein ORF | 86 | MSITGQPHVY KKDTIIRLKP LSLNSNNRSY VFSSSKGNIQ NIINHLNNLN |
| VAT_CAMVS | II | | EIVGRSLLGI WKINSYFGLS KDPSESKSKN PSVFNTAKTI FKSGGVDYSS |
| | Cauliflower mosaic virus (strain | | QLKEIKSLLE AQNTRIKSLE KAIQSLENKI EPEPLTKEEV KELKESINSI |
| | Strasbourg) (CaMV) | | KEGLKNIIG |
| P03553 | Virion-associated protein ORF | 87 | MANLNQIQKE VSEILSDQKS MKADIKAILE LLGSQNPIKE SLETVAAKIV |
| VAP_CAMVD | III | | NDLTKLINDC PCNKEILEAL GNQPKEQLIG QPKEKGKGLN LGKYSYPNYG |
| | Cauliflower mosaic virus (strain | | VGNEELGSSG NPKALTWPFK APAGWPNQY |
| | D/H) (CaMV) | | |
| Q02967 | Virion-associated protein ORF | 88 | MANLNQIQKE VSEILSDQKS MKSDIKAILE LLGSQNPTKE SLEAVAAKIV |
| VAP_CAMVE | III | | NDLTKLINDC PCNKEILEAL GNQPKEQLIE QPKEKGKGLN LGKYSYPNYG |
| | Cauliflower mosaic virus (strain | | VGNEELGSSG NPKALTWPFK APAGWPNQF |
| | BBC) (CaMV) | | |
| P03550 | Aphid transmission protein | 89 | MSITGQPHVY KKDTIIRLKP LSLNSNNRSY VFSSSKGNIQ NIINHLNNLN |
| VAT_CAMVD | ORF II | | KIVGRSLLGI WKINSYFGLS KDPSESKSKN PSVFNTAKTI FKSGGVDYSS |
| | Cauliflower mosaic virus (strain | | QPKEIKSLLE AQNTRIKSLE KAIQSLDEKI EPEPLTKEEV KELKESINSI |
| | D/H) (CaMV) | | KEGLKNIIG |
| Q02966 | Aphid transmission protein | 90 | MRITGQPHVY KKDTIIRLKP LSLNSNNRSY VFSSSKGNIQ NIINHLNNLN |
| VAT_CAMVE | ORF II | | EIVGRSLLGI WKINSYFGLS KDPSESKSKN PSVFNTAKTI FKSGGVDYSS |
| | Cauliflower mosaic virus (strain | | QLKEIKSLLE AQNTRIKNLE KAIQSLDNKI EPEPLTKKEV KELKESINSI |
| | BBC) (CaMV) | | KEGLKNIIG |
| Q01087 | Aphid transmission protein | 91 | MSITGQPHVY KKDTIIRLKP LSLNSNNRSY VLVPQKGNIQ NIINHLNNLN |
| VAT_CAMVW | ORF II | | EIVGRSLLGI WKINSYFGLS KDPSESKSKN PSVFNTAKTI FKSGGVDYS |
| | Cauliflower mosaic virus (strain | | |
| | W260) (CaMV) | | |
| P03555 | Enzymatic polyprotein | 92 | MDHLLLKTQT QIEQVMNVTN PNSIYIKGRL YFKGYKKIEL HCFVDTGASL |
| POL_CAMVC | ORF V | | CIASKFVIPE EHWVNAERPI MVKIADGSSI TISKVCKDID LIIAGEIFKI |
| | Cauliflower mosaic virus (strain | | PTVYQQESGI DFIIGNNFCQ LYEPFIQFTD RVIFTKNKSY PVHITKLTRA |
| | CM-1841) (CaMV) | | VRVGIEGFLE SMKKRSKTQQ PEPVNISTNK IENPLEEIAI LSEGRRLSEE |
| | | | KLFITQQRMQ KIEELLEKVC SENPLDPNKT KQWMKASIKL SDPSKAIKVK |
| | | | PMKYSPMDRE EFDKQIKELL DLKVIKPSKS PHMAPAFLVN NEAEKRRGKK |
| | | | RMVVNYKAMN KATIGDAYNL PNKDELLTLI RGKKIFSSFD CKSGFWQVLL |
| | | | DQESRPLTAF TCPQGHYEWN VVPFGLKQAP SIFQRHMDEA FRVFRKFCCV |
| | | | YVDDILVFSN NEEDHLLHVA MILQKCNQHG IILSKKKAQL FKKKINFLGL |
| | | | EIDEGTHKPQ GHILEHINKF PDTLEDKKQL QRFLGILTYA SDYIPKLAQI |
| | | | RKPLQAKLKE NVPWKWTKED TLYMQKVKKN LQGFPPLHHP LPEEKLIIET |
| | | | DASDDYWGGM LKAIKINEGT NTELICRYAS GSFKAAERNY HSNDKETLAV |
| | | | INTIKKFSIY LTPVHFLIRT DNTHFKSFVN LNYKGDSKLG RNIRWQAWLS |
| | | | HYSFDVEHIK GTDNHFADFL SREFNKVNS |
| Q00962 | Enzymatic polyprotein | 93 | MMNHLLLKTQ TQTEQVMNVT NPNSIYIKGR LYFKGYKKIE LHCFVDTGAS |
| POL_CAMVN | ORF V | | LCIASKFVIP EEHWVNAERP IMVKIADGSS ITISKVCKDI DLIIVGVIFK |
| | Cauliflower mosaic virus (strain | | IPTVYQQESG IDFIIGNNFC QLYEPFIQFT DRVIFTKNKS YPVHIAKLTR |
| | NY8153) (CaMV) | | AVRVGTEGFL ESMKKRSKTQ QPEPVNISTN KIENPLEEIA ILSEGRRLSE |
| | | | EKLFITQQRM QKTEELLEKV CSENPLDPNK TKQWMKASIK LSDPSKAIKV |
| | | | KPMKYSPMDR EEFDKQIKEL LDLKVIKPSK SPHMAPAFLV NNEAENGRGN |
| | | | KRMVVNYKAM NKATVGDAYN LPNKDELLTL IRGKKIFSSF |
| | | | DCKSGFWQVL LDQESRPLTA FTCPQGHYEW NVVPFGLKQA PSIFQRHMDE |
| | | | AFRVFRKFCC VYVDDIVVFS NNEEDHLLHV AMILQKCNQH GIILSKKKAQ |
| | | | LFKKKINFLG LEIDEGTHKP QGHILEHINK FPDTLEDKKQ LQRFLGILTY |
| | | | ASDYIPNLAQ MRQPLQAKLK ENVPWKWTKE DTLYMQKVKK |
| | | | NLQGFPPLHH PLPEEKLIIE TDASDDYWGG MLKAIKINEG TNTELICRYR |
| | | | SGSFKAAERN YHSNDKETLA VINTIKKFSI YLTPVHFLIR TDNTHFKSFV |
| | | | NLNYKGDSKL GRNIRWQAWL SHYSFDVEHI KGTDNHFADF LSREFNKVNS |
| P03547 | Movement protein | 94 | MDLYPEENTQ SEQSQNSENN MQIFKSETSD GFSSDLKISN DQLKNISKTQ |
| MVP_CAMVD | ORF I | | LTLEKEKIFK MPNVLSQVMK KAFSRKNEIL YCVSTKELSV DIHDATGKVY |
| | Cauliflower mosaic virus (strain | | LPLITKEEIN KRLSSLKPEV RRTMSMVHLG AVKILLKAQF RNGIDTPIKI |
| | D/H) (CaMV) | | ALIDDRINSR RDCLLGAAKG NLAYGKFMFT VYPKFGISLN TQRLNQTLSL |
| | | | IHDFENKNLM NKGDKVMTIT YIVGYALTNS HHSIDYQSNA TIELEDVFQE |
| | | | IGNIQQSEFC TIQNDECNWA IDIAQNKALL GAKTKTQIGN SLQIGNIASS |
| | | | SSTENELARV SQNIDLLKNK LKEICGE |
| Q02968 | Movement protein | 95 | MDLYPEENTQ SEQSQNSENN MQIFKSENSD GFSSDLMISN DQLKNISKTQ |
| MVP_CAMVE | ORF I | | LTLEKEKIFK MPNVLSQVMK RAFSRKNEIL YCVSTKELSV DIHDATGKVY |
| | Cauliflower mosaic virus (strain | | LPLITREEIN KRLSSLKPEV RKTMSMVHLG AVKILLKAQF RNGIDTPIKI |
| | BBC) (CaMV) | | ALIDDRINSR RDCLLGAAKG NLAYGKFMFT VYPKFGISLN TQRLNQTLSL |
| | | | IHDFENKNLM NKGDKVMTIT YMVGYALTNS HHSIDYQSNA TIELEDVFQE |
| | | | IGNVBESDFC TIQNDECNWA IDIAQNKALL GAKTKSQIGN NLQIGNSASS |
| | | | SNTENELARV SQNIDLLKNK LKEICGE |
| Q00966 | Movement protein | 96 | MDLYPEEKTQ SKQSHNSENN MQIFKSENSD GFSSDLMISN DQLKNISKTQ |
| MVP_CAMVN | ORF I | | LTLEKEKIFK MPNVLSQVMK KAFSRKNEIL YCVSTKELSV DIHDATGKVY |
| | Cauliflower mosaic virus (strain | | LPLITKEEIN KRLSSLKPEV RKTMSMVHLG AVKILLKAQF RNGIDTPIKI |
| | NY8153) (CaMV) | | ALIDDRINSR RDCLLGAAKG NLAYGKFMFT VYPKFGISLN TQRLNQTLSL |
| | | | IHDFENKNLM NKGDKVMTIT YIVGYALTNS HHSIDYQSNA TIELEDVFQE |
| | | | IGNVQQCDFC TIQNDECNWA IDIAQNKALL GAKTQSQIGN SLQIGNSASS |
| | | | SNTENELARV SQNIDLLKNK LKEICGE |
| Q01089 | Movement protein | 97 | MDLYPEENTQ SEQSHNSENN MQIFKSENSD GFSSDLMISN DQLKNISKTQ |
| MVP_CAMVW | ORF I | | LTLEKEKIFK MPNVLSQVMK KAFSRKNEIL YCVSTKELSV DIHDATGKVY |
| | Cauliflower mosaic virus (strain | | LPLITKEEIN KRLSSLKPEV RRTMSMVHLG AVKILLKAQF RNGIDTPIKI |
| | W260) (CaMV) | | ALIDDRINSR KDCLLGAAKG NLAYGKFMFT VYPKFGISLN TQRLNQTLSL |
| | | | IHDFENKNLM NKGDKVMTIT YIVGYALTNS HHSIDYQSNA TIELEDVFQE |
| | | | IGNVQQSEFC TIQNDECNWA IDIAQNKALL GAKTKSQIGN SLQIGNSASS |
| | | | SNTENELARV SQNIDLLKNK LKEICGE |
| P03552 | Virion-associated protein ORF | 98 | MANLNQIQKE VSEILSDQKS MKSDIKAILE LLGSQNPTKE SLEAVAAKIV |
| VAP_CAMVC | III | | NDLTKLINDC PCNKEILEAL GNQPKEQLIE QPKEKGKGLN LGKYTYPNYG |
| | Cauliflower mosaic virus (strain | | VGNEELGSSG NPKALTWPFK APAGWPNQF |
| | CM-1841) (CaMV) | | |
| Q00967 | Virion-associated protein ORF | 99 | MANLNQIQKE VSEILSDQKS MKSDIKAILE MLGSQNPIKE SLEAVAAKIV |
| VAP_CAMVN | III | | NDLTKLINDC PCNKEILEAL GNQPKEQLIE QPKEKGKGLN LGKYSYPNYG |
| | Cauliflower mosaic virus (strain | | VGNEELGSSG NPKALTWPFK APAGWPNQF |
| | NY8153) (CaMV) | | |
| P03549 | Aphid transmission protein | 100 | MSITGQPHVY KKDTIIRLKP LSLNSNNRSY VFSSSKGNIQ NIINHLNNLN |
| VAT_CAMVC | ORF II | | EIVGRSLLGI WKINSYFGLS KDPSESKSKN PSVFNTAKNI FKSRGVDYSS |
| | Cauliflower mosaic virus (strain | | QLKEVKSLLE AQNTRIKNLE NAIQSLDNKI EPEPLTKEEV KELKESINSI |
| | CM-1841) (CaMV) | | KEGLKNIIG |
| Q00965 | Aphid transmission protein | 101 | MSITGQPHVY KKDTIIRLKP LSLNSNNRSY VFSSSKGNIQ NIINHLNNLN |
| VAT_CAMVN | ORF II | | EIVGRSLLGI WKINSYFGLS KDPSESKSKN PSVFNTAKTI FKSGGVDYSS |
| | Cauliflower mosaic virus (strain | | QLKEIKSLLE AQNTRIKSLE NAIQSLDNKI EPEPLTKEEV KELKESINSI |
| | NY8153) (CaMV) | | KEGLKNIIG |
| P19818 | Aphid transmission protein | 102 | MSITGQPHVY KKDTIIRLKP LSLNSNNRSY VFSSSKGNIQ NIINHLNNLN |
| VAT_CAMVP | ORF II | | EIVGRSLLGI WRINSYFGLS KDPSESKSKN PSVFNTAKTI FKSGGVDYSS |
| | Cauliflower mosaic virus (strain | | QLKEIKSLLE AQNTRIKNLE NAIQSLDNKI QPEPLTKEEV KELKESINSI |
| | PV147) (CaMV) | | KEALKNIIG |

CaMV Peptides:

Movement Protein

| | Predicted | Predicted | Confid. of | SEQ |
|---|---|---|---|---|
| Amino | −logIC50 | IC50 Value | prediction | ID |
| acid groups | (M) | (nM) | (Max = 1) | NO. |
| HLADRB1*0101 | | | | |
| NIDLLKNKL | 9.177 | 0.67 | 0.33 | 103 |
| LIDDRINSR | 8.687 | 2.06 | 0.33 | 104 |
| KILLKAQFR | 8.654 | 2.22 | 0.33 | 105 |
| TENELARVS | 8.441 | 3.62 | 0.29 | 106 |
| ITKEEINKR | 8.44 | 3.63 | 0.29 | 107 |
| HLADRB*0401 | | | | |
| NELARVSQN | 7.206 | 62.23 | 0.33 | 108 |
| VHLGAVKIL | 7.165 | 68.39 | 0.38 | 109 |
| FKMPNVLSQ | 7.121 | 75.68 | 0.29 | 110 |
| YPKFGISLN | 7.097 | 79.98 | 0.38 | 111 |
| VSQNIDLLK | 7.067 | 85.7 | 0.38 | 112 |
| HLADRB*0701 | | | | |
| YALTNSHHS | 7.494 | 32.06 | 0.38 | 113 |
| YCVSTKELS | 7.459 | 34.75 | 0.33 | 114 |
| TENELARVS | 7.367 | 42.95 | 0.38 | 115 |
| MVHLGAVKI | 7.31 | 48.98 | 0.33 | 116 |
| EVRKTMSMV | 7.222 | 59.98 | 0.38 | 117 |
| | Predicted | Predicted | Confidence of | SEQ |
| Amino | −logIC50 | IC50 Value | prediction | ID |
| acid groups | (M) | (nM) | (Max = 1) | NO. |

DNA Binding Protein

| | | | | |
|---|---|---|---|---|
| HLADRB1*0101 | | | | |
| PFKAPAGWP | 8.78 | 1.66 | 0.38 | 118 |
| KIVNDLTKL | 8.484 | 3.28 | 0.33 | 119 |
| DIKAILELL | 8.439 | 3.64 | 0.38 | 120 |
| SLETVAAKI | 8.38 | 4.17 | 0.33 | 121 |
| DLTKLINDC | 8.326 | 4.72 | 0.33 | 122 |
| HLADRB*0401 | | | | |
| EILEALGTQ | 6.927 | 118.3 | 0.29 | 123 |
| FKAPAGWPN | 6.881 | 131.52 | 0.29 | 124 |
| GSQNPIKES | 6.819 | 151.71 | 0.29 | 125 |
| EALGTQPKE | 6.809 | 155.24 | 0.29 | 126 |
| GNPKALTWP | 6.793 | 161.06 | 0.25 | 127 |
| HLADRB*0701 | | | | |
| PKALTWPFK | 7.53 | 29.51 | 0.38 | 128 |
| KGLNLGKYS | 7.439 | 36.39 | 0.38 | 129 |
| PFKAPAGWP | 7.385 | 41.21 | 0.33 | 130 |
| YPNYGVGNE | 7.257 | 55.34 | 0.38 | 131 |
| EALGTQPKE | 7.216 | 60.81 | 0.38 | 132 |

Reverse Transcriptase

| | | | | |
|---|---|---|---|---|
| HLADRB1*0101 | | | | |
| YVDDILVFS | 9.234 | 0.58 | 0.38 | 133 |
| FVDTGASLC | 9.152 | 0.7 | 0.38 | 134 |
| IIETDASDD | 8.959 | 1.1 | 0.29 | 135 |
| FIQFTDRVI | 8.942 | 1.14 | 0.33 | 136 |
| DYIPKLAQI | 8.915 | 1.22 | 0.38 | 137 |
| HLADRB*0401 | | | | |
| VVPFGLKQA | 7.269 | 53.83 | 0.38 | 138 |
| VTNPNSIYI | 7.195 | 63.83 | 0.25 | 139 |
| PLQAKLKEN | 7.183 | 65.61 | 0.29 | 140 |
| HYEWNVVPF | 7.145 | 71.61 | 0.29 | 141 |
| NYKGDSKLG | 7.131 | 73.96 | 0.33 | 142 |
| HLADRB*0701 | | | | |
| YKAMNKATV | 7.754 | 17.62 | 0.38 | 143 |
| EQVMNVTNP | 7.607 | 24.72 | 0.38 | 144 |
| IAKLTRAVR | 7.591 | 25.64 | 0.38 | 145 |
| YPVHIAKLT | 7.529 | 29.58 | 0.33 | 146 |
| GKKRMVVNY | 7.529 | 29.58 | 0.38 | 147 |

Aphid Transmission Protein

| | | | | |
|---|---|---|---|---|
| HLADRB1*0101 | | | | |
| RLKPLSLNS | 9.227 | 0.59 | 0.33 | 148 |
| NIQNIINHL | 8.713 | 1.94 | 0.29 | 149 |
| YKKDTIIRL | 8.446 | 3.58 | 0.38 | 150 |
| IIRLKPLSL | 8.416 | 3.84 | 0.33 | 151 |
| NIINHLNNL | 8.397 | 4.01 | 0.33 | 152 |
| HLADRB*0401 | | | | |
| KSKNPSVFN | 7.381 | 41.59 | 0.33 | 153 |
| IRLKPLSLN | 7.33 | 46.77 | 0.33 | 154 |
| EKAIQSLEN | 6.992 | 101.86 | 0.29 | 155 |
| YVFSSSKGN | 6.961 | 109.4 | 0.38 | 156 |
| QNIINHLNN | 6.919 | 120.5 | 0.29 | 157 |
| HLADRB*0701 | | | | |
| EAQNTRIKS | 8.209 | 6.18 | 0.38 | 158 |
| LNSNNRSYV | 7.434 | 36.81 | 0.38 | 159 |
| YKKDTIIRL | 7.315 | 48.42 | 0.38 | 160 |
| PLSLNSNNR | 7.268 | 53.95 | 0.38 | 161 |
| PEPLTKEEV | 7.224 | 59.7 | 0.38 | 162 |

Capsid Protein

| | | | | |
|---|---|---|---|---|
| HLADRB1*0101 | | | | |
| IIDLTSLPS | 9.436 | 0.37 | 0.38 | 163 |
| ILDRTINRF | 9.134 | 0.73 | 0.38 | 164 |
| LIDDWAAEI | 8.91 | 1.23 | 0.33 | 165 |
| YSLGFAAKI | 8.757 | 1.75 | 0.33 | 166 |
| YINQYLSKI | 8.756 | 1.75 | 0.38 | 167 |
| HLADRB*0401 | | | | |
| MYTMFLGLN | 7.39 | 40.74 | 0.29 | 168 |
| KYKAYKPYK | 6.919 | 120.5 | 0.29 | 169 |
| AKIRMTKLQ | 6.902 | 125.31 | 0.25 | 170 |
| SSEKAHILQ | 6.887 | 129.72 | 0.25 | 171 |
| DGEGPSRYN | 6.887 | 129.72 | 0.33 | 172 |
| HLADRB*0701 | | | | |
| LIRNTRWNR | 7.834 | 14.66 | 0.38 | 173 |
| EANGTSIYS | 7.712 | 19.41 | 0.38 | 174 |
| KIRMTKLQL | 7.425 | 37.58 | 0.38 | 175 |
| EKALTRFRH | 7.302 | 49.89 | 0.38 | 176 |
| EQVIDAMYT | 7.283 | 52.12 | 0.33 | 177 |

Inculsion Body Matrix Protein

| | | | | |
|---|---|---|---|---|
| HLADRB1*0101 | | | | |
| FAKALTSPP | 9.395 | 0.4 | 0.38 | 178 |
| FIQSLLRLN | 8.97 | 1.07 | 0.38 | 178 |
| YLYDLVRKF | 8.936 | 1.16 | 0.38 | 180 |
| NIKDTVSED | 8.87 | 1.35 | 0.33 | 181 |
| NILPKDMNS | 8.758 | 1.75 | 0.29 | 182 |
| HLADRB*0401 | | | | |
| NPLMANILP | 7.344 | 45.29 | 0.25 | 183 |
| VRAKISLAR | 7.164 | 68.55 | 0.33 | 184 |
| PKQKAHWLM | 7.122 | 75.51 | 0.21 | 185 |
| VSKKVVPTE | 7.098 | 79.8 | 0.25 | 186 |
| HTNYYVVYN | 7.068 | 85.51 | 0.21 | 187 |
| HLADRB*0701 | | | | |
| YVVYNGPHA | 7.823 | 15.03 | 0.38 | 188 |
| KKVKDAVKR | 7.777 | 16.71 | 0.33 | 189 |
| KVVPTESKA | 7.745 | 17.99 | 0.38 | 190 |
| PGVAHKKFA | 7.599 | 25.18 | 0.38 | 191 |
| PEKEEAVHS | 7.52 | 30.2 | 0.38 | 192 |

PMMV Protein Sequences:

| Protein | | SEQ ID | |
|---|---|---|---|
| Name | Accession # | NO. | Sequence |
| Replication | NP_619740.1 | 193 | MAYTQQATNAALASTLRGNNPLVNDLANRRLYESAVEQCNAHDRRPKVNFLRSISEEQTLIATKAYPE |
| Associated | | | FQITFYNTQNAVHSLAGGLRSLELEYLMMQIPYGSTTYDIGGNFAAHMFKGRDYVHCCMPNMDLRDV |
| Protein | | | MRHNAQKDSIELYLSKLAQKKKVIPPYQKPCFDKYTDDPQSVVCSKPFQHCEGVSHCTDKVYAVALHS |
| | | | LYDIPADEFGAALLRRNVHVCYAAFHFSENLLLEDSYVSLDDIGAFFSREGDMLNFSFVAESTLNYTHS |
| | | | YSNVLKYVCKTYFPASSREVYMKEFLVTRVNTWFCKFSRLDTFVLYRGVYHRGVDKEQFYSAMEDA |
| | | | WHYKKTLAMMNSERILLEDSSSVNYWFPKMKDMVIVPLFDVSLQNEGKRLARKEVMVSKDFVYTVL |
| | | | NHIRTYQSKALTYANVLSFVESIRSRVIINGVTARSEWDVDKALLQSLSMTFFLQTKLAMLKDDLVVQK |
| | | | FQVHSKSLTEYVWDEITAAFHNCFPTIKERLINKKLITVSEKALEIKVPDLYVTFHDRLVKEYKSSVEMP |
| | | | VLDVKKSLEEAEVMYNALSEISILKDSDKFDVDVFSRMCNTLGVDPLVAAKVMVAVVSNESGLTLTFE |
| | | | RPTEANVALALQPTITSKEEGSLKIVSSDVGESSIKEVVRKSEISMLGLTGNTVSDEFQRSTEIESLQQFH |
| | | | MVSTETIIRKQMHAMVYTGPLKVQQCKNYLDSLVASLSAAVSNLKKIIKDTAAIDLETKEKFGVYDVC |
| | | | LKKWLVKPLSKGHAWGVVMDSDYKCFVALLTYDGENIVCGETWRRVAVSSESLVYSDMGKIRAIRSV |
| | | | LKDGEPHISSAKVTLVDGVPGCGKTKEILSRVNFDEDLVLVPGKQAAEMIRRRANSSGLIVATKENVRT |
| | | | VDSFLMNYGRGPCQYKRLFLDEGLMLHPGCVNFLVGMSLCSEAFVYGDTQQIPYINRVATFPYPKHLS |
| | | | QLEVDAVETRRTTLRCPADITFFLNQKYEGQVMCTSSVTRSVSHEVIQGAAVMNPVSKPLKGKVITFTQ |
| | | | SDKSLLLSRGYEDVHTVHEVQGETFEDVSLVRLTPTPVGIISKQSPHLLVSLSRHTRSIKYYTVVLDAVV |
| | | | SVLRDLECVSSYLLDMYKVDVSTQXQLQIESVYKGVNLFVAAPKTGDVSDMQYYYDKCLPGNSTILNE |
| | | | YDAVTMQIRENSLNVKDCVLDMSKSVPLPRESETTLKPVIRTAAEKPRKPGLLENLVAMIKRNFNSPEL |
| | | | VGVVDIEDTASLVVDKFFDAYLIKEKKKPKNIPLLSRASLERWIEKQEKSTIGQLADFDFIDLPAVDQYR |
| | | | HMIKQQPKQRLDLSIQTEYPALQTIVYHSKKINALFGPVFSELTRQLLETIDSSRFMFYTRKTPTQIEEFFS |
| | | | DLDSNVPMDILELDISKYDKSQNEFHCAVEYEIWKRLGLDDFLAEVWKHGHRKTTLKDYTAGIKTCLW |
| | | | YQRKSGDVTTFIGNTIIIAACLSSMLPMERLIKGAFCGDDSILYFPKGTDFPDIQQGANLLWNFEAKLFRK |
| | | | RYGYFCGRYIIHHDRGCIVYYDPLKLISKLGAKHIKNREHLEEFRTSLCDVAGSLNNCAYYTHLNDAVG |
| | | | EVIKTAPLGSFVYRALVKYLCDKRLFQTLFLE |
| Replication | NP_619741.1 | 194 | MAYTQQATNAALASTLRGNNPLVNDLANRRLYESAVEQCNAHDRRPKVNFLRSISEEQTLIATKAYPE |
| Associated | | | FQITFYNTQNAVHSLAGGLRSLELEYLMMQIPYGSTTYDIGGNFAAHMFKGRDYVHCCMPNMDLRDV |
| Protein | | | MRHNAQKDSIELYLSKLAQKKKVIPPYQKPCFDKYTDDPQSVVCSKPFQHCEGVSHCTDKVYAVALHS |
| | | | LYDIPADEFGAALLRRNVHVCYAAFHFSENLLLEDSYVSLDDIGAFFSREGDMLNFSFVAESTLNYTHS |
| | | | YSNVLKYVCKTYFPASSREVYMKEFLVTRVNTWFCKFSRLDTFVLYRGVYHRGVDKEQFYSAMEDA |
| | | | WHYKKTLAMMNSERILLEDSSSVNYWFPKMKDMVIVPLFDVSLQNEGKRLARKEVMVSKDFVYTVL |
| | | | NHIRTYQSKALTYANVLSFVESIRSRVIINGVTARSEWDVDKALLQSLSMTFFLQTKLAMLKDDLVVQK |
| | | | FQVHSKSLTEYVWDEITAAFHNCFPTIKERLINKKLITVSEKALEIKVPDLYVTFHDRLVKEYKSSVEMP |
| | | | VLDVKKSLEEAEVMYNALSEISILKDSDKFDVDVFSRMCNTLGVDPLVAAKVMVAVVSNESGLTLFE |
| | | | RPTEANVALALQPTITSKEEGSLKIVSSDVGESSIKEVVRKSEISMLGLTGNTVSDEFQRSTEIESLQQFH |
| | | | MVSTETIIRKQMHAMVYTGPLKVQQCKNYLDSLVASLSAAVSNLKKIIKDTAAIDLETKEKFGVYDVC |
| | | | LKKWLVKPLSKGHAWGVVMDSDYKCFVALLTYDGENIVCGETWRRVAVSSESLVYSDMGKIRAIRSV |
| | | | LKDGEPHISSAKVTLVDGVPGCGKTKEILSRVNFDEDLVLVPGKQAAEMIRRRANSSGLIVATKENVRT |
| | | | VDSFLMNYGRGPCQYKRLFLDEGLMLHPGCVNFLVGMSLCSEAFVYGDTQQIPYINRVATFPYPKHLS |
| | | | QLEVDAVETRRTTLRCPADITFFLNQKYEGQVMCTSSVTRSVSHEVIQGAAVMNPVSKPLKGKVITFTQ |
| | | | SDKSLLLSRGYEDVHTVHEVQGETFEDVSLVRLTPTPVGIISKQSPHLLVSLSRHTRSIKYYTVVLDAVV |
| | | | SVLRDLECVSSYLLDMYKVDVSTQ |
| Movement | NP_619742.1 | 195 | MALVVKDDVKISEFINLSAAEKFLPAVMTSVKTVRISKVDKVIAMENDSLSDVNLLKGVKLVKDGYVC |
| Protein | | | LAGLVVSGEWNLPDNCRGGVSVCLVDKRMQRDDEATLGSYRTSAAKKRFAFKLIPNYSITTADAERK |
| | | | VWQVLVNIRGVAMEKGFCPLSLEFVSVCIVHKSNIKLGLREKITSVSEGGPVELTEAVVDEFIESVPMAD |
| | | | RLRKFRNQSKKGSNKYVGKRNDNKGLNKEGKLFDKVRIGQNSESSDAESSSF |
| Coat | NP_619743.1 | 196 | MAYTVSSANQLVYLGSVWADPLELQNLCTSALGNQFQTQQARTTVQQQFSDVWKTIPTATVRFPATG |
| Protein | | | FKVFRYNAVLDSLVSALLGAFDTRNRIIEVENPQNPTTAETLDATRRVDDATVAIRASISNLMNELVRGT |
| | | | GMYNQALFESASGLTWATTP |

PPMV Peptides

| | | | | |
|---|---|---|---|---|
| Amino | Predicted | Predicted | Confidence of | SEQ |
| acid | −logIC50 | IC50 Value | prediction | ID |
| groups | (M) | (nM) | (Max = 1) | NO |

Relication-Associated Protein 1a

| | | | | |
|---|---|---|---|---|
| HLADRB1*0101 | | | | |
| YAVALHSLY | 9.319 | 0.48 | 0.38 | 197 |
| FLQTKLAML | 9.19 | 0.65 | 0.33 | 198 |
| IIKDTAAID | 9.163 | 0.69 | 0.38 | 199 |
| QATNAALAS | 9.16 | 0.69 | 0.33 | 200 |
| MIRRRANSS | 9.072 | 0.85 | 0.29 | 201 |
| HLADRB*0401 | | | | |
| VPLFDVSLQ | 7.427 | 37.41 | 0.38 | 202 |
| YTQQATNAA | 7.422 | 37.84 | 0.33 | 203 |
| KVMVAVVSN | 7.371 | 42.56 | 0.29 | 204 |
| DSLVASLSA | 7.327 | 47.1 | 0.33 | 205 |
| QTLIATKAY | 7.319 | 47.97 | 0.25 | 206 |
| HLADRB*0701 | | | | |
| LIVATKENV | 8.413 | 3.86 | 0.38 | 207 |
| GAALLRRNV | 8.186 | 6.52 | 0.38 | 208 |
| MPVLDVKKS | 8.071 | 8.49 | 0.29 | 209 |
| AKVMVAVVS | 7.973 | 10.64 | 0.38 | 210 |
| DAVETRRTT | 7.909 | 12.33 | 0.38 | 211 |

Relication-Associated Protein 2

| | | | | |
|---|---|---|---|---|
| HLADRB1*0101 | | | | |
| YAVALHSLY | 9.319 | 0.48 | 0.38 | 212 |
| FLQTKLAML | 9.19 | 0.65 | 0.33 | 213 |
| IIKDTAAID | 9.163 | 0.69 | 0.38 | 214 |
| QATNAALAS | 9.16 | 0.69 | 0.33 | 215 |
| MIRRRANSS | 9.072 | 0.85 | 0.29 | 216 |
| HLADRB*0401 | | | | |
| VPLFDVSLQ | 7.427 | 37.41 | 0.38 | 217 |
| YTQQATNAA | 7.422 | 37.84 | 0.33 | 218 |
| KVMVAVVSN | 7.371 | 42.56 | 0.29 | 219 |
| DSLVASLSA | 7.327 | 47.1 | 0.33 | 220 |
| QTLIATKAY | 7.319 | 47.97 | 0.25 | 221 |
| HLADRB*0701 | | | | |
| LIVATKENV | 8.413 | 3.86 | 0.38 | 222 |
| GAALLRRNV | 8.186 | 6.52 | 0.38 | 223 |
| MPVLDVKKS | 8.071 | 8.49 | 0.29 | 224 |
| AKVMVAVVS | 7.973 | 10.64 | 0.38 | 225 |
| DAVETRRTT | 7.909 | 12.33 | 0.38 | 226 |

Movement Protein

| | | | | |
|---|---|---|---|---|
| HLADRB1*0101 | | | | |
| YSITTADAE | 8.95 | 1.12 | 0.33 | 227 |
| YRTSAAKKR | 8.929 | 1.18 | 0.38 | 228 |
| KISEFINLS | 8.825 | 1.5 | 0.33 | 229 |
| FINLSAAEK | 8.643 | 2.28 | 0.33 | 230 |
| SYRTSAAKK | 8.555 | 2.79 | 0.33 | 231 |
| HLADRB*0401 | | | | |
| VCLAGLVVS | 7.372 | 42.46 | 0.33 | 232 |
| VHKSNIKLG | 7.274 | 53.21 | 0.38 | 234 |
| NLLKGVKLV | 7.204 | 62.52 | 0.29 | 235 |
| SGEWNLPDN | 7.161 | 69.02 | 0.29 | 236 |
| ERKVWQVLV | 7.137 | 72.95 | 0.33 | 237 |
| HLADRB*0701 | | | | |
| PAVMTSVKT | 8.309 | 4.91 | 0.38 | 238 |
| EKFLPAVMT | 7.662 | 21.78 | 0.38 | 239 |
| TSVKTVRIS | 7.597 | 25.29 | 0.38 | 240 |
| LPDNCRGGV | 7.567 | 27.1 | 0.38 | 241 |
| GPVELTEAV | 7.52 | 30.2 | 0.38 | 242 |

Relication-Associated Protein 1b

| | | | | |
|---|---|---|---|---|
| HLADRB1*0101 | | | | |
| YYDPLKLIS | 9.635 | 0.23 | 0.33 | 243 |
| FIDLPAVDQ | 9.306 | 0.49 | 0.33 | 244 |
| DIEDTASLV | 9.215 | 0.61 | 0.33 | 245 |
| FVYRALVKY | 9.079 | 0.83 | 0.38 | 246 |
| FFSDLDSNV | 9.029 | 0.94 | 0.33 | 247 |
| HLADRB*0401 | | | | |
| VVLDAVVSV | 7.659 | 21.93 | 0.38 | 248 |
| VYYDPLKLI | 7.366 | 43.05 | 0.38 | 249 |
| VRLTPTPVG | 7.341 | 45.6 | 0.38 | 250 |
| VIQGAAVMN | 7.313 | 48.64 | 0.33 | 251 |
| WYQRKSGDV | 7.285 | 51.88 | 252 | |
| HLADRB*0701 | | | | |
| TVVLDAVVS | 8.395 | 4.03 | 0.33 | 253 |
| KGVNLFVAA | 7.891 | 12.85 | 0.38 | 254 |
| QIRENSLNV | 7.529 | 29.58 | 0.38 | 255 |
| FIDLPAVDQ | 7.495 | 31.99 | 0.38 | 256 |
| FIGNTIIIA | 7.482 | 32.96 | 0.38 | 257 |

Coat Protein

| | | | | |
|---|---|---|---|---|
| HLADRB1*0101 | | | | |
| YTVSSANQL | 9.105 | 0.79 | 0.38 | 258 |
| FRYNAVLDS | 8.598 | 2.52 | 0.29 | 259 |
| RRVDDATVA | 8.557 | 2.77 | 0.33 | 260 |
| NAVLDSLVS | 8.536 | 2.91 | 0.38 | 261 |
| KTIPTATVR | 8.491 | 3.23 | 0.38 | 262 |
| HLADRB*0401 | | | | |
| VRFPATGFK | 7.334 | 46.34 | 0.33 | 263 |
| FRYNAVLDS | 7.148 | 71.12 | 0.38 | 264 |
| VYLGSVWAD | 7.13 | 74.13 | 0.33 | 265 |
| VAIRASISN | 7.087 | 81.85 | 0.29 | 266 |
| VQQQFSDVW | 7.051 | 88.92 | 0.29 | 267 |
| HLADRB*0701 | | | | |
| IPTATVRFP | 7.516 | 30.48 | 0.33 | 268 |
| TLDATRRVD | 7.392 | 40.55 | 0.38 | 269 |
| NAVLDSLVS | 7.358 | 43.85 | 0.33 | 270 |
| QLVYLGSVW | 7.295 | 50.7 | 0.38 | 271 |
| RFPATGFKV | 7.262 | 54.7 | 0.38 | 272 |

Oat Blue Dwarf Virus Protein Sequence:

| | | SEQ | |
|---|---|---|---|
| Protein | | ID | |
| Name | Accession # | NO. | Sequence |
| Capsid | ADD13603.1 | 273 | MSGIHASQVGPPPASDDRTDRQPSLPLAPRLVESSLAVPYVDVPFQWAVASYAGDSAKFLTDDL |
| Protein | | | SGSSHLSRLTIGYRHAELISAELEFAPLAAAFSKPISVTAVWTIASIAPATTTELQYYGGRLLTLGG |
| | | | PVLMGSVTRIPADLTRLNPVIKTAVGFTDCPRFTYSVYANSGSANTPLITVMVRGVIRLSGPSGN |
| | | | TVTATT |
| Replicase- | ADD13602.1 | 274 | MTTYAFHPLLPTPTSFATVTGGGLKDVIETLSSTIHRDTIAAPLMETLASPYRDSLRDFPWAVPAS |
| Associated | 2.1 | | ALPFLQECGITVAGHGFKAHPHPVHKTIETHLLHKVWPHYAQVPSSVLFMKPSKFAKLQRGNA |
| Polyprotein | | | NFSALHNYRLTAKDTPRYPNTSTSLPDTETAFMHDALMYYTPAQIVDLFLSCPKLEKLYASLVV |
| | | | PPESSFTSISLHPDLYRFRFDGDRLIYELEGNPAHNYTQPRSALDWLRTTTIRGPGVSLTVSRLDS |
| | | | WGPCHSLLIQRGIPPMHAEHDSISFRGPRAVAIPEPSSLHQDLRHRLVPEDVYNALFLYVRAVRT |
| | | | LRVTDPAGFVRTQCSKSEYAWVTSSAWDNLAHFALLTAPHRPRTSFYLFSSTFQRLEHWVRHH |
| | | | TFLLAGLTTAFALPPSAWLANLVARTSASHIQGLALARRWLITPPHLFRPPSPPSFALLLQRNSTG |
| | | | PILLRGSRLEFEAFPSLAPQLARRFPFLARLLPQKPINPWIVASLAVAVAIPAASLAVRWFFGPDTP |
| | | | QAMHDRYHTMFHPREWRLTLPRGPISCGRSSFSPLPHPPSPTPAPDSRAGPLQPPSALPSTHEPAP |
| | | | ADLESPAPQAHAPQTEPPSPVIEQEARPDPFPAPAPRPAPTPSASAPSPAPTPSAPEPPSPTASEQAA |
| | | | SLIPAPSSALVVEPSGVVSASSWGATNQPADQVDDSPLARDPSASGPVRFYRDLFPANYAGDSG |
| | | | TFDFRARASGRSPTPYPAMDCLLVATEQATRISREALWDCLTATCPDSFLDPKSIAQHGLSTDHF |
| | | | VILAHRFSLCANFHSAAHVIQLGMADATSTFMINHTAGSAGLPGHFSLRLGDQPRALNGGLAQD |
| | | | LAVAALRFNISGDLLPTRSVHTYRSWPKRAKNLVSNMKNGFDGVMASINPIRPSDAREKIVALD |
| | | | GLLDIAQPRSVRLIHIAGFPGCGKTHPITKLLHTAAFRDFKLAVPTTELRSEWKELMKLSPSQAW |
| | | | RFGTWESSLLKSARILVIDEIYKLPRGYLDLAIHSDSSIEFVIALGDPLQGEYHSTHPSSSNSRLIPE |
| | | | VSHLAPYLDYYCLWSYRVPQDVATFFQVQSHNPALGFARLSKQFPTTGRVLTNSQNSMLTMTQ |
| | | | CGYSAVTIASSQGSTYSGATHIHLDRNSSLLSPSNSLVALTRSRTGVFFSGDPALLNGGPNSNLMF |
| | | | SAFFQGKSRHIRDWFPTLFPTATLLLSPLRQRHNRLTGALAPVEPSHLLLPDLPSLLPLPASGPYS |
| | | | RAFPVRSRFAAAVKPFDRSDVLSWAPIAVGDGETNAPRIDTSFLPETRRPLHFDLPSFRPQAPPPP |
| | | | SDPAPSGTAFEPVYPGETFENLVAHFLPAHDPTDREIHWRGQLSNQFPHIDKEYHLAAQPMTLL |
| | | | APIHDSKHDPTLLAASIQKRLRFRPSASPYRITPRDELLGQLLYESLCRAYHRSPTSTHPFDEALFV |
| | | | ECIDLNEFAQLTSKTQAVIMGNARRSDPDWRWSAVRIFSKTQHKVNEGSIFGAWKACQTLALM |
| | | | HDAVVLLLGPVKKYQRVFDARDRPAHLYIHAGQTPSSMSLWCQTHLTPAVKLANDYTAFDQS |
| | | | QHGEAVVLERKKMERLSIPDHLISLHVYLKTHVETQFGPLTCMRLTGEPGTYDDNTDYNLAVIN |
| | | | LEYAAAHVPTMVSGDDSLLDFEPPRRPEWVAIEPLLALRFKKERGLYATFCGYYASRVGCVRSP |
| | | | IALFAKLAIAVDDSSISDKLAAYLMEFAVGHSLGDSLWSALPLSAVPFQSACFDFFCRRAPRDLK |
| | | | LALHLGEVPETIIQRLSHLSWLSHAVYSLLPSRLRLAILHSSRQHRSLPEDPAVSSLQGELLHTFH |
| | | | APMPSPPSLPLFGGLSPDNILTPHEFRTALYESSAYPTPPNSPTSMSGIHASQVGPPPASDDRTDRQ |
| | | | PSLPLAPRLVESSLAVPYVDVPFQWAVASYAGDSAKFLTDDLSGSSHLSRLTIGYRHAELISAEL |
| | | | EFAPLAAAFSKPISVTAVWTIASIAPATTTELQYYGGRLLTLGGPVLMGSVTRIPADLTRLNPVIK |
| | | | TAVGFTDCPRFTYSVYANSGSANTPLITVMVRGVIRLSGPSGNTVTATT |
| RNA | NP_734079.1 | 275 | LAPAQPSHLLLPDLPSLPPLPASGPYSRSFPVRSRFAAAVKPSDRSDVLSWAPIAVGDGETNAPRI |
| Dependent | | | DTSFLPETRRPLHFDLPSFRPQAPPPPSDPAPSGTAFEPVYPGETEENLVAHFLPAHDPTDREIHW |
| RNA | | | RRQLSNQFPHVDKEYHLAAQPMTLLAPIHDSKHDPTLLAASIQKRLRFRPSASPYRISPRDELLG |
| Polymerase | | | QLLYESLCRAYHRSPTTTHPFDEALFVECIDLNEFAQLTSKTQAVIMGNARRSDPDWRWSAVRI |
| | | | FSKTQHKVNEGSIFGAWKACQTLALMHDAVVLLLGPVKKYQRVFDARDRPAHLYIHAGQTPSS |
| | | | MSLWCQTHLTPAVKLANDYTAFDQSQHGEAVVLERKKMERLSIPDHLISLHVHLKTHVETQFG |
| | | | PLTCMRLTGEPGTYDDNTDYNLAVINLEYAAAHVPTMVSGDDSLLDFEPPRRPEWVAIEPLLAL |
| | | | RFKKERGLYATFCGYYASRVGCVRSPIALFAKLAIAVDDSSISDKLAAYLMEFAVGHSLGDSLW |
| | | | SALPLSAVPFQSACFDFFCRRAPRDLKLALHLGEVPETIIQRLSHLSWLSHAVYSLLPSRLRLAIL |
| | | | HSSRQHRSLPEDPAVSSLQGELLQTFHAPMPSLPSLPLFGG |
| Methyltransferase/ | NP_734078.1 | 276 | MTTYAFHPLLPTPTSFATITGGGLKDVIETLSSTIHRDTIAAPLMETLASPYRDSLRDFPWAVPAS |
| Protease/ | | | ALPFLQECGITVAGHGFKAHPHPVHKTIETHLLHKVWPHYAQVPSSVLFMKPSKFAKLQRGNA |
| Helicase | | | NFSALHNYRLTAKDTPRYPNTSTSLPDTETAFMHDALMYYTPAQIVDLFLSCPKLEKLYASLVV |
| | | | PPESSFTSISLHPDLYRFRFDGDRLIYELEGNPAHNYTQPRSALDWLRTTTIRGPGVSLTVSRLDS |
| | | | WGPCHSLLIQRGIPPMHAEHDSISFRGPRAVAIPEPSSLHQDLRHRLVPEDVYNALFLYVRAVRT |
| | | | LRVTDPAGFVRTQCSKPEYAWVTSSAWDNLAHFALLTAPHRPRTSFYLFSSTFQRLEHWVRHH |
| | | | TFLLAGLTTAFALPPSAWLANLVARASASHIQGLALARRWLITPPHLFRPPPPPSFALLLQRNSTG |
| | | | PVLLRGSRLEFEAFPSLAPQLARRFPFLARLLPQKPIDPWVVASLAVAVAIPAASLAVRWFFGPD |
| | | | TPQAMHDRYHTMFHPREWRLTLPRGPISCGRSSFSPLPHPPSPTPAPDSRAEPLQPPSAPPSTHEP |
| | | | APADLEPQAPPAHAPQTEPPSPVIEQEARPNPLPAPAPLSAPTPSASAPSLAPTPSAPEPPSPTASEQ |
| | | | AASLIPAPSSALVVEPSGVVSASSWGATNQPADQVDDSPLARDPSASGPVRFYRDLFPANYAGD |
| | | | SGTFDFRARASGRSPTPYPAMDCLLVATEQATRISREALWDCLTATCPDSFLDPKSIAQHGLSTD |
| | | | HFVILAHRFSLCANFHSAEHVIQLGMADATSIFMINHTAGSAGLPGHFSLRLGDQPRALNGGLA |
| | | | QDLAVAALRFNISGDLLPTRSVHTYRSWPKRAKNLVSNMKNGFDGVMASINPIRPSDAREKIVA |
| | | | LDGLLDIARPRSVRLIHIAGFPGCGKTHPITKLLHTAAFRDFKLAVPTTELRSEWKELMKLSPSQA |
| | | | WRFGTWESSLLKSARILVIDEIYKLPRGYLDLAIHSDSSIEFVIALGDPLQGEYHSTHPSSSNSRLIP |
| | | | EVSHLAPYLDYYCLWSYRVPQDVAAFFQVQSHNPALGFARLSKQFPTTGRVLTNSQNSMLTMT |
| | | | QCGYSAVTIASSQGSTYSGATHIHLDRNSSLLSPSNSLVALTRSRTGVFFSGDPALLNGGPNSNL |
| | | | MFSAFFQGKSRHIRAWFPTLFPTATLLFSPLRQRHNRLTGA |

Oat Blue Dwarf Virus (OBDV) Peptides

| | | | | |
|---|---|---|---|---|
| Amino | Predicted | Predicted | Confidence of | SEQ |
| acid | −logIC50 | IC50 Value | prediction | ID |
| groups | (M) | (nM) | (Max = 1) | NO |

Capsid Protein

| | | | | |
|---|---|---|---|---|
| HLADRB1*0101 | | | | |
| FLTDDLSGS | 9.31 | 0.49 | 0.38 | 277 |
| PADLTRLNP | 9.024 | 0.95 | 0.38 | 278 |
| FAPLAAAFS | 9.016 | 0.96 | 0.38 | 279 |
| PATTTELQY | 9.01 | 0.98 | 0.38 | 280 |
| FQWAVASYA | 8.813 | 1.54 | 0.33 | 281 |
| HLADRB*0401 | | | | |
| PVLMGSVTR | 7.442 | 36.14 | 0.29 | 282 |
| SGSANTPLI | 7.332 | 46.56 | 0.33 | 283 |
| SVYANSGSA | 7.289 | 51.4 | 0.33 | 284 |
| VWTIASIAP | 7.207 | 62.09 | 0.29 | 285 |
| VIKTAVGFT | 7.196 | 63.68 | 0.38 | 286 |
| HLADRB*0701 | | | | |
| PISVTAVWT | 7.94 | 11.48 | 0.33 | 287 |
| PADLTRLNP | 7.853 | 14.03 | 0.38 | 288 |
| PVIKTAVGF | 7.717 | 19.19 | 0.38 | 289 |
| FQWAVASYA | 7.603 | 24.95 | 0.38 | 290 |
| GPVLMGSVT | 7.57 | 26.92 | 0.38 | 291 |

Replicase Associated Poly Protein a

| | | | | |
|---|---|---|---|---|
| HLADRB1*0101 | | | | |
| YYTPAQIVD | 9.144 | 0.72 | 0.38 | 292 |
| FRDFKLAVP | 9.106 | 0.78 | 0.38 | 293 |
| HIQGLALAR | 9.103 | 0.79 | 0.38 | 294 |
| FALLLQRNS | 9.096 | 0.8 | 0.33 | 295 |
| HRDTIAAPL | 9.051 | 0.89 | 0.38 | 296 |
| HLADRB*0401 | | | | |
| SEQAASLIP | 7.422 | 37.84 | 0.33 | 297 |
| AWLANLVAR | 7.352 | 44.46 | 0.33 | 298 |
| AIPAASLAV | 7.332 | 46.56 | 0.33 | 299 |
| FEAFPSLAP | 7.322 | 47.64 | 0.38 | 300 |
| PRPAPTPSA | 7.315 | 48.42 | 0.33 | 301 |
| HLADRB*0701 | | | | |
| LAVAVAIPA | 8.051 | 8.89 | 0.38 | 302 |
| KPINPWIVA | 8.023 | 9.48 | 0.38 | 303 |
| FALLTAPHR | 7.918 | 12.08 | 0.38 | 304 |
| FAKLQRGNA | 7.887 | 12.97 | 0.38 | 305 |
| LANLVARTS | 7.843 | 14.35 | 0.38 | 306 |

methyltransferase/protease/helicase a

| | | | | |
|---|---|---|---|---|
| HLADRB1*0101 | | | | |
| YYTPAQIVD | 9.144 | 0.72 | 0.38 | 307 |
| FRDFKLAVP | 9.106 | 0.78 | 0.38 | 308 |
| HIQGLALAR | 9.103 | 0.79 | 0.38 | 309 |
| FALLLQRNS | 9.096 | 0.8 | 0.33 | 310 |
| NLVARASAS | 9.076 | 0.84 | 0.33 | 311 |
| HLADRB*0401 | | | | |
| PSLAPTPSA | 7.473 | 33.65 | 0.33 | 312 |
| SEQAASLIP | 7.422 | 37.84 | 0.33 | 313 |
| AWLANLVAR | 7.352 | 44.46 | 0.33 | 314 |
| VRTQCSKPE | 7.341 | 45.6 | 0.25 | 315 |
| AIPAASLAV | 7.332 | 46.56 | 0.33 | 316 |
| HLADRB*0701 | | | | |
| LAVAVAIPA | 8.051 | 8.89 | 0.38 | 317 |
| FALLTAPHR | 7.918 | 12.08 | 0.38 | 318 |
| FAKLQRGNA | 7.887 | 12.97 | 0.38 | 319 |
| KPIDPWVVA | 7.844 | 14.32 | 0.38 | 320 |
| LAQDLAVAA | 7.839 | 14.49 | 0.38 | 321 |

RNA Dependant RNA Pol

| | | | | |
|---|---|---|---|---|
| HLADRB1*0101 | | | | |
| RFRPSASPY | 9.008 | 0.98 | 0.29 | 322 |
| SISDKLAAY | 8.954 | 1.11 | 0.38 | 323 |
| EYHLAAQPM | 8.923 | 1.19 | 0.33 | 324 |
| PAVKLANDY | 8.923 | 1.19 | 0.33 | 325 |
| YIHAGQTPS | 8.854 | 1.4 | 0.38 | 326 |
| HLADRB*0401 | | | | |
| YHLAAQPMT | 7.516 | 30.48 | 0.38 | 327 |
| FRPSASPYR | 7.392 | 40.55 | 0.38 | 328 |
| PSLPPLPAS | 7.346 | 45.08 | 0.29 | 329 |
| DKLAAYLME | 7.339 | 45.81 | 0.25 | 330 |
| FRPQAPPPP | 7.313 | 48.64 | 0.38 | 331 |
| HLADRB*0701 | | | | |
| YPGETFENL | 7.766 | 17.14 | 0.38 | 332 |
| RWSAVRIFS | 7.699 | 20 | 0.38 | 333 |
| PAVKLANDY | 7.679 | 20.94 | 0.38 | 334 |
| YAAAHVPTM | 7.672 | 21.28 | 0.33 | 335 |
| FPVRSRFAA | 7.659 | 21.93 | 0.38 | 336 |

Replicase Associated Poly Protein b

| | | | | |
|---|---|---|---|---|
| HLADRB1*0101 | | | | |
| FPTATLLLS | 9.474 | 0.34 | 0.38 | 337 |
| FLTDDLSGS | 9.31 | 0.49 | 0.38 | 338 |
| TATLLLSPL | 9.04 | 0.91 | 0.33 | 339 |
| FAPLAAAFS | 9.016 | 0.96 | 0.38 | 340 |
| PATTTELQY | 9.01 | 0.98 | 0.38 | 341 |
| HLADRB*0401 | | | | |
| YHLAAQPMT | 7.516 | 30.48 | 0.38 | 342 |
| FRPSASPYR | 7.392 | 40.55 | 0.38 | 343 |
| VYLKTHVET | 7.348 | 44.87 | 0.29 | 345 |
| DKLAAYLME | 7.339 | 45.81 | 0.25 | 346 |
| FRPQAPPPP | 7.313 | 48.64 | 0.38 | 347 |
| HLADRB*0701 | | | | |
| PISVTAVWT | 7.94 | 11.48 | 0.33 | 348 |
| EVSHLAPYL | 7.791 | 16.18 | 0.33 | 349 |
| YPGETFENL | 7.766 | 17.14 | 0.38 | 350 |
| RWSAVRIFS | 7.699 | 20 | 0.38 | 351 |
| PAVKLANDY | 7.679 | 20.94 | 0.38 | 352 |

methyltransferase/protease/helicase b

| | | | | |
|---|---|---|---|---|
| HLADRB1*0101 | | | | |
| FPTATLLFS | 9.283 | 0.52 | 0.38 | 353 |
| GYLDLAIHS | 8.806 | 1.56 | 0.29 | 354 |
| HIHLDRNSS | 8.758 | 1.75 | 0.38 | 355 |
| RNSSLLSPS | 8.755 | 1.76 | 0.33 | 356 |
| TATLLFSPL | 8.732 | 1.85 | 0.33 | 357 |
| HLADRB*0401 | | | | |
| RVLTNSQNS | 7.182 | 65.77 | 0.29 | 358 |
| SHLAPYLDY | 7.172 | 67.3 | 0.25 | 359 |
| TNSQNSMLT | 7.125 | 74.99 | 0.29 | 360 |
| FPTATLLFS | 7.108 | 77.98 | 0.33 | 361 |
| SRLIPEVSH | 7.107 | 78.16 | 0.33 | 362 |
| HLADRB*0701 | | | | |
| EVSHLAPYL | 7.791 | 16.18 | 0.33 | 363 |
| GYLDLAIHS | 7.602 | 25 | 0.38 | 364 |
| YSGATHIHL | 7.507 | 31.12 | 0.38 | 365 |
| YRVPQDVAA | 7.411 | 38.82 | 0.38 | 366 |
| TRSRTGVFF | 7.332 | 46.56 | 0.38 | 367 |

Rice Grassy Stunt Virus Protein Sequence:

| | | SEQ | |
|---|---|---|---|
| Protein | | ID | |
| Name | Accession # | NO. | Sequence |
| RNA | NP_058528.1 | 368 | MNTNCQFSNISYLHNMNNEIVGVERFKYNDVEYDINGSLVDCFYKGAETIPTPSPNLKCFFNALC |
| Polymerase | | | LCLRVESKDYIKVMNKLRNQYYAMSIWTASELKELLRELDPNDSYMATYYSIIHVSICLDICICIH |
| | | | DETWDSHCKTFGDRSKLMIHMKLESRHYEAIDDPTYDYFELSSVLGGYLGSLDDDIDLPSMIELK |
| | | | VETKPLGDVFTERGQWYNSLASLAESNLHQQVPQFNCNLFSSIVRLKKYSRQQEVAMLSLNLGM |
| | | | TIEVRLLSYHENLYSLEGGFKCVGPGNGLLELIYDGSTNKWFFLKISGLLEVDQNYQVLEKVHDL |
| | | | ESLIRQLTQSFVQPSNWYSNKLKMIEKCKTIFPQRREVDYEPFLNKNKLLSLCFLSKELENLLTILL |
| | | | VDNDMVNVGTILKPKIYKYWGQNPELTKKQKHELLDSEGNLWGAVKSGLPVTVLRDDQYDKDF |
| | | | PTLSFSRKTAEFLFTSYDDDIQKLTNPEHSGYDESMYGLYEMHPRLKVPETSEIVSPDETEIVISFEN |
| | | | RFGNRKYHDFPSIPDNRAYSCKISTVKNIVHDFTFALFGDDLDVSFTDAGLFIPGDPDNNKTPDMII |
| | | | KHGEKHYSVIEFTTRNTNMRPDVRSRGWEDKTLKYRDAIHNRRDHFKISIDYYIIVVCQNGVQTN |
| | | | LMNLPTETMDELIYRYKLARQIALQIEQNLEYDIKADQAMKMEISSIKKIIEGIRIHKEDGELDPSK |
| | | | FIKPYTMAHYTKAVGTLESEDYDYLHKLDTYVSNKSMRKMEKLKHLNDVNIRAYRDEIRNESIL |
| | | | MMQSRKMEYESNFIKNEEAYRTSNEASVQLPMLVPKIVRVVGVSNTHEEVRNVVDEIISTSSMSS |
| | | | TEEAWKQGICGFMHYLYEIEDGKSDFSLAMEEPTLSTQMEDDLKKIRNKFNRISMVFDMDDRIDL |
| | | | AKIGINGKKYSKDPEVLAYRNESKKPFSLFTSTDDIERFINEECLQLFTPHDQELDNSVLDLISDSLK |
| | | | IHGCSSQSRLLESLDTYLKSKAYLFTKFVSDLAVELSISVKQNCQPREFIVKRLRDFQVYVLIKSTG |
| | | | SDGKVFFSLLFREDQELSKIINTTFKKVSKLGDRFLYTDFISVNYSKLVNWTRCESLMLSLYAFWR |
| | | | EQYNIPPNIGISSIPDEDFNSDYLKMWANCLLVLLNDKHQTEEVITSTRFIHMEAFVETPNWPKPH |
| | | | KMFEKLSTIPRSRLEVFYIKSAIKLMECYTETPIRLDNSGPMRRWYNIKNPFVTENGSLSNFPNHDV |
| | | | MLSSMYLGYLKNKDEDPEDNASGQLISKILGYEDKLPRGEDKKYLGLEDPPVDQCSTHMYSISLV |
| | | | KRMCDSFLGRLKSETGVSDPKDYLSTLCLEYLSHEFLESFVTLKASSNFSAEYYEYRPNENKRSRP |
| | | | QTVNEDLPKSESNRRNYGRSKVIEKIQTILTKKDPNEKYRLVVDLLKESLEEVEKNACLHVCIFRK |
| | | | NQHGGLREIYVLNIYERIVQKCVEDLARAILSVVPSETMTHPKNKFQIPNKHNIAARKEFGDSYFT |
| | | | VCTSDDASKWNQGHHVSKFITILVRILPKFWHGFIVRALQLWFHKRLFLGDDLLRLFCANDVLNT |
| | | | TDEKVKKVHEVFKGREVAPWMTRGMTYIETESGFMQGILHYISSLFHAIFLEDLAERQKKQLPQ |
| | | | MARIIQPDNESNVIIDCMESSDDSSMMISFSTKSMNDRQTFAMLLLVDRAFSLKEYYGDMLGIYK |
| | | | SIKSTTGTIFMMEFNIEFFFAGDTHRPTIRWVNAALNVSEQETLIASQEEMSNTLKDILEGGGTFYH |
| | | | TFVTQVAQAMLHYRMYGSSVSPLWGSYCSMIKLSKDPALGYFLMDHPMASGLMGFGYNLWKT |
| | | | CKQSFLSVKYADMLNLEFNTENSKRKMTPDIANLGVLSRTTTVGFGNKTKWMKMCDRMHLTD |
| | | | DIFDSIEQNPRILFFHAKNAEEMQQKIAIKMRSPGVMQSLAKTNTLGRRVASSVYFISRNVLFSMS |
| | | | AGVETDEKRKTSIFRELLNSNSNVVSKIGQKEAQIPGVQSLTEEPSDDFYSVEGLREGVIKMVSVL |
| | | | TDLTMEQSERLLSEKFGLTLDDTKLNDWFIDENKLMHKLSKGFGINIHVYISRDPEASFKLCHTFK |
| | | | CLTNSENLYFMLNPNYLLVRRQESSSMSDEHRRQIQYSVAKFIWFGEKDVPAHPKTLKIVWKKY |
| | | | KETWLWLRDTIGDTLVGSPFVSYIQLNNYLSRVSTKGRVLHFVGTMGKASSGNVNLMTLIRNNF |
| | | | SNGIVFSGGFTDVIKKEKTEDYKSLLSNLTMLNQSPLKYEEKLVAMTDLIVDNKDLEYSTSMLGS |
| | | | KRNKLAIIQMFLRTDPDLKFSGDYNTQDAVNLVEHHLGEFDQNLSLGGFRSLIRMGQLVEKELLD |
| | | | SGMGYEELEKNFEDLTINSLSASARRAYCQYIYCDRVLEDAYQQYNKRKPTQKMLLSLELLKAE |
| | | | AANDPTRNWLTMIGHRIVKSSYDLMKLRDEAKYCRRDIMEKIRIGNLGLLGGYVQKQSYNREEK |
| | | | KYFGPGVWRGYLHDVAVQIEVNSDQNMESYIKSVSLSSAMHLSDTIQSLKEWSREHRVGNSHYT |
| | | | MAYGNRDCEMLGRMFEERRVQMSDRDGCPIVLDPKLIIHQPFLSDSECIDITDHSIRLLQECTGER |
| | | | APYTTVLTVHLSKKDVITSELQSQQNVNMIKRLKMDDWLKDWILWRDQRAPTSLFTQMNLGQF |
| | | | PDLVDEKRLKSWCRELFESSLGYQKIVQLSKLSKAARDRLAHDYPESIQEDKEVCEELESMESLLT |
| | | | RISQAYKTIDMTIKDEDLEHLYELARDLAEEQDEIQMEKEAVNVSLFHKMFLSSVRKMDTFMGT |
| | | | DDLRLTMNIIKGESRQKLPASSMHYKRILQFMYDVPDSQFPTYNPPSSRGRGRRGRGRSYMF |
| 18.9K | NP_058527.1 | 369 | MGYYHSKTDNPKLITTKIRKYKVFSIPVKTQVIIITGSTLSLDFFTLQTWIHLQEGFILEMGVRSTNG |
| Protein | 27.1 | | VLKIVNTICQENGKIERDRWDWYGCADSGLRKVHYDEGIARSERTSIRVDIRGTLFVLTVDGHILG |
| | | | VYDVNSCINAINIGLEVLPNSDNTLDFDLIYH |

Rice Grassy Stunt Virus Peptides

| | | | | |
|---|---|---|---|---|
| Amino | Predicted | Predicted | Confidence of | SEQ |
| acid groups | −logIC50 (M) | IC50 Value (nM) | prediction (Max = 1) | ID NO |

RNA Polymerase a

| | | | | |
|---|---|---|---|---|
| HLADRB1*0101 | | | | |
| WYNSLASLA | 9.225 | 0.6 | 0.38 | 370 |
| YRDAIHNRR | 9.181 | 0.66 | 0.38 | 371 |
| YRDEIRNES | 9.075 | 0.84 | 0.29 | 372 |
| ILKPKIYKY | 9.027 | 0.94 | 0.38 | 373 |
| EYDIKADQA | 8.905 | 1.24 | 0.29 | 374 |
| HLADRB*0401 | | | | |
| TNLMNLPTE | 7.454 | 35.16 | 0.25 | 375 |
| QELDNSVLD | 7.452 | 35.32 | 0.33 | 376 |
| VPQFNCNLF | 7.287 | 51.64 | 0.33 | 377 |
| ASLAESNLH | 7.266 | 54.2 | 0.33 | 378 |
| VGPGNGLLE | 7.258 | 55.21 | 0.33 | 379 |
| HLADRB*0701 | | | | |
| KIVRVVGVS | 8.09 | 8.13 | 0.38 | 380 |
| YQVLEKVHD | 7.923 | 11.94 | 0.38 | 381 |
| EEVRNVVDE | 7.674 | 21.18 | 0.38 | 382 |
| PKIVRVVGV | 7.623 | 23.82 | 0.33 | 383 |
| LKHLNDVNI | 7.553 | 27.99 | 0.38 | 384 |

RNA Polymerase c

| | | | | |
|---|---|---|---|---|
| HLADRB1*0101 | | | | |
| DYKSLLSNL | 9.247 | 0.57 | 0.38 | 385 |
| FEDLTINSL | 9.199 | 0.63 | 0.38 | 386 |
| YNTQDAVNL | 9.129 | 0.74 | 0.38 | 387 |
| KYKETWLWL | 9.125 | 0.75 | 0.33 | 388 |
| YIKSVSLSS | 8.873 | 1.34 | 0.38 | 389 |
| HLADRB*0401 | | | | |
| VIKMVSVLT | 7.488 | 32.51 | 0.38 | 390 |
| VNLMTLIRN | 7.398 | 39.99 | 0.33 | 391 |
| QKLPASSMH | 7.377 | 41.98 | 0.29 | 392 |
| QSQQNVNMI | 7.264 | 54.45 | 0.33 | 393 |
| TMLNQSPLK | 7.24 | 57.54 | 0.29 | 394 |
| HLADRB*0701 | | | | |
| HPKTLKIVW | 7.875 | 13.34 | 0.38 | 395 |
| HFVGTMGKA | 7.665 | 21.63 | 0.38 | 396 |
| TTVLTVHLS | 7.6 | 25.12 | 0.38 | 397 |
| KIVQLSKLS | 7.574 | 26.67 | 0.38 | 398 |
| VAVQIEVNS | 7.536 | 29.11 | 0.38 | 399 |

RNA Polymerase b

| | | | | |
|---|---|---|---|---|
| HLADRB1*0101 | | | | |
| YLKMWANCL | 9.609 | 0.25 | 0.33 | 400 |
| YLKSKAYLF | 9.361 | 0.44 | 0.38 | 401 |
| FVSDLAVEL | 9.357 | 0.44 | 0.33 | 402 |
| YLSTLCLEY | 9.278 | 0.53 | 0.29 | 403 |
| FVTLKASSN | 9.227 | 0.59 | 0.38 | 404 |
| HLADRB*0401 | | | | |
| DHPMASGLM | 7.42 | 38.02 | 0.29 | 405 |
| VELSISVKQ | 7.371 | 42.56 | 0.38 | 406 |
| VTLKASSNF | 7.363 | 43.35 | 0.25 | 407 |
| WVNAALNVS | 7.353 | 44.36 | 0.38 | 408 |
| DYLSTLCLE | 7.345 | 45.19 | 0.25 | 409 |
| HLADRB*0701 | | | | |
| LAVELSISV | 7.687 | 20.56 | 0.38 | 410 |
| KQSFLSVKY | 7.637 | 23.07 | 0.38 | 411 |
| FVSDLAVEL | 7.595 | 25.41 | 0.38 | 412 |
| PRSRLEVFY | 7.584 | 26.06 | 0.38 | 413 |
| FISRNVLFS | 7.578 | 26.42 | 0.38 | 414 |

Other Viral Protein

| | | | | |
|---|---|---|---|---|
| HLADRB1*0101 | | | | |
| LITTKIRKY | 9.01 | 0.98 | 0.38 | 415 |
| YYHSKTDNP | 8.943 | 1.14 | 0.33 | 416 |
| HYDEGIARS | 8.588 | 2.58 | 0.33 | 417 |
| KIVNTICQE | 8.47 | 3.39 | 0.33 | 418 |
| KYKVFSIPV | 8.384 | 4.13 | 0.38 | 419 |
| HLADRB*0401 | | | | |
| EVLPNSDNT | 7.398 | 39.99 | 0.25 | 420 |
| VRSTNGVLK | 7.173 | 67.14 | 0.38 | 421 |
| YGCADSGLR | 7.139 | 72.61 | 0.33 | 422 |
| VYDVNSCIN | 7.081 | 82.99 | 0.33 | 423 |
| GVLKIVNTI | 6.951 | 111.94 | 0.25 | 424 |
| HLADRB*0701 | | | | |
| YDVNSCINA | 7.643 | 22.75 | 0.33 | 425 |
| KIVNTICQE | 7.595 | 25.41 | 0.38 | 426 |
| LFVLTVDGH | 7.581 | 26.24 | 0.38 | 427 |
| IPVKTQVII | 7.532 | 29.38 | 0.38 | 428 |
| PVKTQVIII | 7.323 | 47.53 | 0.38 | 429 |

NP_058538.1, NP_058536.1, NP_058528.1, NP_058537.1 >RGSV SEQ ID NO: 440
MALLQKLGSSKVSSKRMSPAMIPLDSINQDLVDPQQEKDAKNKKEGKKKDLDVSMDPLTGKLPLGKKKQVDTGGIAYLENALMQLDLHD
FSFDSIRPRTKTFHMKRQHFKISTVNSRFRLDVEKTGLFSKTLKYSRICTLCLAFLGIKNRAQGTISFTFRDLSYLSENDQIDFKVKNRISKSF
SAIASFPAPIFNDDLGNLICDFEIENASVNGVVIGDLLVLLGIEQSDLPVCYEPQKAKIFEYKPLTEKGLNKISNFAGYVDNVLKAAINHREGE
DDGFSTEGLGVLVHPRVKQIDNSIPIKSLENKPQKMLMRDGSYLDVNPMGKVQFGDGHWANNKEWSELLSEIFSKIRASIDGFANATADL
AAGLEYQAFNPEKILRKLIASSTSLDDFVKDMRDLLVARYTRGTSFLFNAKNSIEKAKDKKKAEAIQVLINRYGVKKNAGDNAVDQATLGR
ISQVLAYMALRVALQITDYHKPIIPLRPISTVDIKNAIIDVVPQFLYLKADQLDSKTNSEAALYVIHLCYQVCVSERIMTKAQKDKHSVHTKS
AMITHCMGFVNLAMDNSSVVSDDKIAGRRMISGPWGLQETALDATGCACIIDVVDFCCRGHKVTDAVAPVRLFRLAIECIKDTADLKDAG
VKLKTLVDKMNTNCQFSNISYLHNMNNEIVGVERFKYNDVEYDINGSLVDCFYKGAETIPTPSPNLKCFFNALCLCLRVESKDYIKVMNKL
RNQYYAMSIWTASELKELLRELDPNDSYMATYYSIIHVSICLDICICIHDETWDSHCKTFGDRSKLMIHMKLESRHYEAIDDPTYDYFELSS
VLGGYLGSLDDDIDLPSMIELKVETKPLGDVFTERGQWYNSLASLAESNLHQQVPQFNCNLFSSIVRLKKYSRQQEVAMLSLNLGMTIEVR
LLSYHENLYSLEGGFKCVGPGNGLLELIYDGSTNKWFFLKISGLLEVDQNYQVLEKVHDLESLIRQLTQSFVQPSNWYSNKLKMIEKCKTIF
PQRREVDYEPFLNKNKLLSLCFLSKELENLLTILLVDNDMVNVGTILKPKIYKYWGQNPELTKKQKHFLLDSEGNLWGAVKSGLPVTVLR
DDQYDKDFPTLSFSRKTAEFLFTSYDDDIQKLTNPEHSGYDESMYGLYEMHPRLKVPETSEIVSPDETEIVISFENRFGNRKYHDFPSIPDN
RAYSCKISTVKNIVHDFTFALFGDDLDVSFTDAGLFIPGDPDNNKTPDMIIKHGEKHYSVIEFTTRNTNMRPDVRSRGWEDKTLKYRDAI
HNRRDHFKISIDYYIIVVCQNGVQTNLMNLPTETMDELIYRYKLARQIALQIEQNLEYDIKADQAMKMEISSIKKIIEGIRIHKEDGELDPSK
FIKPYTMAHYTKAVGTLESEDYDYLHKLDTYVSNKSMRKMEKLKHLNDVNIRAYRDEIRNESILMMQSRKMEYESNFIKNEEAYRTSNE
ASVQLPMLVPKIVRVVGVSNTHEEVRNVVDEIISTSSMSSTEEAWKQGICGFMHYLYEIEDGKSDFSLAMEEPTLSTQMEDDLKKIRNKFN
RISMVFDMDDRIDLAKIGINGKKYSKDPEVLAYRNESKKPFSLFTSTDDIERFINEECLQLFTPHDQELDNSVLDLISDSLKIHGCSSQSRLL
ESLDTYLKSKAYLFTKFVSDLAVELSISVKQNCQPREFIVKRLRDFQVYVLIKSTGSDGKVFFSLLFREDQELSKIINTTFKKVSKLGDRFLYT
DFISVNYSKLVNWTRCESLMLSLYAFWREQYNIPPNIGISSIPDEDFNSDYLKMWANCLLVLLNDKHQTEEVITSTRFIHMEAFVETPNW
PKPHKMFEKLSTIPRSRLEVFYIKSAIKLMECYTETPIRLDNSGPMRRWYNIKNPFVTENGSLSNFPNHDVMLSSMYLGYLKNKDEDPED
NASGQLISKILGYEDKLPRGEDKKYLGLEDPPVDQCSTHMYSISLVKRMCDSFLGRLKSETGVSDPKDYLSTLCLEYLSHEFLESFVTLKASS
NFSAEYYEYRPNENKRSRPQTVNEDLPKSESNRRNYGRSKVIEKIQTILTKKDPNEKYRLVVDLLKESLEEVEKNACLHVCIFRKNQHGGL
REIYVLNIYERIVQKCVEDLARAILSVVPSETMTHPKNKFQIPNKHNIAARKEFGDSYFTVCTSDDASKWNQGHHVSKFITILVRILPKFWH
GFIVRALQLWFHKRLFLGDDLLRLFCANDVLNTTDEKVKKVHEVFKGREVAPWMTRGMTYIETESGFMQGILHYISSLFHAIFLEDLAER
QKKQLPQMARIIQPDNESNVIIDCMESSDDSSMMISFSTKSMNDRQTFAMLLLVDRAFSLKEYYGDMLGIYKSIKSTTGTIFMMEFNIEFFF
AGDTHRPTIRWVNAALNVSEQETLIASQEEMSNTLKDILEGGGTFYHTFVTQVAQAMLHYRMYGSSVSPLWGSYCSMIKLSKDPALGYFL
MDHPMASGLMGFGYNLWKTCKQSFLSVKYADMLNLEFNTENSKRKMTPDIANLGVLSRTTTVGFGNKTKWMKMCDRMHLTDDIFDSI
EQNPRILFFHAKNAEEMQQKIAIKMRSPGVMQSLAKTNTLGRRVASSVYFISRNVLFSMSAGVETDEKRKTSIFRELLNSNSNVVSKIGQK
EAQIPGVQSLTEEPSDDFYSVEGLREGVIKMVSVLTDLTMEQSERLLSEKFGLTLDDTKLNDWFIDENKLMHKLSKGFGINIHVYISRDPEA
SFKLCHTFKCLTNSENLYFMLNPNYLLVRRQESSSMSDEHRRQIQESYKEIQSLFPEETDYLEIESNLSSLNLNMARSGINQRRRVRSQIQL
TGTEQSSTFSVYSVAKFIWFGEKDVPAHPKTLKIVWKKYKETWLWLRDTIGDTLVGSPFVSYIQLNNYLSRVSTKGRVLHFVGTMGKASS
GNVNLMTLIRNNFSNGIVFSGGFTDVIKKEKTEDYKSLLSNLTMLNQSPLKYEEKLVAMTDLIVDNKDLEYSTSMLGSKRNKLAIIQMFLR
TDPDLKFSGDYNTQDAVNLVEHHLGEFDQNLSLGGFRSLIRMGQLVEKELLDSGMGYEELEKNFEDLTINSLSASARRAYCQYIYCDRVLE
DAYQQYNKRKPTQKMLLSLELLKAEAANDPTRNWLTMIGHRIVKSSYDLMKLRDEAKYCRRDIMEKIRIGNLGLLGGYVQKQSYNREEK
KYFGPGVWRGYLHDVAVQIEVNSDQNMESYIKSVSLSSAMHLSDTIQSLKEWSREHRVGNSHYTMAYGNRDCEMLGRMFEFRRVQMSD
RDGCPIVLDPKLIIHQPFLSDSFCIDITDHSIRLLQECTGERAPYTTVLTVHLSKKDVITSELQSQQNVNMIKRLKMDDWLKDWILWRDQR
APTSLFTQMNLGQFPDLVDEKRLKSWCRELFESSLGYQKIVQLSKLSKAARDRLAHDYPESIQEDKEVCEELESMESLLTRISQAYKTIDM
TIKDEDLEHLYELARDLAEEQDEIQMEKEAVNVSLFHKMELSSVRKMDTFMGTDDLRLTMNIIKGESRQKLPASSMHYKRILQFMYDVP
DSQFPTYNPPSSRGRGRRGRGRSYMEMSKSHSDVVGTVSGLNYRLFYDMIPDRISQKLRLREITDPKTCNASKIPLVLICAAEEVSRMDIDH
DKDGYTKVQVKMPEYMKAYLEEMLSASNSTTTGISYSVFLVYMQDKCGDWITEHYLKNVHSMSKQQLHELITGIIETESSDDIEDEHYDD
LICKIPAYVYNIVLRYIDMSGLTT
NP_619743.1, NP_619742.1, NP_619740.1>PMMV SEQ ID NO: 441
MAYTVSSANQLVYLGSVWADPLELQNLCTSALGNQFQTQQARTTVQQQFSDVWKTIPTATVRFPATGFKVFRYNAVLDSLVSALLGAFD
TRNRIIEVENPQNPTTAETLDATRRVDDATVAIRASISNLMNELVRGTGMYNQALFESASGLTWATTPMALVVKDDVKISEFINLSAAEK
FLPAVMTSVKTVRISKVDKVIAMENDSLSDVNLLKGVKLVKDGYVCLAGLVVSGEWNLPDNCRGGVSVCLVDKRMQRDDEATLGSYRTS
AAKKRFAFKLIPNYSITTADAERKVWQVLVNIRGVAMEKGFCPLSLEFVSVCIVHKSNIKLGLREKITSVSEGGPVELTEAVVDEFIESVPM
ADRLRKFRNQSKKGSNKYVGKRNDNKGLNKEGKLFDKVRIGQNSESSDAESSSFMAYTQQATNAALASTLRGNNPLVNDLANRRLYESA
VEQCNAHDRRPKVNFLRSISEEQTLIATKAYPEFQITFYNTQNAVHSLAGGLRSLELEYLMMQIPYGSTTYDIGGNFAAHMFKGRDYVHCC
MPNMDLRDVMRHNAQKDSIELYLSKLAQKKKVIPPYQKPCFDKYTDDPQSVVCSKPFQHCEGVSHCTDKVYAVALHSLYDIPADEFGAA
LLRRNVHVCYAAFHFSENLLLEDSYVSLDDIGAFFSREGDMLNESEVAESTLNYTHSYSNVLKYVCKTYFPASSREVYMKEFLVTRVNTWF
CKFSRLDTFVLYRGVYHRGVDKEQFYSAMEDAWHYKKTLAMMNSERILLEDSSSVNYWFPKMKDMVIVPLEDVSLQNEGKRLARKEVM
VSKDEVYTVLNHIRTYQSKALTYANVLSFVESIRSRVIINGVTARSEWDVDKALLQSLSMTFELQTKLAMLKDDLVVQKFQVHSKSLTEYV
WDEITAAFHNCEPTIKERLINKKLITVSEKALEIKVPDLYVITHDRLVKEYKSSVEMPVLDVKKSLEEAEVMYNALSEISILKDSDKFDVDV
FSRMCNTLGVDPLVAAKVMVAVVSNESGLTLTFERPTEANVALALQPTITSKEEGSLKIVSSDVGESSIKEVVRKSEISMLGLTGNTVSDEF
QRSTEIESLQQFHMVSTETIIRKQMHAMVYTGPLKVQQCKNYLDSLVASLSAAVSNLKKIIKDTAAIDLETKEKEGVYDVCLKKWLVKPLS
KGHAWGVVMDSDYKCEVALLTYDGENIVCGETWRRVAVSSESLVYSDMGKIRAIRSVLKDGEPHISSAKVTLVDGVPGCGKTKEILSRVNF
DEDLVLVPGKQAAEMIRRRANSSGLIVATKENVRTVDSFLMNYGRGPCQYKRLFLDEGLMLHPGCVNELVGMSLCSEAFVYGDTQQIPYI
NRVATFPYPKHLSQLEVDAVETRRTTLRCPADITFELNQKYEGQVMCTSSVTRSVSHEVIQGAAVMNPVSKPLKGKVITFTQSDKSLLLSR
GYEDVHTVHEVQGETFEDVSLVRLTPTPVGIISKQSPHLLVSLSRHTRSIKYYTVVLDAVVSVLRDLECVSSYLLDMYKVDVSTQXQLQIES
VYKGVNLEVAAPKTGDVSDMQYYYDKCLPGNSTILNEYDAVTMQIRENSLNVKDCVLDMSKSVPLPRESETTLKPVIRTAAEKPRKPGLL
ENLVAMIKRNENSPELVGVVDIEDTASLVVDKFFDAYLIKEKKKPKNIPLLSRASLERWIEKQEKSTIGQLADFDFIDLPAVDQYRHMIKQQ
PKQRLDLSIQTEYPALQTIVYHSKKINALFGPVFSELTRQLLETIDSSRFMFYTRKTPTQIEEFFSDLDSNVPMDILELDISKYDKSQNEFHC
AVEYEIWKRLGLDDFLAEVWKHGHRKTTLKDYTAGIKTCLWYQRKSGDVTTFIGNTIIIAACLSSMLPMERLIKGAFCGDDSILYFPKGTD
FPDIQQGANLLWNFEAKLERKRYGYFCGRYIIHHDRGCIVYYDPLKLISKLGAKHIKNREHLEEFRTSLCDVAGSLNNCAYYTHLNDAVGE
VIKTAPLGSFVYRALVKYLCDKRLFQTLFLE
NP_056729.1, NP_056727.1, NP_056725.1, NP_056728.1, NP_056726.1, NP_056724.1>CMV
SEQ ID NO: 442
MENIEKLLMQEKILMLELDLVRAKISLARANGSSQQGDLSLHRETPEKEEAVHSALATFTPSQVKAIPEQTAPGKESTNPLMANILPKDM
NSVQTEIRPVKPSDFLRPHQGIPIPPKPEPSSSVAPLRDESGIQHPHTNYYVVYNGPHAGIYDDWGCTKAATNGVPGVAHKKFATITEARA
AADAYTTSQQTDRLNFIPKGEAQLKPKSFAKALTSPPKQKAHWLMLGTKKPSSDPAPKEISFAPEITMDDFLYLYDLVRKFDGEGDDTMF
TTDNEKISLENFRKNANPQMVREAYAAGLIKTIYPSNNLQEIKYLPKKVKDAVKRFRTNCIKNTEKDIFLKIRSTIPVWTIQGLLHKPRQVI
EIGVSKKVVPTESKAMESKIQIEDLTELAVKTGEQFIQSLLRLNDKKKIFVNMVEHDTLVYSKNIKDTVSEDQRAIETFQQRVISGNLLGFH
CPAICHFIVKIVEKEGGSYKCHHCDKGKAIVEDASADSGPKDGPPPTRSIVEKEDVPTTSSKQVDMSITGQPHVYKKDTHRLKPLSLNSNNR
SYVFSSSKGNIQNIINHLNNLNEIVGRSLLGIWKINSYFGLSKDPSESKSKNPSVFNTAKTIFKSGGVDYSSQLKEIKSLLEAQNTRIKSLEKAI
QSLENKIEPEPLTKEEVKELKESINSIKEGLKNIIGMDHLLLKTQTQTEQVMNVTNPNSIYIKGRLYFKGYKKIELHCFVDTGASLCIASKEVI
PEEHWVNAERPIMVKIADGSSITISKVCKDIDLIIAGEIFRIPTVYQQESGIDFIIGNNECQLYEPFIQFTDRVIFTKNKSYPVHIAKLTRAVRV
GTEGFLESMKKRSKTQQPEPVNISTNKIENPLEEIAILSEGRRLSEEKLFITQQRMQKIEELLEKVCSENPLDPNKTKQWMKASIKLSDPSK
AIKVKPMKYSPMDREEFDKQIKELLDLKVIKPSKSPHMAPAFLVNNEAEKRRGKKRMVVNYKAMNKATVGDAYNLPNKDELLTLIRGKK
IFSSEDCKSGFWQVLLDQESRPLTAFTCPQGHYEWNVVPFGLKQAPSIFQRHMDEAFRVFRKFCCVYVDDILVFSNNEEDHLLHVAMILQ
KCNQHGIILSKKKAQLFKKKINFLGLEIDEGTHKPQGHILEHINKFPDTLEDKKQLQRFLGILTYASDYIPKLAQIRKPLQAKLKENVPWRW
TKEDTLYMQKVKKNLQGFPPLHHPLPEEKLIIETDASDDYWGGMLKAIKINEGTNTELICRYASGSFKAAEKNYHSNDKETLAVINTIKKF
SIYLTPVHFLIRTDNTHFKSFVNLNYKGDSKLGRNIRWQAWLSHYSFDVEHIKGTDNHFADFLSREFNKVNSMANLNQIQKEVSEILSDQ
KSMKADIKAILELLGSQNPIKESLETVAAKIVNDLTKLINDCPCNKEILEALGTQPKEQUEQPKEKGKGLNLGKYSYPNYGVGNEELGSSGN
PKALTWPFKAPAGWPNQFMDLYPEENTQSEQSQNSENNMQIFKSENSDGFSSDLMISNDQLKNISKTQLTLEKEKIFKMPNVLSQVMKK
AFSRKNEILYCVSTKELSVDIHDATGKVYLPLITKEEINKRLSSLKPEVRKTMSMVHLGAVKILLKAQFRNGIDTPIKIALIDDRINSRRDCLL
GAAKGNLAYGKFMFTVYPKFGISLNTQRLNQTLSLIHDFENKNLMNKGDKVMTITYVVGYALTNSHHSIDYQSNATIELEDVFQEIGNVQ
QSEFCTIQNDECNWAIDIAQNKALLGAKTKTQIGNNLQIGNSASSSNTENELARVSQNIDLLKNKLKEICGE
NP_604483.1, NP_604479.1, NP_604477.1, NP_604480.1, NP_604478.1 >BBTV SEQ ID NO: 443
MARYVVCWMFTINNPTTLPVMRDEIKYMVYQVERGQEGTRHVQGYVEMKRRSSLKQMRGFFPGAHLEKRKGSQEEARSYCMKEDTRIE
GPFEFGSFKLSCNDNLFDVIQDMRETHKRPLEYLYDCPNTFDRSKDTLYRVQAEMNKTKAMNSWRTSFSAWTSEVENIMAQPCHRRII
WVYGPNGGEGKTTYAKHLMKTRNAFYSPGGKSLDICRLYNYEDIVIFDIPRCKEDYLNYGLLEEFKNGIIQSGKYEPVLKIVEYVEVIVMAN
FLPKEGIFSEDRIKLVSCMDWAESQFKTCTHGCDWKKISSDSADNRQYVPCVDSGAGRKSPRKVLLRSIEAVFNGSFSGNNRNVRGFLYVS
IRDDDGEMRPVLIVPFGGYGYHNDFYYFEGKGKVECDISSDYVAPGIDWSRDMEVSISNSNNCNELCDLKCYVVCSLRIKEMFRQEMARYP
KKSIKKRRVGRRKYGSKAATSHDYSSSGSILVPENTVKVFRIEPTDKTLPRYFIWKMFMLLVCKVKPGRILHWAMIKSSWEINQPTTCLEA
PGLFIKPEHSHLVKLVCSGELEAGVATGTSDVECLLRKTTVLRKNVTEVDYLYLAFYCSSGVSINYQNRITYHVMEFWESSAMPDDVKREI
KEIYWEDRKKLLFCQKLKSYVRRILVYGDQEDALAGVKDMKTSIIRYSEYLKKPCVVICCVSNKSIVYRLNSMVFFYHEYLEELGGDYSVYQ
DLYCDEVLSSSSTEEEDVGVIYRNVIMASTQEKFSWSDCQQIVISDYDVTLLMALTTERVKLFFEWFLFFGAIFIAITILYILLVLLFEVPRYIK
ELVRCLVEYLTRRRVWMQRTQLTEATGDVEIGRGIVEDRRDQEPAVIPHVSQVIPSQPNRRDDQGRRGNAGPMF
AAL40183.1>Calpain SEQ ID NO: 444
MPTVISASVAPRTAAEPRSPGPVPHPAQSKATEAGGGNPSGIYSAIISRNEPHGVKEKTFEQLHKKCLEKKVLYVDPEFPPDETSLFYSQKF
PIQFVWKRPPEICENPRFIIDGANRTDICQGELGDCWFLAAIACLTLNQHLLFRVIPHDQSFIENYAGIFHFQFWRYGEWVDVVIDDCLPTY
NNQLVFTKSNHRNEFWSALLEKAYAKLHGSYEALKGGNTTEAMEDFTGGVAEFFEIRDAPSDMYKIMKKAIERGSLMGCSIDDGTNMTY
GTSPSGLNMGELIARMVRNMDNSLLQDSDLDPRGSDERPTRTIIPVQYETRMACGLVRGHAYSVTGLDEVPFKGEKVKLVRLRNPWGQV
EWNGSWSDRWKDWSFVDKDEKARLQHQVTEDGEFWMSYEDFIYHFTKLEICNLTADALQSDKLQTWTVSVNEGRWVRGCSAGGCRN
FPDTFWTNPQYRLKLLEEDDDPDDSEVICSFLVALMQKNRRKDRKLGASLFTIGFAIYEVPKEMHGNKQHLQKDFFLYNASKARSKTYIN
MREVSQRFRLPPSEYVIVPSTYEPHQEGEFILRVFSEKRNLSEEVENTISVDRPVKKKKTKPIIFVSDRANSNKELGVDQESEEGKGKTSPD
KQKQSPQPQPGSSDQESEEQQQFRNIFKQIAGDDMEICADELKKVLNTVVNKHKDLKTHGFTLESCRSMIALMDTDGSGKLNLQEFHHL
WNKIKAWQKIFKHYDTDQSGTINSYEMRNAVNDAGEHLNNQLYDIITMRYADKHMNIDEDSFICCFVRLEGMFRAFHAFDKDGDGIIKL
NVLEWLQLTMYA
NP_150634.1>Caspase1 SEQ ID NO: 445
MADKVLKEKRKLFIRSMGEGTINGLLDELLQTRVLNKEEMEKVKRENATVMDKTRALIDSVIPKGAQACQICITYICEEDSYLAGTLGLSA
DQTSGNYLNMQDSQGVLSSFPAPQAVQDNPAMPTSSGSEGNVKLCSLEEAQRIWKQKSAEIYPIMDKSSRTRLALIICNEEFDSIPRRTGA
EVDITGMTMLLQNLGYSVDVKKNLTASDMTTELEAFAHRPEHKTSDSTELVFMSHGIREGICGKKHSEQVPDILQLNAIFNMLNTKNCPS
LKDKPKVIIIQACRGDSPGVVWFKDSVGVSGNLSLPTTEEFEDDAIKKAHIEKDFIAFCSSTPDNVSWRHPTMGSVFIGRLIEHMQEYACSC
DVEEIFRKVRFSFEQPDGRAQMPTTERVTLTRCFYLFPGH
NP_001158286.1>Caspase 2 SEQ ID NO: 446
MWRRKHPRTSGGTRGVLSGNRGVEYGSGRGHLGTFEGRWRKLPKMPEAVGTDPSTSRKMAELEEVTLDGKPLQALRVTDLKAALEQR
GLAKSGQKSALVKRLKGALMLENLQKHSTPHAAFQPNSQIGEEMSQNSFIKQYLEKQQELLRQRLEREAREAAELEEASAESEDEMIHPE
GVASLLPPDFQSSLERPELELSRHSPRKSSSISEEKGDSDDEKPRKGERRSSRVRQARAAKLSEGSQPAEEEEDQETPSRNLRVRADRNLKT
EEEEEEEEEEEEDDEEEEGDDEGQKSREAPILKEFKEEGEEIPRVKPEEMMDERPKTRSQEQEVLERGGRFTRSQEEARKSHLARQQQEK
EMKTTSPLEEEEREIKSSQGLKEKSKSPSPPRLTEDRKKASLVALPEQTASEEETPPPLLTKEASSPPPHPQLHSEEEIEPMEGPAPPVLIQL
SPPNTDADTRELLVSQHTVQLVGGLSPLSSPSDTKAESPAEKVPEESVLPLVQKSTLADYSAQKDLEPESDRSAQPLPLKIEELALAKGITE
ECLKQPSLEQKEGRRASHTLLPSHRLKQSADSSSSRSSSSSSSSSRSRSRSPDSSGSRSHSPLRSKQRDVAQARTHANPRGRPKMGSRSTSES
RSRSRSRSRSASSNSRKSLSPGVSRDSSTSYTETKDPSSGQEVATPPVPQLQVCEPKERTSTSSSSVQARRLSQPESAEKHVTQRLQPERGSP
KKCEAEEAEPPAATQPQTSETQTSHLPESERIHHTVEEKEEVTMDTSENRPENDVPEPPMPIADQVSNDDRPEGSVEDEEKKESSLPKSF
KRKISVVSTKGVPAGNSDTEGGQPGRKRRWGASTATTQKKPSISITTESLKEAVVDLHADDSRISEDETERNGDDGTHDKGLKICRTVTQV
VPAEGQENGQREEEEEEKEPEAEPPVPPQVSVEVALPPPAEHEVKKVTLGDTLTRRSISQQKSGVSITIDDPVRTAQVPSPPRGKISNIVHIS
NLVRPFTLGQLKELLGRTGTLVEEAFWIDKIKSHCFVTYSTVEEAVATRTALHGVKWPQSNPKFLCADYAEQDELDYHRGLLVDRPSETK
TEEQGIPRPLHPPPPPPVQPPQHPRAEQREQERAVREQWAEREREMERRERTRSEREWDRDKVREGPRSRSRSRDRRRKERAKSKEKK
SEKKEKAQEEPPAKLLDDLERKTKAAPCIYWLPLTDSQIVQKEAERAERAKEREKRRKEQEEEEQKEREKEAERERNRQLEREKRREHS
RERDRERERERERDRGDRDRDRERDRERGRERDRRDTKRHSRSRSRSTPVRDRGGRR
NP_004337.2>Caspase3 SEQ ID NO: 447
MENTENSVDSKSIKNLEPKIIHGSESMDSGISLDNSYKMDYPEMGLCIIINNKNFHKSTGMTSRSGTDVDAANLRETERNLKYEVRNKNDL
TREEIVELMRDVSKEDHSKRSSFVCVLLSHGEEGIIFGTNGPVDLKKITNFFRGDRCRSLTGKPKLFIIQACRGTELDCGIETDSGVDDDMA
CHKIPVEADFLYAYSTAPGYYSWRNSKDGSWFIQSLCAMLKQYADKLEFMHILTRVNRKVATEFESFSFDATFHAKKQIPCIVSMLTKELY
FYH
NP_001216.1>Caspase4 SEQ ID NO: 448
MAEGNHRKKPLKVLESLGKDFLTGVLDNLVEQNVLNWKEEEKKKYYDAKTEDKVRVMADSMQEKQRMAGQMLLQTFFNIDQISPNKK
AHPNMEAGPPESGESTDALKLCPHEEFLRLCKERAEEIYPIKERNNRTRLALIICNTEFDHLPPRNGADFDITGMKELLEGLDYSVDVEEN
LTARDMESALRAFATRPEHKSSDSTFLVLMSHGILEGICGTVHDEKKPDVLLYDTIFQIFNNRNCLSLKDKPKVIIVQACRGANRGELWVR
DSPASLEVASSQSSENLEEDAVYKTHVEKDFIAFCSSTPHNVSWRDSTMGSIFITQLITCFQKYSWCCHLEEVFRKVQQSFETPRAKAQMP
TIERLSMTRYFYLFPGN
NP_004338.3>Caspase5 SEQ ID NO: 449
MAEDSGKKKRRKNFEAMFKGILQSGLDNFVINHMLKNNVAGQTSIQTLVPNTDQKSTSVKKDNHKKKTVKMLEYLGKDVLHGVFNYLA
KHDVLTLKEEEKKKYYDTKIEDKALILVDSLRKNRVAHQMFTQTLLNMDQKITSVKPLLQIEAGPPESAESTNILKLCPREEFLRLCKKNH
DEIYPIKKREDRRRLALIICNTKEDHLPARNGAHYDIVGMKRLLQGLGYTVVDEKNLTARDMESVLRAFAARPEHKSSDSTFLVLMSHGIL
EGICGTAHKKKKPDVLLYDTIFQIENNRNCLSLKDKPKVIIVQACRGEKHGELWVRDSPASLALISSQSSENLEADSVCKIHEEKDFIAFCSS
TPHNVSWRDRTRGSIFITELITCFQKYSCCCHLMEIFRKVQKSFEVPQAKAQMPTIERATLTRDFYLFPGN
AAD24962.1>Caspase8 SEQ ID NO: 450
MDFSRNLYDIGEQLDSEDLASLKELSLDYIPQRKQEPIKDALMLFQRLQEKRMLEESNLSFLKELLFRINRLDLLITYLNTRKEEMERELQT
PGRAQISAYRVMLYQISEEVSRSELRSFKFLLQEEISKCKLDDDMNLLDIFIEMEKRVILGEGKLDILKRVCAQINKSLLKIINDYEEFSKERSS
SLEGSPDEFSNGEELCGVMTISDSPREQDSESQTLDKVYQMKSKPRGYCLIINNHNFAKAREKVPKLHSIRDRNGTHLDAGALTTTFEELH
FEIKPHDDCTVEQIYDILKIYQLMDHSNMDCFICCILSHGDKGIIYGTDGQEPPIYELTSQFTGLKCPSLAGKPKVFFIQACQGDNYQKGIPVE
TDSEEQPYLEMDLSSPQTRYIPDEADFLLGMATVNNCVSYRNPAEGTWYIQSLCQSLRERCPRGDDILTILTEVNYEVSNKDDKKNMGKQ
MPQPTFTLRKKLVFPSD
NP_116759.2>Caspase10 SEQ ID NO: 451
MKSQGQHWYSSSDKNCKVSFREKLLIIDSNLGVQDVENLKFLCIGLVPNKKLEKSSSASDVFEHLLAEDLLSEEDPFFLAELLYIIRQKKLLQ
HLNCTKEEVERLLPTRQRVSLERNLLYELSEGIDSENLKDMIFLLKDSLPKTEMTSLSFLAFLEKQGKIDEDNLTCLEDLCKTVVPKLLRNI
EKYKREKAIQIVTPPVDKEAESYQGEEELVSQTDVKTFLEALPQESWQNKHAGSNGNRATNGAPSLVSRGMQGASANTLNSETSTKRAA
VYRMNRNHRGLCVIVNNHSFTSLKDRQGTHKDAEILSHVFQWLGFTVHIHNNVTKVEMEMVLQKQKCNPAHADGDCFVFCILTHGRFG
AVYSSDEALIPIREIMSHETALQCPRLAEKPKLFFIQACQGEEIQPSVSIEADALNPEQAPTSLQDSIPAEADFLLGLATVPGYVSFRHVEEGS
WYIQSLCNHLKKLVPRHEDILSILTAVNDDVSRRVDKQGTKKQMPQPAFTLRKKLVFPVPLDALSL
NP_001020330.1>CD74 SEQ ID NO: 452
MHRRRSRSCREDQKPVMDDQRDLISNNEQLPMLGRRPGAPESKCSRGALYTGFSILVTLLLAGQATTAYFLYQQQGRLDKLTVTSQNLQL
ENLRMKLPKPPKPVSKMRMATPLLMQALPMGALPQGPMQNATKYGNMTEDHVMHLLQNADPLKVYPPLKGSFPENLRHLKNTMETI
DWKVFESWMHHWLLFEMSRHSLEQKPTDAPPKVLTKCQEEVSHIPAVHPGSFRPKCDENGNYLPLQCYGSIGYCWCVFPNGTEVPNTR
SRGHHNCSESLELEDPSSGLGVTKQDLGPVPM
CAG33019.1>FADD SEQ ID NO: 453
MDPFLVLLHSVSSSLSSSELTELKFLCLGRVGKRKLERVQSGLDLFSMLLEQNDLEPGHTELLRELLASLRRHDLLRRVDDFEAGAAAGAA
PGEEDLCAAFNVICDNVGKDWRRLARQLKVSDTKIDSIEDRYPRNLTERVRESLRIWKNTEKENATVAHLVGALRSCQMNLVADLVQEV
QQARDLQNRSGAMSPMSWNSDASTSEAS
AAH12479.1>Fas SEQ ID NO: 454
MLGIWTLLPLVLTSVARLSSKSVNAQVTDINSKGLELRKTVTTVETQNLEGLHHDGQFCHKPCPPGERKARDCTVNGDEPDCVPCQEGKE
YTDKAHESSKCRRCRLCDEGHGLEVEINCTRTQNTKCRCKPNFECNSTVCEHCDPCTKCEHGIIKECTLTSNTKCKEEGSRSNLGWLCLLL
LPIPLIVWVKRKEVQKTCRKHRKENQGSHESPTLNPETVAINLSDVDLSKYITTIAGVMTLSQVKGEVRKNGVNEAKIDEIKNDNVQDTAE
QKVQLLRNWHQLHGKKEAYDTLIKDLKKANLCTLAEKIQTIILKDITSDSENSNFRNEIQSLV
AAO43991.1>FasL SEQ ID NO: 455
MQQPFNYPYPQIYWVDSSASSPWAPPGTVLPCPTSVPRRPGQRRPPPPPPPPPLPPPPPPPPLPPLPLPPLKKRGNHSTGLCLLVMFFMV
LVALVGLGLGMFQLFHLQKELAELRESTSQMHTASSLEKQIGHPSPPPEKKELRKVAHLTGKSNSRSMPLEWEDTYGIVLLSGVKYKKGG
LVINETGLYFVYSKVYFRGQSCNNLPLSHKVYMRNSKYPQDLVMMEGKMMSYCTTGQMWARSSYLGAVFNLTSADHLYVNVSELSLVNF
EESQTFFGLYKL
AAA75490.1>GranB SEQ ID NO: 456
MQPILLLLAFLLLPRADAGEIIGGHEAKPHSRPYMAYLMIWDQKSLKRCGGFLIQDDFVLTAAHCWGSSINVTLGAHNIKEQEPTQQFIPV
KRAIPHPAYNPKNFSNDIMLLQLERKAKRTRAVQPLRLPSNKAQVKPGQTCSVAGWGQTAPLGKHSHTLQEVKMTVQEDRKCESDLRH
YYDSTIELCVGDPEIKKTSFKGDSGGPLVCNKVAQGIVSYGRNNGMPPRACTKVSSFVHWIKKTMKRY
NP_003795.2>Rip1 SEQ ID NO: 457
MQPDMSLNVIKMKSSDFLESAELDSGGFGKVSLCFHRTQGLMIMKTVYKGPNCIEHNEALLEEAKMMNRLRHSRVVKLLGVIIEEGKYSL
VMEYMEKGNLMHVLKAEMSTPLSVKGRIILEHEGMCYLHGKGVIHKDLKPENILVDNDFHIKIADLGLASFKMWSKLNNEEHNELREVD
GTAKKNGGTLYYMAPEHLNDVNAKPTEKSDVYSFAVVLWAIFANKEPYENAICEQQLIMCIKSGNRPDVDDITEYCPREIISLMKLCWEA
NPEARPTFPGIEEKFRPFYLSQLEESVEEDVKSLKKEYSNENAVVKRMQSLQLDCVAVPSSRSNSATEQPGSLHSSQGLGMGPVEESWFAP
SLEHPQEENEPSLQSKLQDEANYHLYGSRMDRQTKQQPRQNVAYNREEERRRRVSHDPFAQQRPYENFQNTEGKGTAYSSAASHGNAV
HQPSGLTSQPQVLYQNNGLYSSHGEGTRPLDPGTAGPRVWYRPIPSHMPSLHNIPVPETNYLGNTPTMPFSSLPPTDESIKYTIYNSTGIQI
GAYNYMEIGGTSSSLLDSTNTNEKEEPAAKYQAIEDNTTSLTDKHLDPIRENLGKHWKNCARKLGFTQSQIDEIDHDYERDGLKEKVYQM
LQKWVMREGIKGATVGKLAQALHQCSRIDLLSSLIYVSQN
NP_003812.1>Rip2 SEQ ID NO: 458
MNGEAICSALPTIPYHKLADLRYLSRGASGTVSSARHADWRVQVAVKHLHIHTPLLDSERKDVLREAEILHKARFSYILPILGICNEPEFLGI
VTEYMPNGSLNELLHRKTEYPDVAWPLRFRILHEIALGVNYLHNMTPPLLHHDLKTQNILLDNEFHVKIADEGLSKWRMMSLSQSRSSK
SAPEGGTIIYMPPENYEPGQKSRASIKHDIYSYAVITWEVLSRKQPFEDVTNPLQIMYSVSQGHRPVINEESLPYDIPHRARMISLIESGWAQ
NPDERPSFLKCLIELEPVLRTFEEITFLEAVIQLKKTKLQSVSSAIHLCDKKKMELSLNIPVNHGPQEESCGSSQLHENSGSPETSRSLPAPQ
DNDELSRKAQDCYFMKLHHCPGNHSWDSTISGSQRAAFCDHKTTPCSSAIINPLSTAGNSERLQPGIAQQWIQSKREDIVNQMTEACLNQ
SLDALLSRDLIMKEDYELVSTKPTRTSKVRQLLDTTDIQGEEFAKVIVQKLKDNKQMGLQPYPEILVVSRSPSLNLLQNKSM
NP_006862.2>Rip3 SEQ ID NO: 459
MSCVKLWPSGAPAPLVSIEELENQELVGKGGFGTVFRAQHRKWGYDVAVKIVNSKAISREVKAMASLDNEFVLRLEGVIEKVNWDQDPK
PALVTKFMENGSLSGLLQSQCPRPWPLLCRLLKEVVLGMFYLHDQNPVLLHRDLKPSNVLLDPELHVKLADEGLSTFQGGSQSGTGSGEP
GGTLGYLAPELFVNVNRKASTASDVYSEGILMWAVLAGREVELPTEPSLVYEAVCNRQNRPSLAELPQAGPETPGLEGLKELMQLCWSSE
PKDRPSFQECLPKTDEVFQMVENNMNAAVSTVKDELSQLRSSNRRESIPESGQGGTEMDGFRRTIENQHSRNDVMVSEWLNKLNLEEPP
SSVPKKCPSLTKRSRAQEEQVPQAWTAGTSSDSMAQPPQTPETSTFRNQMPSPTSTGTPSPGPRGNQGAERQGMNWSCRTPEPNPVTG
RPLVNIYNCSGVQVGDNNYLTMQQTTALPTWGLAPSGKGRGLQHPPPVGSQEGPKDPEAWSRPQGWYNHSGK
NP_008850.1>SerpinB3 SEQ ID NO: 460
MNSLSEANTKFMFDLFQQFRKSKENNIFYSPISITSALGMVLLGAKDNTAQQIKKVLHFDQVTENTTGKAATYHVDRSGNVHHQFQKLLT
EFNKSTDAYELKIANKLFGEKTYLFLQEYLDAIKKFYQTSVESVDFANAPEESRKKINSWVESQTNEKIKNLIPEGNIGSNTTLVLVNAIYFK
GQWEKKFNKEDTKEEKFWPNKNTYKSIQMMRQYTSFHFASLEDVQAKVLEIPYKGKDLSMIVLLPNEIDGLQKLEEKLTAEKLMEWTSL
QNMRETRVDLHLPRFKVEESYDLKDTLRTMGMVDIFNGDADLSGMTGSRGLVLSGVLHKAFVEVTEEGAEAAAATAVVGFGSSPTSTNE
EFHCNHPFLFFIRQNKTNSILFYGRFSSP
NP_002965.1>SerpinB4 SEQ ID NO: 461
MNSLSEANTKFMFDLFQQFRKSKENNIFYSPISITSALGMVLLGAKDNTAQQISKVLHEDQVTENTTEKAATYHVDRSGNVHHQFQKLLT
EFNKSTDAYELKIANKLFGEKTYQFLQEYLDAIKKEYQTSVESTDFANAPEESRKKINSWVESQTNEKIKNLEPDGTIGNDTTLVLVNAIYF
KGQWENKFKKENTKEEKEWPNKNTYKSVQMMRQYNSENFALLEDVQAKVLEIPYKGKDLSMIVLLPNEIDGLQKLEEKLTAEKLMEWT
SLQNMRETCVDLHLPRFKMEESYDLKDTLRTMGMVNIENGDADLSGMTWSHGLSVSKVLHKAFVEVTEEGVEAAAATAVVVVELSSPS
TNEEFCCNHPFLFFIRQNKTNSILFYGRFSSP
NP_004146.1>SerpinB9 SEQ ID NO: 462
METLSNASGTFAIRLLKILCQDNPSHNVFCSPVSISSALAMVLLGAKGNTATQMAQALSLNTEEDIHRAFQSLLTEVNKAGTQYLLRTANR
LFGEKTCQFLSTEKESCLQFYHAELKELSFIRAAEESRKHINTWVSKKTEGKIEELLPGSSIDAETRLVLVNAIYFKGKWNEPFDETYTREM
PFKINQEEQRPVQMMYQEATFKLAHVGEVRAQLLELPYARKELSLLVLLPDDGVELSTVEKSLTFEKLTAWTKPDCMKSTEVEVLLPKFK
LQEDYDMESVLRHLGIVDAFQQGKADLSAMSAERDLCLSKFVHKSFVEVNEEGTEAAAASSCFVVAECCMESGPRECADHPFLFFIRHNR
ANSILFCGRFSSP
NP_005015.1 >SerpinB10 SEQ ID NO: 463
MDSLATSINQFALELSKKLAESAQGKNIFFSSWSISTSLTIVYLGAKGTTAAQMAQVLQFNRDQGVKCDPESEKKRKMEENLSNSEEIHSD
FQTLISEILKPNDDYLLKTANAIYGEKTYAFHNKYLEDMKTYFGAEPQPVNEVEASDQIRKDINSWVERQTEGKIQNLLPDDSVDSTTRMI
LVNALYFKGIWEHQFLVQNTTEKPFRINETTSKPVQMMFMKKKLHIFHIEKPKAVGLQLYYKSRDLSLLILLPEDINGLEQLEKAITYEKL
NEWTSADMMELYEVQLHLPKFKLEDSYDLKSTLSSMGMSDAFSQSKADFSGMSSARNLFLSNVFHKAFVEINEQGTEAAAGSGSEIDIRIR
VPSIEFNANHPFLFFIRHNKTNTILFYGRLCSP
BORFE2 SEQ ID NO: 464
MVTRDVLLAIETHLNQNEKTFVMYELLDPYIPKECEDFLPTLENLHSKRKIIYPILIELMYILQRFDLLRSIFLLDHRFVKDQITSSHWNYISP
YKQLIFSIGQNIDDEDLISIKFISMNYIGKSPSKIKNYLDWVRALEKVAMVGPDNLDLFETLFKQIHRMDIVKMIKNYRTRETLQITL
CrmA SEQ ID NO: 465
MDIFREIASSMKGENVFISPPSISSVLTILYYGANGSTAEQLSKYVEKEADKNKDDISFKSMNKVYGRYSAVFKDSFLRKIGDNFQTVDFTDC
RTVDAINKCVDIFTEGKINPLLDEPLSPDTCLLAISAVYFKAKWLMPFEKEFTSDYPFYVSPTEMVDVSMMSMYGEAFNHASVKESFGNFS
IIELPYVGDTSMVVILPDNIDGLESIEQNLTDTNFKKWCDSMDAMFIDVHIPKFKVTGSYNLVDALVKLGLTEVFGSTGDYSNMCNSDVSV
DAMIHKTYIDVNEEYTEAAAATCALVADCASTVTNEFCADHPFIYVIRHVDGKILFVGRYCSPTTNMHQKRTAMFQDPQERPRKLPQLCT
ELQTTIHDIILECVYCKQQLLRREVYDFAFRDLCIVYRDGNPYAVCDKCLKFYSKISEYRHYCYSLYGTTLEQQYNKPLCDLLIRCINCQKPL
CPEEKQRHLDKKQRFHNIRGRWTGRCMSCCRSSRTRRETQL
314.7kDA1 SEQ ID NO: 466
MSNGAADRARLRHLDHCRQPHCFARDICVFTYFELPEEHPQGPAHGVRITVEKGIDTHLIKFFTKRPLLVEKDQGNTILTLYCICPVPGLH
EDFCCHLCAEFNHL
E314.7kDA2 SEQ ID NO: 467
MKISAVICVLNLIICSGAVPPEEEPNCHPHLSNIKINLSIPHITLRCSFFSTHLTWTFNGKHVTNTDIKFKLHKENITLFQPINLGYYRCSAPP
CTQAFFVAPVIDKRPAPTTAAVTEHITEAVSPSKGTEEIVYFSNFTNHLVLNCSCSNSLISWFANSSLCKTFYQGKLLYSAKLTLCNQSTPSH
LTLLPPEVAGRYFCIGAARTSPCQQHWNLTYCPPPVSPFVINTEYLDYNPLLAYGGLAALILFLISNLFLVQHLYSY
E314.7kDA 3 SEQ ID NO: 468
MLSIFLLFLFSLPSGLYAQTAERPLKVVVEAGHNVTLPHLSGSHQTGHVTWLVETSDYGSASPDNFIFSGQKLCQFTDRTMVWPYYNLHF
NCENYDLNLFWLKVENSAIYNVKNTVNASETNIYYDLRVVQIFPPKCIITSKYLTNDYCHITINCTNSDYPNKVVFNNVSRWYYGYGKGSP
TLPNYFITNFNVSGITKSFNHTYPFNELCDYPTSQSQHSLTHTVSTVIFLGIIGFSILIIIAAFIYLCWHRKSLCVSKTEPLMPIPY
E314.7kDA4 SEQ ID NO: 469
MKTALVLFFMLIPVWASSCQLHKPWNFLDCYTKETNYIGWVYGIMSGLVFVSSVVSLQLYARLNFSWNKYTDDLPEYPNPQDDLPLNIVF
PEPPRPPSVVSYFKFTGEDD
E314.7kDA5 SEQ ID NO: 470
MIEPDLEIDGRITEQRLLTDRARRRQQDQKNKELIDLQTVHQCKKGLFCLVKQATLRYESLPGKEHQLCYTLPTQRQTFTAMVGSVPIKVS
QQAGEQEGSIRCLCDNPECLYTLIKTLCGLRNLLPMN
K13 SEQ ID NO: 471
MATYEVLCEVARKLGTDDREVVLELLNVFIPQPTLAQLIGALRALKEEGRLTFPLLAECLFRAGRRDLLRDLLHLDPRFLERHLAGTMSYF
SPYQLTVLHVDGELCARDIRSLIFLSKDTIGSRSTPQTFLHWVYCMENLDLLGPTDVDALMSMLRSLSRVDLQRQVQTLMGLHLSGPSHS
QHYRHTP
MC159 SEQ ID NO: 472
MSDSKEVPSLPFLRHLLEELDSHEDSLLLFLCHDAAPGCTTVTQALCSLSQQRKLTLAALVEMLYVLQRMDLLKSRFGLSKEGAEQLLGTS
FLTRYRKLMVCVGEELDSSELRALRLFACNLNPSLSTALSESSRFVELVLALENVGLVSPSSVSVLADMLRTLRRLDLCQQLVEYEQQEQAR
YRYCYAASPSLPVRTLRRGHGASEHEQLCMPVQESSDSPELLRTPVQESSSDSPEQTT
p35 SEQ ID NO: 473
MCVIFPVEIDVSQTIIRDCQVDKQTRELVYINKIMNTQLTKPVLMMFNISGPIRSVTRKNNNLRDRIKSKVDEQFDQLERDYSDQMDGFHD
SIKYFKDEHYSVSCQNGSVLKSKFAKILKSHDYTDKKSIEAYEKYCLPKLVDERNDYYVAVCVLKPGFENGSNQVLSFEYNPIGNKVIVPFA
HEINDTGLYEYDVVAYVDSVQFDGEQFEEFVQSLILPSSFKNSEKVLYYNEASKNKSMIYKALEFTTESSWGKSEKYNWKIFCNGFIYDKKS
KVLYVKLHNVTSALNKNVILNTIK
Serp2 SEQ ID NO: 474
MELFKHFLQSTASDVFVSPVSISAVLAVLLEGAKGRTAAQLRLALEPRYSHLDKVTVASRVYGDWRLDIKPKFMQAVRDRFELVNFNHSP
EKIKDDINRWVAARTNNKILNAVNSISPDTKLLIVAAIYFEVAWRNQFVPDFTIEGEFWVTKDVSKTVRMMTLSDDFRFVDVRNEGIKMI
ELPYEYGYSMLVIIPDDLEQVERHLSLMKVISWLKMSTLRYVHLSFPKFKMETSYTLNEALATSGVTDIFAHPNFEDMTDDKNVAVSDIF
HKAYIEVTEFGTTAASCTYGCVTDFGGTMDPVVLKVNKPFIFIIKHDDTFSLLFLGRVTSPNY
UL39.1 SEQ ID NO: 475
MASRPAASSPVEARAPVGGQEAGGPSAATQGEAAGAPLAHGHHVYCQRVNGVMVLSDKTPGSASYRISDNNFVQCGSNCTMIIDGDVVR
GRPQDPGAAASPAPFVAVTNIGAGSDGGTAVVAFGGTPRRSAGTSTGTQTADVPTEALGGPPPPPRFTLGGGCCSCRDTRRRSAVFGGEG
DPVGPAEFVSDDRSSDSDSDDSEDTDSETLSHASSDVSGGATYDDALDSDSSSDDSLQIDGPVCRPWSNDTAPLDVCPGTPGPGADAGGPS
AVDPHAPTPEAGAGLAADPAVARDDAEGLSDPRPRLGTGTAYPVPLELTPENAEAVARFLGDAVNREPALMLEYFCRCAREETKRVPPR
TFGSPPRLTEDDFGLLNYALVEMQRLCLDVPPVPPNAYMPYYLREYVTRLVNGFKPLVSRSARLYRILGVLVHLRIRTREASFEEWLRSKE
VALDFGLTERLREHEAQLVILAQALDHYDCLIHSTPHTLVERGLQSALKYEEFYLKRFGGHYMESVFQMYTRIAGFLACRATRGMRHIALG
REGSWWEMFKFFFHRLYDHQIVPSTPAMLNLGTRNYYTSSCYLVNPQATTNKATLRAITSNVSAILARNGGIGLCVQAFNDSGPGTASVM
PALKVLDSLVAAHNKESARPTGACVYLEPWHTDVRAVLRMKGVLAGEEAQRCDNIFSALWMPDLFFKRLIRHLDGEKNVTWTLFDRDT
SMSLADFHGEEFEKLYQHLEVMGFGEQIPIQELAYGIVRSAATTGSPFVMFKDAVNRHYIYDTQGAAIAGSNLCTEIVHPASKRSSGVCNLG
SVNLARCVSRQTFDFGRLRDAVQACVLMVNIMIDSTLQPTPQCTRGNDNLRSMGIGMQGLHTACLKLGLDLESAEFQDLNKHIAEVMLLS
AMKTSNALCVRGARPFNHFKRSMYRAGRFHWERFPDARPRYEGEWEMLRQSMMKHGLRNSQFVALMPTAASAQISDVSEGFAPLFTN
LFSKVTRDGETLRPNTLLLKELERTFSGKRLLEVMDSLDAKQWSVAQALPCLEPTHPLRRFKTAFDYDQKLLIDLCADRAPYVDHSQSMT
LYVTEKADGTLPASTLVRLLVHAYKRGLKTGMYYCKVRKATNSGVFGGDDNIVCMSCAL
vICA SEQ ID NO: 476
MDDLRDTLMAYGCIAIRAGDFNGLNDFLEQECGTRLHVAWPERCFIQLRSRSALGPFVGKMGTVCSQGAYVCCQEYLHPFGFVEGPGFM
RYQLIVLIGQRGGIYCYDDLRDCIYELAPTMKDFLRHGFRHCDHFHTMRDYQRPMVQYDDYWNAVMLYRGDVESLSAEVTKRGYASYSI
DDPFDECPDTHFAFWTHNTEVMKFKETSFSVVRAGGSIQTMELMIRTVPRITCYHQLLGALGHEVPERKEFLVRQYVLVDTFGVVYGYDP
AMDAVYRLAEDVVMFTCVMGKKGHRNHRFSGRREAIVRLEKTPTCQHPKKTPDPMIMFDEDDDDELSLPRNVMTHEEAESRLYDAITE
NLMHCVKLVTTDSPLATHLWPQELQALCDSPALSLCTDDVEGVRQKLRARTGSLHHFELSYRFHDEDPETYMGFLWDIPSCDRCVRRRR
FKVCDVGRRHIIPGAANGMPPLTPPHAYMNN
UL39.2 SEQ ID NO: 477
MANRPAASALAGARSPSERQEPREPEVAPPGGDHVFCRKVSGVMVLSSDPPGPAAYRISDSSFVQCGSNCSMIIDGDVARGHLRDLEGATS
TGAFVAISNVAAGGDGRTAVVALGGTSGPSATTSVGTQTSGEFLHGNPRTPEPQGPQAVPPPPPPPFPWGHECCARRDARGGAEKDVGA
AESWSDGPSSDSETEDSDSSDEDTGSETLSRSSSIWAAGATDDDDSDSDSRSDDSVQPDVVVRRRWSDGPAPVAFPKPRRPGDSPGNPGL
GAGTGPGSATDPRASADSDSAAHAAAPQADVAPVLDSQPTVGTDPGYPVPLELTPENAEAVARFLGDAVDREPALMLEYFCRCAREESK
RVPPRTFGSAPRLTEDDFGLLNYALAEMRRLCLDLPPVPPNAYTPYHLREYATRLVNGFKPLVRRSARLYRILGVLVHLRIRTREASFEEW
MRSKEVDLDFGLTERLREHEAQLMILAQALNPYDCLIHSTPNTLVERGLQSALKYEEFYLKRFGGHYMESVFQMYTRIAGFLACRATRGM
RHIALGRQGSWWEMFKFFFHRLYDHQIVPSTPAMLNLGTRNYYTSSCYLVNPQATTNQATLRAITGNVSAILARNGGIGLCMQAFNDASP
GTASIMPALKVLDSLVAAHNKQSTRPTGACVYLEPWHSDVRAVLRMKGVLAGEEAQRCDNIFSALWMPDLFFKRLIRHLDGEKNVTWS
LFDRDTSMSLADFHGEEFEKLYEHLEAMGFGETIPIQDLAYAIVRSAATTGSPFIMFKDAVNRHYIYDTQGAAIAGSNLCTEIVHPASKRSS
GVCNLGSVNLARCVSRQTFDFGRLRDAVQACVLMVNIMIDSTLQPTPQCTRGNDNLRSMGIGMQGLHTACLKMGLDLESAEFRDLNTHI
AEVMLLAAMKTSNALCVRGARPFSHFKRSMYRAGRFHWERFSNASPRYEGEWEMLRQSMMKHGLRNSQFIALMPTAASAQISDVSEGF
APLFTNLFSKVTRDGETLRPNTLLLKELERTFGGKRLLDAMDGLEAKQWSVAQALPCLDPAHPLRRFKTAFDYDQELLIDLCADRAPYV
DHSQSMTLYVTEKADGTLPASTLVRLLVHAYKRGLKTGMYYCKVRKATNSGVFAGDDNIVCTSCAL
vIRA SEQ ID NO: 478
MDRQPKVYSDPDNGFFFLDVPMPDDGQGGQQTATTAAGGAFGVGGGHSVPYVRIMNGVSGIQIGNHNAMSIASCWSPSYTDRRRRSYPK
TATNAAADRVAAAVSAANAAVNAAAAAAAAGGGGGANLLAAAVTCANQRGCCGGNGGHSLPPTRAPKTNATAAAAPAVAVASNAKSD
NNHANAASGAGSAAATPAATTSAAAAVENRRPSPSPSTASTAPCDEGSSPRHHRPSHVSVGTQATPSTPIPIPAPRCSTGQQQQQPQAKK
LKPAKADPLLYAATMPPPASVTTAAAAAVAPESESSPAASAPPAAAAMATGGDDEDQSSFSFVSDDVLGEFEDLRIAGLPVRDEMRPPTP
TMTVIPVSRPFRAGRDSGRDALFDDAVESVRCYCHGILGNSRFCALVNEKCSEPAKERMARIRRYAADVTRCGPLALYTAIVSSANRLIQT
DPSCDLDLAECYVETASKRNAVPLSAFYRDCDRLRDAVAAFFKTYGMVVDAMAQRITERVGPALGRGLYSTVVMMDRCGNSFQGREETP
ISVFARVAAALAVECEVDGGVSYKILSSKPVDAAQAFDAFLSALCSFAIIPSPRVLAYAGFGGSNPIFDAVSYRAQFYSAESTINGTLHDICDM
VTNGLSVSVSAADLGGDIVASLHILGQQCKALRPYARFKTVLRIYFDIWSVDALKIFSFILDVGREYEGLMAFAVNTPRIFWDRYLDSSGDK
MWLMFARREAAALCGLDLKSFRNVYEKMERDGRSAITVSPVVWAVCQLDACVARGNTAVVFPHNVKSMIPENIGRPAVCGPGVSVVSGG
FVGCTPIHELCINLENCVLEGAAVESSVDVVLGLGCRFSFKALESLVRDAVVLGNLLIDMTVRTNAYGAGKLLTLYRDLHIGVVGFHAVMN
RLGQKFADMESYDLNQRIAEFIYYTAVRASVDLCMAGADPFPKFPKSLYAAGRFYPDLFDDDERGPRRMTKEFLEKLREDVVKHGIRNAS
FITGCSADEAANLAGTTPGFWPRRDNVFLEQTPLMMTPTKDQMLDECVRSVKIEPHRLHEEDLSCLGENRPVELPVLNSRLRQISKESAT
VAVRRGRSAPFYDDSDDEDEVACSETGWTVSTDAVIKMCVDRQPFVDHAQSLPVAIGFGGSSVELARHLRRGNALGLSVGVYKCSMPPSV
NYR

### Example 6

### Plant Viral Nucleic Acids

Tobacco mosaic virus (genomic DNA, Accession Number: NC_001367.1) (SEQ ID
NO: 430):
GTATTTTTACAACAATTACCAACAACAACAAACAACAAACAACATTACAATTACTATTTACAATTACAAT
GGCATACACACAGACAGCTACCACATCAGCTTTGCTGGACACTGTCCGAGGAAACAACTCCTTGGTCAAT
GATCTAGCAAAGCGTCGTCTTTACGACACAGCGGTTGAAGAGTTTAACGCTCGTGACCGCAGGCCCAAGG
TGAACTTTTCAAAAGTAATAAGCGAGGAGCAGACGCTTATTGCTACCCGGGCGTATCCAGAATTCCAAAT
TACATTTTATAACACGCAAAATGCCGTGCATTCGCTTGCAGGTGGATTGCGATCTTTAGAACTGGAATAT
CTGATGATGCAAATTCCCTACGGATCATTGACTTATGACATAGGCGGGAATTTTGCATCGCATCTGTTCA
AGGGACGAGCATATGTACACTGCTGCATGCCCAACCTGGACGTTCGAGACATCATGCGGCACGAAGGCCA
GAAAGACAGTATTGAACTATACCTTTCTAGGCTAGAGAGAGGGGGGAAAACAGTCCCCAACTTCCAAAAG
GAAGCATTTGACAGATACGCAGAAATTCCTGAAGACGCTGTCTGTCACAATACTTTCCAGACAATGCGAC
ATCAGCCGATGCAGCAATCAGGCAGAGTGTATGCCATTGCGCTACACAGCATATATGACATACCAGCCGA
TGAGTTCGGGGCGGCACTCTTGAGGAAAAATGTCCATACGTGCTATGCCGCTTTCCACTTCTCTGAGAAC
CTGCTTCTTGAAGATTCATACGTCAATTTGGACGAAATCAACGCGTGTTTTTCGCGCGATGGAGACAAGT
TGACCTTTTCTTTTGCATCAGAGAGTACTCTTAATTATTGTCATAGTTATTCTAATATTCTTAAGTATGT
GTGCAAAACTTACTTCCCGGCCTCTAATAGAGAGGTTTACATGAAGGAGTTTTTAGTCACCAGAGTTAAT
ACCTGGTTTTGTAAGTTTTCTAGAATAGATACTTTTCTTTTGTACAAAGGTGTGGCCCATAAAAGTGTAG
ATAGTGAGCAGTTTTATACTGCAATGGAAGACGCATGGCATTACAAAAAGACTCTTGCAATGTGCAACAG
CGAGAGAATCCTCCTTGAGGATTCATCATCAGTCAATTACTGGTTTCCCAAAATGAGGGATATGGTCATC
GTACCATTATTCGACATTTCTTTGGAGACTAGTAAGAGGACGCGCAAGGAAGTCTTAGTGTCCAAGGATT
TCGTGTTTACAGTGCTTAACCACATTCGAACATACCAGGCGAAAGCTCTTACATACGCAAATGTTTTGTC
CTTTGTCGAATCGATTCGATCGAGGGTAATCATTAACGGTGTGACAGCGAGGTCCGAATGGGATGTGGAC
AAATCTTTGTTACAATCCTTGTCCATGACGTTTTACCTGCATACTAAGCTTGCCGTTCTAAAGGATGACT
TACTGATTAGCAAGTTTAGTCTCGGTTCGAAAACGGTGTGCCAGCATGTGTGGGATGAGATTTCGCTGGC
GTTTGGGAACGCATTTCCCTCCGTGAAAGAGAGGCTCTTGAACAGGAAACTTATCAGAGTGGCAGGCGAC
GCATTAGAGATCAGGGTGCCTGATCTATATGTGACCTTCCACGACAGATTAGTGACTGAGTACAAGGCCT
CTGTGGACATGCCTGCGCTTGACATTAGGAAGAAGATGGAAGAAACGGAAGTGATGTACAATGCACTTTC
AGAGTTATCGGTGTTAAGGGAGTCTGACAAATTCGATGTTGATGTTTTTTCCCAGATGTGCCAATCTTTG
GAAGTTGACCCAATGACGGCAGCGAAGGTTATAGTCGCGGTCATGAGCAATGAGAGCGGTCTGACTCTCA
CATTTGAACGACCTACTGAGGCGAATGTTGCGCTAGCTTTACAGGATCAAGAGAAGGCTTCAGAAGGTGC
TTTGGTAGTTACCTCAAGAGAAGTTGAAGAACCGTCCATGAAGGGTTCGATGGCCAGAGGAGAGTTACAA
TTAGCTGGTCTTGCTGGAGATCATCCGGAGTCGTCCTATTCTAAGAACGAGGAGATAGAGTCTTTAGAGC
AGTTTCATATGGCAACGGCAGATTCGTTAATTCGTAAGCAGATGAGCTCGATTGTGTACACGGGTCCGAT
TAAAGTTCAGCAAATGAAAAACTTTATCGATAGCCTGGTAGCATCACTATCTGCTGCGGTGTCGAATCTC
GTCAAGATCCTCAAAGATACAGCTGCTATTGACCTTGAAACCCGTCAAAAGTTTGGAGTCTTGGATGTTG
CATCTAGGAAGTGGTTAATCAAACCAACGGCCAAGAGTCATGCATGGGGTGTTGTTGAAACCCACGCGAG
GAAGTATCATGTGGCGCTTTTGGAATATGATGAGCAGGGTGTGGTGACATGCGATGATTGGAGAAGAGTA
GCTGTCAGCTCTGAGTCTGTTGTTTATTCCGACATGGCGAAACTCAGAACTCTGCGCAGACTGCTTCGAA
ACGGAGAACCGCATGTCAGTAGCGCAAAGGTTGTTCTTGTGGACGGAGTTCCGGGCTGTGGGAAAACCAA
AGAAATTCTTTCCAGGGTTAATTTTGATGAAGATCTAATTTTAGTACCTGGGAAGCAAGCCGCGGAAATG
ATCAGAAGACGTGCGAATTCCTCAGGGATTATTGTGGCCACGAAGGACAACGTTAAAACCGTTGATTCTT
TCATGATGAATTTTGGGAAAAGCACACGCTGTCAGTTCAAGAGGTTATTCATTGATGAAGGGTTGATGTT
GCATACTGGTTGTGTTAATTTTCTTGTGGCGATGTCATTGTGCGAAATTGCATATGTTTACGGAGACACA
CAGCAGATTCCATACATCAATAGAGTTTCAGGATTCCCGTACCCCGCCCATTTTGCCAAATTGGAAGTTG
ACGAGGTGGAGACACGCAGAACTACTCTCCGTTGTCCAGCCGATGTCACACATTATCTGAACAGGAGATA
TGAGGGCTTTGTCATGAGCACTTCTTCGGTTAAAAAGTCTGTTTCGCAGGAGATGGTCGGCGGAGCCGCC
GTGATCAATCCGATCTCAAAACCCTTGCATGGCAAGATCCTGACTTTTACCCAATCGGATAAAGAAGCTC
TGCTTTCAAGAGGGTATTCAGATGTTCACACTGTGCATGAAGTGCAAGGCGAGACATACTCTGATGTTTC
ACTAGTTAGGTTAACCCCTACACCAGTCTCCATCATTGCAGGAGACAGCCCACATGTTTTGGTCGCATTG
TCAAGGCACACCTGTTCGCTCAAGTACTACACTGTTGTTATGGATCCTTTAGTTAGTATCATTAGAGATC
TAGAGAAACTTAGCTCGTACTTGTTAGATATGTATAAGGTCGATGCAGGAACACAATAGCAATTACAGAT
TGACTCGGTGTTCAAAGGTTCCAATCTTTTTGTTGCAGCGCCAAAGACTGGTGATATTTCTGATATGCAG
TTTTACTATGATAAGTGTCTCCCAGGCAACAGCACCATGATGAATAATTTTGATGCTGTTACCATGAGGT
TGACTGACATTTCATTGAATGTCAAAGATTGCATATTGGATATGTCTAAGTCTGTTGCTGCGCCTAAGGA
TCAAATCAAACCACTAATACCTATGGTACGAACGGCGGCAGAAATGCCACGCCAGACTGGACTATTGGAA
AATTTAGTGGCGATGATTAAAAGGAACTTTAACGCACCCGAGTTGTCTGGCATCATTGATATTGAAAATA
CTGCATCTTTAGTTGTAGATAAGTTTTTTGATAGTTATTTGCTTAAAGAAAAAAGAAAACCAAATAAAAA
TGTTTCTTTGTTCAGTAGAGAGTCTCTCAATAGATGGTTAGAAAAGCAGGAACAGGTAACAATAGGCCAG
CTCGCAGATTTTGATTTTGTAGATTTGCCAGCAGTTGATCAGTACAGACACATGATTAAAGCACAACCCA
AGCAAAAATTGGACACTTCAATCCAAACGGAGTACCCGGCTTTGCAGACGATTGTGTACCATTCAAAAAA
GATCAATGCAATATTTGGCCCGTTGTTTAGTGAGCTTACTAGGCAATTACTGGACAGTGTTGATTCGAGC
AGATTTTTGTTTTTCACAAGAAAGACACCAGCGCAGATTGAGGATTTCTTCGGAGATCTCGACAGTCATG
TGCCGATGGATGTCTTGGAGCTGGATATATCAAAATACGACAAATCTCAGAATGAATTCCACTGTGCAGT
AGAATACGAGATCTGGCGAAGATTGGGTTTTGAAGACTTCTTGGGAGAAGTTTGGAAACAAGGGCATAGA
AAGACCACCCTCAAGGATTATACCGCAGGTATAAAAACTTGCATCTGGTATCAAAGAAAGAGCGGGGACG
TCACGACGTTCATTGGAAACACTGTGATCATTGCTGCATGTTTGGCCTCGATGCTTCCGATGGAGAAAAT
AATCAAAGGAGCCTTTTGCGGTGACGATAGTCTGCTGTACTTTCCAAAGGGTTGTGAGTTTCCGGATGTG
CAACACTCCGCGAATCTTATGTGGAATTTTGAAGCAAAACTGTTTAAAAAACAGTATGGATACTTTTGCG
GAAGATATGTAATACATCACGACAGAGGATGCATTGTGTATTACGATCCCCTAAAGTTGATCTCGAAACT
TGGTGCTAAACACATCAAGGATTGGGAACACTTGGAGGAGTTCAGAAGGTCTCTTTGTGATGTTGCTGTT
TCGTTGAACAATTGTGCGTATTACACACAGTTGGACGACGCTGTATGGGAGGTTCATAAGACCGCCCCTC
CAGGTTCGTTTGTTTATAAAAGTCTGGTGAAGTATTTGTCTGATAAAGTTCTTTTTAGAAGTTTGTTTAT
AGATGGCTCTAGTTGTTAAAGGAAAAGTGAATATCAATGAGTTTATCGACCTGACAAAAATGGAGAAGAT
CTTACCGTCGATGTTTACCCCTGTAAAGAGTGTTATGTGTTCCAAAGTTGATAAAATAATGGTTCATGAG
AATGAGTCATTGTCAGAGGTGAACCTTCTTAAAGGAGTTAAGCTTATTGATAGTGGATACGTCTGTTTAG
CCGGTTTGGTCGTCACGGGCGAGTGGAACTTGCCTGACAATTGCAGAGGAGGTGTGAGCGTGTGTCTGGT
GGACAAAAGGATGGAAAGAGCCGACGAGGCCACTCTCGGATCTTACTACACAGCAGCTGCAAAGAAAAGA
TTTCAGTTCAAGGTCGTTCCCAATTATGCTATAACCACCCAGGACGCGATGAAAAACGTCTGGCAAGTTT
TAGTTAATATTAGAAATGTGAAGATGTCAGCGGGTTTCTGTCCGCTTTCTCTGGAGTTTGTGTCGGTGTG
TATTGTTTATAGAAATAATATAAAATTAGGTTTGAGAGAGAAGATTACAAACGTGAGAGACGGAGGGCCC
ATGGAACTTACAGAAGAAGTCGTTGATGAGTTCATGGAAGATGTCCCTATGTCGATCAGGCTTGCAAAGT
TTCGATCTCGAACCGGAAAAAAGAGTGATGTCCGCAAAGGGAAAAATAGTAGTAATGATCGGTCAGTGCC
GAACAAGAACTATAGAAATGTTAAGGATTTTGGAGGAATGAGTTTTAAAAAGAATAATTTAATCGATGAT
GATTCGGAGGCTACTGTCGCCGAATCGGATTCGTTTTAAATATGTCTTACAGTATCACTACTCCATCTCA
GTTCGTGTTCTTGTCATCAGCGTGGGCCGACCCAATAGAGTTAATTAATTTATGTACTAATGCCTTAGGA
AATCAGTTTCAAACACAACAAGCTCGAACTGTCGTTCAAAGACAATTCAGTGAGGTGTGGAAACCTTCAC
CACAAGTAACTGTTAGGTTCCCTGACAGTGACTTTAAGGTGTACAGGTACAATGCGGTATTAGACCCGCT
AGTCACAGCACTGTTAGGTGCATTCGACACTAGAAATAGAATAATAGAAGTTGAAAATCAGGCGAACCCC
ACGACTGCCGAAACGTTAGATGCTACTCGTAGAGTAGACGACGCAACGGTGGCCATAAGGAGCGCGATAA
ATAATTTAATAGTAGAATTGATCAGAGGAACCGGATCTTATAATCGGAGCTCTTTCGAGAGCTCTTCTGG
TTTGGTTTGGACCTCTGGTCCTGCAACTTGAGGTAGTCAAGATGCATAATAAATAACGGATTGTGTCCGT
AATCACACGTGGTGCGTACGATAACGCATAGTGTTTTTCCCTCCACTTAAATCGAAGGGTTGTGTCTTGG
ATCGCGCGGGTCAAATGTATATGGTTCATATACATCCGCAGGCACGTAATAAAGCGAGGGGTTCGAATCC
CCCCGTTACCCCCGGTAGGGGCCCA
Cauliflower Mosaic Virus Sequence (genomic DNA, Accession Number:
NC_001497.1) (SEQ ID NO: 431):
GGTATCAGAGCCATGAATCGGTTTAAGACCAAAACTCAAGAGGGTAAAACCTCACCAAAATACGAAAGAG
TTCTTAACTCTAAAAATAAAAGATCTTTCAAGATCAAACATAGTTCCCTCACACCGGTGACCGACAGGAT
TACCACCGTAAGGTTTCAGAACAACATCGAAAGCGTTTACGCCAACTTCGACTCTCAACTCAAGTCGTCG
TACGATGGTAGATCTAAAAAGATCAAGACTCTAAGCCTTAAAAATCTTAGATGTTACGAAGCCTTCCTCA
GGAAGTACCTTCTGGAACAATAAATCTCTCTGAGAATAGTACTCTATTGAGTATCCACAGGAAAAATAAC
CTTCTGTGTTGAGATGGATTTGTATCCAGAAGAAAATACCCAAAGCGAGCAATCGCAGAATTCTGAAAAT
AATATGCAAATATTTAAATCAGAAAATTCGGATGGATTCTCCTCCGATCTAATGATCTCAAACGATCAAT
TAAAAAATATCTCTAAAACCCAATTAACCTTGGAGAAAGAAAAGATATTTAAAATGCCTAACGTTTTATC
TCAAGTTATGAAAAAAGCGTTTAGCAGGAAAAACGAGATTCTCTACTGCGTCTCGACAAAAGAATTATCA
GTGGACATTCACGATGCCACAGGTAAGGTATATCTTCCCTTAATCACTAAGGAAGAGATAAATAAAAGAC
TTTCCAGCTTAAAACCTGAAGTCAGAAAGACCATGTCCATGGTTCATCTTGGAGCGGTCAAAATATTGCT
TAAAGCTCAATTTCGAAATGGGATTGATACCCCAATCAAAATTGCTTTAATCGATGATAGAATCAATTCT
AGAAGAGATTGTCTTCTTGGTGCAGCCAAAGGTAATCTAGCATACGGTAAGTTTATGTTTACTGTATACC
CTAAGTTTGGAATAAGCCTTAACACCCAAAGACTTAACCAAACCCTAAGCCTTATTCATGATTTTGAAAA
TAAAAATCTTATGAATAAAGGTGATAAAGTTATGACCATAACCTATGTCGTAGGATATGCATTAACTAAT
AGTCATCATAGCATAGATTATCAATCAAATGCTACAATTGAACTAGAAGACGTATTTCAAGAAATTGGAA
ATGTCCAGCAATCTGAGTTCTGTACAATACAGAATGATGAATGCAATTGGGCCATTGATATAGCCCAAAA
CAAAGCCTTATTAGGAGCTAAAACCAAGACTCAAATTGGTAATAACCTTCAAATAGGTAACAGTGCTTCA
TCCTCTAATACTGAAAATGAATTAGCTAGGGTAAGCCAGAACATAGATCTTTTAAAGAATAAATTAAAAG
AAATCTGTGGAGAATAATATGAGCATTACGGGACAACCGCATGTTTATAAAAAAGATACTATTATTAGAC
TAAAACCATTGTCTCTTAATAGTAATAATAGAAGTTATGTTTTTAGTTCCTCAAAAGGGAACATTCAAAA
TATAATTAATCATCTTAACAACCTCAATGAGATTGTAGGAAGAAGCTTACTCGGAATATGGAAGATCAAC
TCATACTTCGGATTAAGCAAAGACCCTTCGGAGTCCAAATCAAAAAACCCGTCAGTTTTTAATACTGCAA
AAACCATTTTTAAGAGTGGGGGGGTTGATTACTCGAGCCAACTAAAGGAAATAAAATCCCTTTTAGAAGC
TCAAAACACTAGAATAAAAAGTCTAGAAAAAGCAATTCAATCCTTAGAAAATAAGATTGAACCAGAGCCC
TTAACTAAAGAGGAAGTTAAAGAGCTAAAAGAATCGATTAACTCGATCAAAGAAGGATTAAAGAATATTA
TTGGCTAAAATGGCTAATCTTAATCAGATCCAAAAAGAAGTCTCTGAAATCCTCAGTGACCAAAAATCCA
TGAAAGCGGATATAAAAGCTATCTTAGAATTATTAGGATCCCAAAATCCTATTAAAGAAAGCTTAGAAAC
CGTTGCAGCAAAAATCGTTAATGACTTAACCAAGCTCATCAATGATTGTCCTTGTAACAAAGAGATATTA
GAAGCCTTAGGTACCCAACCTAAAGAGCAACTAATAGAACAACCTAAAGAAAAAGGTAAAGGCCTTAACT
TAGGAAAATACTCTTACCCCAATTACGGAGTAGGAAATGAAGAATTAGGATCCTCTGGAAACCCTAAAGC
TTTAACCTGGCCCTTCAAAGCTCCAGCAGGATGGCCGAATCAATTTTAGACAGAACCATTAATAGGTTTT
GGTATAATCTGGGAGAAGATTGTCTCTCAGAAAGTCAATTCGATCTTATGATAAGATTGATGGAAGAGTC
CCTTGACGGGGACCAAATTATTGATCTAACCTCTCTACCTAGTGATAATTTGCAGGTTGAACAGGTTATG
ACAACTACCGAAGACTCAATCTCGGAAGAAGAATCAGAATTCCTTCTAGCAATAGGAGAAACATCTGAAG
AAGAAAGCGATTCAGGAGAAGAACCTGAATTCGAGCAAGTTCGAATGGATCGAACAGGAGGAACGGAGAT
TCCAAAAGAAGAAGATGGTGAAGGACCATCTAGATACAATGAGAGAAAGAGAAAGACCCCGGAGGACCGG
TACTTTCCAACTCAACCAAAGACCATTCCAGGACAAAAGCAAACGTCTATGGGAATGCTCAACATTGACT
GCCAAACCAATCGAAGAACTCTAATCGACGACTGGGCAGCAGAAATCGGATTGATAGTCAAGACCAATAG
AGAAGACTATCTCGATCCAGAAACAATTCTACTCTTGATGGAACACAAAACATCAGGAATAGCCAAGGAG
TTAATCCGAAATACAAGATGGAACCGCACTACCGGAGACATCATAGAACAGGTGATCGATGCGATGTACA
CCATGTTCTTAGGACTAAACTACTCCGACAACAAAGTTGCTGAGAAGATTGACGAGCAAGAGAAGGCCAA
GATCAGAATGACCAAGCTCCAGCTCTGCGACATCTGCTACCTTGAGGAATTTACATGTGATTATGAAAAG
AACATGTATAAGACAGAACTGGCGGATTTCCCAGGATATATCAACCAGTACCTGTCAAAAATCCCCATCA
TTGGAGAAAAAGCGTTAACACGCTTTAGGCATGAAGCTAACGGAACCAGCATCTACAGTTTAGGTTTCGC
GGCAAAGATAGTCAAAGAAGAACTATCTAAAATCTGCGACTTATCCAAGAAGCAGAAGAAGTTGAAGAAA
TTCAACAAGAAGTGTTGTAGCATCGGAGAAGCTTCAACAGAATATGGATGCAAGAAGACATCCACAAAGA
AGTATCACAAGAAGCGATACAAGAAAAAATATAAGGCTTACAAACCTTATAAGAAGAAAAAGAAGTTCCG
ATCAGGAAAATACTTCAAGCCCAAAGAAAAGAAGGGCTCAAAGCAAAAGTATTGCCCAAAAGGCAAGAAA
GATTGCAGATGTTGGATCTGCAACATTGAAGGCCATTACGCCAACGAATGTCCTAATCGACAAAGCTCGG
AGAAGGCTCACATCCTTCAACAAGCAGAAAAATTGGGTCTCCAGCCCATTGAAGAACCCTATGAAGGAGT
TCAAGAAGTATTCATTCTAGAATACAAAGAAGAGGAAGAAGAAACCTCTACAGAAGAAAGTGATGGATCA
TCTACTTCTGAAGACTCAGACTCAGACTGAGCAGGTGATGAACGTCACCAATCCCAATTCGATCTACATC
AAGGGAAGACTCTACTTCAAGGGATACAAGAAGATAGAACTTCACTGTTTCGTAGACACGGGAGCAAGCC
TATGCATAGCATCCAAGTTCGTCATACCAGAAGAACATTGGGTCAATGCAGAAAGACCAATTATGGTCAA
AATAGCAGATGGAAGCTCAATCACCATCAGCAAAGTCTGCAAAGACATAGACTTGATCATAGCCGGCGAG
ATATTCAGAATTCCCACCGTCTATCAGCAAGAAAGTGGCATCGATTTCATTATCGGCAACAACTTCTGTC
AGCTGTATGAACCATTCATACAGTTTACGGATAGAGTTATCTTCACAAAGAACAAGTCTTATCCTGTTCA
TATTGCGAAGCTAACCAGAGCAGTGCGAGTAGGCACCGAAGGATT TCTTGAATCAATGAAGAAACGTTCA
AAAACTCAACAACCAGAGCCAGTGAACATTTCTACAAACAAGATAGAAAATCCACTAGAAGAAATTGCTA
TTCTTTCAGAGGGGAGGAGGTTATCAGAAGAAAAACTCTTTATCACTCAACAAAGAATGCAAAAAATCGA
AGAACTACTTGAGAAAGTATGTTCAGAAAATCCATTAGATCCTAACAAGACTAAGCAATGGATGAAAGCT
TCTATCAAGCTCAGCGACCCAAGCAAAGCTATCAAGGTTAAACCCATGAAGTATAGCCCAATGGATCGCG
AAGAATTTGACAAGCAAATCAAAGAATTACTGGACCTAAAAGTCATCAAGCCCAGTAAAAGCCCTCACAT
GGCACCAGCCTTCTTGGTCAACAATGAAGCCGAGAAGCGAAGAGGAAAGAAACGTATGGTAGTCAACTAC
AAAGCTATGAACAAAGCTACTGTAGGAGATGCCTACAATCTTCCCAACAAAGACGAGTTACTTACACTCA
TTCGAGGAAAGAAGATCTTCTCTTCCTTCGACTGTAAGTCAGGATTCTGGCAAGTTCTGCTAGATCAAGA
ATCAAGACCTCTAACGGCATTCACATGTCCACAAGGTCACTACGAATGGAATGTGGTCCCTTTCGGCTTA
AAGCAAGCTCCATCCATATTCCAAAGACACATGGACGAAGCATTTCGTGTGTTCAGAAAGTTCTGTTGCG
TTTATGTCGACGACATTCTCGTATTCAGTAACAACGAAGAAGATCATCTACTTCACGTAGCAATGATCTT
ACAAAAGTGTAATCAACATGGAATTATCCTTTCCAAGAAGAAAGCACAACTCTTCAAGAAGAAGATAAAC
TTCCTTGGTCTAGAAATAGATGAAGGAACACATAAGCCTCAAGGACATATCTTGGAACACATCAACAAGT
TCCCCGATACCCTTGAAGACAAGAAGCAACTTCAGAGATTCTTAGGCATACTAACATATGCCTCGGATTA
CATCCCGAAGCTAGCTCAAATCAGAAAGCCTCTGCAAGCCAAGCTTAAAGAAAACGTTCCATGGAGATGG
ACAAAAGAGGATACCCTCTACATGCAAAAGGTGAAGAAAAATCTGCAAGGATTTCCTCCACTACATCATC
CCTTACCAGAGGAGAAGCTGATCATCGAGACCGATGCATCAGACGACTACTGGGGAGGTATGTTAAAAGC
TATCAAAATTAACGAAGGTACTAATACTGAGTTAATTTGCAGATACGCATCTGGAAGCTTTAAAGCTGCA
GAAAAGAATTACCACAGCAATGACAAAGAGACATTGGCGGTAATAAATACTATAAAGAAATTTAGTATTT
ATCTAACTCCTGTTCATT TTCTGATTAGGACAGATAATACTCATTTCAAGAGTTTCGTTAATCTCAATTA
CAAAGGAGATTCGAAACTTGGAAGAAACATCAGATGGCAAGCATGGCTTAGCCACTATTCATTTGATGTT
GAACACATTAAAGGAACCGACAACCACTTTGCGGACTTCCTTTCAAGAGAATTCAATAAGGTTAATTCCT
AATTGAAATCCGAAGATAAGATTCCCACACACTTGTGGCTGATATCAAAAGGCTACTGCCTATTTAAACA
CATCTCTGGAGACTGAGAAAATCAGACCTCCAAGCATGGAGAACATAGAAAAACTCCTCATGCAAGAGAA
AATACTAATGCTAGAGCTCGATCTAGTAAGAGCAAAAATAAGCTTAGCAAGAGCTAACGGCTCTTCGCAA
CAAGGAGACCTCTCTCTCCACCGTGAAACACCGGAAAAAGAAGAAGCAGTTCATTCTGCACTGGCTACTT
TTACGCCATCTCAAGTAAAAGCTATTCCAGAGCAAACGGCTCCTGGTAAAGAATCAACAAATCCGTTGAT
GGCTAATATCTTGCCAAAAGATATGAATTCAGTTCAGACTGAAATTAGGCCCGTAAAGCCATCGGACTTC
TTACGTCCACATCAGGGAATTCCAATCCCACCAAAACCTGAACCTAGCAGTTCAGTTGCTCCTCTCAGAG
ACGAATCGGGTATTCAACACCCTCATACCAACTACTACGTCGTGTATAACGGACCTCATGCCGGTATATA
CGATGACTGGGGTTGTACAAAGGCAGCAACAAACGGTGTTCCCGGAGTTGCGCATAAGAAGTTTGCCACT
ATTACAGAGGCAAGAGCAGCAGCTGACGCGTATACAACAAGTCAGCAAACAGATAGGTTGAACTTCATCC
CCAAAGGAGAAGCTCAACTCAAGCCCAAGAGCTTTGCGAAGGCCTTAACAAGCCCACCAAAGCAAAAAGC
CCACTGGCTCATGCTAGGAACTAAAAAGCCCAGCAGTGATCCAGCCCCAAAAGAGATCTCCTTTGCCCCA
GAGATCACAATGGACGACTTCCTCTATCTCTACGATCTAGTCAGGAAGTTCGACGGAGAAGGTGACGATA
CCATGTTCACCACTGATAATGAGAAGATTAGCCTTTTCAATTTCAGAAAGAATGCTAACCCACAGATGGT
TAGAGAGGCTTACGCAGCAGGTCTCATCAAGACGATCTACCCGAGCAATAATCTCCAGGAGATCAAATAC
CTTCCCAAGAAGGTTAAAGATGCAGTCAAAAGATTCAGGACTAACTGCATCAAGAACACAGAGAAAGATA
TATTTCTCAAGATCAGAAGTACTATTCCAGTATGGACGATTCAAGGCTTGCTTCACAAACCAAGGCAAGT
AATAGAGATTGGAGTCTCTAAAAAGGTAGTTCCCACTGAATCAAAGGCCATGGAGTCAAAGATTCAAATA
GAGGACCTAACAGAACTCGCCGTAAAGACTGGCGAACAGTTCATACAGAGTCTCTTACGACTCAATGACA
AGAAGAAAATCTTCGTCAACATGGTGGAGCACGACACGCTTGTCTACTCCAAAAATATCAAAGATACAGT
CTCAGAAGACCAAAGGGCAATTGAGACTTTTCAACAAAGGGTAATATCCGGAAACCTCCTCGGATTCCAT
TGCCCAGCTATCTGTCACTTTATTGTGAAGATAGTGGAAAAGGAAGGTGGCTCCTACAAATGCCATCATT
GCGATAAAGGAAAGGCCATCGTTGAAGATGCCTCTGCCGACAGTGGTCCCAAAGATGGACCCCCACCCAC
GAGGAGCATCGTGGAAAAAGAAGACGTTCCAACCACGTCTTCAAAGCAAGTGGATTGATGTGATATCTCC
ACTGACGTAAGGGATGACGCACAATCCCACTATCCTTCGCAAGACCCTTCCTCTATATAAGGAAGTTCAT
TTCATTTGGAGAGGACACGCTGAAATCACCAGTCTCTCTCTACAAATCTATCTCTCTCTATAATAATGTG
TGAGTAGTTCCCAGATAAGGGAATTAGGGTTCTTATAGGGTTTCGCTCATGTGTTGAGCATATAAGAAAC
CCTTAGTATGTATTTGTATTTGTAAAATACTTCTATCAATAAAATTTCTAATTCCTAAAACCAAAATCCA
GTACTAAAATCCAGATCTCCTAAAGTCCCTATAGATCTTTGTGGTGAATATAAACCAGACACGAGACGAC
TAAACCTGGAGCCCAGACGCCGTTTGAAGCTAGAAGTACCGCTTAGGCAGGAGGCCGTTAGGGAAAAGAT
GCTAAGGCAGGGTTGGTTACGTTGACTCCCCCGTAGGTTTGGTTTAAATATCATGAAGTGGACGGAAGGA
AGGAGGAAGACAAGGAAGGATAAGGTTGCAGGCCCTGTGCAAGGTAAGACGATGGAAATTTGATAGAGGT
ACGTTACTATACTTATACTATACGCTAAGGGAATGCTTGTATTTACCCTATATACCCTAATGACCCCTTA
TCGATTTAAAGAAATAATCCGCATAAGCCCCCGCTTAAAAAATT
Tomato mosaic virus (genomic DNA, Accession Number: NC_002692.1) (SEQ ID
NO: 432):
GTATTTTTACAACAATTACCAACAACAACAACAAACAACAACAACATTACATTTTACATTCTACAACTAC
AATGGCATACACACAAACAGCCACATCGTCCGCTTTGCTTGAGACCGTCCGAGGTAACAATACCTTGGTC
AACGATCTTGCAAAGCGGCGTCTATATGACACAGCGGTAGATGAATTTAATGCTAGGGACCGCAGGCCTA
AAGTCAATTTTTCCAAAGTAGTAAGCGAAGAACAGACGCTTATTGCAACCAAAGCCTACCCAGAATTCCA
AATTACATTCTACAACACGCAGAATGCTGTGCATTCCCTTGCAGGCGGTCTCCGATCATTAGAATTGGAA
TATCTGATGATGCAAATTCCCTACGGATCATTGACATATGATATCGGAGGTAATTTTGCATCTCATCTGT
TCAAAGGGCGAGCATACGTTCACTGCTGTATGCCGAATCTAGATGTCCGCGACATAATGCGGCACGAGGG
CCAAAAGGACAGTATTGAACTATACCTTTCTAGGCTCGAGAGGGGCAACAAACATGTCCCAAACTTCCAA
AAGGAAGCTTTCGACAGATACGCTGAAATGCCAAACGAAGTAGTCTGTCACGATACTTTCCAAACGTGTA
GGCATTCTCAAGAATGTTACACGGGAAGAGTGTATGCTATTGCTTTGCATAGTATATACGATATACCTGC
CGACGAGTTCGGCGCGGCACTGCTGAGAAAGAATGTACATGTATGTTATGCCGCTTTCCACTTTTCCGAG
AATTTACTTCTCGAAGATTCACACGTCAACCTCGATGAGATCAATGCATGTT TCCAAAGAGATGGAGACA
GGTTGACTTTTTCCTTTGCATCTGAGAGTACTCTTAATTATAGTCATAGTTATTCTAATATTCTTAAGTA
TGTTTGCAAAACTTACTTCCCAGCCTCTAATAGAGAGGTTTACATGAAGGAGTTTTTAGTAACTAGAGTT
AATACCTGGTTTTGTAAATTTTCTAGAATAGATACTTTCTTATTGTACAAAGGTGTAGCGCATAAGGGTG
TAGATAGTGAGCAGTTTTACAAGGCTATGGAAGACGCATGGCACTACAAAAAGACTCTTGCGATGTGCAA
CAGTGAAAGAATCTTGTTAGAGGATTCTTCATCAGTTAATTACTGGTTTCCAAAAATGAGGGATATGGTG
ATAGTTCCACTATTTGACATATCTCTCGAGACTAGTAAAAGAACACGCAAAGAGGTCTTAGTTTCAAAGG
ACTTTGTTTATACAGTGTTAAATCACATTCGTACGTACCAGGCCAAAGCGCTTACTTACTCCAACGTGTT
ATCTTTCGTCGAATCAATTCGTTCGAGAGTGATCATTAACGGGGTTACTGCCAGGTCTGAGTGGGATGTC
GATAAATCATTATTACAGTCCTTGTCGATGACGTTCTTCCTACATACCAAGCTTGCCGTTCTGAAAGACG
ATCTTTTGATTAGCAAGTTTGCACTTGGACCAAAAACTGTCTCACAACATGTGTGGGATGAGATTTCCCT
AGCTTTCGGCAATGCTTTCCCATCGATCAAGGAAAGATTGATAAACCGGAAACTGATCAAAATTACGGAG
AATGCGTTAGAGATCAGGGTGCCCGATCTTTATGTCACTTTCCATGATAGGTTAGTTTCTGAGTACAAAA
TGTCAGTGGACATGCCGGTGCTAGACATTAGGAAAAAGATGGAAGAAACTGAGGAAATGTACAATGCACT
GTCCGAACTGTCTGTACTTAAAAATTCAGACAAGTTCGATGTTGATGTTTTTTCCCAGATGTGCCAATCT
TTAGAAGTTGATCCAATGACTGCAGCAAAGGTAATAGTAGCAGTTATGAGCAACGAGAGTGGTCTTACTC
TCACGTTTGAACAGCCCACCGAAGCTAATGTTGCGCTAGCATTGCAAGATTCTGAAAAGGCTTCTGATGG
GGCGTTGGTAGTTACCTCAAGAGATGTTGAGGAACCGTCCATAAAGGGTTCGATGGCCCGTGGTGAGTTA
CAATTGGCCGGATTATCTGGCGACGTTCCTGAATCTTCATACACTAGGAGCGAGGAGATTGAGTCTCTCG
AGCAGTTTCATATGGCAACAGCTAGTTCGTTAATTCATAAGCAGATGTGTTCGATCGTGTACACGGGCCC
TCTTAAAGTTCAACAAATGAAAAACTTTATAGACAGCCTGGTAGCCTCGCTCTCTGCTGCGGTGTCGAAT
CTAGTGAAGATCCTAAAAGATACAGCCGCGATTGACCTTGAAACTCGTCAAAAGTTCGGAGTTCTGGATG
TTGCTTCGAAAAGGTGGCTAGTTAAACCATCCGCAAAGAACCATGCATGGGGGGTTGTTGAGACTCATGC
GAGGAAATATCACGTCGCATTACTGGAGCACGATGAATTTGGCATTATTACGTGCGATAACTGGCGACGG
GTGGCTGTGAGTTCTGAGTCGGTAGTATATTCTGATATGGCTAAACTCAGGACTCTGAGAAGATTGCTCA
AAGATGGAGAACCACACGTTAGTTCAGCAAAGGTGGTTTTGGTGGATGGCGTTCCAGGGTGCGGGAAGAC
AAAGGAAATTCTTTCGAGAGTTAATTTCGAAGAAGATCTAATTCTTGTCCCTGGTCGTCAAGCTGCCGAG
ATGATCAGAAGAAGAGCTAATGCGTCGGGCATAATAGTGGCTACAAAGGATAATGTGCGCACCGTCGATT
CATTTTTGATGAATTACGGGAAAGGGGCACGCTGTCAGTTCAAAAGATTGTTCATAGACGAAGGTTTGAT
GCTGCATACTGGTTGTGTGAATTTCTTGGTTGAAATGTCTCTGTGCGATATTGCATATGTTTATGGAGAC
ACCCAACAAATTCCGTACATCAACAGAGTAACTGGTTTCCCGTACCCTGCGCACTTTGCAAAATTGGAGG
TCGACGAAGTCGAAACAAGAAGAACTACTCTTCGCTGTCCGGCTGATGTCACACACTTCCTAAATCAAAG
GTATGAAGGACACGTAATGTGCACGTCTTCTGAAAAGAAATCAGTTTCCCAGGAAATGGTTAGTGGGGCT
GCGTCTATCAATCCTGTGTCCAAGCCGCTTAAAGGGAAAATTTTGACTTTCACACAGTCTGACAAGGAGG
CCCTTCTCTCAAGGGGCTACGCAGATGTCCATACTGTACATGAGGTACAAGGTGAGACTTATGCAGACGT
ATCGTTAGTTCGACTAACACCTACGCCTGTATCTATCATCGCAAGAGACAGTCCGCATGTTCTGGTCTCG
TTGTCAAGACACACAAAATCCCTAAAGTACTACACCGTTGTGATGGATCCTTTAGTTAGTATCATTAGAG
ATTTAGAACGGGTTAGTAGTTACTTATTAGACATGTACAAAGTAGATGCAGGTACTCAATAGCAATTACA
GGTCGACTCTGTGTTTAAAAATTTCAATCTTTTTGTAGCAGCTCCAAAGACTGGAGATATATCTGATATG
CAATTTTACTATGATAAGTGTCTTCCTGGGAACAGCACGTTGTTGAACAACTACGACGCTGTTACCATGA
AATTGACTGACATTTCTCTGAATGTCAAAGATTGCATATTAGATATGTCTAAGTCTGTAGCTGCTCCGAA
AGATGTCAAACCAACTTTAATACCGATGGTACGAACGGCGGCAGAAATGCCTCGCCAGACTGGACTGTTG
GAAAATCTAGTTGCGATGATTAAAAGAAATTTTAATTCACCAGAGTTGTCCGGAGTAGTTGATATTGAAA
ATACTGCATCTTTAGTGGTAGATAAGTTTTTTGATAGTTATTTACTTAAGGAAAAAAGAAAACCAAACAA
AAATTTTTCACTGTTTAGTAGAGAGTCTCTCAATAGGTGGATAGCAAAGCAAGAACAAGTCACAATTGGT
CAGTTGGCCGATTTTGATTTTGTGGATCTTCCAGCCGTTGATCAGTACAGGCATATGATTAAAGCGCAAC
CGAAGCAGAAACTGGATCTGTCAATTCAGACAGAATATCCAGCGTTGCAAACGATTGTGTATCATTCAAA
GAAAATCAACGCAATATTTGGTCCTCTTTTCAGTGAGCTTACAAGGCAATTACTTGACAGTATTGACTCA
AGCAGATTCTTGTTCTTTACGAGAAAGACACCGGCTCAGATCGAAGATTTCTTCGGAGATCTAGACAGTC
ATGTCCCAATGGACGTACTTGAGTTGGATGTTTCGAAGTATGATAAGTCTCAAAACGAGTTTCATTGTGC
TGTTGAGTACGAAATCTGGAGGAGACTGGGTCTGGAGGATTTCTTGGCAGAAGTGTGGAAACAAGGGCAT
AGAAAAACCACCCTGAAAGATTACACTGCTGGTATAAAAACGTGTTTATGGTACCAGAGAAAGAGTGGTG
ATGTTACAACTTTTATCGGTAATACCGTCATCATTGCTTCGTGTCTTGCATCAATGCTCCCGATGGAAAA
ATTGATAAAAGGAGCCTTCTGCGGAGATGACAGTTTGTTGTACTTTCCTAAGGGTTGTGAGTATCCCGAT
ATACAACAAGCTGCCAATCTAATGTGGAATTTTGAGGCCAAACTGTTCAAGAAGCAATATGGGTACTTCT
GCGGGAGGTACGTGATTCATCACGATAGAGGTTGCATAGTATACTACGACCCTTTGAAGCTGATTTCGAA
ACTTGGTGCTAAACACATCAAGGATTGGGATCATTTGGAGGAGTTCAGAAGATCCCTCTGTGATGTTGCT
GAGTCGTTGAACAATTGCGCGTATTACACACAATTGGACGACGCTGTTGGGGAGGTTCATAAAACCGCCC
CACCTGGTTCGTTTGTTTATAAGAGTTTAGTTAAGTATTTGTCAGATAAAGTTTTGTTTAGAAGTTTATT
TCTTGATGGCTCTAGTTGTTAAAGGTAAGGTAAATATTAATGAGTTTATCGATCTGTCAAAGTCTGAGAA
ACTTCTCCCGTCGATGTTCACGCCTGTAAAGAGTGTTATGGTTTCAAAGGTTGATAAGATTATGGTCCAT
GAAAATGAATCATTGTCTGAAGTAAATCTCTTAAAAGGTGTAAAACTTATAGAAGGTGGGTATGTTTGCT
TAGTCGGTCTTGTTGTGTCCGGTGAGTGGAATTTACCAGATAATTGCCGTGGTGGTGTGAGTGTCTGCAT
GGTTGACAAGAGAATGGAAAGAGCGGACGAAGCCACACTGGGGTCATATTACACTGCTGCTGCTAAAAAG
CGGTTTCAGTTTAAAGTGGTCCCAAATTACGGTATTACAACAAAGGATGCAGAAAAGAACATATGGCAGG
TCTTAGTAAATATTAAAAATGTAAAAATGAGTGCGGGCTACTGCCCTTTGTCATTAGAATTTGTGTCTGT
GTGTATTGTTTATAAAAATAATATAAAATTGGGTTTGAGGGAGAAAGTAACGAGTGTGAACGATGGAGGA
CCCATGGAACTTTCAGAAGAAGTTGTTGATGAGTTCATGGAGAATGTTCCAATGTCGGTTAGACTCGCAA
AGTTTCGAACCAAATCCTCAAAAAGAGGTCCGAAAAATAATAATAATTTAGGTAAGGGGCGTTCAGGCGG
AAGGTCTAAACCAAAAAGTTTTGATGAAGTTGAAAAAGAGTTTGATAATTTGATTGAAGATGAAGCCGAG
ACGTCGGTCGCGGATTCTGATTCGTATTAAATATGTCTTACTCAATCACTTCTCCATCGCAATTTGTGTT
TTTGTCATCTGTATGGGCTGACCCTATAGAATTGTTAAACGTTTGTACAAATTCGTTAGGTAACCAGTTT
CAAACACAGCAAGCAAGAACTACTGTTCAACAGCAGTTCAGCGAGGTGTGGAAACCTTTCCCTCAGAGCA
CCGTCAGATTTCCTGGCGATGTTTATAAGGTGTACAGGTACAATGCAGTTTTAGATCCTCTAATTACTGC
GTTGCTGGGGGCTTTCGATACTAGGAATAGAATAATCGAAGTAGAAAACCAGCAGAGTCCGACAACAGCT
GAAACGTTAGATGCTACCCGCAGGGTAGACGACGCTACGGTTGCAATTCGGTCTGCTATAAATAATTTAG
TTAATGAACTAGTAAGAGGTACTGGACTGTACAATCAGAATACTTTTGAAAGTATGTCTGGGTTGGTCTG
GACCTCTGCACCTGCATCTTAAATGCATAGGTGCTGAAATATAAATTTTGTGTTTCTAAAACACACGTGG
TACGTACGATAACGTACAGTGTTTTTCCCTCCACTTAAATCGAAGGGTAGTGTCTTGGAGCGCGCGGAGT
AAACATATATGGTTCATATATGTCCGTAGGCACGTAAAAAAGCGAGGGATTCGAATTCCCCCGGAACCCC
CGGTTGGGGCCCA
Pepper mild mottle virus (genomic DNA, Accession Number: NC_003630.1) (SEQ ID
NO: 433):
GTAAATTTTTCACAATTTAACAACAACAACACAAACAACAAACAACATTACAAACAAAATACAACTACAA
TGGCTTACACACAACAAGCTACCAACGCCGCATTAGCAAGTACTCTCCGAGGGAATAACCCCTTGGTGAA
CGATCTTGCTAATCGGAGACTGTACGAATCAGCGGTCGAACAATGCAATGCACATGACCGCAGGCCCAAG
GTTAATTTTTTAAGGTCGATAAGCGAAGAGCAGACGCTTATCGCAACTAAGGCCTACCCTGAGTTCCAAA
TCACGTTCTACAACACGCAGAACGCTGTGCACAGTCTCGCAGGTGGACTTCGGTCTTTGGAACTAGAATA
CTTGATGATGCAGATCCCCTACGGTTCAACGACATATGATATCGGGGGAAATTTTGCTGCTCACATGTTT
AAAGGTCGTGACTACGTTCATTGCTGCATGCCTAACATGGACTTACGTGACGTCATGCGTCACAATGCTC
AAAAGGATAGCATTGAACTGTACCTTTCAAAGCTTGCGCAAAAGAAAAAGGTAATACCGCCATATCAAAA
GCCATGCTTTGATAAATACACGGACGATCCGCAATCAGTAGTGTGCTCGAAACCTTTTCAGCACTGCGAA
GGCGTTTCGCACTGCACGGATAAAGTATACGCTGTCGCTTTGCACAGTTTATACGACATTCCAGCAGATG
AATTTGGGGCAGCACTTCTGAGGAGAAATGTTCATGTCTGCTATGCTGCCTTCCACTTTTCTGAGAATCT
TCTTTTAGAAGATTCGTATGTCAGTCTTGACGACATAGGCGCTTTCTTCTCGAGAGAGGGCGATATGTTG
AACTTTTCTTTTGTAGCAGAGAGTACTTTAAATTATACTCATTCCTATAGTAATGTGCTTAAGTATGTGT
GTAAGACTTACTTCCCCGCTTCTAGTAGAGAAGTGTACATGAAGGAGTTTTTGGTAACTAGGGTAAATAC
TTGGTTTTGTAAGTTTTCAAGGTTAGATACCTTTGTACTATATAGAGGTGTATACCACAGAGGTGTAGAC
AAGGAGCAATTTTACAGTGCAATGGAAGATGCTTGGCATTACAAAAAGACTTTGGCGATGATGAATAGCG
AAAGAATCCTCTTAGAGGATTCATCGTCTGTTAATTATTGGTTTCCAAAGATGAAAGATATGGTGATAGT
ACCTTTGTTCGACGTATCTTTACAGAACGAGGGGAAAAGGTTAGCAAGAAAGGAGGTCATGGTCAGCAAG
GACTTCGTTTATACTGTGCTTAATCATATTCGCACATACCAGTCGAAAGCGCTTACTTACGCCAATGTAT
TATCGTTCGTTGAGTCGATAAGATCAAGAGTGATAATCAATGGGGTGACTGCGCGCTCAGAGTGGGATGT
GGATAAGGCTTTGTTGCAGTCCCTGTCAATGACTTTTTTCTTGCAGACCAAATTGGCCATGCTCAAGGAT
GACCTCGTGGTTCAGAAATTCCAAGTGCATTCCAAATCGCTCACTGAATATGTCTGGGATGAGATTACTG
CTGCTTTTCACAATTGTTTTCCTACAATCAAGGAGAGGTTGATTAACAAGAAACTCATAACTGTTTCGGA
AAAGGCTCTTGAAATTAAAGTACCTGATTTGTATGTAACTTTCCACGATAGATTGGTTAAGGAGTACAAG
TCTTCGGTGGAAATGCCGGTACTGGACGTTAAAAAGAGCTTGGAAGAAGCAGAAGTGATGTACAATGCTT
TGTCAGAAATCTCAATTCTTAAAGACAGTGACAAGTTTGATGTTGATGTTTTTTCCCGGATGTGTAATAC
ATTAGGCGTAGATCCATTGGTGGCAGCAAAGGTAATGGTAGCTGTGGTTTCAAATGAGAGTGGTTTGACC
TTAACGTTTGAGAGGCCTACCGAAGCAAATGTCGCACTTGCATTGCAACCGACAATTACATCAAAGGAGG
AAGGTTCGTTGAAGATTGTGTCGTCAGACGTAGGTGAGTCCTCAATCAAGGAAGTGGTTCGAAAATCAGA
GATTTCTATGCTTGGTCTAACAGGCAACACAGTGTCCGATGAGTTCCAAAGAAGTACAGAAATCGAGTCG
TTGCAGCAGTTCCATATGGTATCCACAGAGACGATTATCCGTAAACAGATGCATGCGATGGTGTATACTG
GTCCGCTAAAAGTTCAACAATGCAAGAACTATTTAGACAGCCTGGTAGCCTCGCTCTCTGCTGCGGTATC
AAACCTGAAGAAGATAATCAAAGACACAGCTGCTATAGATCTCGAGACTAAGGAAAAATTTGGAGTCTAC
GACGTGTGCCTTAAGAAATGGTTGGTGAAACCTCTATCAAAAGGACATGCTTGGGGTGTGGTGATGGACT
CAGACTATAAGTGCTTTGTTGCGCTTCTCACATACGATGGCGAGAACATTGTGTGCGGAGAGACATGGCG
TAGAGTCGCAGTGAGCTCCGAATCTTTGGTGTATTCAGATATGGGGAAGATAAGAGCTATACGCTCTGTG
CTTAAAGACGGTGAACCCCATATAAGCAGTGCAAAGGTTACACTTGTTGATGGTGTTCCTGGTTGCGGAA
AGACAAAGGAGATTCTTTCGAGGGTCAACTTTGACGAAGATCTAGTTCTGGTACCAGGAAAACAGGCTGC
TGAAATGATAAGAAGAAGGGCAAACAGTTCTGGTTTAATCGTGGCGACCAAGGAGAATGTAAGGACGGTA
GACTCTTTCTTAATGAATTACGGTCGAGGTCCGTGCCAATACAAAAGGCTGTTTCTGGATGAAGGTCTAA
TGTTACACCCTGGTTGTGTTAATTTTCTGGTTGGCATGTCTCTATGCTCCGAGGCTTTTGTTTATGGAGA
CACCCAGCAGATTCCTTACATCAACAGAGTTGCAACTTTTCCCTATCCTAAGCATTTGAGTCAACTCGAG
GTCGATGCTGTTGAAACTCGCAGAACAACGTTGCGGTGTCCAGCTGATATCACCTTCTTCTTGAATCAGA
AGTACGAAGGGCAAGTTATGTGCACATCAAGTGTTACACGCTCGGTGTCACACGAGGTCATCCAAGGTGC
AGCGGTAATGAATCCAGTGTCTAAACCACTTAAAGGGAAGGTGATTACATTCACTCAGTCAGACAAGTCA
TTGCTGCTCTCGAGGGGTTACGAAGATGTGCATACCGTTCATGAGGTGCAAGGGGAAACGTTTGAAGACG
TCTCACTAGTGAGGCTGACGCCAACACCCGTGGGAATAATTTCAAAGCAGAGTCCGCACCTGTTGGTCTC
ATTGTCTAGGCATACAAGGTCGATCAAATATTACACAGTTGTGCTAGATGCAGTCGTTTCAGTGCTTAGA
GATCTGGAGTGTGTGAGTAGTTACCTGTTAGATATGTACAAAGTTGATGTGTCGACTCAATAGCAATTAC
AGATAGAATCGGTGTACAAAGGTGTTAACCTTTTCGTCGCAGCACCAAAAACAGGAGATGTTTCTGACAT
GCAATATTATTACGACAAGTGTTTGCCGGGAAACAGTACTATACTCAATGAGTATGATGCTGTAACTATG
CAAATACGAGAGAATAGTTTGAATGTCAAGGATTGTGTGTTGGATATGTCGAAATCGGTGCCTCTTCCGA
GAGAATCTGAGACGACATTGAAACCTGTGATCAGGACTGCTGCTGAAAAACCTCGAAAACCTGGATTGTT
GGAAAATTTGGTCGCGATGATCAAAAGAAATTTCAACTCTCCCGAATTAGTAGGGGTTGTTGACATCGAA
GACACCGCTTCTCTAGTAGTAGATAAGTTTTTTGATGCATACTTAATTAAAGAAAAGAAAAAACCAAAAA
ATATACCTCTGCTTTCAAGGGCGAGTTTGGAAAGATGGATCGAAAAGCAAGAGAAGTCAACAATTGGCCA
GTTGGCTGATTTTGACTTTATTGATTTACCAGCCGTTGATCAATACAGGCACATGATCAAGCAGCAGCCG
AAACAGCGTTTGGATCTTAGTATTCAAACTGAATACCCGGCTTTGCAAACTATTGTGTATCATAGCAAGA
AAATCAATGCGCTTTTTGGTCCTGTATTTTCAGAATTAACAAGACAGCTGCTAGAGACAATTGACAGTTC
AAGATTCATGTTTTATACAAGGAAAACGCCTACACAGATCGAAGAATTTTTCTCAGATCTGGACTCTAAT
GTTCCTATGGACATATTAGAGCTAGACATTTCCAAGTATGACAAATCACAGAACGAATTTCATTGTGCAG
TCGAGTATGAGATTTGGAAAAGGTTAGGCTTAGACGATTTCTTGGCTGAAGTTTGGAAACACGGGCATCG
GAAGACAACGTTGAAAGACTACACAGCCGGAATAAAAACGTGTTTGTGGTACCAGAGGAAAAGCGGTGAT
GTCACCACATTCATTGGAAACACGATCATTATTGCTGCATGTCTGTCCTCTATGCTACCGATGGAGAGAT
TGATTAAAGGTGCCTTTTGTGGTGATGATAGTATATTATACTTTCCAAAGGGCACTGATTTCCCCGATAT
TCAACAGGGCGCAAACCTTCTCTGGAATTTTGAAGCCAAGTTGTTTAGGAAGAGATATGGTTACTTTTGC
GGTAGGTACATAATTCACCATGACAGAGGCTGTATTGTATATTATGACCCTCTAAAATTGATCTCGAAAC
TCGGTGCAAAACACATCAAGAATAGAGAACATTTAGAGGAATTTAGGACCTCTCTTTGTGATGTTGCTGG
GTCGTTGAACAATTGTGCGTACTATACACATTTGAACGACGCTGTCGGTGAGGTTATTAAGACCGCACCT
CTTGGTTCGTTTGTTTATAGAGCATTAGTTAAGTACTTGTGTGATAAAAGGTTATTTCAAACATTGTTTT
TGGAGTAAATGGCGTTAGTAGTCAAGGACGACGTTAAGATTTCTGAGTTCATCAATTTGTCTGCCGCTGA
GAAATTCTTACCTGCTGTTATGACTTCGGTCAAGACGGTACGAATTTCGAAAGTTGACAAAGTGATTGCA
ATGGAAAACGATTCGTTATCCGATGTGAATTTGCTTAAAGGTGTAAAGCTTGTTAAGGATGGTTATGTGT
GTTTAGCAGGGTTAGTTGTGTCCGGGGAGTGGAACCTACCCGACAACTGCAGAGGTGGAGTAAGCGTTTG
TTTGGTTGATAAGAGAATGCAAAGAGATGACGAAGCAACACTTGGATCTTATAGAACCAGTGCAGCTAAG
AAACGATTTGCCTTCAAATTGATCCCGAATTATAGCATTACTACCGCCGATGCTGAGAGAAAAGTTTGGC
AAGTTTTAGTTAATATTAGAGGTGTTGCCATGGAAAAGGGTTTCTGTCCTTTATCTTTGGAGTTTGTCTC
AGTTTGTATTGTACACAAATCCAATATAAAATTAGGCTTGAGAGAGAAAATTACTAGTGTGTCAGAAGGA
GGACCCGTTGAACTTACAGAAGCAGTCGTTGATGAGTTCATCGAATCAGTTCCAATGGCTGACAGATTAC
GTAAATTTCGCAATCAATCTAAGAAAGGAAGTAATAAGTATGTAGGTAAGAGAAATGATAATAAGGGTTT
GAATAAGGAAGGGAAGCTGTTTGATAAGGTTAGAATTGGGCAGAACTCGGAGTCATCGGACGCCGAGTCT
TCTTCGTTTTAACTATGGCTTACACAGTTTCCAGTGCCAATCAATTAGTGTATTTAGGTTCTGTATGGGC
TGATCCATTAGAGTTACAAAATCTGTGTACTTCGGCGTTAGGCAATCAGTTTCAAACACAACAGGCTAGA
ACTACGGTTCAACAGCAGTTCTCTGATGTGTGGAAGACTATTCCGACCGCTACAGTTAGATTTCCTGCTA
CTGGTTTCAAAGTTTTCCGATATAATGCCGTGCTAGATTCTCTAGTGTCGGCACTTCTCGGAGCCTTTGA
TACTAGGAACAGGATAATAGAAGTTGAAAATCCGCAAAATCCTACAACTGCCGAGACGCTTGATGCGACG
AGGCGGGTAGACGATGCGACGGTGGCCATTAGGGCCAGTATAAGTAACCTCATGAATGAGTTAGTTCGTG
GCACGGGAATGTACAATCAAGCTCTGTTCGAGAGCGCGAGTGGACTCACCTGGGCTACAACTCCTTAAAC
ATGATGGCATAAATAAGTTGAACGAACATTAAACGTCCGTGGCGAGTACGATAACTCGTAGTGTTTTTCC
CTCCACTTAAATCGAAGGGTTGTCGTTGGGATGGAACGCAATTAAATACATGTGTGACGTGTATTTGCGA
ACGACGTAATTATTTTTCAGGGGTTCGAATCCCCCCCGAACCGCGGGTAGCGGCCCA
Citrus yellow mosaic virus (genomic DNA, Accession Number: NC_003382.1) (SEQ ID
NO: 434):
TGGTATCAGAGCTTGGTTATGTTCTTACAACGATGGGAGCTTAAGTTCTTCCATTAGGTCTGAGGAAAGA
GTTGGTTGTATGGTGTGTTTAGTTCCTATCTGTATTGTTATTCCTGTGTTCATGATATAGAAAACGATCA
TCGCGAAAAGGGTGAAGGCACTATATCTGGCAGCGAGAGGAGAGTAAGTCCAGTGAAACCCTTCGCATGA
CGCTAAAGGTGATCTAATCTATGTCTAGAATTTGGGAAGAAGCAATACAGAAATGGTATGAGACATCCCA
TACAGCTAATCTCGAGTACTTAGATCTAGCTTCAAAACCAAAAGTTTCCAATTCAGAAATTTCACACAAC
CTTGCTGTAGTTTATGATCGTTTGAATCTGTTTAGCCGTGTCTCTATTAAAAATTTCAAAAGTATCCAAG
AAACCTTAGAAAAACAAGACCTTAGAATTCGAAAGCTTGAGTCTAGTTTGAAAACCTTAACCAGTGAGTT
TATAGCCCATAAACCTTTGTCCAAAAGTGAGGTAAAAGCCTTAGTCACAGAAATTGCCAAACAGCCAAAG
CTTGTTGAAGCACAGGCCCTTCAGTTGACCGAGTCTCTTAACCAAAAGCTTGATAGGGTTGAAACCCTAA
TAGCTAAGGTTGAACGGTGGGTTCATTCATGACCTACCAGAATACTGAAAAGACTCCTACATACAAAAGA
GCTTTAGAAGCAACCGAGCCTATCAACAGTCCCGCCCTAGGTTTTATAAATCCAGAAGATTATTCAGGAG
GCATTACTGGTACGAAGGCTTTGATTAAGCAAAACAACCTGCTCATTCAACTTGTGGTGGAACTTTCTGT
CAACGTCAACAGCTTATCTGAACAGGTTGCTCAACTTACAAGGCAACTTGGAAAGCAACCCCAGCAAGGC
TCATCAACAGCAACCTTACCTGACGATTTGGTTGACAAACTCAAGAACCTTTCCTTAGGTACTGAGAAAA
AGAAGGAGAAGCGTGGTACCTTCTACGCTTACAAAGACCCATACCTGATCTACAAGGAAGAGGTAGAAAA
GTTAAAGAAGCAACAACAATGAGTACCAGTCGTGCTCGTACAGTTATAGAGCAACTCCCTCCGGCTACAA
CAGCTCGGGTGGAAGAAAGGGATAATACTCCCCTCTATGATGACCAAATCAGAGATTATAGGCAGTGGCA
GCGGCGGCGGCACAACATGGGGCGGAGATGGAATCAGTTGATAGGACGACCCTACAATCAGACCTTGGAA
CAGGTTGTGGACCCTGAAGTAGCTTTACAGCTATCAATGCAGGAGCGTGCCAGACTAGTACCAGCAGAGG
TACTTTACAGATCAAGAACTGATGATCGGCACCATCAAGTCTACATTCACAAGTCAGAGGAGGCTATCCT
TTGTGTAGATGGTGATCAAGTTGACCGGTTACTAATTCAACCGGAAAGTGCTGAACAGTTAAGCAGGAGC
GGTATGTCCTTCATTCATATGGGCATAGTTCAGGTTCGGATCCAGATCTTACACAGACAGCATGAGGGAA
CAACAGCCCTTGTGGTGTTTAGAGACAATCGGTGGCAAGGAGACCAGTCAATATTTGCCACCATGGAGCT
GGATTTAACTAAAGGTATGCAGATGGTGTACATAATCCCGGACACCATGATGACAGTCAGAGACTTCTGC
CGGAATGTTCAAATTTCCATATTAACAAAAGGATATGGGAATTGGCAGAATGGCGAGGCAAATCTGCTTG
TTACAAGGGGAATTGTTGGACGGTTATCAAATACCCCTAATGTGGCCTTTGCCTATCAGATCCAAAATGT
TACCGACTACTTGGTCAGTCATGGAATTCAGGCCCTGCCAGGACGGCGATATTCTACTGCAGATATACAG
GGCCAACAATGGTTCCTAAGACCATCCAATATCCCAGCAGTCCCAATGGCCCCCACCAACGTGGATACAA
GAAACATGATTGATGGATCTATTTCTCTTAGATTCAACAGTTACCAACCAGCTCCAGATCCAACCCCTGT
TGCTTATAATCAGCATGATGAGGAAGTACCCCCTGATGAAGATGAAGAGCAGATCCGTAATCATACCATC
GCTTTATGGCGGGAAGATGACGAGGTATGGGATACACTTGGTGAACCTTCGGGCAAATTTGATTTTTATG
TCCGTTATACTCGACCTGCACATGCTCTACAAGATCCTGCTCATATTGTTGCTACTGGATGGGATGACCT
TGACAATGATCCATCCACCTCAAGTCCTTCTAATAATATCCTTACTTACCTCACCCCTTCTTCCTCTTCT
GATGAGGATGATGACATGTCCTATCTCCAATACCTTGCTCAACAATCACCTGTTCCTTCTCCTACACAGG
ATTTCACCAATCCTTTTTCGGAAGGTGGTGGGGAATCTACCTACCCTTACCCCTCATTTCAACCACCATT
CGACCTTCAATCAGACGACTCATATGGTACTTTGGCAACTTGGAGTGAATATGATGCTATGAGTCAATCA
AACAGTCCTTCATCACACTCAGATGCTATTCAACATCTTAGTTTCCAGCACCCAAGTGCAGATACTGTCC
TTGATTTTGACAGATATTCTTTTACAACAAGTGAGGATGACGTGGTTCAATCAGCCTGGATATCTGAAAA
TCTATTTCGTGAAAACACCGGAAACGGTGAAGTTCACAATCTTGTTCCACCTAGACCGGACACCCCTCGG
GGTGATGAGGTCAAAGGAACTCAGGAATCCATGGCCCATACTGTTGCAGTAACCACAGAGGAATCAAAAC
ATGAGGCTGAATTTGACTATCCGGCTTTTGCCAGATTACAAGCCCATGAAGAGTCAGGGCGGCCCAAACC
CAAAACTGAGAAAGTCTTATCCTCAGCAATTTCTTCATATACCCCACCAACGGATACTGCAATGACACCT
GTTGCGTACCCCCCAGCCCAAAATATAGCCAGCCCAAGTTACAATCCAAGCCCACAAATGCCCATGTTCG
AAGGGTATTATCCCAAAAGGCCAAATTTTAAGAGGGATAATCATGCCTTTATCAGTCTTCCCTCGGCCCA
ACAAAATACTGGGGCTTTATTCATTATGCCTCAACAAATTGGCCTGTTTCATGAGGTTTTTACTTCATGG
GAAGCTATAACAAAGGCCTATGTTGCTCAACAGGGTATCACAGACCCAAGGGATAAAGCCGAGTTCATTG
AAAACATGTTGGGTCCAACAGAAAAGATAATTTGGACTCAATGGCGTATGGGCTACGCCGATGAATATGA
GAACCTTGTTACAACTGCTGATGGTCGTGAGGGTACTCAAAATATACTCTCTCAGATGCGAAGGGTCTTT
TCCTTAGAAGATCCAACCACAGGTTCAACTGCAGTCCAAGATGAAGCCTACAGAGACTTGGAGAGGCTTA
CTTGTGATTCTGTCAAGCATATAGTCCAATACTTAAATGACTTTATGCGGATTGCAGCAAAGACTGGGCG
CATGTTCATAGGCCCAGAATTGAGTGAAAAGTTATGGCTTAAAATGCCAGGTGACCTAGGCCAAAGAATG
AAGAAGGCCTATGAAGAAAAACATCCAGGGAACATTGTTGGTGTTTGCCCTCGGATTCTGTTTGCTTATA
AGTACCTTGAAGGCGAATGCAAAGATGCAGCGTTCAGACGCTCCCTGAAAAATCTATCCTTTTGCAGCTC
AATCCCTATCCCAGGCTATTACGGTGGTAAAAGTGGAGAGAAACGTTATGGTGTAAGGCGCACAACCACT
TATAAGGGAAAGCCTCATAGCACCCATGCAAGGATTGAAAAGACAAAACATTTGCGCAATAAAAAGTGCA
AGTGTTATCTGTGTGGGGAAGAAGGTCACTTCGCCCGGGAATGTCCAAATGACCGGCGAAATGTGAAACG
AGTTGCAATGTTCGAAGGTTTAGACCTCCCAGATGACTGTGAGATAGTCTCCATCGATGAAGGTGATCCA
GATAGTGATGCAATCTTCAGTATTTCCGAAGGAGAAGAAGCTGGAACTCTTGAAGAACAATGTTTTGTGT
TCCAGGAAGAATGCAATGGAACATATTGGCTTGGTAAAAGAGGTGGATACCAGGATCTCGTGCAAATCTC
TAAGGAGATCTACTATTGCCAGCATGAATGGGAGGAGAATCAACCCATTAATGATCCAGCACATGTTCGG
TGTTACCCTTGTAAAAGGGAAACCACTCAGAGAGCTCGCTTACATTGCAAGCTATGCCACATAACATCTT
GCCTTATGTGTGGCCCCACCTATTTCAACAAAAAGATTACTGTCCAGCCAATGCCTCAAGCACCCTTCAA
CCAAAAGGGATTGTTACAGCAACAGCAGGAGTACATCGCCTGGTGCAATAATGAAATTGCCAGGTTAAAG
GAAGAAGTTGCTTTTTACAAGCAGCTCGCCCAGGAGAGAGAATTGCAGTTGCAACTTGAGCAATCAAGGA
AGGAGCTAGCAGGAGTAGACTCTCGCAGGCGAAAAGACAAAGGAATAGTAATCGATGAAGGGTCATGCTA
CTTCAATCCTGAAGAAACAACCAGGATAATTGCTCACGGTGACACACAAGTTACCAAAACTCGACCAGTT
AAGAATATGCTCTACAACATGGATGTGCGAATGGAAATTCCAGGCATCCCAGCTTTTACAGTAAAGGCGA
TTCTTGACACAGGAGCAACAACCTGCTGTATTGACAGCAGAAGTGTACCAAAAGATGCCCTTGAAGAGAA
TTCATTTGTGGTAAATTTCTCAGGCATCAATTCCAAGCAACAAGTCAAGCAGAAGCTTAAAACTGGAAAA
ATGTTCATCAATGAGCATTACTTCCGGATCCCATATTGTTACAGCTTTGAGATGCAAATTGGTGATGGCA
TCCAACTTATCCTTGGGTGCAACTTTATACGAAGTATGTATGGTGGTGTACGATTAGAAGGTAATACTAT
AACCTTCTACAAGCAGATAACAAGTATCAACACCAGGCTTGCTGCACCTCTCCTTAAGCAAGAAGAAGAG
GAGAAAGAAGAAGAACTCAACCTGGAAGAGCACAGGTTGATTCAAGAAATGGTTGCATACTCCACTGAGC
GGCCATTTGTTCAATTCCAACAAAAGTTTGCAGGGCTTATTCAAGACTTAAAAGCCCAGGGATACATTGG
GGAAGAGCCTATGAAGTATTGGGCCAAAAACCAAGTTGTTTGCCATCTGGACATTAAAAACCCAGATATG
GTAATTGAAGATCGCCCACTGAAGCATGTGACACCCCAGATGGAAGAAAGCTTTCGCAAGCATGTGGAAG
CCCTGTTAAAAATAGGAGCAATCCGGCCCAGTAAAAGTCGGCACAGAACCACAGCTATAATAGTCAACTC
TGGAACCAGCATAGACCCTATTACAGGGAAGGAGGTTAAGGGAAAGGAGCGAATGGTCTTTAACTATAAA
AGGTTAAATGACCTAACTAATAAAGATCAGTACAGCCTTCCTGGAATCCAGACGATCCTGCAGAGATTAA
AGGGGAGCACAATATTTTCCAAATTCGACCTAAAAAGTGGCTTTCATCAGGTAGCAATGCATCCAGATTC
AATAGAATGGACAGCTTTTTGGGTGCCCAGCGGTCTTTATGAATGGTTAGTTATGCCATTCGGATTAAAG
AATGCTCCAGCAATTTTTCAAAGGAAAATGGATCACTGTTTCAAAGGCACGGAGGCCTTTATTGCCGTCT
ACATCGACGACATCCTAGTATTCTCAAAGACTGAACAGGATCATGAGAAGCATTTACAGATTATGCTCGC
TATCTGTCAAAAGAATGGGCTTATCCTAAGCCCAACAAAGATGAAAATTGCCCAAGCTGAAATTGAATTC
CTTGGGGCAATCATTCACAAAGGGCTTATCAAGTTGCAGCCCCACATTGTTCAAAAGTTGCTCACTTTTA
CCAATAAGCAACTTGAGGAGGTTAAAGGGCTTAGATCATGGCTAGGCCTGCTAAACTATGCAAGGAGCTA
TATTCCCCATATGGGCCGTCTACTTAGCCCATTATATGCCAAAGTCAGCCCAACTGGTGAGCGGAGAATG
AACAGACAAGATTGGGCCCTGATTGACAAAATAAGAGCCCAAGTCCAAAATCTACCAGCCCTGGAATTAC
CACCTGCAGACTGTTTCATCATCATCGAAACGGATGGATGCATGGATGGTTGGGGAGGTGTCTGCAAATG
GAAAGTAGCGCAATACGACCCTCGAAGTTCAGAAAGGGTTTGTGCTTATGCAAGTGGGAAGTTCAACCCA
CCAAAGTCAACAATTGATGCGGAGATACATGCAGTGATGAACAGCCTCAACAACTTCAAAATCTATTACC
TAGACAAGTCCAGTTTATGTTTGAGGACTGACTGTCAAGCTATTATTAGCTTCTTTAATAAGTCCAATGT
TAACAAACCGTCTAGGGTTAGATGGATTGCTTTCACAGATTTCCTTACTGGTCTAGGAATCCCTGTAAAT
ATAGAGCACATAGATGGAAAAAATAACCATCTGGCTGATGCTCTGTCCAGATTAGTAACTGGTTTTGTTT
TTGCAGAACCACAATGTCAAGACAAGTTCCAGGACGATTTAGGGAAATTGGAAGCAGCTCTTCAGGAGAA
GAAAGAGGCTCCGCAAGCAATGCACGTAGAATATGTCTCCCTGTTGATCAGATCAGCGGACCGCATTACC
CGCTCGCTCTGCTTTATGAGGGACTCGTCTCACAGCAGAATTTACTCATGCAGGCCAGGCAAAGAACCAA
TGAAGGCCTTAATCTGCGAACAGAAGTCATGCCAATCCAAAGGCGACTTAGGGAATACGAGGACTGTGCA
CTCCAAGAGTGCATTCAATCAGCAAGACAACTGGTGGCCCTCCACCAGCACAAACTCGCTTACATCAGAA
GCAAAGCTACAAGGGACAACGCATATGCCGATAGGCTACCCACATGCAATCGGGACCACGAGCAACTGTG
TGAAGTGGTCGAGCTATTAGAAGGAATCTCGGAAAGAATCAGCGATACAGCTGTCTAGGACAGCTGGCTT
CAATTATGGAGCGTGATGGACCCCCCCGCAATAATCCAAAGTTTGGTGTGCTTTTAGTAGTGCGTCTTTA
TGGACCACTACTTTATTGTAATAATCGATGCTTTTTGTAGTGCGCTCTTCGTGCGCTCTACTTTATGCTT
TTGCTTTTGTAAGTGCGCTGTAAGTGCGCCTGTCTTTCTTCAGATGCTTATCCTTTAAGCATCTTTTGCT
TTTTGCGTGGCATCCTTTAGTTCACAATTTAAAGAATGACGATGGGGCCCAAGATGTGCACCCGGTTCTC
TAAATTGCCTATATAAGGATATGCCATAGCCTTGTTTTTGCAAGTCAGGAATACCTGAGCATAACTTGGC
TAAGCAAAAGTTTGTAAGTGTTCTAAGCTTTCATTTGTAAACTTTCTGTTTGGTTTTAATAAAATCTCTC
GTCAATCGTTGTGAACATATATTGTTTGTTTGTATTGTTGTATCTTATTTGTTGTGGTGATAATGGTAA
Oat blue dwarf virus (genomic DNA, Accession Number: NC_001793.1) (SEQ ID
NO: 435):
GTGTCCCAGTGTCATTATTCCGCTCAGTTTCAGATCTGCCGGAATTCTCCAAGCATCCCGCCCCAAAAGC
CGGCTGCTTAAAATCTGATCTTCTCCATCTTGTCAAGTGTCGTTATGACCACATACGCCTTCCACCCGCT
GCTCCCCACCCCGACCTCCTTCGCCACTATCACTGGGGGTGGTTTGAAGGATGTTATCGAAACCCTCTCG
TCCACCATCCACAGAGACACGATCGCAGCACCCCTCATGGAGACCCTCGCCTCGCCTTACCGAGACTCCC
TTCGCGACTTCCCTTGGGCCGTCCCCGCCTCCGCCCTGCCCTTCCTCCAGGAATGTGGCATCACGGTCGC
CGGCCACGGTTTCAAAGCTCATCCCCACCCTGTCCACAAAACCATCGAGACCCACCTCCTCCACAAGGTT
TGGCCTCACTATGCCCAAGTCCCTTCTTCCGTCCTCTTCATGAAGCCCTCGAAGTTCGCCAAACTCCAGC
GGGGCAACGCCAACTTCTCCGCACTCCACAACTATCGCCTCACCGCCAAAGACACCCCGCGGTATCCTAA
CACTTCAACCTCTCTCCCCGACACCGAGACCGCCTTCATGCATGACGCCCTCATGTATTACACCCCCGCT
CAAATTGTTGACCTGTTCCTTTCCTGCCCGAAGCTCGAGAAACTGTACGCCTCCCTTGTCGTCCCCCCCG
AGTCCTCCTTCACCTCTATCTCTCTCCATCCAGATCTTTACCGCTTTCGCTTTGACGGGGACCGTTTGAT
TTATGAGTTGGAGGGCAACCCCGCCCACAACTACACCCAACCTCGATCCGCCCTCGACTGGCTCCGCACA
ACCACCATCCGCGGACCAGGCGTTTCTCTCACCGTGTCCAGGCTCGACTCGTGGGGTCCCTGCCATTCCC
TCCTCATCCAGCGCGGCATTCCCCCCATGCACGCCGAGCACGACTCCATCTCGTTCAGGGGTCCACGCGC
CGTCGCCATTCCCGAGCCCTCCTCCCTCCACCAGGATCTGCGCCACCGTCTCGTTCCAGAGGACGTGTAT
AACGCCCTCTTCCTCTACGTCCGCGCTGTCCGCACGCTCCGCGTAACCGATCCCGCCGGCTTTGTCCGCA
CCCAGTGCTCTAAGCCCGAGTACGCTTGGGTCACTTCCTCCGCTTGGGACAACTTGGCCCACTTCGCCCT
CCTCACCGCTCCACACCGGCCCCGCACCTCGTTCTACCTATTCTCCTCTACCTTCCAGCGCCTTGAGCAC
TGGGTCCGCCATCACACCTTCCTCCTCGCCGGCCTCACCACAGCCTTTGCTCTCCCGCCGTCTGCCTGGC
TCGCGAACCTCGTCGCCCGCGCCTCCGCTTCACACATCCAAGGCCTCGCGCTAGCCCGCCGGTGGCTCAT
CACTCCCCCTCATCTCTTCCGCCCCCCTCCACCCCCAAGCTTCGCTCTTCTTCTCCAGCGCAACTCCACC
GGCCCGGTCCTTCTCCGTGGCTCCCGCCTCGAGTTTGAGGCCTTCCCTTCTCTCGCCCCACAACTCGCCC
GTCGCTTTCCATTCCTCGCTCGCCTTCTCCCCCAGAAACCCATCGACCCCTGGGTCGTCGCGAGCCTCGC
TGTCGCCGTTGCTATACCCGCCGCCTCCCTCGCCGTTCGCTGGTTCTTCGGCCCCGACACCCCCCAAGCC
ATGCACGACCGATACCACACCATGTTCCACCCCAGAGAGTGGCGCCTCACCCTGCCCAGGGGCCCCATCT
CATGTGGCCGCTCCAGCTTCTCCCCCCTTCCCCACCCACCTTCGCCCACTCCCGCTCCCGACTCCCGAGC
TGAACCCCTCCAGCCACCCTCCGCTCCACCCTCGACCCACGAGCCGGCTCCCGCCGATCTCGAGCCCCAA
GCTCCTCCGGCCCACGCCCCCCAGACCGAGCCTCCGAGTCCCGTGATCGAGCAAGAAGCGCGTCCGAATC
CCCTTCCCGCTCCTGCCCCGCTTTCTGCTCCCACCCCCTCCGCTTCCGCGCCTTCACTTGCCCCAACACC
CTCGGCCCCCGAGCCTCCCTCGCCGACCGCTTCCGAGCAGGCCGCGTCCCTCATCCCTGCTCCCTCTTCC
GCCCTCGTCGTGGAGCCATCCGGCGTCGTCTCTGCCTCATCTTGGGGCGCCACCAACCAGCCGGCCGATC
AAGTCGATGACTCCCCTCTCGCTCGCGATCCCAGCGCCTCCGGCCCCGTCCGCTTCTATCGAGACCTCTT
CCCCGCCAACTACGCGGGTGATTCCGGCACCTTCGACTTCCGCGCCCGCGCCTCAGGCCGCTCTCCCACC
CCATACCCCGCCATGGATTGCCTCCTCGTCGCCACCGAGCAAGCCACCCGCATCTCTCGAGAGGCCCTCT
GGGACTGCCTCACAGCCACCTGCCCCGACTCATTCCTCGACCCCAAGAGCATCGCCCAGCATGGCCTCAG
CACCGATCACTTCGTCATCCTCGCTCATCGCTTTTCCCTATGTGCCAACTTCCACTCCGCCGAGCACGTC
ATTCAGCTCGGGATGGCCGATGCCACCTCCATTTTCATGATCAACCACACGGCTGGCTCCGCGGGCCTCC
CGGGCCACTTCTCCCTCCGCCTGGGTGACCAGCCCCGTGCCCTCAACGGTGGCCTCGCTCAGGACCTCGC
CGTCGCCGCCCTCCGATTCAACATCTCCGGTGATCTCCTCCCAACCCGATCCGTTCACACTTACAGGTCT
TGGCCAAAGCGCGCCAAGAACCTTGTGTCCAACATGAAGAACGGCTTTGACGGAGTCATGGCCAGCATCA
ACCCGATCCGACCCAGCGATGCTCGCGAGAAGATCGTCGCCCTCGACGGTCTCCTAGACATTGCCCGACC
CCGATCCGTCCGCCTCATCCACATTGCTGGTTTCCCAGGCTGCGGAAAAACACATCCGATCACCAAGCTC
CTCCACACCGCCGCCTTCCGCGACTTCAAACTCGCCGTCCCGACCACCGAGCTCCGGTCTGAGTGGAAAG
AGCTCATGAAGCTCTCACCCTCTCAGGCCTGGCGCTTCGGCACCTGGGAGTCCTCCCTTCTCAAGAGCGC
CAGGATCCTCGTGATCGATGAGATCTACAAGTTGCCCCGAGGGTACCTCGACCTAGCCATCCACTCCGAC
TCGTCCATCGAGTTTGTTATCGCCCTGGGAGATCCTCTGCAAGGCGAGTATCACTCCACTCATCCCAGCT
CCTCCAACTCTCGCCTCATTCCCGAAGTCAGCCATCTCGCTCCCTACCTCGACTACTACTGCCTCTGGAG
TTACCGCGTCCCCCAAGACGTCGCCGCTTTCTTCCAGGTTCAGAGCCACAACCCTGCTCTCGGGTTTGCC
CGTCTCTCGAAGCAGTTTCCCACGACCGGGCGCGTCCTCACCAACTCACAGAACTCGATGCTTACCATGA
CGCAGTGCGGCTACTCTGCCGTCACCATTGCCTCAAGCCAGGGTTCCACCTACAGCGGCGCCACGCACAT
CCACCTTGACCGCAACTCATCGCTCCTCTCCCCTTCGAACTCCCTCGTCGCCCTCACTCGCTCGAGAACC
GGCGTGTTCTTCTCCGGGGACCCTGCTCTTCTCAACGGTGGTCCCAACTCCAACCTCATGTTCTCTGCCT
TCTTTCAGGGCAAGTCTCGCCACATTCGCGCCTGGTTCCCCACCCTTTTCCCTACGGCCACTCTCCTCTT
CTCCCCCCTCCGCCAACGCCACAACCGCCTCACTGGCGCCCTCGCTCCCGCCCAACCTTCCCACCTCCTG
CTCCCTGACCTTCCGAGCCTCCCTCCTCTCCCCGCCTCCGGTCCCTACTCCCGCTCATTCCCAGTTCGAT
CTCGCTTCGCCGCGGCCGTCAAGCCTTCCGACCGGTCAGACGTCCTCTCGTGGGCCCCTATCGCCGTCGG
TGACGGGGAAACCAACGCCCCTCGCATTGACACCTCCTTCCTGCCCGAAACTCGCCGCCCGCTTCATTTT
GATCTTCCCTCGTTCCGCCCCCAAGCCCCACCGCCTCCCTCTGACCCAGCCCCTTCTGGGACCGCCTTTG
AGCCCGTTTACCCCGGCGAAACCTTCGAAAATTTGGTCGCCCACTTCCTTCCGGCTCACGACCCCACTGA
CCGCGAAATCCACTGGCGTCGGCAGCTTTCCAACCAGTTTCCCCATGTCGATAAGGAGTACCACCTCGCG
GCTCAGCCAATGACGCTCCTCGCTCCCATCCACGACTCCAAGCACGACCCCACCCTCCTTGCCGCCTCCA
TCCAGAAACGACTTCGATTTCGACCCTCCGCCTCTCCCTACCGAATCTCCCCTCGTGACGAGCTGCTTGG
CCAGCTCCTCTACGAGAGTCTCTGCCGCGCGTATCATCGTTCCCCAACCACCACCCACCCTTTCGATGAG
GCCCTCTTCGTCGAGTGTATCGACCTGAACGAATTCGCTCAACTCACCAGCAAAACTCAGGCCGTCATCA
TGGGCAACGCCCGCCGCTCTGACCCAGACTGGCGCTGGTCCGCCGTCCGGATCTTCAGCAAAACCCAGCA
CAAGGTCAACGAAGGTTCGATCTTTGGAGCCTGGAAAGCTTGCCAGACCCTCGCTCTCATGCACGACGCC
GTCGTTCTGCTCCTTGGCCCCGTCAAGAAGTATCAACGCGTCTTCGATGCTCGAGACCGCCCCGCCCACC
TCTACATCCACGCCGGCCAGACGCCCTCTTCCATGAGCCTGTGGTGCCAGACCCACCTCACCCCCGCTGT
CAAGCTCGCGAACGACTACACCGCTTTCGACCAGTCTCAGCATGGCGAGGCCGTCGTCCTCGAGAGAAAG
AAGATGGAACGCCTTTCCATCCCGGATCACCTCATCTCCCTCCACGTTCACCTTAAGACCCATGTCGAAA
CCCAGTTTGGCCCTCTCACCTGCATGCGCCTAACCGGCGAGCCTGGCACCTACGACGACAACACTGACTA
TAACCTCGCCGTCATCAACCTCGAGTACGCGGCTGCCCACGTCCCGACCATGGTCTCGGGCGACGATTCA
CTCCTTGACTTCGAGCCCCCACGCCGCCCAGAGTGGGTCGCCATCGAACCTCTTTTAGCCCTCCGCTTCA
AGAAGGAGCGCGGTCTGTATGCCACCTTCTGCGGCTACTACGCCTCGCGAGTTGGCTGCGTCCGATCTCC
CATCGCCCTCTTCGCTAAGCTCGCCATCGCCGTCGACGACTCATCCATCTCCGACAAGCTCGCCGCATAC
CTCATGGAGTTCGCGGTCGGTCACTCTCTCGGCGACTCTCTTTGGTCCGCCCTCCCCCTGTCCGCCGTCC
CCTTTCAGTCAGCCTGTTTCGATTTCTTCTGCCGCCGCGCTCCCCGCGATCTAAAGCTCGCCCTTCACCT
GGGCGAAGTCCCTGAAACCATCATCCAACGCCTCTCCCACCTCTCCTGGCTATCCCACGCCGTCTACAGC
CTCCTCCCATCTCGCCTTCGCCTCGCCATCCTTCACAGCTCACGCCAGCACCGTTCCCTCCCCGAAGACC
CAGCCGTTTCTTCGCTTCAGGGTGAATTGCTTCAGACGTTCCATGCTCCAATGCCCTCTCTCCCTTCACT
CCCACTCTTCGGCGGTCTATCTCCCGACAACATCCTCACTCCCCACGAGTTCCGCACCGCCCTCTACGAA
AGCTCCGCCTACCCTACTCCTCCCAACTCTCCGACCTCCATGTCAGGAATCCATGCCTCGCAAGTTGGTC
CGCCCCCCGCCAGCGATGATCGCACTGACCGCCAGCCTTCTCTTCCTCTTGCTCCTCGTATTGTGGAGAG
CTCTCTCGCCGTGCCGCACGTCGACGTCCCGTTCCAATGGGCCGTCGCGTCGTACGCCGGAGACTCCGCC
AAGTTCCTCACCGACGACCTCTCAGGATCCTCTCACCTGAGCCGCCTCACCATCGGCTATCGCCACGCCG
AGCTCATCTCCGCCGAGCTCGAGTTCGCCCCCCTTGCCGCCGCCTTCGCCAAGCCCATCTCCGTCACCGC
CGTCTGGACCATAGCCTCCATCGCCCCAGCCACCACCACCGAGCTCCAGTACTACGGTGGCCGACTCCTC
ACCCTCGGAGGCCCCGTCCTCATGGGCTCCGTCACCCGCATCCCAGCCGACCTCACCCGCCTCAACCCCG
TCATCAAGACCGCCGTGGGCTTCACTGACTGCCCCCGCTTCACCTACTCCGTCTATGCCAACGGCGGGTC
CGCCAACACTCCTCTCATCACCGTCATGGTGCGAGGAGTTATCCGCCTCTCCGGCCCTTCGGGCAACACC
GTCACCGCCACCTAAGCCCTCTCACCGGTTTCAACAGGAGTTTCTTCCTCGTTCTTCTCCTGACGACCAA
TGAACGTTGCTTATCCCCCCTTCACATCCCTCCGTTTCCCCCTCCGTTTTCCTCTCTGTTCCATTCCCCC
TCTCCCTCCCCGTCTCAGCAATGAGTAAGGTTCCAGGTCGATTCAAAGACCTGATGGGATTTTCCTCGG
Rice grassy stunt virus (RNA 1, Accession Number: N NC_002323.1) (SEQ ID
NO: 436):
ACACAAAGTCCTGGACAACAAAAACAAAAAAACTCTTTCATCAATATTTCGTTTCTCTTAAGTATTAACT
TTAAATATAATTATAAAGATTGTGTATTCTTCAACGACAGAGGAGTTCTCTATCTACTTTATAACAGTTT
TATTAAAGTTTGTTCTTGCGATAGTATGGGTTACTATCACTCCAAGACTGATAATCCAAAATTGATAACT
ACAAAAATAAGGAAGTACAAAGTATTCTCAATTCCTGTTAAAACTCAGGTTATCATCATTACTGGATCGA
CTCTCTCATTAGACTTCTTTACACTACAAACATGGATACACCTCCAAGAGGGTTTTATCTTAGAAATGGG
TGTTAGATCTACAAATGGTGTGCTGAAAATAGTTAACACTATTTGCCAAGAGAATGGGAAGATAGAGCGT
GATAGGTGGGATTGGTACGGTTGTGCGGATAGTGGTTTGCGTAAGGTTCATTATGATGAAGGGATAGCTA
GATCTGAGAGAACAAGCATAAGGGTTGATATTCGAGGTACCTTATTTGTATTGACTGTAGATGGGCACAT
ACTTGGGGTGTATGATGTTAATAGCTGTATCAATGCCATAAATATTGGTTTGGAAGTTTTGCCAAATTCA
GATAACACGCTGGATTTTGATTTAATATATCACTAGGAAAATACTTATATTAAAGGTAGATATTAATTAA
ATATCGGATATGGGCCGAAGCCCATATATCCAATCAAATGTCCAATATTCTCTAGCATAATCCAAACACA
CAAACTAGAACATGTATGACCTACCTCTACCCCTCCTTCCTCTCCCTCTTGAAGAAGGCGGGTTATAAGT
AGGAAACTGTGAATCAGGCACATCATACATGAATTGTAGAATCCTTTTGTAGTGCATTGAACTCGCTGGC
AGTTTCTGTCGACTTTCACCTTTAATTATATTCATAGTTAATCTCAAATCATCTGTTCCCATGAATGTAT
CCATTTTCCTAACTGAAGATAAGAACATTTTATGAAAGAGAGAAACATTAACTGCCTCTTTCTCCATTTG
GATTTCGTCTTGCTCCTCAGCAAGATCTCTAGCCAACTCATACAGGTGTTCCAAGTCTTCATCTTTTATT
GTCATATCAATAGTTTTATATGCTTGGCTAATCCTGGTTAATAGTGACTCCATACTTTCCAACTCTTCAC
AAACTTCCTTGTCTTCTTGAATACTCTCAGGATAATCATGAGCTAACCTATCTCTTGCTGCCTTGCTTAG
CTTAGATAGTTGAACTATCTTTTGGTAGCCTAATGATGATTCAAAAAGTTCTCTGCACCAACTCTTCAGT
CTTTTCTCATCAACTAGATCTGGAAACTGACCCAAATTCATCTGTGTGAATAAACTAGTGGGTGCTCTCT
GGTCTCTCCACAATATCCAGTCTTTAAGCCAATCATCCATTTTGAGTCTTTTTATCATATTCACATTCTG
CTGTGACTGTAGCTCACTAGTGATCACATCTTTCTTTGAAAGATGAACAGTCAACACTGTTGTGTAAGGT
GCTCTCTCACCAGTGCACTCTTGAAGCAATCTTATAGAGTGATCAGTTATGTCAATACAGAATGAGTCTG
ACAAAAATGGCTGGTGAATTATCAACTTGGGATCTAGAACTATTGGACATCCATCTCTATCACTCATTTG
TACCCTTCGAAATTCAAACATCCTCCCAAGCATTTCACAGTCTCTGTTACCATATGCCATAGTGTAGTGG
GAATTTCCCACTCGATGTTCTCTTGACCATTCCTTCAATGATTGTATAGTATCTGATAGGTGCATTGCAC
TAGATAGACTAACACTTTTGATGTAAGACTCCATATTTTGATCAGAGTTTACTTCAATCTGAACTGCTAC
ATCATGTAGATAACCTCTCCAAACACCTGGTCCGAAGTACTTCTTTTCCTCCCTGTTATATGATTGCTTT
TGGACGTAGCCACCTAGTAGACCTAGATTACCAATTCTTATCTTCTCCATAATGTCTCTCCTACAGTATT
TAGCTTCATCCCTCAGTTTCATTAGGTCATAACTACTCTTAACAATCCTGTGACCAATCATTGTCAACCA
GTTCCTCGTTGGATCATTAGCTGCTTCTGCTTTTAGTAATTCTAAGCTTAGCAGCATTTTTTGTGTTGGT
TTTCTTTTGTTATACTGTTGATATGCATCCTCTAGAACCCTATCACAGTATATGTACTGACAATATGCTC
TTCTAGCAGAAGCACTGAGACTATTTATAGTGAGATCTTCAAAGTTTTTCTCCAGCTCCTCATATCCCAT
ACCACTGTCAAGCAACTCTTTCTCCACTAATTGCCCCATTCTTATTAAGGACCTGAAACCACCAAGAGAA
AGGTTTTGATCAAACTCGCCAAGATGATGTTCAACCAAATTCACTGCATCCTGAGTGTTGTAGTCTCCAG
AGAACTTCAGGTCAGGATCTGTTCTCAAGAACATTTGTATTATAGCCAGCTTGTTCCTTTTGGAACCTAA
CATAGATGTTGAGTATTCTAGATCCTTATTATCAACTATGAGATCTGTCATTGCAACTAGCTTCTCCTCA
TATTTTAAAGGTGATTGGTTTAACATTGTTAAGTTAGATAGTAGAGATTTATAATCCTCAGTTTTTTCTT
TCTTTATCACATCTGTAAAACCTCCAGAGAAAACTATTCCATTGGAGAAATTATTTCTGATAAGTGTCAT
TAGGTTGACGTTTCCTGAGGAAGCCTTGCCCATAGTTCCTACAAAATGCAGAACTCTCCCCTTCGTGCTC
ACTCTAGATAGGTAGTTATTAAGCTGAATGTAGCTAACAAATGGTGATCCTACTAAGGTGTCTCCGATTG
TGTCTCTGAGCCAGAGCCAGGTCTCTTTGTATTTTTTCCAAACTATCTTTAATGTTTTGGGGTGTGCTGG
TACATCCTTTTCTCCGAACCATATGAACTTTGCAACAGAATACACTGAGAATGTTGAACTCTGTTCTGTT
CCAGTCAATTGAATCTGTGATCTCACCCTTCTCCTTTGATTGATTCCACTCCTAGCCATATTGAGATTTA
GGCTGGAGAGATTGGATTCTATTTCTAAATAATCCGTCTCTTCTGGGAACAATGACTGTATCTCTTTGTA
TGATTCTTGTATTTGTCTTCTATGTTCATCAGACATACTGCTAGACTCTTGTCTTCTAACTAATAAGTAA
TTTGGATTTAGCATGAAGTACAGATTCTCAGAATTTGTCAGGCACTTAAAGGTGTGACATAATTTAAAGC
TTGCTTCAGGGTCTCTGCTTATATACACGTGTATGTTAATTCCGAATCCTTTGGACAATTTATGCATTAA
CTTATTTTCGTCAATGAACCAGTCATTCAGTTTAGTATCATCAAGAGTCAATCCAAACTTTTCAGACAAC
AGCCTCTCTGATTGTTCCATGGTTAGGTCAGTTAAAACTGAAACCATCTTTATAACACCCTCTCTCAGTC
CTTCTACTGAATAAAAATCATCACTGGGCTCTTCTGTTAAAGATTGGACGCCAGGAATCTGAGCTTCTTT
CTGGCCAATCTTACTTACCACATTGGAGTTGCTGTTTAGTAATTCTCTGAAAATAGAGGTCTTTCTCTTC
TCATCTGTTTCTACACCAGCAGACATGCTGAACAATACATTTCTAGATATAAAATACACTGAGGATGCAA
CTCTTCTTCCAAGAGTATTTGTCTTTGCTAAGCTTTGCATAACACCTGGGCTTCTCATCTTGATAGCAAT
TTTCTGCTGCATTTCTTCTGCATTCTTAGCATGGAAAAATAGTATTCTAGGATTTTGCTCGATAGAATCA
AAGATATCATCTGTTAAGTGCATCCTATCACACATCTTCATCCATTTGGTCTTGTTGCCGAATCCAACAG
TTGTAGTCCTAGAGAGTACTCCTAGATTAGCAATATCAGGTGTCATCTTCCTCTTTGAATTTTCAGTATT
GAATTCCAGATTTAGCATGTCTGCATATTTCACTGATAAGAATGACTGCTTGCATGTTTTCCATAGATTA
TATCCAAACCCCATCAACCCAGATGCCATTGGGTGGTCCATTAGAAAGTATCCAAGAGCTGGATCTTTTG
ACAACTTAATCATCGAACAGTAGCTGCCCCATAGAGGTGATACTGAAGACCCATACATCCTATAGTGTAA
CATTGCTTGAGCAACTTGAGTTACGAAAGTGTGATAGAACGTCCCTCCACCTTCCAATATATCCTTAAGA
GTGTTTGACATCTCCTCTTGACTAGCGATCAAGGTTTCTTGCTCAGAGACATTCAGTGCAGCATTCACCC
ATCTAATTGTTGGTCTATGGGTGTCTCCTGCAAAGAAAAACTCAATATTAAATTCCATCATAAATATTGT
TCCGGTTGTTGATTTTATAGATTTGTAGATACCTAACATATCCCCATAGTATTCTTTCAGGGAGAATGCT
CTATCTACCAACAGAAGCATTGCAAATGTTTGCCTGTCATTCATAGATTTAGTTGAGAATGATATCATCA
TTGAGCTATCATCTGATGACTCCATGCAATCTATAATAACATTAGACTCATTATCTGGTTGTATTATCCT
AGCCATCTGTGGGAGTTGTTTCTTTTGCCTCTCTGCCAAATCCTCTAGGAATATAGCATGAAACAGAGAG
CTGATATAATGCAGTATACCTTGCATAAATCCTGATTCTGTTTCTATATAAGTCATACCTCTTGTCATCC
AGGGAGCAACCTCTCTTCCTTTAAAAACCTCATGAACTTTCTTGACCTTTTCATCAGTAGTATTTAGTAC
ATCATTTGCACAAAAGAGTCTAAGTAAGTCATCACCCAAGAATAATCTTTTGTGAAACCATAATTGTAAT
GCCCTAACTATGAAGCCGTGCCAGAATTTTGGTAGAATCCTAACTAATATGGTTATAAACTTTGAGACAT
GGTGACCTTGATTCCATTTTGATGCATCATCACTGGTACACACAGTAAAGTAGCTATCACCAAATTCCTT
TCTTGCTGCAATATTGTGTTTATTTGGAATTTGAAATTTGTTCTTAGGATGGGTCATAGTCTCACTAGGG
ACGACTGATAATATAGCTCTGGCAAGATCTTCAACACATTTTTGTACAATCCTCTCATATATATTTAAGA
CATAAATCTCCCTAAGCCCTCCATGCTGGTTCTTCCTGAAAATACACACATGCAGGCAAGCATTCTTTTC
AACCTCCTCCAGAGACTCTTTGAGAAGATCAACAACTAACCTATACTTTTCATTAGGATCCTTTTTAGTT
AAGATAGTTTGTATTTTTTCTATAACTTTCGACCTACCATAGTTTCTCCTGTTAGATTCTGATTTAGGTA
GATCTTCGTTAACGGTCTGCGGTCTACTTCTTTTATTTTCATTAGGTCTGTACTCATAGTACTCAGCCGA
AAAATTGGATGATGCTTTTAGGGTCACAAAAGACTCTAAAAACTCATGTGACAGATACTCTAGACATAAA
GTGCTCAGGTAATCCTTAGGATCACTAACTCCTGTCTCGCTTTTCAATCTTCCCAGAAAACTATCACACA
TCCTTTTAACCAGAGATATAGAATACATGTGGGTACTACACTGATCAACTGGAGGGTCTTCTAATCCCAA
ATACTTCTTATCTTCACCTCTGGGCAGCTTGTCCTCATACCCTAATATCTTAGATATGAGTTGCCCTGAT
GCATTATCTTCTGGATCCTCATCTTTGTTCTTGAGATACCCTAAATACATGCTACTTAGCATTACATCAT
GATTTGGAAAATTGGACAGTGAGCCATTTTCAGTTACAAAGGGATTTTTTATGTTATACCATCTCCTCAT
TGGTCCACTGTTGTCCAATCTAATGGGAGTCTCTGTGTAGCATTCCATCAATTTAATGGCAGACTTTATG
TAGAATACTTCCAATCTAGATCTTGGTATTGTTGATAGTTTTTCAAACATCTTATGAGGTTTAGGCCAGT
TAGGAGTCTCCACAAATGCCTCCATATGTATGAATCTTGTACTAGTGATGACTTCCTCAGTCTGGTGCTT
ATCATTTAGGAGTACCAATAAACAGTTCGCCCACATCTTGAGATAATCAGAGTTGAAATCTTCATCTGGT
ATACTGGAGATACCAATATTTGGTGGGATATTATATTGCTCTCTCCAGAAAGCATATAAACTCAACATTA
GAGATTCACATCTAGTCCAATTGACCAGTTTAGAATAGTTCACTGATATGAAATCAGTGTAGAGGAACCT
ATCTCCTAGTTTTGATACTTTCTTGAAAGTAGTGTTTATAATTTTGCTTAACTCCTGATCCTCTCTAAAA
AGTAAAGAAAAGAAAACTTTACCATCACTCCCAGTTGATTTGATGAGAACGTACACTTGGAAATCCCTCA
ATCTTTTCACAATAAACTCTCTTGGTTGACAGTTCTGCTTGACAGAAATTGATAGTTCAACAGCCAAATC
TGATACAAACTTAGTGAATAGGTATGCCTTGCTCTTGAGATATGTGTCTAAGGACTCCAACAGTCTAGAT
TGAGAAGAACAACCATGAATCTTAAGTGAATCACTTATTAGGTCTAACACACTATTATCTAATTCTTGAT
CATGAGGTGTAAACAATTGGAGACACTCTTCATTTATAAACCTCTCAATGTCATCAGTGGATGTGAATAA
GGAGAAGGGTTTCTTTGACTCATTTCTGTAAGCCAGTACCTCAGGATCCTTACTATATTTCTTACCATTT
ATTCCTATCTTTGCTAGATCTATCCTATCATCCATGTCAAACACCATCGAAATTCTGTTGAATTTATTCC
TAATCTTTTTGAGATCATCCTCCATTTGAGTGGAAAGAGTTGGTTCTTCCATTGCAAGAGAGAAATCCGA
TTTACCATCTTCAATCTCATATAGATAATGCATGAAACCACAAATGCCTTGTTTCCAAGCTTCTTCTGTT
GAACTCATTGAAGAAGTACTAATGATTTCATCAACTACATTTCTGACCTCCTCATGTGTGTTGCTTACTC
CCACAACTCGCACAATCTTGGGAACTAACATTGGAAGCTGAACAGATGCCTCGTTGGATGTTCTGTAAGC
TTCTTCATTTTTTATAAAGTTGGATTCATATTCCATTTTCCTTGATTGCATCATCAATATGGATTCATTT
CTTATTTCATCTCTATAGGCTCTTATATTTACATCATTAAGATGCTTCAGTTTCTCCATCTTCCTCATGG
ATTTATTGGAGACGTAAGTATCAAGTTTGTGTAAGTAATCATAATCTTCAGATTCTAGTGTCCCAACAGC
TTTAGTATAATGAGCCATGGTATATGGTTTAATAAATTTACTGGGATCCAATTCTCCATCTTCCTTATGT
ATTCTTATTCCTTCTATAATCTTCTTTATAGACGAGATCTCCATTTTCATCGCTTGGTCAGCCTTTATGT
CATATTCTAAATTTTGCTCAATTTGTAGGGCTATCTGTCTTGCTAGTTTATATCTATAGATCAATTCATC
CATTGTCTCTGTGGGGAGATTCATCAGGTTAGTTTGAACGCCATTTTGACACACTACGATTATATAGTAA
TCTATGCTAATCTTGAAGTGGTCTCTCCTATTGTGAATAGCATCTCTGTACTTGAGAGTTTTATCTTCCC
AACCTCTGCTTCTTACATCTGGTCTCATATTAGTGTTTCTAGTAGTGAACTCAATAACACTGTAATGTTT
TTCCCCATGTTTTATTATCATGTCAGGGGTCTTATTATTGTCTGGATCTCCAGGTATAAAGAGACCAGCA
TCAGTGAAAGAGACATCTAGATCATCACCAAACAGGGCAAAAGTGAAGTCATGGACAATGTTTTTCACTG
TGCTTATTTTGCATGAGTAAGCTCTGTTGTCGGGTATTGAAGGAAAGTCATGATACTTCCTATTACCAAA
TCTATTTTCAAAGCTGATCACAATTTCAGTTTCATCAGGAGAAACAATCTCACTAGTTTCTGGCACTTTC
AACCTTGGATGCATCTCATACAATCCATACATAGATTCATCATAACCACTATGTTCAGGATTTGTTAACT
TCTGTATATCATCATCGTAACTCGTGAACAGGAATTCTGCTGTTTTCCTGCTGAAGCTAAGAGTGGGGAA
ATCCTTATCATATTGGTCATCTCTGAGAACTGTCACTGGCAAACCACTTTTTACAGCACCCCATAGGTTT
CCTTCAGAGTCCAATAAAAAATGTTTCTGCTTCTTAGTAAGTTCAGGATTCTGACCCCAGTATTTATAAA
TTTTTGGTTTTAATATCGTCCCAACATTGACCATATCATTATCCACTAGGAGTATAGTGAGCAGATTTTC
CAACTCTTTTGATAGGAAACAAAGTGATAACAATTTATTCTTGTTCAAAAACGGTTCATAGTCCACTTCT
CTGCGCTGTGGAAATATTGTTTTACACTTCTCAATCATTTTCAGTTTGTTACTATACCAATTACTAGGTT
GTACAAATGACTGTGTTAGTTGTCTTATTAGTGACTCCAAGTCATGCACTTTTTCTAACACCTGATAATT
CTGATCAACTTCAAGTAGTCCTGAAATCTTTAGGAAAAACCACTTGTTCGTTGAGCCATCATAAATAAGC
TCTAATAAGCCATTCCCTGGCCCTACACACTTGAAACCACCTTCTAAACTATACAAGTTCTCATGATATG
ACAACAAGCGAACTTCTATAGTCATTCCCAAATTGAGTGACAACATTGCTACCTCTTGCTGCCTAGAATA
CTTCTTCAGTCTTACAATGGAAGAAAATAAGTTGCAGTTAAATTGAGGCACTTGCTGATGTAAATTACTT
TCAGCAAGACTAGCAAGAGAATTGTACCACTGGCCTCTCTCAGTGAAAACATCTCCCAATGGTTTTGTCT
CAACCTTCAGCTCAATCATGCTAGGTAGATCTATGTCATCATCTAAACTACCTAGATATCCACCTAATAC
TGAAGATAGCTCAAAGTAGTCATATGTTGGGTCATCTATGGCTTCATAGTGCCTACTTTCCAGTTTCATG
TGAATCATCAACTTACTCCTATCACCGAATGTTTTACAATGGCTGTCCCATGTTTCATCATGAATACATA
TACATATGTCTAGACATATTGAAACATGAATGATTGAATAATAAGTAGCCATATAAGAATCATTTGGATC
CAGTTCTCGTAACAACTCCTTCAACTCAGAAGCTGTCCATATACTCATGGCATAGTACTGATTCCTCAGC
TTGTTCATCACCTTGATGTAATCCTTACTCTCCACTCTTAAACATAAACATAAGGCATTGAAGAAACATT
TTAGATTTGGAGAGGGAGTCGGTATTGTTTCAGCACCTTTATAGAAGCAATCCACCAGGCTACCGTTGAT
ATCATATTCGACATCATTGTACTTAAACCTCTCTACACCTACTATTTCATTGTTCATATTATGTAAGTAG
GAAATATTAGAGAATTGACAGTTTGTATTCATGTTAGCTAGTGAGAGGTATACAACAATGACAAAACCAA
CCAGATGATATGGTGTGGACAATATTCTAGAGATATTATTATAATGTAATTAAGAATAAGAAATTAACTA
ATAAATAAATGCAATAATTAATAAAATTATATTACTGAAAAAGTATTCCCTGAATATTATGCTATTTGTT
CGTTTTTCTAATTTTGTCCAGACTTTGTGT
Oat chlorotic stunt virus (genomic DNA, Accession Number: NC_003633.1) (SEQ ID
NO: 437):
TTAAATCGTCCCGATTTAGCAAGCCATGGCTCTTTATCCGTCTCAAGATGTCTTGGCCCTCACTCAGTGG
GGTGCCAAATGGCTCAAGTTCGGTTTCAACATGGTTGTCGGTAACACACCCGAGGCGCAGTTTGCCCAAG
GAACTCCTCACGGCGTTTGATACATGTAATGTGGCTCCCGAAGCACTTTTGGTGTTGCGGTCCACATCGT
TGATGATACTTGAGGAAACCTGTGTGGTTGTGGGTGCGGCAGAGATGCCCACCGCTGAGGATAACTCTGG
TCGGGAGTTGTTCATTGGCTCCAACGGTGACCCGATGGAAAGGAAAACCCGCACGGCGCACCATGCCATC
AAGAAGACCGTGCGCATCAAGAAAGGGCATCGCACAACCTTCGCCATGACTGTGGCGAACGGGGCGTATG
TCAAGTTTGGTGCCCGTCCATTGACGGAGGCAAATGTGCTGGTCGTGCGTAAATGGATCGTTAAGCTTAT
TGCTGACGAGTACAAGGATTTGCGGGTGTGCGACCAGGCACTGGTTATAGACCGTGCCACGTTCCTATCA
TTCATTCCTACCATGGCGTGGAATAACTATAAGTTTATCTTCCACGGTAAGAATGCCGTCACAGATCGCG
TGGCGGGAGAGAACCTGTTTTCCCGGATCGCCCAATGGGCGAATCCAGGGAAATAGGGGTGCCCAGTAGT
CGTCACAGGGCAGGGATGCGTCATTAGCCGCGCTCCCGATTGTGCCCAGTTGCGTGTGAAGAGGCTATTG
GGAGTCACAAAGAACCGGACATGTATGCGTGTGTCTGGGGTTTCCCCTAACATCCAAATCATCCCGTTCA
ATAACGACATCACGACTCTGGAGAGGGCCATAAAAGAGAGGGTGTTCTTTGTCAAAAACCTCGACAAGGG
ATCGCCCACCAAATTTGTCTCCCCTCCCAGACCTGCGCCTGGTGTGTTTGCCCAGAGATTGTCAAATACG
TTGGGACTGTTAGTACCTTTTCTTCCCTCGACCGCTCCGATGTCACATCAGCAATTTGTTGATAGCACGC
CGAGCCGCAAGAGGAAGGTGTACCAACAGGCTCTCGAGGATATCAGTTGTCATGGGCTGAACCTCGAGAC
AGACAGCAAGGTGAAGGTGTTTGTGAAATACGAGAAAACCGACCATACATCCAAGGCAGATCCAGTGCCG
CGGGTGATTTCTCCCCGTGATCCTAAGTACAACCTGGCGCTCGGCAGGTATCTTAGGCCCATGGAAGAAC
GAATATTCAAGGCGCTTGGCAAATTATTCGGCCATCGCACCGTCATGAAAGGTATGGATACCGATGTGAC
GGCTAGGGTGATCCAGGAGAAATGGAACATGTTCAACAAGCCTGTAGCTATAGGCTTGGATGCGTCTAGA
TTTGACCAGCATGTTTCACTGGAAGCGCTTGAATTTGAGCATTCAGTGTACCTCAAGTGTGTGCGCAGGA
TGGTGGACAAGCGTAAGCTTGGCAACATCCTGCGACATCAACTTCTAAACAAATGTTACGGCAACACGCC
TGATGGCGCGGTGTCGTACACCATTGAGGGTACACGAATGAGTGGGGACATGAACACATCCCTAGGTAAT
TGCGTTTTGATGTGTATGATGATCCACGCTTATGGTTTGCATAAGAGTGTCAACATACAACTGGCGAACA
ATGGGGATGATTGTGTCGTGTTTCTGGAGCAATCCGATTTGGCCACCTTCTCAGAAGGCTTGTTTGAATG
GTTCCTAGAAATGGGATTCAACATGGCCATCGAGGAGCCCTCCTACGAACTGGAGCATATCGAGTTTTGT
CAGTGCAGGCCGGTGTTTGATGGTGTTAAATACACCATGTGCCGGAACCCCCGCACTGCCATTGCTAAAG
ATAGCGTGTATCTGAAACACGTTGATCAGTTCGTCACATATTCTAGCTGGCTGAATGCCGTGGGGACAGG
TGGGTTGGCGCTGGCGGGTGGTTTGCCCATCTTTGATGCGTTTTACACCTGTTATAAGCGTAACAGCAAC
TCCCACTGGTTCAGTGGCCGGAAAGGAAGGTTGAAAACCCTGTCAAGTGTTGATGATTCGCTCCCCTGGT
TCATGCGCGAGCTTGGACTGAAAGGGAAAAGGTCGTCAGCCGAGCCGTTACCAGCGTCTCGTGCCAGCTT
TTACCTCGCATGGGGGGTCACCCCCTGTGAGCAGTTGGAGCTTGAGAAATATTACAAATCGTTCAAACTG
GACACGTCCACATTGCTTGAGGAGCATTTGTGGCAGCCTCGCGGGGTGTTTCCCGATGAGGATTGAGCAC
ATTGTGGAAGAAGGTCACCACATTAAATCCACCCTTTACCATGGGGCTTGTCGTTAAATTGCCAAAACCA
ATTTGATGGGCTGATATAGATGCCAAGAGACTGCACGGCATACTACGTCGACAAGTGAACAGTCCCGTTG
TGTTGCGGGATCCCATACTAACAATCGTTCCTATGACTCTGAACTTACGTAAGGTACCAGCATACCTACC
AGGCAAAGTTGACGGAGCGCTCACTAATTTGGTGCACGCCGCCGTTGACCACGTGGTTCCTGGATTAGGC
AAAGCAGAGAAAGCTGCGGCAGTGTACAATATCAAACAGGTCGTTAAGAAACTCGGTACATACACCGAGC
AAGGCGTCAAGAAAATCGCAAAGAAAACGTTGGGTGAGTTGGGTTATCTCAATTACACCCCATCGTCACA
TCTTGGCATGGCTATAACCGGTCGAGGTACAAAACAAATCAATATGTCTCGCAGCACAAATGCTGGCGGT
TTTGCCCTCGGTGGCACCACCGCAGCGCCAGTGTCCATATCCCGCAATATCAACCGCCGCTCCAAGCCCA
GCATTAAGATGATGGGTGATGCGGTGGTTATCTCGCACAGTGAAATGTTGGGTGCCATTAATTCTGGCAC
CCCTTCATCGAATGTCACCGCTTTCCGTTGCACTGGCTACCGAGCTAATCCTGGGATGTCAACTATCTTC
CCTTGGCTGTCTGCAACTGCCGTTAATTACGAGAAGTACAAATTTCGTAGGCTCAGCTTCACTCTTGTCC
CGTTGGTTTCTACCAATTATAGCGGAAGAATAGGAGTTGGGTTTGATTACGATTCGTCTGACCTCGTACC
TGGCAACAGACAGGAATTTTATGCTCTCTCAAACCATTGTGAGAATATGCCGTGGCAGGAAAGCACTGTG
GAGATTAAATGTGATAATGCGTACCGATTCACTGGCACTCATGTTGCAGCGGACAATAAGCTGATTGACC
TCGGCCAAGTCGTGGTGATGTCTGATTCTGTGTCCAATGGTGGCACTATTTCCGCTGCGTTGCCGCTTTT
CGACCTGATAGTCAATTATACTGTGGAGCTGATTGAACCTCAACAAGCCTTGTTTTCATCCCAACTGTAT
AGTGGTTCTACCACTTTTACCTCTGGGATACCACTTGGCACAGGTGCTGATACCACAACTGTGGTCGGTC
CCACTGTTGTAAACTCCACAACTGTCACGAACTGTGTGGTCACCTTCAAGCTGCCCGCTGGGGTGTTTGA
GGTGTCATATTTCATTGCCTGGTCCACAGGAACCGCTGCTGTTGTGCCCACTGTTCCCACTACTGGGGCT
GGGTCCAAGTTGTCGAACACATCCACTGGCTCCAACTCTTATGGGGTCTGTTTCATAAACAGCCCCGTTG
AGTGTGATCTGTTGCTTACGGCAACGGTACTGCTTATAATTCCAACCTTACCAAGTTCAACGTGTGTGTT
TCACGCACCTGCTCGCAGGTGTACAACGCCTATGTGTCATAGGTTGCTAACGTCTCTTGCTGGCTGAGAC
ATTAATAAATGGATCCAGTAGGTCGTCAAAGCAAACCAACAAGGCTTGCCGGGGTGGATGCGTAGCGCAG
CATGTCTGTGTTGGTACGGCCACACCCGGAGGGACCTCACCTTGTAGGCAGGAGTACACGACTGTTTTCT
TTATTGTTGCTCACAATGGAAAATACAAAAATAGGCTTATCACCATGATGGACACGCCAAAATATTCCAG
CCCTGGCGAGTCGCGGTCGCAATCCGCAGTTTATTAAAACCCTTCGGGGTGGGC
Rice stripe virus (RNA 1, Accession Number: NC_003755.1) (SEQ ID NO: 438):
ACACATAGTCAGAGGAAAAAATAATTTTGATTTTGTTTTCCACAAAAGAATTGAAGGATGACGACACCAC
CTCTCGTTATACCCTTGCATGTTCATGGCAGGTCTTATGAACTGTTGGCGGGGTATCATGAAGTTGATTG
GCAGGAGATAGAAGAGTTGGAAGAAACAGATGTCAGAGGAGATGGATTTTGTCTTTATCATTCCATACTA
TATAGTATGGGCCTGAGCAAGGAGAACTCTCGCACCACTGAATTTATGATAAAGCTACGATCGAATCCAG
CCATCTGCCAGCTGGATCAAGAAATGCAACTGAGCCTTATGAAGCAGCTTGATCCAAATGACTCATCAGC
CTGGGGTGAAGATATAGCAATTGGGTTTATAGCTATAATATTGAGAATTAAGATAATTGCTTACCAGACA
GTTGATGGGAAGTTGTTTAAGACTATTTATGGTGCTGAGTTTGAGAGTACTATTAGAATTAGGAACTATG
GGAATTACCACTTCAAGTCACTTGAGACAGATTTTGATCATAAAGTAAAGCTCAGATCAAAAATTGAAGA
ATTCTTGAGAATGCCAGTGGAAGACTGTGAATCCATATCCTTGTGGCATGCATCTGTTTACAAGCCTATA
GTATCTGATAGCCTTTCTGGACACAAGAGCTTTAGTAATGTGGATGAATTGATAGGTAGCATAATATCCA
GCATGTATAAGATCATGGACAATGGTGATCAATGTTTTCTTTGGAGTGCAATGAGAATGGTAGCCAGACC
CTCTGAAAAACTATATGCCCTTGCAGTGTTTTTGGGATTCAATCTTAAGTTCTATCATGTGAGGAAAAGA
GCTGAAAAATTGACGGCAAAACTTGAGAGTGATCATACTAATTTGGGAGTGAAGCTGATTGAGGTATATG
AAGTTTCTGAGCCAACCAGATCTACCTGGGTCCTGAAACCAGGAGGGAGCAGAATAACTGAAACAAGAAA
TTTTGTGATTGAGGAGATAATAGATAACAGGCGCTCTCTGGAGAGCTTATTTGTGTCAAGCAGTGAGTAT
CCTGCAGAGTTATGTTCCCAGAAACTTAGTGCCATCAAAGACAGAATAGCACTAATGTTTGGCTTTATCA
ACAGAACCCCTGAAAACAGTGGGAGGGAACTCTACATAAACACATACTATCTGAAGAGGATCTTACAGGT
GGAAAGAAATGTAATTAGAGATTCTTTAAGATCACAGCCTGCTGTGGGGATGATCCAGATAATCAGATTA
CCAACAGCATTTGGTACATACAACCCGGAAGTGGGCACTCTGTTGTTAGCCCAAACTGGACTAATCTATA
GACTTGGCACCACAACTAGAGTGCAGATGGAGGTCAGGAGATCTCCCTCTGTTATTTCAAGATCTCATAA
GATCACTAGTTTTCCGGAGACACAAAAACATAACAACAATTTGTATGATTATGCACCCAGAACACAGGAG
ACATTTTATCACCCAAATGCTGAGATCTATGAGGCTGTTGATGTAAAGACTCCTAGTGTTATTACAGAGA
TTGTTGATAATCATATAGTGATAAAATTGAACACTGATGATAAGGGTTGGTCAGTCAGTGATTCGATAAA
GCAAGATTTTGTATATCGGAAGAGACTAATGGATGCAAAGAATATTGTTCATGACTTTGTTTTTGATATC
TTATCAACTGAGACTGACAAGAGCTTTAAGGGTGCTGACTTATCTATAGGAGGAATCTCAGATAACTGGT
CACCAGATGTCATTATATCAAGAGAAAGTGATCCACAGTATGAAGATATCGTTGTCTATGAGTTCACAAC
AAGGTCCACTGAGTCTATAGAATCTCTACTAAGATCAGTAGAGGTTAAAAGCTTACGATATAAAGAAGCA
ATTCAGGAAAGAGCCATCACATTAAAGAAGAGAATATCGTATTACACAATATGTGTCAGTCTAGATGCTG
TAGCCACAAATCTGCTATCACTTCCTGCTGATGTCTGCAGAGAACTAATAATTCGTTTAAGAGTTGCTAA
TCAGGTGAAGATCCAGCTAGCTGATAACGATATCAATCTTGACTCTGCCACTTTGCTAGCACCTGACATT
TACAGAATAAAGGAAATGTTTAGGGAAAGTTTCCCAAATAATAAATTTATACATCCTATTACTAAGGAAA
TGTATGAGCATTTTGTCAATCCAATGATTTCAGGAGAAAAAGACTATGTTGCCAATTTAAAGAGCATAAT
AGACAAAGAGACCAGAGATGAGCAGAGAAAGAATTTAGAGAGTCTGAAAGTTGTGGATGGGAAAAAGTAC
ACAGAGAGAAAAGCAGAAACTGCTCTGAATGAGATGTCACAAGCAGAAGAGCATTATAGAAGCTATTTTG
AAAATGACAATTTTAGGTCCACACTAAAAGCTCCAGTCCAACTTCCCTTAATCATACCGGATGTGTCAAG
TCAGGACAATCAATTCTCAAACAAGGAACTATCTGATAGGATACGGAAGAAGCCGATCGACCACCCTATT
TACAACATCTGGGATCAAGCAGTTAATAAGAGAAATTGCTCGATTGCACTCGGCCATTTGGACGAGCTAG
AAATATCTATGCTAGAAGGACAAGTGGCTAAGAAAGTGGAGGAATCTTATAAGAAAGATAGGAGTCAGTA
CAACAGGACAACTCTGCTAACTAATATGAAGGAGGACATCTACTTGGCTGAAAGGGGGATAAATGCTAAG
AAGAGGTTGGAAGAACCAGATGTGAAATTTTATCGAGATCAGTCTAAGAGGCCTTTTCATCCTTTTGTGA
GTGAAACCAGAGACATAGAGCAGTTCACTCAGAAAGAGTGCCTGGAACTCAATGAAGAGTCAGGACACTG
CTCGCTGATAAATGTAGAGGATCTAGTGTTATCTGCTCTAGAGTTGCATGAGGTAGGTGATTTAGAACAC
TTATGGAACAACATAAAAGCTCATTCTAAAACAAAGTTTGCATTATATGCTAAGTTTATCTCTGATCTTG
CCACCGAGCTAGCCATTTCATTATCCCAGAATTGCAAAGAAGACACCTATGTGGTTAAGAAACTCAGAGA
TTTTAGCTGCTACGTACTCATTAAACCAGTAAACTTAAAGAGTAATGTGTTCTTCTCTTTATACATACCT
TCTAATATTTATAAGTCACACAACACAACTTTCAAGACTCTGATAGGCAGTCCAGAATCAGGGTATATGA
CTGATTTCGTCTCTGCTAATGTGAGCAAGTTAGTGAATTGGGTTAGATGTGAAGCTATGATGCTAGCACA
AAGAGGTTTCTGGCGAGAATTTTATGCTGTGGCCCCTAGCATTGAGGAACAAGATGGAATGGCGGAGCCA
GACTCAGTATGTCAGATGATGAGTTGGACACTCCTCATATTACTAAACGACAAGCATCAGTTAGAAGAGA
TGATCACAGTGTCTAGGTTTGTCCATATGGAAGGCTTTGTAACTTTTCCTGCATGGCCTAAACCTTATAA
AATGTTTGATAAATTATCAGTAACTCCGAGGTCTAGGTTAGAATGTCTAGTCATAAAGAGGCTCATTATG
CTAATGAAGCATTATTCAGAAAATCCCATTAAATTTATGATAGAAGACGAGAAGAAAAAGTGGTTTGGAT
TCAAAAATATGTTCTTGCTTGATTGTAATGGTAAACTTGCTGATTTATCTGATCAGGATCAAATGCTTAA
TCTCTTTTATCTTGGGTATCTAAAGAACAAAGATGAGGAGGTCGAAGACAATGGCATGGGTCAACTATTG
ACTAAAATCCTGGGCTTTGAGAGTGCCATGCCAAAGACAAGAGACTTCTTGGGTATGAAAGATCCTGAGT
ATGGTACAATCAAGAAGCATGAGTTCTCCATAAGCTATGTGAAGGACCTCTGTGATAAATTCTTAGACAG
ATTAAAAAAGACACACGGAATCAAAGATCCAATTACTTATTTGGGCGACAAGATAGCTAAATTCCTTAGC
ACTCAGTTTATTGAGACGATGGCATCTTTGAAGGCATCATCTAACTTCTCAGAGGATTACTATTTATACA
CACCCAGTAGAAGACTAAAAAACCAGGAGCAATCTAGAAGTAAACATGTAATAGACGCCGGTGGGAATAT
ATCTGCTAGTGTCAAAGGTAAGCTGTATCATAGAAGCAAAGTAATTGAGAAGCTCACAACCCTAATTAAA
GACGAAACACCAGGAAAAGAACTGAAAATAGTGGTAGATCTCTTACCGAAGGCTATGGAAGTCCTAAACA
AAAATGAATGTATGCACATTTGTATTTTCAAGAAGAATCAGCATGGAGGCCTTAGAGAAATATATGTTCT
TAATATCTTTGAAAGAATAATGCAGAAGACAGTGGAAGATTTCTCTAGAGCCATTCTAGAATGCTGTCCT
AGTGAGACAATGACATCCCCGAAAAACAAGTTTAGAATACCTGAATTGCACAACATGGAAGCAAGGAAAA
CTCTAAAAAATGAGTATATGACAATATCTACTAGTGATGATGCATCGAAATGGAATCAAGGTCACTATGT
ATCTAAATTCATGTGTATGCTATTGAGGCTCACTCCAACATATTATCATGGCTTCTTAGTTCAGGCTCTT
CAACTATGGCATCATAAGAAGATATTCCTAGGAGACCAGCTGTTGCAATTATTTAATCAAAATGCTATGC
TAAATACCATGGACACAACCCTCATGAAAGTCTTTCAAGCCTACAAAGGGGAGATTCAAGTGCCTTGGAT
GAAGGCAGGTAGATCCTACATAGAGACTGAGACAGGTATGATGCAGGGAATTCTCCACTATACTAGCTCT
CTATTCCATGCTATCTTCTTGGACCAACTGGCTGAAGAGTGTAGAAGAGATATAAATAGAGCAATTAAGA
CAATAAATAATAAAGAAAATGAGAAGGTGTCATGTATAGTGAACAATATGGAAAGTTCTGACGATAGTAG
CTTCATTATTAGTATTCCCAATTTCAAAGAGAATGAAGCAGCACAATTGTACCTGCTCTGTGTGGTTAAC
TCTTGGTTCAGAAAGAAAGAGAAGCTTGGAACTTATCTTGGGATATATAAATCTCCAAAGAGTACAACTC
AGACATTGTTTGTGATGGAATTCAACTCAGAATTCTTCTTTTCTGGTGATGTTCACAGGCCAACTTTTAG
GTGGGTCAATGCAGCAGTGCTAATAGGAGAGCAAGAGACATTGTCTGGTATACAGGAAGAGTTGTCAAAT
ACATTGAAGGATGTAATAGAAGGTGGAGGAACATATGCCCTCACTTTTATAGTGCAAGTTGCTCAAGCTA
TGATACACTATAGAATGCTGGGCAGTAGTGCTTCATCAGTGTGGCCAGCATATGAAACTCTTCTGAAAAA
CTCATATGATCCTGCACTTGGCTTCTTCCTAATGGATAATCCTAAATGTGCTGGCTTGTTGGGATTCAAC
TATAATGTTTGGATTGCCTGTACGACGACACCTTTGGGAGAGAAGTATCATGAGATGATACAAGAAGAAA
TGAAGGCTGAGTCTCAGAGCTTAAAATCAGTAACAGAAGATACAATTAACACGGGATTAGTTTCACGAAC
AACTATGGTGGGCTTTGGAAACAAGAAAAGATGGATGAAACTCATGACCACACTGAATCTGAGTGCAGAT
GTGTATGAAAAGATAGAAGAGGAGCCAAGAGTGTACTTTTTCCACGCAGCAACAGCTGAACAAATAATTC
AGAAAATTGCTATTAAAATGAAGAGTCCCGGTGTGATACAGTCACTGTCTAAAGGAAACATGCTGGCAAG
GAAGATAGCGTCAAGTGTATTCTTCATATCTAGACATATAGTCTTCACAATGTCCGCTTATTATGATGCA
GACCCTGAGACAAGGAAAACATCACTGCTGAAGGAGTTGATTAATAGCTCTAAAATACCTCAGAGACATG
ACTATCTGCAGGAACCGCATACATTGAAGCCAACTAAAGTTGAAGTTGATGAGGACAGCTGGGAATTCAA
GTCAGCAAAAGAGGAATGCGTTAGAGTGCTAAAACAAAGAATCAAAATACACACTGGGAGAGAAGAGAGA
TCTATTAGTCTTTTGTTTGAAAATATGGCTAAGTCAATGATTGGGAGGTGCACGGACCAGTATGATGTTA
GAGAAAATGTTTCCATTCTAGCATGTGCACTGAAAATGAACTATTCTATATTCAAGAAGGATGCTGCACC
CAATAGGTATCTCCTTGATGAGAAGAACCTTGTATACCCACTGATTGGAAAGGAAGTATCTGTTTATGTT
AAGTCTGACAAAGTACATATTGAAATATCTGAGAAGAAAGAAAGGCTATCAACCAAATTATTTAATATAG
ATAAAATGAAGGATATAGAAGAGACTCTCTCACTACTGTTTCCTAGTTATGGAGATTACTTATCCTTGAA
AGAAACAATTGACCAAGTAACTTTCCAATCTGCCATACACAAAGTCAACGAGAGAAGAAGAGTTAGGGCA
GATGTGCACTTAACAGGGACAGAAGGATTTTCTAAGTTGCCAATGTATACAGCAGCTGTCTGGGCCTGGT
TTGATGTGAAGACTATCCCTGCACATGACAGCATTTATAGAACTATCTGGAAAGTCTACAAAGAACAATA
CTCCTGGTTGTCAGATACACTGAAAGAGACAGTGGAGAAGGGACCATTTAAAACAGTACAAGGTGTGGTT
AACTTCATTTCTAGAGCTGGTGTGAGATCGAGAGTCGTCCATCTAGTAGGGTCATTTGGTAAGAATGTCA
GGGGTAGCATAAATCTGGTGACGGCAATAAAAGATAACTTTAGCAACGGACTAGTTTTCAAAGGGAATAT
ATTCGATATCAAGGCAAAGAAAACTAGAGAAAGTTTGGATAACTACTTGTCAATCTGCACCACTCTGTCT
CAGGCACCTATCACTAAGCATGATAAGAACCAGATTTTGCGCTCTCTTTTCGTCAGTGGTCCAAGAATCC
AGTATGTGTCATCACAGTTTGGATCAAGAAGAAACAGGATGTCAATATTACAAGAAGTCGTGGCAGATGA
TCCAACTCTACATTGGCCTGACCAAGACACAAGTCAGAAACAGCTAGAAGACAAATTCAGAGAACTAGCA
CACAAGGAGCTCCCATTTCTAACAGAGAAGGTGTTTCACGATTATCTGGAAAAGATAGAGCAGCTAATGA
AGGAGAACACTCATCTAGGTGGTAGGGATGTTGATGCTAGCAAAACCCCATATGTGCTTGCCAGAGCAAA
TGATATTGAAATACATTGTTATGAGTTGTGGAGAGAGTATGATGAGGATGAAGATGAAGCATACCAGGCT
TATTGCAGTGAAGTGGAGGCTGCTATGGATCAAGAGAAACTTAATGCTCTAATAGAGAGATACCATGTAG
ACCCTAAAGCAAACTGGATTCAAATGTTAATGAATGGTGAGATTGAAACAGTTGAAGAGCTGAACAAGCT
TGACAAGGGGTTTGAGAGCCACAGACTTGCTCTAGTCGAAAGAATTAGGGTGGGGAAACTTGGAATTTTA
GGCAGTTACACCAAGTGTCAACAGAGAATTGAGGAGCTAGATGGTGAAGGTAATAAGACTCATAGATACA
CAGGAGAAGGGATATGGAGAGGTTCATTCGATGATTCCGATGTTTGCATAGTTGTCCAAGACCTGAAGAA
GACAAGAGAGAGTTACTTAAAATGTGTCGTTTTTTCCAAAGTGTCAGATTATAAAGTCTTGATGGGCCAT
CTGAAGACATGGTGCAGGGAACACCATATTAGTAATGATGAGTTTCCTACCTGTACTCAGAAAGAGCTTT
TAAGCTATGGTGTCACCAAGAGTTCAGTTCTATTGTACAAGATGAATGGAATGAAAATGTTGAGGAACAT
GGAAAAAGGTATTCCTCTGTACTGGAATCCTAGCTTGTCAACTAGAAGCCAAACTTATATCAACTGGCTT
GCTGTTGATATCACAGATCATAGCTTACGGCTTAGGAACAGAACTGTTGAGAATGGGAGAGTTGTAAATC
AAACAATCATGGTTGTTCCTCTGTACAAAACTGATGTGCAGATATTCAAAACATCTCCTGTAGATCTTGA
GCAAGATGTGCAGAATGATAGACTTAAGCTATTATCAGTAACGAAAGCTGGGGAGTTGAGATGGCTTCAA
GATTGGATAATGTGGAGATCATCTGCTGTAGACGATTTGAACATACTAAACCAGGTTAGAAGAAATAAGG
CTGCAAGGGATCATTTTAATGCTAAACCAGAGTTCAAAAAATGGATAAAAGAGCTGTGGGACTATGCACT
TGACACCACACTAATCAATAAGAAAGTCTTCATAACTACACAAGGATCAGAGTCACAGAGCACAGTTTCT
TCAGGAGATAGCGACAGTGCAGTGGCACCTTTAACTGATGAGGCAGTGGATGAGATTCATGATCTCTTAG
ACAAAGAGTTAGAAAAGGGCACCTTAAAACAGATCATCCATGATGCAACCATCGATGCCCAGCTTGATAT
CCCTGCTATAGAGAGCTTCCTGGCTGAAGAAATGGAGGTGTTCAAGAGTAGCTTAGCCAAGAGCCACCCT
CTTCTACTAAATTATGTTAGGTACATGATTCAAGAGATAGGTGTGACCAACTTCAGATCATTGATTGATA
GCTTTAATCAGAAAGATCCCTTGAAAAGTGTGTCTCTAAGCATCCTAGACTTGAAAGAAGTGTTCAAGTT
TGTGTACCAGGACATAAATGATGCCTATTTTGTTAAACAGGAAGAAGACCATAAGTTCGATTTCTGAGAA
GTCCTCTTCAACAAAGGGACTGCAGCACAAACACAAGTCCAGACACCATTGAAATCCATACAAATATTTC
ACGTTTTATCCCTTATGACTTAGATTTTCAATAATTAATTATATAAACAAAAACATTTTGTTTTCCTCTG
GACTTTGTGT

| Protein | | SEQ ID | |
|---|---|---|---|
| Name | Accession # | NO. | Sequence |
| HIV | NC_001802.1 | 479 | GGTCTCTCTGGTTAGACCAGATCTGAGCCTGGGAGCTCTCTGGCTAACTAGGGAACCCACTGCTTAAGCC |
| | | | CAATAAAGCTTGCCTTGAGTGCTTCAAGTAGTGTGTGCCCGTCTGTTGTGTGACTCTGGTAACTAGAGATC |
| | | | CCTCAGACCCTTTTAGTCAGTGTGGAAAATCTCTAGCAGTGGCGCCCGAACAGGGACCTGAAAGCGAAAGG |
| | | | GAAACCAGAGGAGCTCTCTCGACGCAGGACTCGGCTTGCTGAAGCGCGCACGGCAAGAGGCGAGGGGCGGC |
| | | | GACTGGTGAGTACGCCAAAAATTTTGACTAGCGGAGGCTAGAAGGAGAGAGATGGGTGCGAGAGCGTCAG |
| | | | TATTAAGCGGGGGAGAATTAGATCGATGGGAAAAAATTCGGTTAAGGCCAGGGGGAAAGAAAAAATATAA |
| | | | ATTAAAACATATAGTATGGGCAAGCAGGGAGCTAGAACGATTCGCAGTTAATCCTGGCCTGTTAGAAACAT |
| | | | CAGAAGGCTGTAGACAAATACTGGGACAGCTACAACCATCCCTTCAGACAGGATCAGAAGAACTTAGATCA |
| | | | TTATATAATACAGTAGCAACCCTCTATTGTGTGCATCAAAGGATAGAGATAAAAGACACCAAGGAAGCTTT |
| | | | AGACAAGATAGAGGAAGAGCAAAACAAAAGTAAGAAAAAAGCACAGCAAGCAGCAGCTGACACAGGACAC |
| | | | AGCAATCAGGTCAGCCAAAATTACCCTATAGTGCAGAACATCCAGGGGCAAATGGTACATCAGGCCATATC |
| | | | ACCTAGAACTTTAAATGCATGGGTAAAAGTAGTAGAAGAGAAGGCTTTCAGCCCAGAAGTGATACCCATGT |
| | | | TTTCAGCATTATCAGAAGGAGCCACCCCACAAGATTTAAACACCATGCTAAACACAGTGGGGGGACATCAA |
| | | | GCAGCCATGCAAATGTTAAAAGAGACCATCAATGAGGAAGCTGCAGAATGGGATAGAGTGCATCCAGTGC |
| | | | ATGCAGGGCCTATTGCACCAGGCCAGATGAGAGAACCAAGGGGAAGTGACATAGCAGGAACTACTAGTACC |
| | | | CTTCAGGAACAAATAGGATGGATGACAAATAATCCACCTATCCCAGTAGGAGAAATTTATAAAAGATGGA |
| | | | TAATCCTGGGATTAAATAAAATAGTAAGAATGTATAGCCCTACCAGCATTCTGGACATAAGACAAGGACCA |
| | | | AAGGAACCCTTTAGAGACTATGTAGACCGGTTCTATAAAACTCTAAGAGCCGAGCAAGCTTCACAGGAGGT |
| | | | AAAAAATTGGATGACAGAAACCTTGTTGGTCCAAAATGCGAACCCAGATTGTAAGACTATTTTAAAAGCA |
| | | | TTGGGACCAGCGGCTACACTAGAAGAAATGATGACAGCATGTCAGGGAGTAGGAGGACCCGGCCATAAGGC |
| | | | AAGAGTTTTGGCTGAAGCAATGAGCCAAGTAACAAATTCAGCTACCATAATGATGCAGAGAGGCAATTTT |
| | | | AGGAACCAAAGAAAGATTGTTAAGTGTTTCAATTGTGGCAAAGAAGGGCACACAGCCAGAAATTGCAGGG |
| | | | CCCCTAGGAAAAAGGGCTGTTGGAAATGTGGAAAGGAAGGACACCAAATGAAAGATTGTACTGAGAGACA |
| | | | GGCTAATTTTTTAGGGAAGATCTGGCCTTCCTACAAGGGAAGGCCAGGGAATTTTCTTCAGAGCAGACCAG |
| | | | AGCCAACAGCCCCACCAGAAGAGAGCTTCAGGTCTGGGGTAGAGACAACAACTCCCCCTCAGAAGCAGGAG |
| | | | CCGATAGACAAGGAACTGTATCCTTTAACTTCCCTCAGGTCACTCTTTGGCAACGACCCCTCGTCACAATAA |
| | | | AGATAGGGGGGCAACTAAAGGAAGCTCTATTAGATACAGGAGCAGATGATACAGTATTAGAAGAAATGAG |
| | | | TTTGCCAGGAAGATGGAAACCAAAAATGATAGGGGGAATTGGAGGTTTTATCAAAGTAAGACAGTATGAT |
| | | | CAGATACTCATAGAAATCTGTGGACATAAAGCTATAGGTACAGTATTAGTAGGACCTACACCTGTCAACAT |
| | | | AATTGGAAGAAATCTGTTGACTCAGATTGGTTGCACTTTAAATTTTCCCATTAGCCCTATTGAGACTGTAC |
| | | | CAGTAAAATTAAAGCCAGGAATGGATGGCCCAAAAGTTAAACAATGGCCATTGACAGAAGAAAAAATAAA |
| | | | AGCATTAGTAGAAATTTGTACAGAGATGGAAAAGGAAGGGAAAATTTCAAAAATTGGGCCTGAAAATCCA |
| | | | TACAATACTCCAGTATTTGCCATAAAGAAAAAAGACAGTACTAAATGGAGAAAATTAGTAGATTTCAGAG |
| | | | AACTTAATAAGAGAACTCAAGACTTCTGGGAAGTTCAATTAGGAATACCACATCCCGCAGGGTTAAAAAAG |
| | | | AAAAAATCAGTAACAGTACTGGATGTGGGTGATGCATATTTTTCAGTTCCCTTAGATGAAGACTTCAGGAA |
| | | | GTATACTGCATTTACCATACCTAGTATAAACAATGAGACACCAGGGATTAGATATCAGTACAATGTGCTTC |
| | | | CACAGGGATGGAAAGGATCACCAGCAATATTCCAAAGTAGCATGACAAAAATCTTAGAGCCTTTTAGAAA |
| | | | ACAAAATCCAGACATAGTTATCTATCAATACATGGATGATTTGTATGTAGGATCTGACTTAGAAATAGGGC |
| | | | AGCATAGAACAAAAATAGAGGAGCTGAGACAACATCTGTTGAGGTGGGGACTTACCACACCAGACAAAAA |
| | | | ACATCAGAAAGAACCTCCATTCCTTTGGATGGGTTATGAACTCCATCCTGATAAATGGACAGTACAGCCTA |
| | | | TAGTGCTGCCAGAAAAAGACAGCTGGACTGTCAATGACATACAGAAGTTAGTGGGGAAATTGAATTGGGC |
| | | | AAGTCAGATTTACCCAGGGATTAAAGTAAGGCAATTATGTAAACTCCTTAGAGGAACCAAAGCACTAACAG |
| | | | AAGTAATACCACTAACAGAAGAAGCAGAGCTAGAACTGGCAGAAAACAGAGAGATTCTAAAAGAACCAGT |
| | | | ACATGGAGTGTATTATGACCCATCAAAAGACTTAATAGCAGAAATACAGAAGCAGGGGCAAGGCCAATGG |
| | | | ACATATCAAATTTATCAAGAGCCATTTAAAAATCTGAAAACAGGAAAATATGCAAGAATGAGGGGTGCCC |
| | | | ACACTAATGATGTAAAACAATTAACAGAGGCAGTGCAAAAAATAACCACAGAAAGCATAGTAATATGGGG |
| | | | AAAGACTCCTAAATTTAAACTGCCCATACAAAAGGAAACATGGGAAACATGGTGGACAGAGTATTGGCAA |
| | | | GCCACCTGGATTCCTGAGTGGGAGTTTGTTAATACCCCTCCCTTAGTGAAATTATGGTACCAGTTAGAGAA |
| | | | AGAACCCATAGTAGGAGCAGAAACCTTCTATGTAGATGGGGCAGCTAACAGGGAGACTAAATTAGGAAAA |
| | | | GCAGGATATGTTACTAATAGAGGAAGACAAAAAGTTGTCACCCTAACTGACACAACAAATCAGAAGACTG |
| | | | AGTTACAAGCAATTTATCTAGCTTTGCAGGATTCGGGATTAGAAGTAAACATAGTAACAGACTCACAATAT |
| | | | GCATTAGGAATCATTCAAGCACAACCAGATCAAAGTGAATCAGAGTTAGTCAATCAAATAATAGAGCAGT |
| | | | TAATAAAAAAGGAAAAGGTCTATCTGGCATGGGTACCAGCACACAAAGGAATTGGAGGAAATGAACAAGT |
| | | | AGATAAATTAGTCAGTGCTGGAATCAGGAAAGTACTATTTTTAGATGGAATAGATAAGGCCCAAGATGAA |
| | | | CATGAGAAATATCACAGTAATTGGAGAGCAATGGCTAGTGATTTTAACCTGCCACCTGTAGTAGCAAAAGA |
| | | | AATAGTAGCCAGCTGTGATAAATGTCAGCTAAAAGGAGAAGCCATGCATGGACAAGTAGACTGTAGTCCA |
| | | | GGAATATGGCAACTAGATTGTACACATTTAGAAGGAAAAGTTATCCTGGTAGCAGTTCATGTAGCCAGTGG |
| | | | ATATATAGAAGCAGAAGTTATTCCAGCAGAAACAGGGCAGGAAACAGCATATTTTCTTTTAAAATTAGCA |
| | | | GGAAGATGGCCAGTAAAAACAATACATACTGACAATGGCAGCAATTTCACCGGTGCTACGGTTAGGGCCGC |
| | | | CTGTTGGTGGGCGGGAATCAAGCAGGAATTTGGAATTCCCTACAATCCCCAAAGTCAAGGAGTAGTAGAAT |
| | | | CTATGAATAAAGAATTAAAGAAAATTATAGGACAGGTAAGAGATCAGGCTGAACATCTTAAGACAGCAGT |
| | | | ACAAATGGCAGTATTCATCCACAATTTTAAAAGAAAAGGGGGGATTGGGGGGTACAGTGCAGGGGAAAGA |
| | | | ATAGTAGACATAATAGCAACAGACATACAAACTAAAGAATTACAAAAACAAATTACAAAAATTCAAAATT |
| | | | TTCGGGTTTATTACAGGGACAGCAGAAATCCACTTTGGAAAGGACCAGCAAAGCTCCTCTGGAAAGGTGAA |
| | | | GGGGCAGTAGTAATACAAGATAATAGTGACATAAAAGTAGTGCCAAGAAGAAAAGCAAAGATCATTAGGG |
| | | | ATTATGGAAAACAGATGGCAGGTGATGATTGTGTGGCAAGTAGACAGGATGAGGATTAGAACATGGAAAA |
| | | | GTTTAGTAAAACACCATATGTATGTTTCAGGGAAAGCTAGGGGATGGTTTTATAGACATCACTATGAAAG |
| | | | CCCTCATCCAAGAATAAGTTCAGAAGTACACATCCCACTAGGGGATGCTAGATTGGTAATAACAACATATT |
| | | | GGGGTCTGCATACAGGAGAAAGAGACTGGCATTTGGGTCAGGGAGTCTCCATAGAATGGAGGAAAAAGAG |
| | | | ATATAGCACACAAGTAGACCCTGAACTAGCAGACCAACTAATTCATCTGTATTACTTTGACTGTTTTTCAG |
| | | | ACTCTGCTATAAGAAAGGCCTTATTAGGACACATAGTTAGCCCTAGGTGTGAATATCAAGCAGGACATAAC |
| | | | AAGGTAGGATCTCTACAATACTTGGCACTAGCAGCATTAATAACACCAAAAAAGATAAAGCCACCTTTGCC |
| | | | TAGTGTTACGAAACTGACAGAGGATAGATGGAACAAGCCCCAGAAGACCAAGGGCCACAGAGGGAGCCACA |
| | | | CAATGAATGGACACTAGAGCTTTTAGAGGAGCTTAAGAATGAAGCTGTTAGACATTTTCCTAGGATTTGGC |
| | | | TCCATGGCTTAGGGCAACATATCTATGAAACTTATGGGGATACTTGGGCAGGAGTGGAAGCCATAATAAGA |
| | | | ATTCTGCAACAACTGCTGTTTATCCATTTTCAGAATTGGGTGTCGACATAGCAGAATAGGCGTTACTCGAC |
| | | | AGAGGAGAGCAAGAAATGGAGCCAGTAGATCCTAGACTAGAGCCCTGGAAGCATCCAGGAAGTCAGCCTAA |
| | | | AACTGCTTGTACCAATTGCTATTGTAAAAAGTGTTGCTTTCATTGCCAAGTTTGTTTCATAACAAAAGCCT |
| | | | TAGGCATCTCCTATGGCAGGAAGAAGCGGAGACAGCGACGAAGAGCTCATCAGAACAGTCAGACTCATCAA |
| | | | GCTTCTCTATCAAAGCAGTAAGTAGTACATGTAATGCAACCTATACCAATAGTAGCAATAGTAGCATTAGT |
| | | | AGTAGCAATAATAATAGCAATAGTTGTGTGGTCCATAGTAATCATAGAATATAGGAAAATATTAAGACAA |
| | | | AGAAAAATAGACAGGTTAATTGATAGACTAATAGAAAGAGCAGAAGACAGTGGCAATGAGAGTGAAGGAG |
| | | | AAATATCAGCACTTGTGGAGATGGGGGTGGAGATGGGGCACCATGCTCCTTGGGATGTTGATGATCTGTAG |
| | | | TGCTACAGAAAAATTGTGGGTCACAGTCTATTATGGGGTACCTGTGTGGAAGGAAGCAACCACCACTCTAT |
| | | | TTTGTGCATCAGATGCTAAAGCATATGATACAGAGGTACATAATGTTTGGGCCACACATGCCTGTGTACCC |
| | | | ACAGACCCCAACCCACAAGAAGTAGTATTGGTAAATGTGACAGAAAATTTTAACATGTGGAAAAATGACA |
| | | | TGGTAGAACAGATGCATGAGGATATAATCAGTTTATGGGATCAAAGCCTAAAGCCATGTGTAAAATTAAC |
| | | | CCCACTCTGTGTTAGTTTAAAGTGCACTGATTTGAAGAATGATACTAATACCAATAGTAGTAGCGGGAGAA |
| | | | TGATAATGGAGAAAGGAGAGATAAAAAACTGCTCTTTCAATATCAGCACAAGCATAAGAGGTAAGGTGCA |
| | | | GAAAGAATATGCATTTTTTTATAAACTTGATATAATACCAATAGATAATGATACTACCAGCTATAAGTTG |
| | | | ACAAGTTGTAACACCTCAGTCATTACACAGGCCTGTCCAAAGGTATCCTTTGAGCCAATTCCCATACATTA |
| | | | TTGTGCCCCGGCTGGTTTTGCGATTCTAAAATGTAATAATAAGACGTTCAATGGAACAGGACCATGTACAA |
| | | | ATGTCAGCACAGTACAATGTACACATGGAATTAGGCCAGTAGTATCAACTCAACTGCTGTTAAATGGCAGT |
| | | | CTAGCAGAAGAAGAGGTAGTAATTAGATCTGTCAATTTCACGGACAATGCTAAAACCATAATAGTACAGCT |
| | | | GAACACATCTGTAGAAATTAATTGTACAAGACCCAACAACAATACAAGAAAAAGAATCCGTATCCAGAGA |
| | | | GGACCAGGGAGAGCATTTGTTACAATAGGAAAAATAGGAAATATGAGACAAGCACATTGTAACATTAGTA |
| | | | GAGCAAAATGGAATAACACTTTAAAACAGATAGCTAGCAAATTAAGAGAACAATTTGGAAATAATAAAAC |
| | | | AATAATCTTTAAGCAATCCTCAGGAGGGGACCCAGAAATTGTAACGCACAGTTTTAATTGTGGAGGGGAAT |
| | | | TTTTCTACTGTAATTCAACACAACTGTTTAATAGTACTTGGTTTAATAGTACTTGGAGTACTGAAGGGTCA |
| | | | AATAACACTGAAGGAAGTGACACAATCACCCTCCCATGCAGAATAAAACAAATTATAAACATGTGGCAGA |
| | | | AAGTAGGAAAAGCAATGTATGCCCCTCCCATCAGTGGACAAATTAGATGTTCATCAAATATTACAGGGCTG |
| | | | CTATTAACAAGAGATGGTGGTAATAGCAACAATGAGTCCGAGATCTTCAGACCTGGAGGAGGAGATATGA |
| | | | GGGACAATTGGAGAAGTGAATTATATAAATATAAAGTAGTAAAAATTGAACCATTAGGAGTAGCACCCAC |
| | | | CAAGGCAAAGAGAAGAGTGGTGCAGAGAGAAAAAAGAGCAGTGGGAATAGGAGCTTTGTTCCTTGGGTTC |
| | | | TTGGGAGCAGCAGGAAGCACTATGGGCGCAGCCTCAATGACGCTGACGGTACAGGCCAGACAATTATTGTC |
| | | | TGGTATAGTGCAGCAGCAGAACAATTTGCTGAGGGCTATTGAGGCGCAACAGCATCTGTTGCAACTCACAG |
| | | | TCTGGGGCATCAAGCAGCTCCAGGCAAGAATCCTGGCTGTGGAAAGATACCTAAAGGATCAACAGCTCCTG |
| | | | GGGATTTGGGGTTGCTCTGGAAAACTCATTTGCACCACTGCTGTGCCTTGGAATGCTAGTTGGAGTAATAA |
| | | | ATCTCTGGAACAGATTTGGAATCACACGACCTGGATGGAGTGGGACAGAGAAATTAACAATTACACAAGCT |
| | | | TAATACACTCCTTAATTGAAGAATCGCAAAACCAGCAAGAAAAGAATGAACAAGAATTATTGGAATTAGA |
| | | | TAAATGGGCAAGTTTGTGGAATTGGTTTAACATAACAAATTGGCTGTGGTATATAAAATTATTCATAATG |
| | | | ATAGTAGGAGGCTTGGTAGGTTTAAGAATAGTTTTTGCTGTACTTTCTATAGTGAATAGAGTTAGGCAGG |
| | | | GATATTCACCATTATCGTTTCAGACCCACCTCCCAACCCCGAGGGGACCCGACAGGCCCGAAGGAATAGAA |
| | | | GAAGAAGGTGGAGAGAGAGACAGAGACAGATCCATTCGATTAGTGAACGGATCCTTGGCACTTATCTGGG |
| | | | ACGATCTGCGGAGCCTGTGCCTCTTCAGCTACCACCGCTTGAGAGACTTACTCTTGATTGTAACGAGGATT |
| | | | GTGGAACTTCTGGGACGCAGGGGGTGGGAAGCCCTCAAATATTGGTGGAATCTCCTACAGTATTGGAGTCA |
| | | | GGAACTAAAGAATAGTGCTGTTAGCTTGCTCAATGCCACAGCCATAGCAGTAGCTGAGGGGACAGATAGGG |
| | | | TTATAGAAGTAGTACAAGGAGCTTGTAGAGCTATTCGCCACATACCTAGAAGAATAAGACAGGGCTTGGA |
| | | | AAGGATTTTGCTATAAGATGGGTGGCAAGTGGTCAAAAAGTAGTGTGATTGGATGGCCTACTGTAAGGGA |
| | | | AAGAATGAGACGAGCTGAGCCAGCAGCAGATAGGGTGGGAGCAGCATCTCGAGACCTGGAAAAACATGGA |
| | | | GCAATCACAAGTAGCAATACAGCAGCTACCAATGCTGCTTGTGCCTGGCTAGAAGCACAAGAGGAGGAGGA |
| | | | GGTGGGTTTTCCAGTCACACCTCAGGTACCTTTAAGACCAATGACTTACAAGGCAGCTGTAGATCTTAGCC |
| | | | ACTTTTTAAAAGAAAAGGGGGGACTGGAAGGGCTAATTCACTCCCAAAGAAGACAAGATATCCTTGATCTG |
| | | | TGGATCTACCACACACAAGGCTACTTCCCTGATTAGCAGAACTACACACCAGGGCCAGGGGTCAGATATCC |
| | | | ACTGACCTTTGGATGGTGCTACAAGCTAGTACCAGTTGAGCCAGATAAGATAGAAGAGGCCAATAAAGGA |
| | | | GAGAACACCAGCTTGTTACACCCTGTGAGCCTGCATGGGATGGATGACCCGGAGAGAGAAGTGTTAGAGTG |
| | | | GAGGTTTGACAGCCGCCTAGCATTTCATCACGTGGCCCGAGAGCTGCATCCGGAGTACTTCAAGAACTGCT |
| | | | GACATCGAGCTTGCTACAAGGGACTTTCCGCTGGGGACTTTCCAGGGAGGCGTGGCCTGGGCGGGACTGGG |
| | | | GAGTGGCGAGCCCTCAGATCCTGCATATAAGCAGCTGCTTTTTGCCTGTACTGGGTCTCTCTGGTTAGACC |
| | | | AGATCTGAGCCTGGGAGCTCTCTGGCTAACTAGGGAACCCACTGCTTAAGCCTCAATAAAGCTTGCCTTGA |
| | | | GTGCTTC |
| BBTVR | NC_003479.1 | 480 | AGATGTCCCGAGTTAGTGCGCCACGTAAGCGCTGGGGCTTATTATTACCCCCAGCGCTCGGG |
| | | | ACGGGACATTTGCATCTATAAATAGACCTCCCCCCTCTCCATTACAAGATCATCATCGACGAC |
| | | | AGAATGGCGCGATATGTGGTATGCTGGATGTTCACCATCAACAATCCCACAACACTACCAGT |
| | | | GATGAGGGATGAGATAAAATATATGGTATATCAAGTGGAGAGGGGACAGGAGGGTACTCGT |
| | | | CATGTGCAAGGTTATGTCGAGATGAAGAGACGAAGCTCTCTGAAGCAGATGAGAGGCTTCTT |
| | | | CCCAGGCGCACACCTTGAGAAACGAAAGGGAAGCCAAGAAGAAGCGCGGTCATACTGTATG |
| | | | AAGGAAGATACAAGAATCGAAGGTCCCTTCGAGTTTGGTTCATTTAAATTGTCATGTAATGA |
| | | | TAATTTATTTGATGTCATACAGGATATGCGTGAAACGCACAAAAGGCCTTTGGAGTATTTATA |
| | | | TGATTGTCCTAACACCTTCGATAGAAGTAAGGATACATTATACAGAGTACAAGCAGAGATGA |
| | | | ATAAAACGAAGGCGATGAATAGCTGGAGAACTTCTTTCAGTGCTTGGACATCAGAGGTGGAG |
| | | | AATATCATGGCGCAGCCATGTCATCGGAGAATAATTTGGGTCTATGGCCCAAATGGAGGAGA |
| | | | AGGAAAGACAACGTATGCAAAACATCTAATGAAGACGAGAAATGCGTTTTATTCTCCAGGAG |
| | | | GAAAATCATTGGATATATGTAGACTGTATAATTACGAGGATATTGTTATATTTGATATTCCAA |
| | | | GATGCAAAGAGGATTATTTAAATTATGGGTTATTAGAGGAATTTAAGAATGGAATAATTCAA |
| | | | AGCGGGAAATATGAACCCGTTTTGAAGATAGTAGAATATGTCGAAGTCATTGTAATGGCTAA |
| | | | CTTCCTTCCGAAGGAAGGAATCTTTTCTGAAGATCGAATAAAGTTGGTTTCTTGCTGAACAAG |
| | | | TAATGACTTTACAGCGCACGCTCCGACAAAAGCACACTATGACAAAAGTACGGGTATCTGAT |
| | | | TGGGTTATCTTAACGATCTAGGGCCGTAGGCCCGTGAGCAATGAACGGCGAGATC |
| BBTVN | NC_003476.1 | 481 | AGCACGGGGGACTATTATTACCCCCCGTGCTCGGGACGGGACATGACGTCAGCAAGGATTAT |
| | | | AATGGGCTTTTTATTAGCCCATTTATTGAATTGGGCCGGGTTTTGTCATTTTACAAAAGCCCG |
| | | | GTCCAGGATAAGTATAATGTCACGTGCCGAATTAAAAGGTTGCTTCGCCACGAAGAAACCTA |
| | | | ATTTGAGGTTGCGTATTCAATACGCTACCGAATATCTATTAATATGTGAGTCTCTGCCGAAAA |
| | | | AAATCAGAGCGAAAGCGGAAGGCAGAAGCGATGGATTGGGCGGAATCACAATTCAAGACCT |
| | | | GTACTCATGGATGCGATTGGAAGAAGATATCATCGGATTCAGCCGATAATCGACAATATGTA |
| | | | CCATGCGTCGATTCTGGAGCTGGAAGAAAGTCGCCTCGCAAGGTACTTCTTAGATCTATTGA |
| | | | AGCTGTGTTTAACGGAAGCTTCAGCGGAAATAATAGGAATGTTCGTGGATTTCTCTACGTATC |
| | | | GATCAGAGACGATGACGGAGAAATGCGTCCAGTACTCATAGTACCATTCGGAGGATATGGAT |
| | | | ATCATAATGATTTTTATTATTTCGAAGGGAAGGGGAAAGTTGAATGTGATATATCATCAGATT |
| | | | ATGTTGCGCCAGGAATAGATTGGAGCAGAGACATGGAAGTTAGTATTAGTAACAGCAACAA |
| | | | CTGTAATGAATTATGTGATCTGAAGTGTTATGTTGTTTGTTCGTTAAGAATCAAGGAATAAAA |
| | | | GTTGTGCTGTAATGTTAATTAATAAAACGTATATTTGGGAAATTGATAGTTGTATAAAACATA |
| | | | CAACACACTATGAAATACAAGACGCTATGACAAATGTACGGGTATCTGAATGAGTTTTAGTA |
| | | | TCGCTTAAGGGCCGCAGGCCCGTTAAAAATAATAATCGAATTATAAACGTTAGATAATAATC |
| | | | AGAGATAGGTGATCAGATAATATAAACATAAACGAAGTATATGCCGGTACAATAATAAAAT |
| | | | AAGTAATAACAAAAAAAATATGTATACTAATCTCTGATTGGTTCAGGAGAAAGGCCCACCAA |
| | | | CTAAAAGGTGGGGAGAATGTCCCGATGACGTA |
| BBTVM | 003474.1 | 482 | AGCGCTGGGGCTTATTATTACCCCCAGCGCTCGGGACGGGACATCACGTGCGTCAACAAATGCACGTGACT |
| | | | GATATAAGGGACATAACGGGTTTAGATAACGGTTTATGCGGATTAGAATATAACGTCACGTGTGAAAGCC |
| | | | GAAAGGCACGTGACGAAGACAAATGGATTGAATAAACATTTGACGTCCGGTAGCTTCCGAAGGAAGTAAG |
| | | | CTTCGCGGCGAAGCAAACCATTTATATATTTGCGTAGGCTTGCGGCCTATAAATAGGACGCAGCTAAATGG |
| | | | CATTAACAACAGAGCGGGTGAAACTATTCTTTGAATGGTTTCTGTTCTTTGGAGCAATATTTATTGCGATT |
| | | | ACAATATTATATATATTGTTGGTTTTGCTCTTTGAGGTACCCAGGTATATTAAGGAGCTCGTGAGGTGTTT |
| | | | GGTAGAATACCTGACCAGACGACGTGTATGGATGCAGAGGACGCAGTTGACGGAGGCAACTGGAGATGTA |
| | | | GAGATCGGCAGAGGTATTGTGGAAGACAGACGAGATCAAGAACCGGCTGTCATACCACATGTATCTCAGGT |
| | | | AATCCCTTCTCAACCAAATAGAAGGGATGATCAAGGAAGACGAGGAAACGCTGGACCTATGTTCTAATACA |
| | | | CGGTATATTAATATACGAAATATAAATGGGTATTGATGTAAATGATCATACATAATATATGTATGATAAT |
| | | | GAAACATATTGTAATATGTGAATTGTAAACGAGAGTTGTATGTATAAAACATACAACACGCTATGAAATA |
| | | | CAAGACGCTATGACAAAAGTACTGGTATATGATTAGGTATCCTAACGATCTAGGGCCGAAGGCCCGTGAGC |
| | | | AATATGCGTCGAAATAATGTTTAACAAACAAATATACATGATACGGATAGTTGAATACATAAACAACGAG |
| | | | GTATACAATACAACAAACTGTTGTAAAGAAATAAAAAATAAGAAGAGATAGTATATTTGTGTTGGATAAG |
| | | | CCTTGCAACCACCACTTTAGTGGTGGGCCAGATGTCCCGAGTTAGTGCGCCACGTA |
| BBTVC | NC_003477.1 | 483 | AGCGCTGGGGCTTATTATTACCCCCAGCGCTCGGGACGGGACATCACGTGCAACTAACAGACGCACGTGAG |
| | | | AATGCAGTAGCTTGCAGCGAAAGATAGACGTCAACATCAATAAAGAAGAAGGAATATTCTTTGCTTCGGC |
| | | | ACGAAGCAAAGGGTATAGATATTTGTTCGAGATGCGAAAATGGAGGCTATTTAAACCTGATGGTTTTGTG |
| | | | ATTTCCGAAATCACTCGTCGGAAGAGAAATGGAGTTCTGGGAATCGTCTGCCATGCCTGACGATGTCAAGA |
| | | | GAGAGATTAAGGAAATATATTGGGAAGATCGGAAGAAACTTCTGTTCTGTCAGAAGTTGAAGAGCTATGT |
| | | | CAGAAGGATTCTTGTTTATGGAGATCAAGAGGATGCCCTTGCCGGAGTGAAGGATATGAAGACTTCTATTA |
| | | | TTCGCTATAGCGAATACTTGAAGAAACCATGTGTGGTAATTTGTTGTGTTAGCAATAAATCAATTGTGTAT |
| | | | AGGTTAAACAGCATGGTGTTCTTTTATCATGAATACCTTGAAGAACTAGGTGGTGATTACTCAGTATATCA |
| | | | AGATCTCTATTGTGATGAGGTACTCTCTTCTTCATCGACAGAGGAAGAAGATGTAGGAGTAATATATAGG |
| | | | AATGTTATCATGGCATCGACACAAGAGAAGTTCTCTTGGAGTGATTGTCAGCAGATAGTTATATCAGACTA |
| | | | TGATGTAACATTACTCTAATGTAATATCCATTATCATCAATAAAATAATGGAATGTTGATTATGTATTTA |
| | | | TCATAAATACATAATGGTATACGTATAGCATAAAATACATTAACCAACATACAACACACTATAAAATACA |
| | | | ACACACTATAACAAATGTACGGGTATTTGATTGGGCTATATTAACCCCTTAAGGGCCGAAGGCCCGTTTAA |
| | | | ATATGTGTTGGACGAAGTCCAAACACAAAAAAGTAAGCAGAACAACGGAATAATATGAGCTGGCAACGTA |
| | | | GGGTCCATGTCCCGAGTTAGTGCGCCACGTA |
| BBTVU3 | NC_003475.1 | 484 | GGCGCTGGGGCTTATTATTACCCCCAGCGCCGGGACGGGACATGGGCTTTTTAAATGGGCTTTGCGAGTTT |
| | | | GAACAGTTCAGTATCTTCGTTATTGGGCCAACCCGGCCCAATAATTAAGAGAACGTGTTCAAATTCGTGGT |
| | | | ATGACCGAAGGTCAAGGTAACCGGTCAACATTATTCTGGCTTGCGCAGCAAGATACACGAATTAATTTATT |
| | | | AATTCGTAGGACACGTGGACGGACCGAAATACTCTTGCATCTCTATAAATACCCTAATCCTGTCAAGGATA |
| | | | ATTGCTCTCTCTCTTCTGTCAAGGTGGTTGTGCTGAGGCGGAAGATCGCCAGCGGCGATCGTCGGAACGAC |
| | | | CTGCATCTAGAGAGGCGGCGAGGAAACTACGAAGCGTATATCGGGTATTTATAGACTTATAGCGTAGCTAG |
| | | | AAGTATACACTGTACAGATATTGTATCTTGTAAATTACGAAGCAATTCGTATTTGATATTAATAAAACAA |
| | | | CTGGGTTTGTTAATGTTTACATTAACTAGTATCTTATATGTACAAATTAAAATACAGTATACGGAACGTAT |
| | | | ACTAACGTAAAAATTAAATGATAGGCGAAGCATGATTAACAGGTGTTTAGGTATAATTAACATAATTATG |
| | | | AGAAGTAATAATAATACGGAAAATGAATAAGTATGAGGTGAAAGAGGAGATATTAGAATATTTAAAAACC |
| | | | CAATTATATTATTTTGGAACGAAATACAACACGCTATGAAATACAAGACGCTATGACAAATGTACGGGAA |
| | | | TATGATTGTGTATCTTAACGTATAAGGGCCGCAGGCCCGTCAAGTTGAATGAACGGTCCAGATTAATTCCT |
| | | | TAGCGACGAAGAAAGGAATCTTAAAGGGGACCACATTAAAGACAGCTGTCATTGATTAAATAAATAATAT |
| | | | AATAACCAAAAGACCTTTGTACCCTTCCTAATGATGACGTATAGGGGTGTCCCGATGTAATTTAACATAGC |
| | | | TCTGAAAAGAGATATGGGCCGTTGGATGCCTCCATCGGACGATGGAGGTTGAATGAACTTCTGCTGACGTA |
| BBTVS | NC_003473.1 | 485 | AGCGCTGGGGACTATTATTACCCCCAGCGCTCGGGACGGGACATGGGCTAATGGATTGTGGATATAGGGCC |
| | | | CAAAGGGCCCGTTTAGATGGGTTTTGGGCTCATGGGCTTTATCCAGAAGACCAAAAACAGGCGGGAACCGT |
| | | | CCCAAATTCAAACTTCGATTGCTTGCCCTGCAACGCATCTAGAAGTCTATAAATACCAGTGTCTAGATAGA |
| | | | TGTTCAGACAAGAAATGGCTAGGTATCCGAAGAAATCCATCAAGAAGAGGCGGGTTGGGCGCCGGAAGTA |
| | | | TGGCAGCAAGGCGGCAACGAGCCACGACTACTCGTCGTCAGGGTCAATATTGGTTCCTGAAAACACCGTCA |
| | | | AGGTATTTCGGATTGAGCCTACTGATAAAACATTACCCAGATATTTTATCTGGAAAATGTTTATGCTTCTT |
| | | | GTGTGCAAGGTGAAGCCCGGAAGAATACTTCATTGGGCTATGATCAAGAGTTCTTGGGAAATCAACCAGCC |
| | | | GACAACCTGTCTGGAAGCCCCAGGTTTATTTATTAAACCTGAACACAGCCATCTGGTTAAACTGGTATGTA |
| | | | GTGGGGAACTTGAAGCAGGAGTCGCAACAGGAACATCAGATGTTGAATGTCTTTTGAGGAAGACAACCGT |
| | | | GTTGAGGAAGAATGTAACAGAGGTGGATTATTTATATTTGGCATTCTATTGTAGTTCTGGAGTAAGTATA |
| | | | AACTACCAGAACAGAATTACATATCATGTTTGATATGTTTATGTAAACATAAACTATTGTATGGAATGAA |
| | | | ATCCAAATAACATACAACACGCTATGAAATACAAGACGCTATGACAAAAGTACTGGTATATGATTAGGTA |
| | | | TCCTAACGATCTAGGGCCGAAGGCCCGTGAGCAATATGCGTCGAAATAATGTTTAACAAACAAATATACAT |
| | | | GATACGGATAGTTGAATACATAAACAACGAGGTATACAATACAACAAACTGTTGTAAAGAAATAAAAAAT |
| | | | AAGAAGAGAGAGTATATTTGTGTCGGATAAGCATCACACCCACCACTTTAGTGGTGGGCCAGATGTCCCGA |
| | | | GTTAGTGCGCCACGTA |

Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

What is claimed is:

1. A vaccine, comprising:
an isolated plant viral antigen, wherein the plant viral antigen is immunogenic, and a pharmaceutically acceptable carrier.

2. The vaccine of claim 1, wherein the plant viral antigen is an immunogenic peptide, and optionally further comprising an adjuvant.

3. The vaccine of claim 1, wherein the plant viral antigen is a nucleic acid comprising at least one gene encoding a plant viral peptide and optionally further comprising:
a replication defective vector comprising the nucleic acid, and/or
wherein the gene is operably linked to a heterologous promoter and transcription terminator, the replication defective vector is optionally an adenoviral vector.

4. The vaccine of claim 1, wherein the plant viral antigen is a plant virus selected from the group consisting of Maize chlorotic mottle virus; Maize rayado fino virus; Oat chlorotic stunt virus; Chayote mosaic tymovirus; Grapevine asteroid mosaic-associated virus; Grapevine fleck virus; Grapevine Red Globe virus; Grapevine rupestris vein feathering virus; Melon necrotic spot virus; Physalis mottle tymovirus; Prunus necrotic ringspot; Nigerian tobacco latent virus; Tobacco mild green mosaic virus; Tobacco mosaic virus; Tobacco necrosis virus; Eggplant mosaic virus; Kennedya yellow mosaic virus; Lycopersicon esculentum TVM viroid; Oat blue dwarf virus; Obuda pepper virus; Olive latent virus 1; Paprika mild mottle virus; PMMV; Tomato mosaic virus; Turnip vein-clearing virus; Carnation mottle virus; Cocksfoot mottle virus; Galinsoga mosaic virus; Johnsongrass chlorotic stripe mosaic virus; Odontoglossum ringspot virus; Ononis yellow mosaic virus; Panicum mosaic virus; Poinsettia mosaic virus; Pothos latent virus; and Ribgrass mosaic virus.

5. The vaccine of claim 1, further comprising an agent selected from:
a TLR agonist,
a CLIP inhibitor, wherein the CLIP inhibitor is optionally FRIMAVLAS (SEQ ID NO. 439),
a fatty acid metabolism inhibitor, and/or
an autophagy inhibitor.

6. A method of modulating gastrointestinal plant viral levels in a subject, comprising:
administering to the subject an amount of a plant virus vaccine effective to modulate the plant virus levels in the gastrointestinal tract of the subject, wherein the plant virus vaccine is optionally a vaccine of claim 1.

7. The method of claim 6, wherein the levels of plant virus in the gastrointestinal system of the subject corresponding to the plant virus vaccine are decreased in the gastrointestinal system of the subject relative to the levels that are observed in the absence of the administration of the plant virus vaccine, optionally, wherein the levels of plant virus in the gastrointestinal system of the subject are measured in a fecal or blood sample.

8. A method, comprising:
administering to a subject at risk of having a plant virus associated cancer, a plant virus vaccine in an effective amount to inhibit infection with the plant virus in the subject, wherein the plant virus vaccine is optionally a vaccine of claim 1.

9. The method of claim 8, wherein the subject has been exposed to a plant virus.

10. A method for treating a subject, comprising:
administering an anti-viral compound to the subject, wherein the subject has a disease associated with a plant virus, in an effective amount to reduce infection with the plant virus in the subject.

11. The method of claim 10, further comprising administering an agent selected from:
a TLR agonist, wherein the TLR agonist optionally is TLR3 agonist such as poly(I:C), a TLR7 agonist, a TLR8 agonist or a TLR9 agonist such as a CpG oligonucleotide,
a CLIP inhibitor, wherein the CLIP inhibitor is optinally FRIMAVLAS (SEQ ID NO. 439),
a fatty acid metabolism inhibitor, and/or
an autophagy inhibitor.

12. A method, comprising:
determining whether a subject having a virally caused disease, such as cancer, has been exposed to a plant virus that causes the disease, and treating the subject with a compound that is a plant defense mechanism against the plant virus in an effective amount to reduce infection of the subject with the plant virus.

13. The method of claim 12, wherein the compound is a naturally occurring substance found in a plant susceptible to the plant virus or is an analog, homolog, or derivative thereof and is optionally selected from the group consisting of flavonoids, anthocyanins, phytoalexins, medicarpin, rishitin, camalexin, capsaisin, glucosinolate, defensins, alpha-amylase, protease inhibitors, lignin and furanocoumarins.

14. The method of claim 12, wherein the step of determining whether the subject has been exposed to the plant virus involves analyzing a biological sample of the subject for the presence of the plant virus, wherein the biological sample optionally is a fecal sample.

15. A method for silencing plant virus gene expression in a mammal needing relief from the gene expression, comprising:
administering to the mammal an inhibitory nucleic acid that targets the genome of an essential plant virus in an effective amount to reduce infection of the mammal with the plant virus.

16. The method of claim 15, wherein the inhibitory nucleic acid comprises:
a) a double stranded nucleic acid of 15 to 30 nucleotides in length,
b) a first nucleotide sequence that targets the genome of the essential plant virus and a second nucleotide that is a complement of the first nucleotide sequence, and/or
c) a nucleotide sequence having sufficient complementarity to a target sequence of about 15 to about 30 contiguous nucleotides in an RNA of a virus for the inhibitory nucleic acid to direct cleavage of the RNA via RNA interference, wherein the virus is selected from the group consisting of Maize chlorotic mottle virus; Maize rayado fino virus; Oat chlorotic stunt virus; Chayote mosaic tymovirus; Grapevine asteroid mosaic-associated virus; Grapevine fleck virus; Grapevine Red Globe virus; Grapevine rupestris vein feathering virus; Melon necrotic spot virus; Physalis mottle tymovirus; Prunus necrotic ringspot; Nigerian tobacco latent virus; Tobacco mild green mosaic virus; Tobacco mosaic virus; Tobacco necrosis virus; Eggplant mosaic virus; Kennedya yellow mosaic virus; Lycopersicon esculentum TVM viroid; Oat blue dwarf virus; Obuda pepper virus; Olive latent virus 1; Paprika mild mottle virus; PMMV; Tomato mosaic virus; Turnip vein-clearing virus; Carnation mottle virus; Cocksfoot mottle virus; Galinsoga mosaic virus; Johnsongrass chlorotic stripe mosaic virus; Odontoglossum ringspot virus; Ononis yellow mosaic virus; Panicum mosaic virus; Poinsettia mosaic virus; Pothos latent virus; and Ribgrass mosaic virus, wherein the target sequence is in a gene essential for infectivity or replication of the virus, wherein the gene essential for infectivity or replication of the virus is optionally selected from the group consisting of plant virus genome-linked protein (VPg), VPg-Pro, the 3′UTR, the 5′ UTR, zinc finger region of the capsid protein, and tRNA like domain.

17. A composition comprising: a vector comprising a nucleic acid encoding an inhibitory nucleic acid that targets the genome of an essential plant virus operably linked to a mammalian promoter.

18. A method, comprising:
performing a physical analytical step on a biological sample, optionally a fecal sample, of a subject,
identifying the presence of plant virus in the biological sample based on the physical analytical step, and
determining a course of treatment for the subject based on the presence of the plant virus, wherein the presence of the plant virus is indicative of a predisposition to cancer.

19. The method of claim 18, wherein the plant virus is selected from the group consisting of tobacco mosaic virus, Maize chlorotic mottle virus; Maize rayado fino virus; Oat chlorotic stunt virus; Chayote mosaic tymovirus; Grapevine asteroid mosaic-associated virus; Grapevine fleck virus; Grapevine Red Globe virus; Grapevine rupestris vein feathering virus; Melon necrotic spot virus; Physalis mottle tymovirus; Prunus necrotic ringspot; Nigerian tobacco latent virus; Tobacco mild green mosaic virus; Tobacco necrosis virus; Eggplant mosaic virus; Kennedya yellow mosaic virus; Lycopersicon esculentum TVM viroid; Oat blue dwarf virus; Obuda pepper virus; Olive latent virus 1; Paprika mild mottle virus; PMMV; Tomato mosaic virus; Turnip vein-clearing virus; Carnation mottle virus; Cocksfoot mottle virus; Galinsoga mosaic virus; Johnsongrass chlorotic stripe mosaic virus; Odontoglossum ringspot virus; Ononis yellow mosaic virus; Panicum mosaic virus; Poinsettia mosaic virus; Pothos latent virus; and Ribgrass mosaic virus.

20. The method of claim 18, further comprising analyzing the status of inflammation in the subject.

21. The method of claim 18, wherein the course of treatment is the administration of a plant virus vaccine, optionally the plant virus vaccine claim 1.

22. A method for treating a plant virus associated cancer, comprising:
administering to a subject having a plant virus associated cancer an anti-viral compound in an effective amount to treat the cancer, wherein the anti-viral compound is a compound that interferes with viral synthesis.

23. The method of claim 22, wherein the anti-viral compound is selected from:
a) an inhibitor of plant specific RNA dependent RNA polymerase,
b) an inhibitor that is an RNA dependent RNA polymerase antagonist,
c) an RNA dependent RNA polymerase antagonist that is an inhibitory peptide, such as an antibody,
d) an RNA dependent RNA polymerase antagonist that is an inhibitory nucleic acid, and/or
e) an inhibitory nucleic acid that is an siRNA.

24. A method for identifying an anti-cancer agent, comprising:
performing a physical analytical step on a plant to determine a plant defense mechanism for preventing infection with a plant virus,
identifying an association of the plant virus with a mammalian cancer, and
selecting the plant defense mechanism as an anti-cancer agent for the mammalian cancer.

25-29. (canceled)
